(19) United States
(12) Patent Application Publication
(10) Pub. No.: US 2023/0265157 A1
(43) Pub. Date: Aug. 24, 2023

(54) MHC MULTIMER EXPRESSION CONSTRUCTS AND USES THEREOF

(71) Applicant: Repertoire Immune Medicines, Inc., Cambridge, MA (US)

(72) Inventors: Joanna Swain, Concord, MA (US); Michael Birnbaum, Somerville, MA (US)

(21) Appl. No.: 18/012,573

(22) PCT Filed: Jun. 23, 2021
(86) PCT No.: PCT/US2021/038724

§ 371 Date: Dec. 22, 2022

Related U.S. Application Data
(60) Provisional application No. 63/043,316, filed on Jun. 24, 2020.

(51) Int. Cl. C07K 14/74; C07K 14/005; C07K 14/47; C07K 14/36; G01N 33/569

(52) U.S. Cl. CPC C07K 14/70539; C07K 14/005; C07K 14/4703; C07K 14/36; G01N 33/56977; C07K 2319/03; C07K 2319/02

(57) ABSTRACT

MHC multimer expression constructs are provided that contiguously encode an MHC-binding peptide, MHC molecule chains and a multimerization domain in the construct such that expression in a host cell results in production of peptide-loaded MHC (pMHC) multimers by the host cell. The multimers can further comprise oligonucleotide barcodes. Peptide exchange can be performed with a plurality of pMHC multimers to create pMHC multimer libraries. Methods of making and using the pMHC multimers and libraries are also provided. Peptide-loaded MHC Class I and MHC Class II multimers, and libraries thereof, are provided.

### RELATED APPLICATIONS

This application claims priority to U.S. Provisional Application No. 63/043,316 filed Jun. 24, 2020, the entire contents of which is hereby incorporated by reference.

### BACKGROUND

Identification of peptides recognized by individual T cells is important for the understanding and treatment of immune-related diseases, as well as vaccine development for prevention of diseases. Techniques for the detection of antigen-responsive T cells exploit the interaction between a given TCR and its peptide-MHC (pMHC) recognition motif. The ability to prepare soluble MHC molecules allowed for the preparation of soluble peptide-MHC complexes, which then can be made into multimeric complexes. T cell detection using multimerized pMHC molecules has become the preferred method for detecting antigen-specific T cells in a wide variety of research and clinical situations.

MHC multimers have been used for detection of antigen-responsive T cells since Altman et al. (Science 274:94-96, 1996) showed that tetramerization of peptide-loaded MHC class I (pMHCI) molecules provided sufficient stability to T cell receptor (TCR)-pMHC interactions, allowing detection of fluorescently-labeled MHC multimer-binding T cells using flow cytometry. However, since MHC Class I molecules are largely unstable when they are not part of a complex with peptide, pMHCI-based technologies were initially restricted by the tedious production of molecules in which each peptide required an individual folding and purification procedure (Bakker et al., Curr. Opin. Immunol. 17:428-433, 2005).

More recently, a variety of MHCI molecules with covalently linked peptides have been reported (e.g., reviewed by Goldberg et al., J. Cell. Mol. Med. 15:1822-1832, 2011). Several types of pMHCI microarrays systems also have been developed, but most work has focused on optimizing the supporting surface and modifying the conditions applied during binding and/or washing. The use of these systems is also limited due to poor detection limits and low reproducibility compared to existing cytometry-based analyses. For example, a general limitation to such array-based strategies is the propensity of a given T cell to pursue all potential pMHCI interactions displayed on a given array. As a consequence, the frequency of antigen-responsive T cells in the cell preparations typically needs to be >0.1% to allow a robust readout.

MHCI multimers, and libraries thereof, have been prepared using biotinylated peptide-MHCI monomers that then associate with the biotin-binding site on streptavidin to form tetramers (see e.g., Leisner et al., PLoS One 3(2):e1678, 2008). For the creation of MHC Class I libraries, approaches have been described in which oligonucleotide barcode labels have been conjugated to the streptavidin. However, existing strategies involve complex and/or costly approaches that limit the facile production of large libraries. For example, in one approach, individual streptavidin precursors must be barcoded individually by overlap extension PCR prior to tetramerization of biotinylated peptide-HLA monomers (Zhang et al., Nature Biotech. 2018; doi:10.1038.nbt.4282). In another approach, streptavidin-conjugated dextran, which is a costly reagent, is used to create a dextramer to which both the biotinylated peptide-HLA monomers and the biotinylated barcode oligonucleotide are complexed (Bentzen et al., Nature Biotech. 34:10: 1037-1045, 2016) via the streptavidin conjugated to the dextran backbone.

Similar to the approach with pMHCI tetramers, soluble MHC class II molecules also have been used to prepare pMHCII tetramers, which have been used in the study of the antigenic specificity of CD4+ T helper cells (as reviewed in, for example, Nepom et al. (2002) Arthrit. Rheumat. 46:5-12; Vollers and Stern (2008) Immunol. 123:305-313; Cecconi et al. (2008) Cytometry 73A:1010-1018). Typically to prepare pMHCII multimers, soluble biotinylated MHCII α/β dimers are recombinantly expressed and then tetramerized by binding to streptavidin or avidin through their biotin-binding sites. Fluorescent labeling of the streptavidin or avidin then allows for isolation of T cells that bind the pMHCII multimers by flow cytometry. With regard to antigenic peptide loading of the MHCII molecules, in one approach, a peptide is attached to the MHCII α/β dimers covalently. Some groups have generated pMHCII loaded with a covalent but cleavable “stuffer” peptide that can be exchanged with a peptide of interest under acidic conditions (Day et al., J Clin Invest. 2003; 112(6):831-842).

In an alternative approach, “empty” MHCII α/β dimers are prepared and then loaded with soluble MHCII-binding peptides (see e.g., Novak et al. (1999) J. Clin. Invest. 104:63-67; Nepom et al. (2002) Arthrit. Rheumat. 46:5-12; Macaubus et al. (2006) J. Immunol. 176:5069-5077). While this approach allows for greater diversity of peptide loading onto the MHCII α/β dimers, the ability to recombinantly express stable “empty” MHCII α/β dimers is limited, thus again hampering the preparation of large scale pMHCII multimer libraries. For example, production of “empty” MHCII α/β dimers by refolding from E. coli inclusion bodies or by insect cell or mammalian cell expression has been reported, but with yields that are too low to support high throughput methods (reviewed in Vollers and Stern (2008) Immunology 123: 305-313).

Accordingly, there remains a need for efficient and cost effective methods of generating peptide-MHC libraries, including barcoded libraries, which may be utilized in a variety of methods, for example, screening of T cell specificity for analyses of T cell recognition, for example, at genome-wide levels rather than analyses restricted to a selection of model antigens.

### SUMMARY

The present disclosure provides methods for producing barcoded, peptide loaded MHC (pMHC) multimers (e.g., tetramers), including libraries thereof, using a recombinant genetic engineering approach involving expression of an MHC multimer expression construct in a host cell. The methods provide high protein yields of pMHC multimers within a short time period using efficient reaction conditions that allow for ease of peptide exchange and barcode labeling of the multimers to thereby allow for efficient preparation of large pMHC multimer libraries. Accordingly, the compositions and methods described herein are suitable for routine laboratory research, as well as large scale industrial and clinical applications, in all circumstances where pMHC multimers are useful. In one embodiment, the pMHC multimer is a pMHC Class I (pMHCI) multimer, which is useful for analysis of CD8+ T cell antigen recognition. In another embodiment, the pMHC multimer is a pMHC Class II (pMHCII) multimer, which is useful for analysis of CD4+ T cell antigen recognition.

The MHC multimer expression constructs of the disclosure encode a fusion polypeptide comprising an MHC-binding peptide, the MHC molecule chains and a multimerization domain. Typically, the regions of the construct encoding the MHC-binding peptide, the MHC molecule chains and the multimerization domain regions are separated by intervening linker sequences within the expression construct. Additionally, typically the linker that is operatively linked to the MHC-binding peptide is a cleavable linker such that upon cleavage of the linker, the MHC binding peptide is released from the fusion polypeptide. Release of this “placeholder” MHC-binding peptide thus allows for peptide exchange (e.g., with “rescue” peptides that bind to the same MHC molecule), thereby allowing for the preparation of libraries of peptide-bound-MHC multimers. Moreover, the MHC multimers of the disclosure can be labeled with individual identifiers, such as oligonucleotide barcodes, to facilitate identification of library members. For example when the multimerization domain is streptavidin, since the biotin-binding sites within streptavidin are not being used for multimerization of the MHC monomers, these biotin-binding sites are available for easy labeling using biotinylated oligonucleotide barcodes.

Accordingly, in one aspect, the disclosure pertains to a method of producing a Major Histocompatibility Complex (MHC) multimer, the method comprising:
(a) providing an MHC multimer expression construct comprising a nucleic acid encoding (i) an MHC-binding peptide operatively linked to a cleavage site; (ii) a first MHC subunit; (iii) a second MHC subunit; and (iv) a multimerization domain;
(b) introducing the MHC multimer expression construct into a host cell; and
(c) expressing the MHC multimer in the host cell.

In another aspect, the disclosure pertains to an isolated Major Histocompatibility Complex (MHC) multimer expression construct, the construct comprising a nucleic acid encoding (i) an MHC-binding peptide operatively linked to a cleavage site; (ii) a first MHC subunit; (iii) a second MHC subunit; and (iv) a multimerization domain.

In one embodiment of the methods and compositions of the disclosure, the first MHC subunit is a beta2-microglobulin chain, the second MHC subunit is an MHC Class I alpha chain and the MHC-binding peptide is an MHC Class I binding peptide. In another embodiment, the first MHC subunit is an MHC Class I alpha chain, the second MHC subunit is a beta2-microglobulin chain and the MHC-binding peptide is an MHC Class I binding peptide. In one embodiment, the MHC Class I binding peptide is a CMV pp65 peptide comprising the amino acid sequence NLVPMVATV (SEQ ID NO: 4). In one embodiment, the MHC Class I binding peptide is a peptide comprising an amino acid sequence selected from the group consisting of SEQ ID NOs: 204-223 and 267-320. In one embodiment, the MHC Class I alpha chain is an HLA-A*02:01 polypeptide comprising the amino acid sequence shown in SEQ ID NO: 5 or 321. In other embodiments, the MHC Class I alpha chain comprises an amino acid sequence shown selected from the group of sequences shown in SEQ ID NOs: 76-141. In one embodiment, the beta2-microglobulin chain comprises an amino acid sequence shown in SEQ ID NO: 143.

In another embodiment of the methods and compositions of the disclosure, the first MHC subunit is an MHC Class II alpha chain, the second MHC subunit is an MHC Class II beta chain and the MHC-binding peptide is an MHC Class II binding peptide. In another embodiment, the first MHC subunit is an MHC Class II beta chain, the second MHC subunit is an MHC Class II alpha chain and the MHC-binding peptide is an MHC Class II binding peptide. In one embodiment, the MHC Class II binding peptide is a CLIP peptide comprising the amino acid sequence KPVSKMRMATPLLMQA (SEQ ID NO: 224). In one embodiment, the MHC Class II alpha chain comprises an amino acid sequence shown selected from the group of sequences shown in SEQ ID NOs: 174, 190, 192, 194 and 196. In one embodiment, the MHC Class II beta chain comprises an amino acid sequence shown selected from the group of sequences shown in SEQ ID NOs: 175-189, 191, 193, 195 and 197-203.

In one embodiment of the methods and compositions of the disclosure, the MHC multimer expression construct encodes a linker between the first MHC subunit and the second MHC subunit, such as a (G₄S)₄ linker. In one embodiment, the MHC multimer expression construct encodes a linker between (i) the first and second MHC subunits and (ii) the multimerization domain, such as a (GS)₂AG₂SGSG₃S linker.

In one embodiment of the methods and compositions of the disclosure, the cleavage site operatively linked to the MHC-binding peptide is a Factor Xa cleavage site (e.g., comprising the amino acid sequence shown in SEQ ID NO: 235).

In one embodiment of the methods and compositions of the disclosure, the multimerization domain comprises streptavidin. Suitable streptavidin sequences are provided herein.

In one embodiment of the methods and compositions of the disclosure, the MHC multimer expression construct further encodes a signal peptide, such as an Ig Kappa chain V-III region CLL signal peptide.

In one embodiment of the methods and compositions of the disclosure, the MHC multimer expression construct further encodes an expression tag, such as an expression tag selected from the group consisting of 6×His tag, FLAG tag, V5 tag, Myc tag, protein C tag and combinations thereof.

In one embodiment of the methods and compositions of the disclosure, the MHC multimer expression construct comprises a nucleic acid encoding, from 5′ to 3′: an optional signal peptide—an MHC-binding peptide—a cleavage site—a first MHC subunit—a linker—a second MHC subunit—a linker—and a multimerization domain. Other suitable 5′ to 3′ configurations of the MHC multimer expression construct are described herein. In one embodiment, the MHC multimer expression construct comprises a nucleic acid encoding from 5′ to 3′: a signal peptide—an MHC Class I binding peptide—a Factor Xa cleavage site—beta2-microglobulin-a linker—an MHC Class I alpha chain—a linker—and streptavidin. In one embodiment, the MHC multimer expression construct encodes an amino acid sequence shown in SEQ ID NO: 3. In one embodiment, the MHC multimer expression construct comprises the nucleotide sequence shown in SEQ ID NO: 1.

In one embodiment, the MHC multimer further comprises an oligonucleotide barcode, such as a biotin-conjugated oligonucleotide barcode.

In one embodiment of the methods and compositions of the disclosure, the host cell is a mammalian host cell, such as a human embryonic kidney (HEK) cell line (e.g., a 293-derived cell line).

In one embodiment of the method of producing the MHC multimer, the MHC multimer is secreted from the host cell into cell culture medium (e.g., cell supernatant). In one embodiment, when the multimerization domain is streptavidin or avidin, the cell culture medium lacks biotin and the method further comprises incubating the MHC multimer with a biotin-conjugated oligonucleotide barcode, to thereby label the MHC multimers through the biotin-binding sites on streptavidin or avidin.

In one embodiment of the method of producing the MHC multimer, the method further comprising incubating the MHC multimer produced by the host cell with an agent that cleaves the cleavage site operatively linked to the MHC-binding peptide, to thereby release the MHC-binding peptide from its covalent conjugation to the recombinant MHC multimer fusion polypeptide. Following peptide cleavage (e.g., with Factor Xa), the method can further comprise incubating the MHC multimer with at least one MHC-binding rescue peptide such that peptide exchange occurs between the (original) MHC-binding peptide and the MHC-binding rescue peptide. In one embodiment, the MHC multimers are incubated with a plurality of MHC-binding rescue peptides thereby to produce a library of peptide-bound MHC multimers.

In one embodiment of the MHC multimer expression constructs of the disclosure, the expression construct is a plasmid. Host cell compositions transfected with an expression construct of the disclosure are also provided. In one embodiment, the host cell is a mammalian host cell, such as a human embryonic kidney (HEK) cell line (e.g., a 293-derived cell line) or a CHO cell line. In another embodiment, the host cell is a eukaryotic host cell such as the Drosophila cell line S2.

Isolated supernatants comprising a recombinant MHC multimer are also provided, wherein the supernatant can be isolated from culture medium of the host cells of the disclosure. In one embodiment, when the multimerization domain is streptavidin or avidin, the culture medium lacks biotin and the supernatant further comprises a biotin-conjugated oligonucleotide barcode, such that the MHC multimers are labeled with the oligonucleotide barcodes through the biotin-binding sites on streptavidin or avidin.

In one embodiment, a supernatant of the disclosure comprising MHC multimers can further comprise an agent that cleaves the cleavage site (e.g., Factor Xa for cleavage at a Factor Xa site within the multimer). In certain embodiments, MHC multimers are purified, or semi-purified, from the supernatant before cleavage with the cleaving agent (e.g., protease).

In one embodiment, following cleavage of the supernatant, or MHC multimers purified therefrom, with the cleaving agent, the supernatant or purified MHC multimers can be incubated with at least one MHC-binding rescue peptide such that peptide exchange occurs between the MHC-binding peptide released by cleavage and the MHC-binding rescue peptide.

In one embodiment, a plurality of MHC-binding rescue peptides is used such that following peptide exchange a library of peptide-bound MHC multimers is obtained (e.g., is contained in the supernatant). Accordingly, in another aspect, the disclosure pertains to a polypeptide library comprising a plurality of peptide loaded MHC (pMHC) multimers, wherein each of the pMHC multimers comprises two or more pMHC monomers conjugated to a multimerization domain, wherein the polypeptide library is prepared according to the methods of the disclosure. In one embodiment, the library comprises pMHCI multimers. In another embodiment, the library comprises pMHCII multimers.

In yet another aspect, the disclosure pertains to a method of isolating pMHC-multimer bound lymphocytes, the method comprising:
(a) contacting a plurality of lymphocytes with the library of pMHC multimers prepared according to the methods of the disclosure, thereby to produce a corresponding plurality of lymphocytes each bound to a pMHC-multimer; and
(b) isolating a pMHC-multimer-bound lymphocyte.
   In one embodiment, the pMHC-multimer-bound lymphocyte is isolated using a capture support.
   In another embodiment, the pMHC-multimer-bound lymphocyte is isolated by cell sorting, e.g., by fluorescent activated cell sorting (FACS) using an appropriate fluorescent secondary antibody. In one embodiment, the pMHC multimers are pMHCI multimers. In another embodiment, the pMHC multimers are pMHCII multimers. In various embodiments, the lymphocyte is a T cell, B cell or NK cell.

In yet another aspect, the disclosure pertains to a method of identifying a lymphocyte bound to an pMHC multimer, the method comprising:
(a) contacting a plurality of lymphocytes with the library of pMHC multimers prepared according to the methods of the disclosure;
(b) compartmentalizing a lymphocyte of the plurality of lymphocytes bound to a pMHC multimer of the library in a single compartment, wherein the pMHC multimer comprises a unique identifier; and
(c) determining the unique identifier for the pMHC bound to the compartmentalized lymphocyte.
   In one embodiment, the pMHC multimers are pMHCI multimers. In another embodiment, the pMHC multimers are pMHCII multimers. In various embodiments, the lymphocyte is a T cell, B cell or NK cell.

For a fuller understanding of the nature and advantages of the present disclosure, reference should be had to the ensuing detailed description taken in conjunction with the accompanying figures. The present disclosure is capable of modification in various respects without departing from the present disclosure. Accordingly, the figures and description of these embodiments are not restrictive.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic diagram of a representative example of an A*02:01-NLV pMHC I multimer expression construct.

FIG. 2 shows an anti-FLAG Western blot analysis of supernatants from host cells 6 days post-transfection with candidate pMHCI tetramer constructs.

FIG. 3A-3B show SDS-PAGE gels of purified A*02:01-NLV pMHCI tetramers from host cells transfected with candidate pMHCI tetramer constructs, comparing samples that were reduced/boiled, non-reduced/non-boiled or non-reduced/non-boiled and barcode-labeled. FIG. 3A shows results using a 4-12% Bis-Tris polyacrylamide gel. FIG. 3B shows non-reduced, non-boiled results using a 3-8% Tris-Acetate polyacrylamide gel.

FIG. 4A-4B are bar graphs of results of fluorescent staining experiments for antigen-specific CD8+ T cells stained with A*02:01-NLV pMHCI tetramers exchanged with the indicated peptide epitopes, confirming Factor Xa digestion and peptide exchange. FIG. 4A shows percent tetramer binding. FIG. 4B shows mean fluorescence intensity (MFI).

FIGS. 5A-5F are graphs of results of Differential Scanning Fluorimetry (DSF) experiments for pMHCI tetramers exchanged with the indicated peptide epitopes, confirming Factor Xa digestion and peptide exchange. FIG. 5A shows results for MART-1 peptide-exchanged tetramers, FIG. 5B shows results for HPV peptide-exchanged tetramers, FIG. 5C shows results for HSV peptide-exchanged tetramers, FIG. 5D shows results for WT-1 peptide-exchanged tetramers, FIG. 5E shows results for control tetramers subjected to Factor Xa digestion but in the absence of peptide and FIG. 5F shows results for untreated control tetramers.

FIGS. 6A-6I show graphs of analytical size-exclusion chromatography results for pMHCI tetramers to evaluate stability under different conditions. FIG. 6A shows baseline control results at time 0, FIG. 6B shows results for incubation at 4° C. for 1 day, FIG. 6C shows results for incubation at 4° C. for 2 days, FIG. 6D shows results for incubation at 4° C. for 4 days, FIG. 6E shows results for incubation at 4° C. for 7 days, FIG. 6F shows results for incubation at 4° C. for 13 days, FIG. 6G shows results after one round of freeze/thaw, FIG. 6H shows results after two rounds of freeze/thaw and FIG. 6I shows results for incubation at 30° C. for 24 hours.

FIGS. 7A-7D show graphs of analytical size-exclusion chromatography results for pMHCI tetramers to evaluate stability during and after peptide exchange. FIG. 7A shows baseline control results before Factor Xa cleavage and peptide exchange. FIG. 7B shows results after Factor Xa cleavage and exchange of the peptide. FIG. 7C shows results after Factor Xa cleavage and exchange of the peptide plus one round of freeze/thaw. FIG. 7D shows Factor Xa enzyme alone.

FIGS. 8A-8C show MFI results of fluorescent staining experiments for antigen-specific CD8+ T cells stained with titrations of A*02:01-NLV pMHCI tetramers exchanged with the indicated peptide epitopes, confirming Factor Xa digestion and peptide exchange. FIG. 8A shows a titration of tetramers that were untreated, digested with Factor Xa only, or exchanged with 2 different concentrations of excess WT-1 peptide, on WT-1-expanded CD8+ T cells. FIG. 8B shows a titration of tetramers that were untreated, digested with Factor Xa only, or exchanged with excess WT-1 peptide, on NLV-expanded CD8+ T cells. FIG. 8C shows a titration of tetramers that were untreated, digested with Factor Xa only, or exchanged with excess MART1-1 peptide, on MART-1-expanded CD8+ T cells.

FIG. 9A-C show results of peptide exchange with an MHCI construct containing a Y84A mutation in the HLA sequence. FIG. 9A is a schematic diagram of a representative example of an A*02:01-NLV pMHC I multimer expression construct with a Y84A mutation in the HLA heavy chain. FIG. 9B and FIG. 9C are bar graphs of MFI results of fluorescent staining experiments with WT-1-expanded and NLV-expanded CD8+ T cells stained with the Y84A variant of A*02:01-NLV pMHCI tetramers untreated (UT), digested with Factor Xa, or exchanged with WT-1 peptide, confirming Factor Xa digestion and peptide exchange.

FIG. 10A-D show anti-FLAG Western blot analysis of supernatants from host cells 6 days post-transfection with candidate pMHCI tetramer constructs corresponding to 56 different MHC Class I alleles with the indicated linked peptides.

FIG. 11 shows a plot of W6/32 ELISA analysis of supernatants from host cells 6 days post-transfection with candidate pMHCI tetramer constructs corresponding to 51 different MHC Class I alleles with the indicated linked peptides.

### DETAILED DESCRIPTION

### Definitions

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, an “altered peptide ligand” or “APL” refers to an altered or mutated version of a peptide ligand, such as an MHC binding peptide. The altered or mutated version of the peptide ligand contains at least one structural modification (e.g., amino acid substitution) as compared to the peptide ligand from which it is derived. For example, a panel of APLs can be prepared by systematic or random mutation of a known MHC binding peptide, to thereby create a pool of APLs that can be used as a library of MHC binding peptides for loading onto MHC Multimers as described herein.

As used herein, the term “and/or” when used in the context of a list of entities, refers to the entities being present singly or in any possible combination or subcombination.

The term “antigenic determinant” or “epitope” refers to a site on an antigen to which the variable domain of a T-cell receptor, an MHC molecule or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given TCR or antibody (i.e., epitope mapping) are well known in the art and include, for example, immunoblotting and immunoprecipitation assays, wherein overlapping or contiguous peptides from the antigen are tested for reactivity with the given TCR or immunoglobulin. Methods of determining spatial conformation of epitopes include techniques in the art and those described herein, for example, x-ray crystallography nuclear magnetic resonance, cryogenic electron microscopy (cryo-EM), hydrogen deuterium exchange mass spectrometry (HDX-MS), and site-directed mutagenesis (see, e.g., Epitope Mapping Protocols in Methods in Molecular Biology, Vol. 66, G. E. Morris, Ed. (1996)).

The term “avidity” as used herein, refers to the binding strength of as a function of the cooperative interactivity of multiple binding sites of a multivalent molecule (e.g., a soluble multimeric pMHC-immunoglobulin protein) with a target molecule. A number of technologies exist to characterize the avidity of molecular interactions including switchSENSE and surface plasmon resonance (Gjelstrup et al., J. Immunol. 188:1292-1306, 2012); Vorup-Jensen, Adv. Drug. Deliv. Rev. 64:1759-1781, 2012).

As used herein a “barcode”, also referred to as an oligonucleotide barcode, is a short nucleotide sequence (e.g., about 1, 2, 3, 4, 5, 6, 7, 8, 9, 10, 11, or 12 nucleotides long) that identifies a molecule to which it is conjugated. Barcodes can be used, for example, to identify molecules in a reaction mixture. Barcodes uniquely identify the molecule to which it is conjugated, for example, by performing reverse transcription using primers that each contain a “unique molecular identifier” barcode. In other embodiment, primers can be utilized that contain “molecular barcodes” unique to each molecule. The process of labeling a molecule with a barcode is referred to herein as “barcoding.” A “DNA barcode” is a DNA sequence used to identify a target molecule during DNA sequencing. In some embodiments, a library of DNA barcodes is generated randomly, for example, by assembling oligos in pools. In other embodiments, the library of DNA barcodes is rationally designed in silico and then manufactured.

“Binding affinity” generally refers to the strength of the sum total of noncovalent interactions between a single binding site of a molecule (e.g., a TCR, pMHC) and its binding partner. Unless indicated otherwise, as used herein, “binding affinity” refers to intrinsic binding affinity which reflects a 1:1 interaction between members of a binding pair (e.g., TCR and antigen). The affinity of a molecule X for its partner Y can generally be represented by the dissociation constant (Kd). For example, the Kd can be about 200 nM, 150 nM, 100 nM, 60 nM, 50 nM, 40 nM, 30 nM, 20 nM, 10 nM, 8 nM, 6 nM, 4 nM, 2 nM, 1 nM, or stronger, including up to 20 μM. Affinity can be measured by common methods known in the art, including those described herein. Low-affinity TCRs generally bind antigen slowly and tend to dissociate readily, whereas high-affinity TCRs generally bind antigen faster and tend to remain bound longer. A variety of methods of measuring binding affinity are known in the art, any of which can be used for purposes of the present disclosure.

As used herein, the terms “carrier” and “pharmaceutically acceptable carrier” includes any and all solvents, dispersion media, coatings, antibacterial and antifungal agents, isotonic and absorption delaying agents, and the like that are physiologically compatible.

As used herein, the term “cleavage site” or “cleavable moiety” refers to a site, a motif or sequence that is cleavable, such as by an enzyme (e.g., a protease) or by particular reaction conditions. In some embodiments, the cleavage moiety comprises a protein, e.g., enzymatic, cleavage site. In some embodiments, the cleavage moiety comprises a chemical cleavage site, e.g., through exposure to oxidation/reduction conditions, light/sound, temperature, pH, pressure, etc.

As used herein, the term “cross-linking unit” can refer to a molecule that links to another (same or different) molecule. In some embodiments, the cross-linking unit is a monomer. In some embodiments, the cross-link is a chemical bond. In some embodiments, the cross-link is a covalent bond. In some embodiments, the cross-link is an ionic bond. In some embodiments, the cross-link alters at least one physical property of the linked molecules, e.g., a polymer's physical property.

As used herein, the term “endoprotease” refers to a protease that cleaves a peptide bond of a non-terminal amino acid.

As used herein, the term “epitope” (as in “peptide epitope”) refers to a portion of an antigen (e.g., antigenic protein) that binds to (interacts with or is recognized by) an immune receptor. Thus, a T cell receptor recognizes and binds to an MHC molecule complexed with (loaded with) a peptide epitope.

The terms “exchangeable pMHC polypeptide”, “exchangeable pMHC multimers”, and “placeholder-peptide loaded MHC polypeptide”, which are used interchangeably herein, refer to MHC monomers and MHC multimers, comprising a placeholder peptide in the binding groove of the MHC polypeptide, and are also referred to as “p*MHC” monomers or multimers. “Exchangeable” refers to the property of a p*MHC monomer or p*MHC multimer allowing for the exchange of the placeholder peptide with an antigenic peptide. In one embodiment, the exchangeable pMHC or p*MHC polypeptide comprises an MHC Class I molecule with an MHC Class I-binding peptide in the binding groove of the MHC Class I molecule. In another embodiment, the exchangeable pMHC or p*MHC polypeptide comprises an MHC Class II molecule with an MHC Class II-binding peptide in the binding groove of the MHC Class II molecule.

As used herein, the term “expression construct” refers to a vector designed for gene expression, e.g., in a host cell. An expression vector promotes the expression (i.e., transcription/translation) of an encoded polypeptide (e.g., fusion polypeptide). Typically, the vector is a plasmid, although other suitable vectors, including viral and non-viral vectors are also encompassed by the term “expression construct.”

A “fusion protein” or “fusion polypeptide” as used interchangeably herein refers to a recombinant protein prepared by linking or fusing two polypeptides into a single protein molecule.

The term “isolated” as applied to MHC monomers herein refers to an MHC glycoprotein, which is in other than its native state, for example, not associated with the cell membrane of a cell that normally expresses MHC. This term embraces a full length subunit chain, as well as a functional fragment of the MHC monomer. A functional fragment is one comprising an antigen binding site and sequences necessary for recognition by the appropriate T cell receptor. It typically comprises at least about 60-80%, typically 90-95% of the sequence of the full-length chain. An “isolated” MHC subunit component may be recombinantly produced or solubilized from the appropriate cell source. In one embodiment, the “isolated” MHC monomer is an MHC Class I monomer, such as a soluble form of the MHC Class I heavy chain (α chain) associated with β2-microglobulin. In another embodiment, the “isolated” MHC monomer is an MHC Class II monomer, such as a soluble form of the MHC Class II a/P chains.

As used herein, the term “identifier” refers to a readable representation of data that provides information, such as an identity, that corresponds with the identifier.

As used herein, the terms “linked,” “conjugated,” “fused,” or “fusion,” are used interchangeably when referring to the joining together of two more elements or components or domains, by whatever means including recombinant or chemical means.

As used herein, the term “linker sequence” refers to a nucleotide sequence, and corresponding encoded amino acid sequence, within an expression construct that serves to link or separate two polypeptides, such as two polypeptide domains of a fusion protein. For example, an intervening linker sequence can serve to provide flexibility and/or additional space between the two polypeptides that flank the linker.

As used herein, the terms “operatively linked” and “operably linked” are used interchangeably to describe configurations between sequences within an expression construct that allow for particular operations to carried out. For example, when a regulatory sequence is “operatively linked” to a coding sequence within an expression construct, the regulatory sequence operates to regulate the expression of the coding sequence. Similarly, when a cleavage sequence (site) is “operatively linked” to a peptide sequence within an expression construct, cleavage at the cleavage sequence operates to cleave the peptide sequence away from the rest of the polypeptide encoded by the expression construct.

The term “Major Histocompatibility Complex” or “MHC” refers to genomic locus containing a group of genes that encode the polymorphic cell-membrane-bound glycoproteins known as MHC classical class I and class II molecules that regulate the immune response by presenting peptides of fragmented proteins to circulating cytotoxic and helper T lymphocytes, respectively. In humans this group of genes is also called the “human leukocyte antigen” or “HLA” system. Human MHC class I genes encode, for example, HLA-A, HL-B and HLA-C molecules. HLA-A is one of three major types of human MHC class I cell surface receptors. The others are HLA-B and HLA-C. The HLA-A protein is a heterodimer, and is composed of a heavy a chain and smaller R chain. The α chain is encoded by a variant HLA-A gene, and the R chain is an invariant β2 microglobulin (β2m) polypeptide. The β2 microglobulin polypeptide is coded for by a separate region of the human genome. HLA-A*02 (A*02) is a human leukocyte antigen serotype within the HLA-A serotype group. The serotype is determined by the antibody recognition of the α2 domain of the HLA-A α-chain. For A*02, the α chain is encoded by the HLA-A*02 gene and the R chain is encoded by the B2M locus. Human MHC class II genes encode, for example, HLA-DPA1, HLA-DPB1, HLA-DQA1, HLA-DQB1, HLA-DRA and HLA-DRB1. The complete nucleotide sequence and gene map of the human major histocompatibility complex is publicly available (e.g., The MHC sequencing consortium, Nature 401:921-923, 1999).

As used herein, the terms “MHC molecule” and “MHC protein” are used herein to refer to the polymorphic glycoproteins encoded by the MHC class I and MHC class II genes, which are involved in the presentation of peptide epitopes to T cells. The terms “MHC class I” or “MHC I” are used interchangeably to refer to protein molecules comprising an a chain composed of three domains (α1, α2 and α3), and a second, invariant β2-microglobulin. The α3 domain is transmembrane, anchoring the MHC class I molecule to the cell membrane. Antigen-derived peptide epitopes, which are located in the peptide-binding groove, in the central region of the α1/α2 heterodimer. MHC Class I molecules such as HLA-A are part of a process that presents short polypeptides to the immune system. These polypeptides are typically 9-11 amino acids in length and originate from proteins being expressed by the cell. MHC class I molecules present antigen to CD8+ cytotoxic T cells. The terms “MHC class II” and “MHC II” are used interchangeably to refer to protein molecules containing an a chain with two domains (α1 and α2) and a β chain with two domains (β1 and β2). The peptide-binding groove is formed by the α1/β1 heterodimer. MHC class II molecules present antigen to specific CD4+ T cells. Antigens delivered endogenously to APCs are processed primarily for association with MHC class I. Antigens delivered exogenously to APCs are processed primarily for association with MHC class II.

As used herein, MHC proteins (MHC Class I or Class II proteins) also includes MHC variants which contain amino acid substitutions, deletions or insertions and yet which still bind MHC peptide epitopes (MHC Class I or MHC Class II peptide epitopes). The term also includes fragments of all these proteins, for example, the extracellular domain, which retain peptide binding.

The term “MHC protein” also includes MHC proteins of non-human species of vertebrates. MHC proteins of non-human species of vertebrates play a role in the examination and healing of diseases of these species of vertebrates, for example, in veterinary medicine and in animal tests in which human diseases are examined on an animal model, for example, EAE (experimental autoimmune encephalomyelitis) in mice (Mus musculus), which is an animal model of the human disease multiple sclerosis. Non-human species of vertebrates are, for example, and more specifically mice (Mus musculus), rats (Rattus norvegicus), cows (Bos taurus), horses (Equus equus) and green monkeys (Macaca mulatta). MHC proteins of mice are, for example, referred to as H-2-proteins, wherein the MHC class I proteins are encoded by the gene loci H2K, H2L and H2D and the MHC class II proteins are encoded by the gene loci H2I.

A “peptide free MHC polypeptide” or “peptide free MHC multimer” as used herein refers to an MHC monomer or MHC multimer which does not contain a peptide in binding groove of the MHC polypeptide. Peptide free MHC monomers and multimers are also referred to as “empty”. In one embodiment, the peptide free MHC polypeptide or multimer is an MHC Class I polypeptide or multimer. In another embodiment, the peptide free MHC polypeptide or multimer is an MHC Class II polypeptide or multimer.

As used herein, the term “multimer” refers to a plurality of units. In some embodiments, the multimer comprises one or more different units. In some embodiments, the units in the multimer are the same. In some embodiments, the units in the multimer are different. In some embodiments, the multimer comprises a mixture of units that are the same and different.

The terms “peptide epitope”, “MHC peptide epitope”, “MHC peptide antigen” and “MHC ligand” are used interchangeably herein and refer to an MHC ligand that can bind in the peptide binding groove of an MHC molecule. The peptide epitope can typically be presented by the MHC molecule. A peptide epitope typically has between 8 and 25 amino acids that are linked via peptide bonds. The peptide can contain modification such as, but not limited to, the side chains of the amino acid residues, the presence of a label or tag, the presence of a synthetic amino acid, a functional equivalent of an amino acid, or the like. Typical modifications include those as produced by the cellular machinery, such as glycan addition and phosphorylation. However, other types of modification are also within the scope of the disclosure.

As used herein, the terms “peptide exchange” refers to a competition assay wherein a placeholder peptide is removed and replaced by a “exchanged peptide” (or “exchange peptide epitope”) also referred to herein as a “rescue peptide” (or “rescue peptide epitope”) or “competitor peptide” (or “competitor peptide epitope). Typically, peptide exchange occurs under conditions in which the placeholder peptide is released by cleavage of the peptide or under suitable conditions allowing rescue peptides to compete for binding to the binding pocket of an MHC monomer or multimer. For example, peptide exchange can be accomplished by, for example, temperature-induced exchange, UV-induced exchange, dipeptide-induced exchange, or other exchange methods known in the art, and disclosed herein.

As used herein, the term “peptide library” refers to a plurality of peptides. In some embodiments, the library comprises one or more peptides with unique sequences. In some embodiments, each peptide in the library has a different sequence. In some embodiments, the library comprises a mixture of peptides with the same and different sequences.

As used herein, the term “high diversity peptide library” refers to a peptide library with a high degree of peptide variety. For example, a high diversity peptide library comprises about 10³, about 10⁴, about 10⁵, about 10⁶, about 10⁷, about 10⁸, about 10⁹, about 10¹⁰, about 10¹¹, about 1012, about 1013, about 1014, about 1015, about 1016, about 1017, about 1018, about 1019, about 10²⁰, or more different peptides.

As used herein, the term “library peptide” refers to a single peptide in the library.

As used herein, the terms “placeholder peptide” or “exchangeable peptide” are used interchangeably to refer to a peptide or peptide-like compound that binds with sufficient affinity to an MHC protein (e.g., MHCI or MHCII protein) and which causes or promotes proper folding of the MHC protein from the unfolded state or stabilization of the folded MHC protein. The placeholder peptide can subsequently be exchanged with a different peptide of interest (referred to as an exchange peptide or rescue peptide). This exchange can be accomplished by, for example, UV-induced exchange, dipeptide-induced exchange, temperature-induced exchange, or other exchange methods known in the art.

The terms “polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The terms “isolated protein” and “isolated polypeptide” are used interchangeably to refer to a protein (e.g., a soluble, multimeric protein) which has been separated or purified from other components (e.g., proteins, cellular material) and/or chemicals. Typically, a polypeptide is purified when it constitutes at least 60 (e.g., at least 65, 70, 75, 80, 85, 90, 92, 95, 97, or 99) % by weight of the total protein in the sample.

As used herein, the term “protein folding” refers to spatial organization of a peptide. In some embodiments, the amino acid sequence influences the spatial organization or folding of the peptide. In some embodiments, a peptide may be folded in a functional conformation. In some embodiments, a folded peptide has one or more biological functions. In some embodiments, a folded peptide acquires a three-dimensional structure.

As used herein, the term “N-terminus amino acid residue” refers to one or more amino acids at the N-terminus of a polypeptide.

As used herein, the terms “small ubiquitin-like modifier moiety” or “SUMO domain” or “SUMO moiety” are used interchangeably and refer to a specific protease recognition moiety.

As used herein, the term “tag” refers to an oligonucleotide component, generally DNA, that provides a means of addressing a target molecule (e.g., an MHC Multimer) to which it is joined. For example, in some embodiments, a tag comprises a nucleotide sequence that permits identification, recognition, and/or molecular or biochemical manipulation of the molecule to which the tag is attached (e.g., by providing a unique sequence, and/or a site for annealing an oligonucleotide, such as a primer for extension by a DNA polymerase, or an oligonucleotide for capture or for a ligation reaction). The process of joining the tag to the target molecule is sometimes referred to herein as “tagging” and a target molecule that undergoes tagging or that contains a tag is referred to as “tagged” (e.g., a “tagged MHC Multimer”).” A tag can be a barcode, an adapter sequence, a primer hybridization site, or a combination thereof.

The term “T cell” refers to a type of white blood cell that can be distinguished from other white blood cells by the presence of a T cell receptor on the cell surface. There are several subsets of T cells, including, but not limited to, T helper cells (a.k.a. T_{H} cells or CD4⁺ T cells) and subtypes, including T_{H}1, T_{H}2, T_{H}3, T_{H}17, T_{H}9, and T_{FH} cells, cytotoxic T cells (a.k.a T_{C} cells, CD8+ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺FOXP3⁺T_{reg} cells, CD4⁺FOXP3⁻T_{reg} cells, Tr1 cells, Th3 cells, and T_{reg}17 cells, natural killer T cells (a.k.a. NKT cells), mucosal associated invariant T cells (MAITs), and gamma delta T cells (γδ T cells), including Vγ9/Vβ2 T cells. The term “T cell cytotoxicity” includes any immune response that is mediated by CD8+ T cell activation.

As used herein, the phrase “T cell receptor” and the term “TCR” refer to a surface protein of a T cell that allows the T cell to recognize an antigen and/or an epitope thereof, typically bound to one or more major histocompatibility complex (MHC) molecules. A TCR functions to recognize an antigenic determinant and to initiate an immune response. Typically, TCRs are heterodimers comprising two different protein chains. In the vast majority of T cells, the TCR comprises an alpha (α) chain and a beta (β) chain. Each chain comprises two extracellular domains: a variable (V) region and a constant (C) region, the latter of which is membrane-proximal. The variable domains of α-chains and of β-chains consist of three hypervariable regions that are also referred to as the complementarity determining regions (CDRs). The CDRs, in particular CDR3, are primarily responsible for contacting antigens and thus define the specificity of the TCR, although CDR1 of the α-chain can interact with the N-terminal part of the antigen, and CDR1 of the β-chain interacts with the C-terminal part of the antigen. Approximately 5% of T cells have TCRs made up of gamma and delta (γ/δ) chains. All numbering of the amino acid sequences and designation of protein loops and sheets of the TCRs is according to the IMGT numbering scheme (IMGT, the international ImMunoGeneTics information system@imgt.cines.fr; http://imgt.cines.fr; Lefranc et al., (2003) Dev Comp Immunol 27:55 77; Lefranc et al. (2005) Dev Comp Immunol 29:185-203).

As used herein, the terms “soluble T-cell receptor” and “sTCR” refer to heterodimeric truncated variants of TCRs, which comprise extracellular portions of the TCR α-chain and 3-chain (e.g., linked by a disulfide bond), but which lack the transmembrane and cytosolic domains of the full-length protein. The sequence (amino acid or nucleic acid) of the soluble TCR α-chain and β-chains may be identical to the corresponding sequences in a native TCR or may comprise variant soluble TCR α-chain and β-chain sequences, as compared to the corresponding native TCR sequences. The term “soluble T-cell receptor” as used herein encompasses soluble TCRs with variant or non-variant soluble TCR α-chain and β-chain sequences. The variations may be in the variable or constant regions of the soluble TCR α-chain and β-chain sequences and can include, but are not limited to, amino acid deletion, insertion, substitution mutations as well as changes to the nucleic acid sequence, which do not alter the amino acid sequence. Variants retain the binding functionality of their parent molecules.

As used herein, a “TCR/pMHC complex” refers to a protein complex formed by binding between T cell receptor (TCR), or soluble portion thereof, and a peptide-loaded MHC molecule. Accordingly, a “component of a TCR/pMHC complex” refers to one or more subunits of a TCR (e.g., Vα, Vβ, Cα, Cβ), or to one or more subunits of an MHC or pMHC class I or II molecule.

As used herein, the term “unbiased” refers to lacking one or more selective criteria.

### Overview

This disclosure provides methods and compositions for the high-throughput generation of libraries containing peptide-loaded MHC (pMHC) multimers containing a plurality of unique peptides in the MHC binding groove and having oligonucleotide barcode labeling to facilitate identification of library members. In the methods provided herein, a recombinant expression construct is used that contiguously encodes all of the components of the MHC multimer in a single construct such that upon expression in a host cell, the MHC multimer is produced and self-assembles. These components include an MHC-binding peptide, MHC molecule chains (alpha chain and beta2-microglobulin for MHC Class I; alpha chain and beta chain for MHC Class II) and a multimerization domain. Upon expression, multimerization mediated by the multimerization domain occurs such that a multimer is produced that contains a plurality of MHC monomers, with the peptide-binding groove of each monomer being occupied by the MHC-binding peptide. This MHC binding peptide can be released from the multimer through digestion at a cleavage site such that peptide exchange can be carried out, e.g., with a panel of rescue peptide epitopes that bind the same MHC molecule, to thereby prepare pMHC libraries. Moreover, a binding site on the multimerization domain (e.g., the biotin-binding site of streptavidin or avidin) can be used for labeling the MHC multimers with unique identifiers (e.g., biotinylated oligonucleotide barcodes).

The libraries of pMHC multimers provided herein are useful in a range of therapeutic, diagnostic, and research applications, essentially in any situation in which pMHC multimers are useful. For example, pMHC multimers as described herein can be used in a variety of methods, for example, to identify and isolate specific T-cells in a wide array of applications. In one embodiment, the pMHC multimers are pMHC Class I multimers, which are useful for determining the antigenic specificity of CD8+ T cells (e.g., cytotoxic T cells). In another embodiment, the pMHC multimers are pMHC Class II multimers, which are useful for determining the antigenic specificity of CD4+ T cells (e.g., helper T cells).

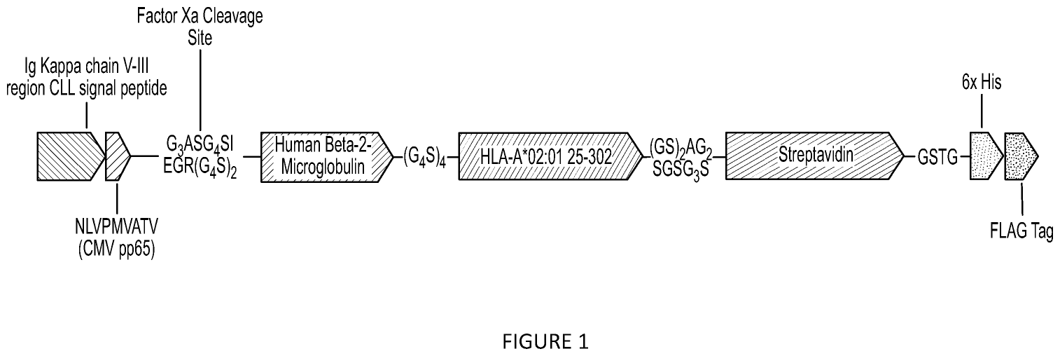

While prior approaches for making pMHC multimers involve expression and purification of the pMHC monomer and multimerization domain components separately, followed by assemblage of the multimer extracellularly, the present disclosure provides a single expression construct that encodes all the necessary components of the pMHC multimers, including the MHC-binding peptide, the MHC molecule chains and the multimerization domain, such that self-assembly of the MHC multimer occurs following host cell expression. A non-limiting representative example of an MHC (class I) multimer expression construct is shown schematically in FIG. 1. This schematic illustrates the contiguous coding region contained in the vector, which encodes all necessary components of the MHC multimer. Linker sequences typically are interspersed between the sequences of the functional components (i.e., MHC-binding peptide, the MHC molecule chains and the multimerization domain). Additionally, the N-terminus typically encodes a signal sequence to facilitate secretion of the MHC multimer from the host cells. Still further, the N- or C-terminus of the encoded fusion polypeptide can include one or more tags (e.g., affinity tags) to facilitate detection of the MHC multimer following expression, by standard techniques. Various components and aspects of the disclosure are described in further detail in the subsections below.

### I. MHC Multimer Polynucleotides, Expression Vectors and Host Cells

As described in Example 1 and illustrated in FIG. 1, MHC multimer expression constructs can be designed that encode all necessary functional components of the MHC multimer such that the multimer self-assembles upon expression in a host cell. These functional components include: an MHC-binding peptide (also abbreviated herein as “PEP”; e.g., an exchangeable “placeholder” peptide), the MHC molecule chains (abbreviated herein as “MHC”) and a multimerization domain (abbreviated herein as “MD”). Typically, linker sequences are interspersed between the functional components, with the MHC-binding peptide being operatively linked to a linker sequence that comprises a cleavage site (e.g., an enzyme recognition site), to facilitate cleavage of the placeholder peptide from the MHC multimer, such as to carry out peptide exchange. Suitable linker sequences and cleavage sites are known in the art and are described further herein, including GS linkers and protease recognition sites. Expression and screening of MHC multimer expression constructs in mammalian host cells is described in detail in Example 2.

In one embodiment, the 5′ to 3′ configuration of the expression construct is: 5′-PEP-MHC-MD-3′. For example, the 5′ to 3′ configuration can comprise: 5′-signal sequence-peptide-cleavage site-first MHC domain-linker-second MHC domain-linker-multimerization domain-3′. In one embodiment, the 5′ to 3′ configuration can comprise: 5′-signal sequence-peptide-cleavage site-MHCI alpha chain-linker-β2-microglobulin chain linker-multimerization domain-3′. In one embodiment, the 5′ to 3′ configuration can comprise: 5′-signal sequence-peptide-cleavage site-β2-microglobulin chain-linker-MHCI alpha chain-linker-multimerization domain-3′. In one embodiment, the 5′ to 3′ configuration can comprise: 5′-signal sequence-peptide-cleavage site-MHCII alpha chain linker-MHCII beta chain-linker-multimerization domain-3′. In one embodiment, the 5′ to 3′ configuration can comprise: 5′-signal sequence-peptide-cleavage site-MHCII beta chain-linker-MHCII alpha chain-linker-multimerization domain-3′.

In another embodiment, the 5′ to 3′ configuration of the expression construct is: 5′-PEP-MD-MHC-3′. For example, the 5′ to 3′ configuration can comprise: 5′-signal sequence-peptide-cleavage site-multimerization domain-linker-first MHC domain-linker-second MHC domain-3′. In one embodiment, the 5′ to 3′ configuration can comprise: 5′-signal sequence-peptide-cleavage site-multimerization domain-linker-MHCI alpha chain-linker-β2-microglobulin chain-3′. In one embodiment, the 5′ to 3′ configuration can comprise: 5′-signal sequence-peptide-cleavage site-multimerization domain-linker-β2-microglobulin chain-linker-MHCI alpha chain-3′. In one embodiment, the 5′ to 3′ configuration can comprise: 5′-signal sequence-peptide-cleavage site-multimerization domain-MHCII alpha chain-linker-MHCII beta chain-3′. In one embodiment, the 5′ to 3′ configuration can comprise: 5′-signal sequence-peptide-cleavage site-multimerization domain-linker-MHCII beta chain-linker-MHCII alpha chain-3′.

In another embodiment, the 5′ to 3′ configuration of the expression construct is: 5′-MHC-MD-PEP-3′. For example, the 5′ to 3′ configuration can comprise: 5′-signal sequence-first MHC domain-linker-second MHC domain-linker-multimerization domain-cleavage site-peptide-3′. In one embodiment, the 5′ to 3′ configuration can comprise: 5′-signal sequence-MHCI alpha chain-linker-β2-microglobulin chain-linker-multimerization domain-cleavage site-peptide-3′. In one embodiment, the 5′ to 3′ configuration can comprise: 5′-signal sequence-β2-microglobulin chain-linker-MHCI alpha chain-linker-multimerization domain-cleavage site-peptide-3′. In one embodiment, the 5′ to 3′ configuration can comprise: 5′-signal sequence-MHCII alpha chain-linker-MHCII beta chain-linker-multimerization domain-cleavage site-peptide-3′. In one embodiment, the 5′ to 3′ configuration can comprise: 5′-signal sequence-MHCII beta chain linker-MHCII alpha chain-linker-multimerization domain-peptide-cleavage site-3′.

In another embodiment, the 5′ to 3′ configuration of the expression construct is: 5′-MD MHC-PEP-3′. For example, the 5′ to 3′ configuration can comprise: 5′-signal sequence multimerization domain-linker-first MHC domain-linker-second MHC domain-cleavage site-peptide-3′. In one embodiment, the 5′ to 3′ configuration can comprise: 5′-signal sequence-multimerization domain-linker-MHCI alpha chain-linker-β2-microglobulin chain-cleavage site-peptide-3′. In one embodiment, the 5′ to 3′ configuration can comprise: 5′-signal sequence-multimerization domain-linker-β2-microglobulin chain linker-MHCI alpha chain-cleavage site-peptide-3′. In one embodiment, the 5′ to 3′ configuration can comprise: 5′-signal sequence-multimerization domain-linker-MHCII alpha chain-linker-MHCII beta chain-cleavage site-peptide-3′. In one embodiment, the 5′ to 3′ configuration can comprise: 5′-signal sequence-multimerization domain-linker-MHCII beta chain-linker-MHCII alpha chain-cleavage site-peptide-3′.

In certain embodiments, the expression construct includes a signal sequence operatively linked at the N-terminal end of the coding region, such that the encoded fusion polypeptide is transcribed with a signal sequence to thereby facilitate secretion of the MHC multimer from the host cell (e.g., into the cell culture medium such that the MHC multimers can be recovered from the cellular supernatant). Suitable linker sequences and cleavage sites are known in the art and are described further herein. In one embodiment, the signal sequence is a heterologous signal sequence (i.e., the signal sequence is not a native MHC signal sequence). In one embodiment, the signal sequence is from an Ig supergroup member. In one embodiment, the signal sequence is an immunoglobulin chain signal sequence. In one embodiment, the signal sequence is an Ig Kappa chain V-III region CLL signal peptide, e.g., having the sequence MEAPAQLLFLLLLWLPDTTG (SEQ ID NO: 255). Other suitable signal sequences include a human CD4 signal peptide, e.g., having the sequence MNRGVPFRHLLLVLQLALLPAAT (SEQ ID NO: 256), a mouse Ig kappa chain V-III region signal peptide, e.g., having the sequence METDTLLLWVLLLWVPGSTG (SEQ ID NO: 257), a mouse H-2Kb signal peptide, e.g., having the sequence MVPCTLLLLLAAALAPTQTRA (SEQ ID NO: 258), a human serum albumin signal peptide, e.g., having the sequence MKWVTFISLLFLFSSAYS (SEQ ID NO: 259), a human IL-2 signal peptide, e.g., having the sequence MYRMQLLSCIALSLALVTNS (SEQ ID NO: 260), a human HLA-A*02:01 signal peptide, e.g., having the sequence MAVMAPRTLLLLLSGALALTQTWA (SEQ ID NO: 261) and a human b2m signal peptide, e.g., having the sequence MSRSVALAVLALLSLSGLEA (SEQ ID NO: 262). In another embodiment, the signal sequence is a homologous signal sequence, i.e., the signal sequence is a native MHC signal sequence (e.g., from an MHC class I alpha chain, a beta-2 immunoglobulin, or an MHC class II alpha or beta chain).

In certain embodiments, the expression construct includes at least one tag sequence, most typically as at the C-terminal end of the coding region, although inclusion of a tag at the N-terminal end (alternative to or in addition to the C-terminal end) is also encompassed. Suitable tag sequences are known in the art and described further herein.

In one embodiment, the MHC multimer is an MHC Class I multimer, in which case the expression construct encodes an MHCI-binding peptide (e.g., “placeholder” peptide), the MHCI alpha chain and beta2-microglobulin and a multimerization domain.

In one embodiment, the MHC multimer is an MHC Class II multimer, in which case the expression construct encodes an MHCII-binding peptide (e.g., “placeholder” peptide), the MHCII alpha chain and beta chain and a multimerization domain.

The present disclosure encompasses nucleic acid sequences encoding any of the proteins (e.g., MHC multimer polypeptides) described herein. In one embodiment, the nucleic acid sequence is incorporated into a vector, such as a plasmid vector, a viral vector or a non-viral vector. The vector is selected to be suitable for use in the intended host cell (i.e., the vector incudes all necessary transcriptional regulatory elements to allow for expression of the encoded MHC multimer polypeptide in the host cell). Suitable vectors, including transcriptional regulatory elements for use in various host cells, including mammalian host cells, are well established in the art.

As appreciated by those skilled in the art, because of third base degeneracy, almost every amino acid can be represented by more than one triplet codon in a coding nucleotide sequence. In addition, minor base pair changes may result in a conservative substitution in the amino acid sequence encoded but are not expected to substantially alter the biological activity of the gene product. Therefore, a nucleic acid sequence encoding a protein described herein may be modified slightly in sequence and yet still encode its respective gene product.

Nucleic acids encoding any of the various proteins or polypeptides described herein may be synthesized chemically or prepared through standard recombinant DNA techniques. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA, 100(2):438-442 (Jan. 21, 2003); Sinclair et al., Protein Expr. Purif., 26(I):96-105 (October 2002); Connell, N.D., Curr. Opin. Biotechnol., 12(5):446-449 (October 2001); Makrides et al., Microbiol. Rev., 60(3):512-538 (September 1996); and Sharp et al., Yeast, 7(7):657-678 (October 1991).

General techniques for nucleic acid manipulation are described in, for example, Sambrook et al., Molecular Cloning: A Laboratory Manual, 2nd Edition, Vols. 1-3, Cold Spring Harbor Laboratory Press (1989), or Ausubel, F. et al., Current Protocols in Molecular Biology, Green Publishing and Wiley-Interscience, New York (1987) and periodic updates, herein incorporated by reference. Generally, the DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding site, and sequences that control the termination of transcription and translation. The ability to replicate in a host, usually conferred by an origin of replication, and a selection gene to facilitate recognition of transformants is additionally incorporated.

In one embodiment, the vector is designed for expression in a mammalian host cell. In one embodiment, the mammalian host cells are human host cells. In one embodiment, the human host cells are human embryonic kidney (HEK) cells. In one embodiment, the HEK cells are 293 cells or are a 293-derived HEK strain. Such HEK cells are commercially available in the art, a non-limiting example of which is the Expi293F™ cell line (Fisher ThermoScientific). In yet another embodiment, the mammalian host cell is a CHO cell line.

When mammalian host cells are used, typically the signal sequence used in the expression construct is derived from a mammalian protein. Furthermore, the transcriptional regulatory sequences used in the vector are selected for their effectiveness in mammalian host cell expression.

Other expression systems include stable Drosophila cell transfectants and baculovirus infected insect-cells suitable for expression of proteins.

For prokaryotic host cells that do not recognize and process a native signal sequence, the signal sequence is substituted by a prokaryotic signal sequence selected, for example, from the group of the alkaline phosphatase, penicillinase, 1 pp, or heat-stable enterotoxin II leaders.

For yeast secretion the native signal sequence may be substituted by, e.g., a yeast invertase leader, a factor leader (including Saccharomyces and Kluyveromyces alpha-factor leaders), or acid phosphatase leader, the C. albicans glucoamylase leader, or the signal sequence described in U.S. Pat. No. 5,631,144. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression and cloning vectors usually contain a promoter that is recognized by the host organism and is operably linked to the nucleic acid encoding the MHC multimer described herein. Promoters suitable for use with prokaryotic hosts include the phoA promoter, beta-lactamase and lactose promoter systems, alkaline phosphatase, a tryptophan (trp) promoter system, and hybrid promoters such as the tan promoter. However, other known bacterial promoters are suitable. Promoters for use in bacterial systems also will contain a Shine-Dalgarno (S.D.) sequence operably linked to the DNA encoding the protein described herein. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding protein described herein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the peptide-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of mRNA encoding the protein described herein. One useful transcription termination component is the bovine growth hormone polyadenylation region. See WO 94/11026 and the expression vector disclosed therein.

The recombinant DNA can also include any type of protein tag sequence that may be useful for purifying the protein. Examples of protein tags include, but are not limited to, a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, New York (1985)), the relevant disclosure of which is hereby incorporated by reference.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow et al. (Bio/Technology, 6:47 (1988)). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides described herein would make expression in E. coli as the preferred method for expression. The protein is then purified from culture media or cell extracts.

The host cells used to produce the proteins of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma)) are suitable for culturing the host cells. In addition, many of the media described in Ham et al., Meth. Enzymol., 58:44 (1979), Barites et al., Anal. Biochem., 102:255 (1980), U.S. Pat. Nos. 4,767,704, 4,657,866, 4,927,762, 4,560,655, 5,122,469, 6,048,728, 5,672,502, or U.S. Pat. No. RE 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins described herein can also be produced using cell-free translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Proteins described herein can also be produced by chemical synthesis (e.g., by the methods described in Solid Phase Peptide Synthesis, 2nd Edition, The Pierce Chemical Co., Rockford, Ill. (1984)). Modifications to the protein can also be produced by chemical synthesis.

The proteins of the present invention can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, get filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified polypeptide is preferably at least 85% pure, or preferably at least 95% pure, and most preferably at least 98% pure. Regardless of the exact numerical value of the purity, the polypeptide is sufficiently pure for its intended use.

The amino acid sequences of numerous MHC Class I and Class II proteins are known in the art (described further below), and the genes have been cloned; therefore, the MHC molecule sequences can be incorporated into the expression constructs of the disclosure. Methods for the recombinant expression and purification of MHCI monomers have been extensively described (e.g., Altman et al., Curr. Protoc. Enz. 17.3.1-17.2-44, 2016). For example, the MHCI heavy chain and β2-microglobulin have been expressed in separate cells, and isolated by purification and then refolded in vitro. The amino acid sequences of numerous MHC Class II proteins, including human MHCII, also are known in the art (described further below), and the genes have been cloned. Therefore, the alpha and beta chain monomers can be expressed using recombinant methods. Methods for the expression and purification of MHCII molecules have been extensively described (e.g., Crawford et al. (1998) Immunity, 8:675-682; Novak et al. (1999) J. Clin. Invest., 104:R63-R67; Nepom et al. (2002) Arthrit. Rheum., 46:5-12; Day et al. (2003) J. Clin. Invest., 112:831-842; Vollers and Stern (2008) Immunol., 123:305-313; Cecconi et al. (2008) Cytometry, 73A:1010-1018, the entire contents of each of which is hereby incorporated by reference).

MHC polypeptide chains have been expressed in E. coli, where MHC polypeptide chains accumulate as insoluble inclusion bodies in the bacterial cell. In vitro refolding occurs in a refolding buffer where the polypeptides are added by e.g. dialysis or dilution. Refolding buffers can be any buffer wherein the MHC polypeptide chains and peptide are allowed to reconstitute the native trimer fold. The buffer may contain oxidative and/or reducing agents thereby creating a redox buffer system helping the MHC proteins to establish the correct fold. Examples of suitable refolding buffers include but are not limited to Tris-buffer, CAPS buffer, TAPs buffer, PBS buffer, other phosphate buffer, carbonate buffer and Ches buffer. Chaperone molecules or other molecules improving correct protein folding may also be added and likewise agents increasing solubility and preventing aggregate formation may be added to the buffer. Examples of such molecules include but is not limited to Arginine, GroE, HSP70, HSP90, small organic compounds, DnaK, CIpB, proline, glycinbetaine, glycerol, tween, salt, PLURONIC™

Once expressed the MHC multimers of the disclosure can be purified directly from MHC multimer expressing cells, or supernatants thereof. In one embodiment, the MHC multimers are secreted from the host cells, e.g., through the use of a signal peptide. Alternatively, MHC multimers may be expressed on the surface of cells, and are then isolated by disruption of the cell membrane using, e.g., detergent followed by purification of the MHC multimers. In some embodiments, MHC multimers are expressed into the periplasm and expressing cells are lysed and released MHC multimers purified. Alternatively, MHC multimers may be purified from the supernatant of cells secreting expressed proteins into culture supernatant. Methods for purifying MHC multimers are well known in the art, for example, via the use of affinity tags together with affinity chromatography, beads coated with ant-tag and/or other techniques involving immobilization of MHC multimer to affinity matrix; size exclusion chromatography using, e.g., gel filtration, ion exchange or other methods able to separate MHC molecules from cells and/or cell lysates.

In some embodiments, recombinant expression of MHC multimers allows for introduction of modifications into the MHC monomers. For example, recombinant techniques provide methods for carboxy terminal truncation which deletes the hydrophobic transmembrane domain. The carboxy termini can also be arbitrarily chosen to facilitate the conjugation of ligands or labels, for example, by introducing cysteine and/or lysine residues into the molecule. The synthetic gene will typically include restriction sites to aid insertion into expression vectors and manipulation of the gene sequence. The genes encoding the appropriate monomers are then inserted into expression vectors, expressed in an appropriate host, such as mammalian cells, E. coli, yeast, insect, or other suitable cells, and the recombinant proteins are obtained.

### II. MHC Polypeptides

A. MHC Class I Polypeptides

The Class I histocompatibility ternary complex consists of three parts associated by noncovalent bonds. The MHCI heavy chain is a polymorphic transmembrane glycoprotein of about 45 kDa consisting of three extracellular domains, each containing about 90 amino acids (α1 at the N-terminus, α2 and α3), a transmembrane domain of about 40 amino acids and a cytoplasmic tail of about 30 amino acids. The α1 and α2 domains of the MHCI heavy chain contain two segments of alpha helix that form a peptide-binding groove or cleft. A short peptide of about 8-10 amino acids binds noncovalently (“fits”) into this groove between the two alpha helices. The α3 domain of the MHCI heavy chain is proximal to the plasma membrane. The MHCI heavy chain is non-covalently bound to a 32 microglobulin (β2m) polypeptide, forming a ternary complex. In MHCI, the binding groove is closed at both ends by conserved tyrosine residues leading to a size restriction of the bound peptides to usually 8-10 residues with its C-terminal end docking into the F-pocket.

The disclosure provides a multimeric protein comprising a two or more MHCI or MHCI-like polypeptides. The MHCI molecule can suitably be a vertebrate MHC molecule such as a human, a mouse, a rat, a porcine, a bovine or an avian MHC molecule.

In some embodiments, the multimeric MHCI multimers described herein, the MHC molecule is a human MHC class I protein: HLA-A, HLA-B or HLA-C. In some embodiments, the multimer comprises MHC Class I like molecules (including non-classical MHC Class I molecules) including, but not limited to, CD1d, HLA E, HLA G, HLA F, HLA H, MIC A, MIC B, ULBP-1, ULBP-2, and ULBP-3. The amino acid sequences of the MHCI heavy chains, β2m polypeptides and of MHC Class I like molecules from a variety of vertebrate species are known in the art and publicly available.

In some embodiments, the MHCI heavy chain alpha domain is human, and comprise, for example, an MHCI heavy chain alpha domain(s) from a human MHC Class I molecule(s) selected from the group consisting of HLA-A*01:01, HLA-A*03:01, HLA-A*11:01, HLA-A*24:02, HLA-B*07:02, HLA-C*04:01, HLA-C*07:02, HLA-B*08:01, HLA-B*35:01, HLA-B*57:01, HLA-B*57:03, HLA-E, HLA-C*16:01, HLA-C*08:02, HLA-C*07:01, HLA-C*05:01, HLA-B*44:02, HLA-A*29:02, HLA-B*44:03, HLA-C*03:04, HLA-B*40:01, HLA-C*06:02, HLA-B*15:01, HLA-C*03:03, HLA-A*30:01, HLA-B*13:02, HLA-C*12:03, HLA-A*26:01, HLA-B*38:01, HLA-B*14:02, HLA-A*33:01, HLA-A*23:01, HLA-A*25:01, HLA-B*18:01, HLA-B*37:01, HLA-B*51:01, HLA-C*14:02, HLA-C*15:02, HLA-C*02:02, HLA-B*27:05, HLA-A*31:01, HLA-A*30:02, HLA-B*42:01, HLA-C*17:01, HLA-B*35:02, HLA-B*39:06, HLA-C*03:02, HLA-B*58:01, HLA-A*33:03, HLA-A*68:02, HLA-C*01:02, HLA-C*07:04, HLA-A*68:01, HLA-A*32:01, HLA-B*49:01, HLA-B*53:01, HLA-B*50:01, HLA-A*02:05, HLA-B*55:01, HLA-B*45:01, HLA-B*52:01, HLA-C*12:02, HLA-B*35:03, HLA-B*40:02, HLA-B*15:03 and/or HLA-A*74:01. The full-length amino acid sequences (including signal sequence and transmembrane domain) of these MHCI molecules are shown in SEQ ID NOs: 10-75, respectively. The amino acid sequences of soluble forms of these MHCI molecules (lacking signal sequence and transmembrane domain) are shown in SEQ ID NOs: 76-141, respectively.

In some embodiments, the pMHCI multimers described herein comprises the α1 and α2 domains of an MHCI heavy chain. In some embodiments, the compound described herein comprises the α1, α2, and α3 domains of an MHCI heavy chain.

In some embodiments, the two or more pMHCI or pMHCI-like polypeptides in the multimer comprises a 02-microglobulin polypeptide, e.g., a human 02-microglobulin. In some embodiments, the 02-microglobulin is wild-type human 02-microglobulin. In some embodiments, the 02-microglobulin comprises an amino acid sequence that is at least 80, 85, 90, 95, or 99% identical to the amino acid sequence of the human β2 microglobulin, the full-length sequence of which is shown in SEQ ID NO: 142 (UniProt Id. No. P61769). Alternatively, the human β2-microglobulin polypeptide used in the pMHCI multimer can comprise or consist of the amino acid sequence shown in SEQ ID NO: 143.

In some embodiments, the multimeric protein comprises a soluble MHCI polypeptide. In some embodiments the MHC-multimeric protein comprises a soluble MHCI a domain and a β2-microglobulin polypeptide. In some embodiments, the soluble MHCI protein comprises the MHCI heavy chain α1 domain and the MHCI heavy chain α2 domain.

Alternatively, in some embodiments, the MHCI monomer is a fusion protein comprising a β2m polypeptide or functional fragment thereof covalently linked to the MHCI heavy chain or functional fragment thereof. In some embodiments the carboxy (—COOH) terminus of β2m is covalently linked to the amino (—NH₂) terminus of the MHCI heavy chain.

In some embodiments, the MHC monomers comprise one or more linkers between the individual components of the MHCI monomer. In some embodiments, the MHCI monomer comprises a heavy chain fused with β2m through a linker. In some embodiments, the linker between the heavy chain and β2m is a flexible linker, e.g., made of glycine and serine. In some embodiments, the flexible linker between the heavy chain and β2m is between 5-20 residues long. In other embodiments, the linker between the heavy chain and β2m is rigid with a defined structure, e.g. made of amino acids like glutamate, alanine, lysine, and leucine. In one embodiment, the linker is a (G₄S)₄ linker (SEQ ID NO: 233).

B. MHC Class II Polypeptides

MHC class II molecules are heterodimers composed of an α chain and a β chain, both of which are encoded by the MHC. The alpha chain is comprised of α1 and α2 domains. The beta chain is comprised of β 1 and β 2 domains. The α1 and β1 domains of the chains interact noncovalently to form a membrane-distal peptide-binding domain, whereas the α2 and β2 domains form a membrane-proximal immunoglobulin-like domain. The antigen binding groove, where a peptide epitope binds, is made up of two α-helices and a β-sheet. Since the antigen binding groove of MHC class II molecules is open at both ends, the groove can accommodate longer peptide epitopes than MHC class I molecules. Peptide epitopes presented by MHC class II molecules typically are about 15-24 amino acid residues in length.

The disclosure provides a multimeric protein comprising two or more MHCII or MHCII-like polypeptides. The MHCII molecule can suitably be a vertebrate MHCII molecule such as a human, a mouse, a rat, a porcine, a bovine or an avian MHCII molecule.

In some embodiments, the multimeric MHCII multimers described herein, the MHC molecule is a human MHC class II protein: HLA-DR, HLA-DQ, HLA-DX, HLA-DO, HLA-DZ, and HLA-DP. The amino acid sequences of the MHCII α and β chains from a variety of vertebrate species, including humans, are known in the art and publicly available.

In some embodiments, the human MHCII molecule is of an allotype selected from the group consisting of DRB1*0101 (see, e.g., Cameron et al. (2002) J. Immunol. Methods, 268:51-69; Cunliffe et al. (2002) Eur. J. Immunol., 32:3366-3375; Danke et al. (2003) J. Immunol., 171:3163-3169), DRB1*1501 (see, e.g., Day et al. (2003) J. Clin. Invest, 112:831-842), DRB5*0101 (see, e.g., Day et al., ibid), DRB1*0301 (see, e.g., Bronke et al. (2005) Hum. Immunol., 66:950-961), DRB1*0401 (see, e.g., Meyer et al. (2000) PNAS, 97:11433-11438; Novak et al. (1999) J. Clin. Invest, 104:R63-R67; Kotzin et al. (2000) PNAS, 97:291-296), DRB1*0402 (see, e.g., Veldman et al. (2007) Clin. Immunol., 122:330-337), DRB1*0404 (see, e.g., Gebe et al. (2001) J. Immunol. 167:3250-3256), DRB1*1101 (see, e.g., Cunliffe, ibid; Moro et al. (2005) BMC Immunol., 6:24), DRB1*1302 (see, e.g., Laughlin et al. (2007) Infect. Immunol. 75:1852-1860), DRB1*0701 (see, e.g., Danke, ibid), DQA1*0102 (see, e.g., Kwok et al. (2000) J. Immunol., 164:4244-4249), DQB1*0602 (see, e.g., Kwok, ibid), DQA1*0501 (see, e.g., Quarsten et al. (2001) J. Immunol., 167:4861-4868), DQB1*0201 (see, e.g., Quarsten, ibid), DPA1*0103 (see, e.g., Zhang et al. (2005) Eur. J. Immunol, 35:1066-1075; Yang et al. (2005) J. Clin. Immunol., 25:428-436), and DPB1*0401 (see, e.g., Zhang, ibid; Yang, ibid).

In some embodiments, the MHCII molecule is human, and comprise, for example, an MHCII alpha and beta chains selected from the group consisting of HLA-DRA*01:01, HLA-DRB1*01:01, HLA-DRB1*01:02, HLA-DRB1*03:01, HLA-DRB1*04:01, HLA-DRB1*04:04, HLA-DRB1*07:01, HLA-DRB1*08:01, HLA-DRB1*10:01, HLA-DRB1*11:01, HLA-DRB1*11:04, HLA-DRB1*13:01, HLA-DRB1*13:02, HLA-DRB1*14:01, HLA-DRB1*15:01, HLA-DRB1*15:03, HLA-DQA1*01:01, HLA-DQB1*05:01, HLA-DQA1*01:02, HLA-DQB1*06:02, HLA-DQA1*03:01, HLA-DQB1*03:02, HLA-DQA1*05:01, HLA-DQB1*02:01, HLA-DQB1*03:01, HLA-DQB1*03:03, HLA-DQB1*04:02, HLA-DQB1*05:03, HLA-DQB1*06:03 and HLA-DQB1*06:04. The full-length amino acid sequences (including signal sequence and transmembrane domain) of these MHCII chains are shown in SEQ ID NOs: 144-173, respectively. The amino acid sequences of soluble forms of these MHCII chains (lacking signal sequence and transmembrane domain) are shown in SEQ ID NOs: 174-203, respectively. MHC Class II alpha chain sequences are shown in SEQ ID NOs: 144, 160, 162, 164 and 166 (full-length sequences) and 174, 190, 192, 194 and 196 (soluble sequences). MHC Class II beta chain sequences are shown in SEQ ID NOs: 145-159, 161, 163, 165 and 167-173 (full-length sequences) and 175-189, 191, 193, 195 and 197-203 (soluble sequences).

In certain embodiments, an additional amino acid sequence can be appended to the C-terminal sequence of the alpha or beta chain of the MHCII molecule, for example for purposes of labeling and/or for attaching a moiety that mediates attachment (e.g., conjugation) to the multimerization domain. For example, an avitag (that mediates binding through the biotin binding site of Sav) can be appended, such as an avitag with a Myc tag (SEQ ID NO: 244), an avitag with a Myc tag and a His tag (SEQ ID NO: 245) or an avitag with a His tag and a FLAG tag (SEQ ID NO: 246).

In certain embodiments, heterodimerization pairs can be appended to the C-terminal sequence of the alpha and/or beta chains of the MHCII molecule. Non-limiting examples of such heterodimerization pair sequences include Fos and Jun (e.g., having the amino acid sequences shown in SEQ ID NOs: 247 and 248, respectively), acidic and basic leucine zippers (e.g., having the amino acid sequences shown in SEQ ID NOs: 249 and 250, respectively), knob and hole sequences (e.g., having the amino acid sequences shown in SEQ ID NOs: 251 and 252, respectively) for knobs-into-holes technology or spytag and spycatcher sequences (e.g., having the amino acid sequences shown in SEQ ID NOs: 253 and 254, respectively).

Typically, an MHCII-binding placeholder peptide is encoded in the expression construct adjacent to the coding sequences of the MHCII chains such that the placeholder peptide and a digestible linker are encoded in the construct (e.g., upstream of (N-terminally)) and in operative linkage with the coding sequences for the MHCII chain. In certain embodiments, an expression tag is also encoded upstream or downstream of the placeholder peptide. Non-limiting examples of such tags include a FLAG tag (e.g., having the amino acid sequence shown in SEQ ID NO: 238), a 6×His tag (e.g., having the amino acid sequence shown in SEQ ID NO: 239), a V5 tag (e.g., having the amino acid sequence shown in SEQ ID NO: 240), a Strep-Tag (e.g., having the amino acid sequence shown in SEQ ID NO: 241) and/or a Protein C tag (e.g., having the amino acid sequence shown in SEQ ID NO: 242).

In some embodiments, the pMHCII multimers described herein comprise the α1 and α2 domains of an MHCII alpha chain and the β1 and β2 domains of an MHCII beta chain. In some embodiments, the multimer described herein comprises only the α1 and β1 domains of an MHCII heavy chain. In other embodiments, the pMHCII multimers comprise an alpha-chain and a beta-chain combined with a peptide. Other embodiments include an MHCII molecule comprised only of alpha-chain and beta-chain (so-called “empty” MHC II without loaded peptide), a truncated alpha-chain (e.g. the α1 domain) combined with full-length beta-chain, either empty or loaded with a peptide, a truncated beta-chain (e.g. the β1 domain) combined with a full-length alpha-chain, either empty or loaded with a peptide, or a truncated alpha-chain combined with a truncated beta-chain (e.g. α1 and β1 domain), either empty or loaded with a peptide.

In some embodiments, the multimeric protein comprises a soluble MHCII polypeptide. In some embodiments the MHC-multimeric protein comprises a soluble MHCII lacking transmembrane and intracellular domains.

### III. Placeholder Peptides

A. MHC Class I Placeholder Peptides

In the methods and constructs provided herein, the MHC multimer expression construct encodes an MHC-binding peptide that binds to the MHC molecule also encoded by the construct such that upon expression in a host cell, MHC molecules loaded with peptide (e.g., a placeholder peptide) are expressed by the host cell. For MHCI multimers MHCI monomers are expressed such that they are loaded with a placeholder peptide to facilitate proper folding of the MHCI monomers to produce placeholder-peptide loaded MHCI (p*MHCI) within the multimers. Examples of placeholder peptides and methods of inducing folding MHCI heavy chains and 02-microglobulin in vitro in the presence of a placeholder peptide have been described in the art (e.g., Bakker et al., PNAS 105:3825-3830, 2008; Rodenko et al., Nat. Prot. 1: 1120-1132, 2006).

In some embodiments, the placeholder peptide is an HLA-A, HLA-B or HLA-C peptide. In some embodiments, the placeholder peptide is an HLA-A1 peptide (e.g., A*1:01 binding peptide). In some embodiments, the placeholder peptide is an HLA-A2 peptide (e.g., A*02:01 or A*02:05 binding peptide). In other embodiments, the placeholder peptide is an HLA-A3 peptide (e.g., A*03:01 binding peptide), an HLA-A11 peptide (e.g., A*11:01 binding peptide), an HLA-A23 peptide (e.g., A*23:01 binding peptide), an HLA-A24 peptide (e.g., A*24:02 binding peptide), an HLA-A26 peptide (e.g., A*26:01 binding peptide), an HLA-A30 peptide (e.g., A*30:01 binding peptide), an HLA-A31 peptide (e.g., A*31:01 binding peptide), an HLA-A32 peptide (e.g., A*32:01 binding peptide), an HLA-A33 peptide (e.g., A*33:01 binding peptide), an HLA-A68 peptide (e.g., A*68:02 binding peptide), an HLA-A74 peptide (e.g., A*74:01 binding peptide), an HLA-B7 peptide (e.g., B*7:02 binding peptide), an HLA-B8 peptide (e.g., B*08:01 binding peptide), an HLA-B13 peptide (e.g., B*13:02 binding peptide), an HLA-B14 peptide (e.g., B*14:02 binding peptide), an HLA-B15 peptide (e.g., B*15:01 or B*15:03 binding peptide), an HLA-B18 peptide (e.g., B*18:01 binding peptide), an HLA-B27 peptide (e.g., B*27:05 binding peptide), an HLA-B35 peptide (e.g., B*35:01, B*35:02 or B*35:03 binding peptide), an HLA-B37 peptide (e.g., B*37:01 binding peptide), an HLA-B38 peptide (e.g., B*38:01 binding peptide), an HLA-B39 peptide (e.g., B*39:06 binding peptide), an HLA-B40 peptide (e.g., B*40:01 or B*40:02 binding peptide), an HLA-B42 peptide (e.g., B*42:01 binding peptide), an HLA-B44 peptide (e.g., B*44:02 or B*44:03 binding peptide), an HLA-B45 peptide (e.g., B*45:01 binding peptide), an HLA-B50 peptide (e.g., B*50:01 binding peptide), an HLA-B51 peptide (e.g., B*51:01 binding peptide), an HLA-B52 peptide (e.g., B*52:01 binding peptide), an HLA-B53 peptide (e.g., B*53:01 binding peptide), an HLA-B55 peptide (e.g., B*55:01 binding peptide), an HLA-B57 peptide (e.g., B*57:01 or B*57:03 binding peptide), an HLA-B58 peptide (e.g., B*58:01 binding peptide), an HLA-C1 peptide (e.g., C*01:02 binding peptide), an HLA-C3 peptide (e.g., C*03:03 or C*03:04 binding peptide), an HLA-C4 peptide (e.g., C*04:01 binding peptide), an HLA-C5 peptide (e.g., C*05:01 binding peptide), an HLA-C6 peptide (e.g., C*06:02 binding peptide), an HLA-C7 peptide (e.g., C*07:01, C*07:02 or C*07:04 binding peptide), an HLA-C8 peptide (e.g., C*08:01 or C*08:02 binding peptide), an HLA-C12 peptide (e.g., C*12:02 binding peptide), an HLA-C14 peptide (e.g., C*14:02 binding peptide) or an HLA-C15 peptide (e.g., C*15:02 binding peptide). In some embodiments, the placeholder peptide is an HLA-E-binding peptide. In some embodiments, the placeholder peptide is a synthetic peptide. Non-limiting examples of peptides that bind HLA-A, B, C and E alleles as indicated above are shown in SEQ ID NOs: 204-223 and 267-320.

In some embodiments, the affinity of the placeholder peptide for the binding groove of MHCI is lower than the rescue peptide(s). In some embodiments, the affinity of the placeholder peptide for the MHCI binding groove is about 10-fold lower than the rescue peptide(s). In some embodiments, the affinity of the place holder peptide for the binding groove of MHCI is higher than the rescue peptide(s); however, the placeholder peptide can still be replaced by the rescue peptide by use of an excess concentration of the rescue peptide.

In some embodiments, the placeholder peptide is thermolabile. In some embodiments, the placeholder peptide is thermolabile at a temperature between about 30-37° C. In some embodiments, the placeholder peptide is labile at a temperature at or above 30° C., at or above 32° C., at or above 34° C., at or above 35° C., at or above 36° C., or at about 37° C. Thermal labile placeholder peptides and methods of identifying and producing thermal labile placeholder peptides have been described (e.g., WO 93/10220; WO 2005/047902; US 2008/0206789; Luimstra et al., Curr. Protoc. Immunol. 126(1):e85, 2019; Luimstra et al., J. Exp. Med. 215(5):1493-1504, 2018).

In some embodiments the placeholder peptide is labile at an acidic pH. In some embodiments, the placeholder peptide is labile between about pH 2.5 and 6.5. In some embodiments, the placeholder peptide is labile at a pH of about 2.5-6.0, 3.0-6.0, 3.0-6.5, 3.5-6.0 3.5-6.5, 4.0-6.0, 4.0-6.5, 4.5-6.0, 4.5-6.5, 5.0-6.0, 5.0-6.5, 5.0, 5.5., 6.0 or 6.5. In some embodiments, the placeholder peptide is labile at a basic pH. In some embodiments, the placeholder peptide is labile between about pH 9-11. In some embodiments, the placeholder peptide is labile at or above pH 9, at or above pH 9.5, at or about pH 10, at or about pH 10.5, or at or about pH 11. Methods of generating and using pH sensitive placeholder peptides are publicly available, for example, as described in WO 93/10220; US 2008/0206789; and Cameron et al., J. Immunol. Meth. 268:51-59.

In some embodiments, the placeholder peptide comprises a cleavable moiety. Various types of cleavable moieties are known in the art and include, for example, moieties that are cleaved by photoirradiation, enzymes, nucleophilic or electrophilic agents, reducing and oxidizing reagents (e.g., reviewed in Leriche et al., Biorg. Med. Chem. 20(2):571-582, 2012).

In some embodiments, the MHCI molecule is an HLA-A*02:01 molecule and the peptide is an HLA-A*02:01-restricted peptide. In one embodiment, the HLA-A*02:01-restricted peptide is a CMV pp65 peptide epitope. In one embodiment, the CMV pp65 peptide epitope comprises the amino acid sequence NLVPMVATV (SEQ ID NO: 4). In some embodiments, the CMV pp65 peptide epitope consists of the amino acid sequence NLVPMVATV (SEQ ID NO: 4). Other HLA-A*02:01-restricted peptide sequences include the MART-1 sequence EAAGIGILTV (SEQ ID NO: 6) or its heteroclitic variant ELAGIGILTV (SEQ ID NO: 322), the HPV sequence YMLDLQPETT (SEQ ID NO: 7), the HSV sequence SLPITVYYA (SEQ ID NO: 8) and the WT-1 sequence RMFPNAPYL (SEQ ID NO: 9).

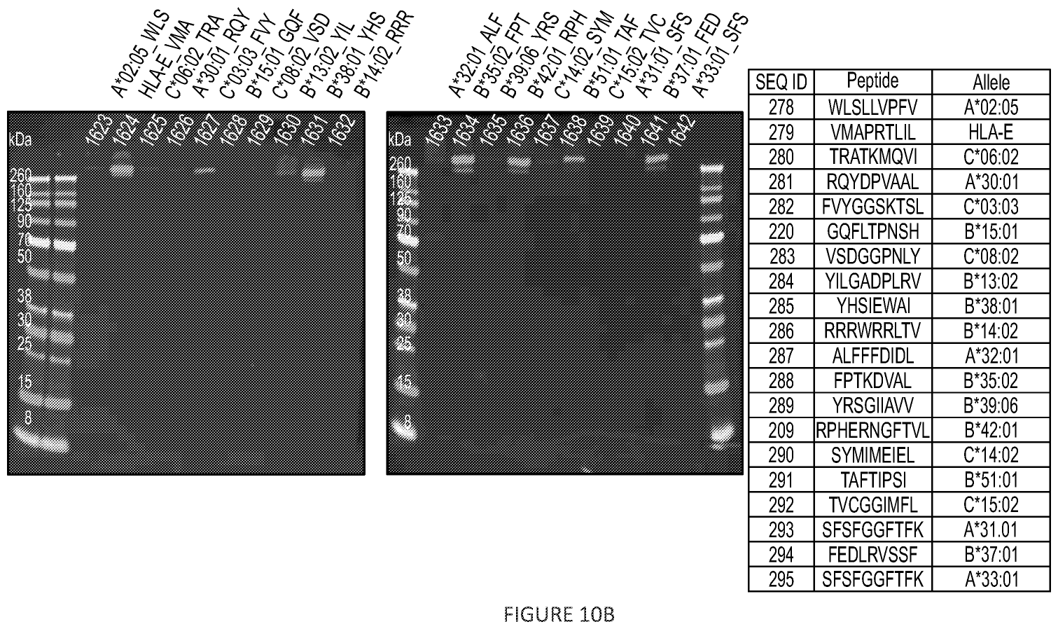

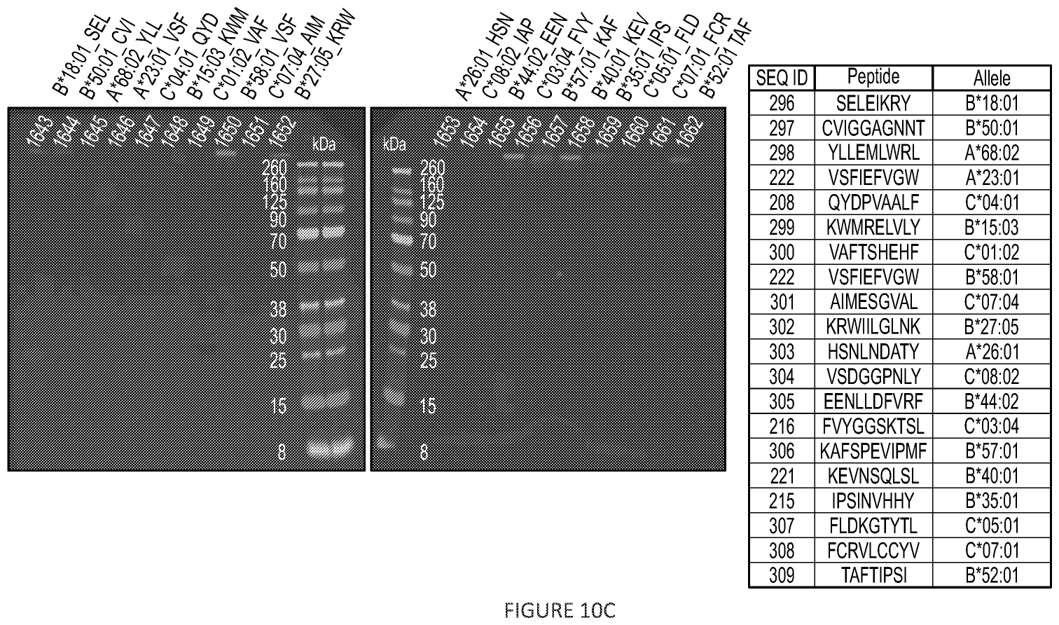

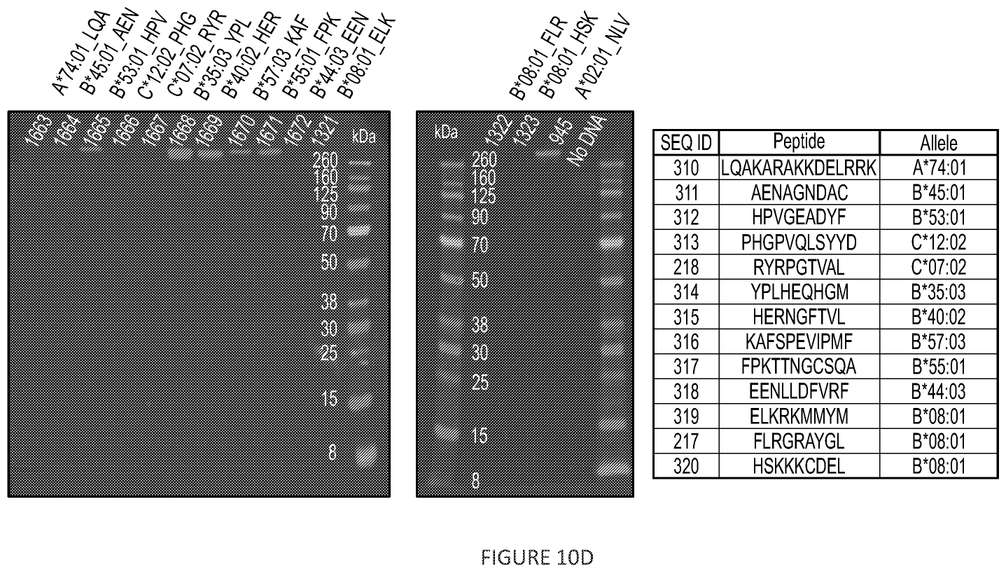

In other embodiments, the HLA-A2 placeholder peptide is p*A02:01, KILGFVFTV (SEQ ID NO: 211) or GILGFVFTL (SEQ ID NO: 204). In yet other embodiments, the MHCI/placeholder peptide combination can be selected from the group consisting of p*A1:01, VTEHDTLLY (SEQ ID NO: 212); p*A3:01, TVRSHCVSK (SEQ ID NO:213); p*A11:01, TTFLQTMLR (SEQ ID NO: 214); p*A24:02, RYPLTFGWCF (SEQ ID NO: 207); p*B7:02, RPHERNGFTVL (SEQ ID NO: 210); p*B35:01, IPSINVHHY (SEQ ID NO: 215); p*C3:04, FVYGGSKTSL (SEQ ID NO: 216), p*B8:01, FLRGRAYGL (SEQ ID NO: 217); p*C7:02, RYRPGTVAL (SEQ ID NO: 218); p*C4:01, QYDPVAALF (SEQ ID NO: 219); p*B15:01, GQFLTPNSH (SEQ ID NO: 220); p*B40:01, KEVNSQLSL (SEQ ID NO: 221); p*B58:01, VSFIEFVGW (SEQ ID NO: 222); and p*C8:02, IAPWYAFAL (SEQ ID NO: 223). Sequences of non-limiting examples MHCI-binding peptides are shown in SEQ ID NOs: 204-223 and 267-320, as well as FIG. 10A-D.

In some embodiments, the placeholder peptide comprises a chemoselective moiety. In some embodiments, the chemoselective moiety comprises a sodium dithionite sensitive azobenzene linker, wherein the azobenzene comprises at least one aromatic group comprising an electron-donor group and is located between two amino acid residues. Azobenzine linkers and methods for chemoselective peptide exchange are known in the art, for example, as described in U.S. Pat. No. 10,400,024.

In some embodiments, the placeholder peptide comprises a cleavable moiety that is cleaved upon exposure to an aminopeptidase. In some embodiments, the cleavage of the amino acid residue occurs via the use of a methionine aminopeptidase. The methionine aminopeptidase can cleave a methionine from a peptide when the amino acid residue at position two is, for example, glycine, alanine, serine, cysteine, or proline. In some embodiments, the cleavable moiety comprises a thrombin cleavage domain.

In some embodiments, the placeholder peptide is a dipeptide. In some embodiments, the dipeptide binds to the F pocket of the MHCI binding groove. In some embodiments, the second amino acid of the dipeptide is hydrophobic. In some embodiments, the dipeptide is selected from the group consisting of glycyl-leucine (GL), glycyl-valine (GV), glycyl-methionine (GM), glycyl-cyclohexylalanine (GCha), glycyl-homoleucine (GHle) and glycyl-phenylalanine (GF). Methods for producing and using dipeptides as placeholder peptides are publicly available, for example, as described in Saini et al. (PNAS 112:202-207, 2015).

In some embodiments, the placeholder peptide further comprises a fluorescent label. In some embodiments, the fluorescent label is attached to a cysteine residue in the placeholder peptide.

B. MHC Class II Placeholder Peptides

In the methods and constructs provided herein, the MHCII monomers are expressed such that they are loaded with a placeholder peptide to facilitate proper folding of the MHCII monomers to produce placeholder-peptide loaded MHCII (p*MHCII) within the multimers. In various embodiments, the placeholder peptide is peptide that binds HLA-DR, HLA-DQ, HLA-DX, HLA-DO, HLA-DZ or HLA-DP. In some embodiments, the placeholder peptide is a synthetic peptide.

In some embodiments, the affinity of the placeholder peptide for the binding groove of MHCII is lower than the rescue peptide(s). In some embodiments, the affinity of the placeholder peptide for the MHCII binding groove is about 10-fold lower than the rescue peptide(s).

In some embodiments, the placeholder peptide is thermolabile. In some embodiments, the placeholder peptide is thermolabile at a temperature between about 30-37° C. In some embodiments, the placeholder peptide is labile at a temperature at or above 30° C., at or above 32° C., at or above 34° C., at or above 35° C., at or above 36° C., or at about 37° C. Thermal labile placeholder peptides and methods of identifying and producing thermal labile placeholder peptides have been described (e.g., WO 93/10220; WO 2005/047902; US 2008/0206789; Luimstra et al., Curr. Protoc. Immunol. 126(1):e85, 2019; Luimstra et al., J. Exp. Med. 215(5):1493-1504, 2018).

In some embodiments the placeholder peptide is labile at an acidic pH. In some embodiments, the placeholder peptide is labile between about pH 2.5 and 6.5. In some embodiments, the placeholder peptide is labile at a pH of about 2.5-6.0, 3.0-6.0, 3.0-6.5, 3.5-6.0 3.5-6.5, 4.0-6.0, 4.0-6.5, 4.5-6.0, 4.5-6.5, 5.0-6.0, 5.0-6.5, 5.0, 5.5., 6.0 or 6.5. In some embodiments, the placeholder peptide is labile at a basic pH. In some embodiments, the placeholder peptide is labile between about pH 9-11. In some embodiments, the placeholder peptide is labile at or above pH 9, at or above pH 9.5, at or about pH 10, at or about pH 10.5, or at or about pH 11. Methods of generating and using pH sensitive placeholder peptides are publicly available, for example, as described in WO 93/10220; US 2008/0206789; and Cameron et al., J. Immunol. Meth. 268:51-59.

In some embodiments, the placeholder peptide comprises a cleavable moiety. Various types of cleavable moieties are known in the art and include, for example, moieties that are cleaved by photoirradiation, enzymes, nucleophilic or electrophilic agents, reducing and oxidizing reagents (e.g., reviewed in Leriche et al., Biorg. Med. Chem. 20(2):571-582, 2012).

In one embodiment, the placeholder peptide is fused to a degradation tag and peptide exchange is promoted by proteolysis in the presence of a corresponding protease (the digests the degradation tag) along with the presence of the rescue peptide(s).

In some embodiments, the cleavable placeholder peptide is a photocleavable peptide, e.g., cleaved upon exposure to UV light. For example, the placeholder peptide can comprise one or more photocleavable non-natural amino acids. MHCII-binding photocleavable peptides, e.g., that incorporate the UV-sensitive amino acid analog 3-amino-3-(2-nitrophenyl)-propionate have been described (see e.g., Negroni and Stern (2018) PLos One, 13(7):e0199704).

In one embodiment, the MHCII placeholder peptide is a CLIP peptide, such as having the amino acid sequence KPVSKMRMATPLLMQA (SEQ ID NO: 224). Additional suitable CLIP peptides (or CLIP peptide variants) include those having the amino acid sequence RMATPLLMQALPMGAL (SEQ ID NO: 323) or the amino acid sequence LMQALPMGALPQGP (SEQ ID NO: 324). In one embodiment, the CLIP peptide is cleavable. In one embodiment, the MHCII monomers are synthesized with the cleavable CLIP peptide covalently attached, such as by synthesis of single-chain MHC class II chain-peptide complexes, directed by engineering peptide-specific complementary DNA (cDNA) sequences proximal to the beta-chain cDNA (see e.g., Day et al. (2003) J. Clin. Invest., 112:831-842). Cleavage of the covalent linkage between the CLIP peptide (as the placeholder peptide) and MHCII thus allows for peptide exchange with other MHCII-binding peptides.

Other MHCII binding peptides have been described in the art that can be used as placeholder peptides, based on appropriate pairing of an MHCII molecule and its known MHCII binding peptide. Non-limiting examples of known MHCII molecule/MHCII binding peptide pairs include: DRA1*0101/DRB1*0401 and the immunodominant peptide of hemagglutinin, HA₃₀₇₋₃₁₉ (see Novak et al. (1999) J. Clin. Invest., 104:R63-R67) and HLA-DR*1101 and tetanus-toxoid (TT)-derived p2 peptide (TT830-844) having the amino acid sequence QIYKANSKFIGITEL (SEQ ID NO: 225) (see Cecconi et al. (2008) Cytometry, 73A:1010-1018).

### IV. Multimerization Domains

Multimerization domains for use in producing the pMHC multimers provided herein include proteins, polypeptide or other multimeric moieties suitable for the coexpression with two or more pMHC monomers, which do not interfere with binding of the pMHC polypeptides to cells. In some embodiments, the multimerization domain comprises protein subunits. In some embodiments, the multimerization domain is a homomultimer of protein subunits. In some embodiments, the multimerization domain is a heteromultimer of protein subunits. In some embodiments, the multimer is a dimer, trimer, tetramer, pentamer, hexamer, octamer decamer or dodecamer. In one preferred embodiment, the pMHC multimer is a tetramer.

Examples of suitable binding entities are streptavidin (SA) and avidin and derivatives thereof, biotin, immunoglobulins, antibodies (monoclonal, polyclonal, and recombinant), antibody fragments and derivatives thereof, leucine zipper domain of AP-1 (jun and fos), hexa-his (metal chelate moiety), hexa-hat GST (glutathione S-transferase) glutathione affinity, Calmodulin-binding peptide (CBP), Strep-tag®, Cellulose Binding Domain, Maltose Binding Protein, S-Peptide Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, VSV Epitope, lectins that mediate binding to a diversity of compounds, including carbohydrates, lipids and proteins, e. g., Con A (Canavaliaensiformis) or WGA (wheat germ agglutinin) and tetranectin or Protein A or G (antibody affinity) or coiled-coil polypeptides e.g. leucine zipper. Combinations of such binding entities are also included.

In some embodiments, the multimerization domain is a tetramer of streptavidin (SA or SAv) or a derivative thereof. In some embodiments, the multimerization domain is tetrameric streptavidin. In some embodiments, the tetramer comprises Strep-tactin®, an engineered form of streptavidin that binds an engineered peptide sequence referred to as Strep-tag®. Strep-tag® and Strep-tactin® are described in U.S. Pat. Nos. 5,506,121 and 6,103,493, respectively, and are commercially available from a number of sources.

To attach MHC monomers to streptavidin non-covalently via the biotin-binding site of SAv, an avitag can be incorporated into MHC monomer, for example at the C-terminal end, such that the MHC monomer can be biotinylated through the avitag. Non-limiting examples of avitag sequences include SEQ ID NO: 244 (avitag with Myc tag), SEQ ID NO: 245 (avitag with Myc tag and 6×His tag) and SEQ ID NO: 246 (avitag with 6×His Tag and FLAG tag).

In one embodiment, the multimerization domain comprises full-length streptavidin. In another embodiment, the multimerization domain comprises a natural streptavidin core polypeptide. In another embodiment, the multimerization domain comprises a recombinant streptavidin core polypeptide, such as STV25 or STV13 (e.g., as described in Sano et al. (1995) J. Biol. Chem. 270:28204-28209). Accordingly, as used herein, the term “streptavidin” is intended to encompass the full-length protein as well as core portions thereof, including but not limited to the following representative sequences:

| | |
|---|---|
| Full | DPSKDSKAQVSAAEAGITGTWYNQLGSTFIVTAGADGALTG |
| length | TYESAVGNAESRYVLTGRYDSAPATDGSGTALGWTVAWKNN |
| SA | YRNAHSATTWSGQYVGGAEARINTQWLLTSGTTEANAWKST |
| | LVGHDTFTKVKPSAASIDAAKKAGVNNGNPLDAVQQ |
| | (SEQ ID NO: 263) |
| Natural | AEAGITGTWYNQLGSTFIVTAGADGALTGTYESAVGNAESR |
| Core SA | YVLTGRYDSAPATDGSGTALGWTVAWKNNYRNAHSATTWSG |
| | QYVGGAEARINTQWLLTSGTTEANAWKSTLVGHDTFTKVKP |
| | SAAS (SEQ ID NO: 264) |
| STV25 | MEAGITGTWYNQLGSTFIVTAGADGALTGTYESAVGNAESR |
| | YVLTGRYDSAPATDGSGTALGWTVAWKNNYRNAHSATTWSG |
| | QYVGGAEARINTQWLLTSGTTEANAWKSTLVGHDTFTKVKP |
| | SAA (SEQ ID NO: 265) |
| STV13 | MGITGTWYNQLGSTFIVTAGADGALTGTYESAVGNAESRYV |
| | LTGRYDSAPATDGSGTALGWTVAWKNNYRNAHSATTWSGQY |
| | VGGAEARINTOWLLTSGTTEANAWKSTLVGHDTFTKV |
| | (SEQ ID NO: 266) |

### V. Peptide Linkers and Tags

A. Peptide Linkers

In certain embodiments, the expression construct encoding the MHC multimers encodes one or more peptide linkers, located for example in between the domain-encoding regions of the expression construct. The term “peptide linker” denotes a linear amino acid chain of natural and/or synthetic origin. The linker has the function to ensure that polypeptides conjugated to each other can perform their biological activity by allowing the polypeptides to fold correctly and to be presented properly. The peptide linker may contain repetitive amino acid sequences or sequences of naturally occurring polypeptides. In some embodiments, the peptide linker has a length of from 2 to 50 amino acids. In some embodiments, the peptide linker is between 3 and 30 amino acids, between 5 to 25 amino acids, between 5 to 20 amino acids, or between 10 and 20 amino acids.

In some embodiments, the peptide linker is rich in glycine, glutamine, and/or serine residues. These residues are arranged e.g. in small repetitive units of up to five amino acids. This small repetitive unit may be repeated for one to five times. At the amino- and/or carboxy-terminal ends of the multimeric unit up to six additional arbitrary, naturally occurring amino acids may be added. Other synthetic peptidic linkers are composed of a single amino acid, which is repeated between 10 to 20 times and may comprise at the amino- and/or carboxy-terminal end up to six additional arbitrary, naturally occurring amino acids. All peptidic linkers can be encoded by a nucleic acid molecule and therefore can be recombinantly expressed. As the linkers are themselves peptides, the polypeptide connected by the linker are connected to the linker via a peptide bond that is formed between two amino acids.

Suitable peptide linkers are well known in the art, and are disclosed in, e.g., US2010/0210511 US2010/0179094, and US2012/0094909, which are herein incorporated by reference in its entirety. Other linkers are provided, for example, in U.S. Pat. Nos. 5,525,491; Alfthan et al., Protein Eng., 1995, 8:725-731; Shan et al., J. Immunol., 1999, 162:6589-6595; Newton et al., Biochemistry, 1996, 35:545-553; Megeed et al.; Biomacromolecules, 2006, 7:999-1004; and Perisic et al., Structure, 1994, 12:1217-1226; each of which is incorporated by reference in its entirety.

In some embodiments, the polypeptide linker is synthetic. As used herein, the term “synthetic” with respect to a polypeptide linker includes peptides (or polypeptides) which comprise an amino acid sequence (which may or may not be naturally occurring) that is linked in a linear sequence of amino acids to a sequence (which may or may not be naturally occurring) to which it is not naturally linked in nature. For example, the polypeptide linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). Polypeptide linkers may be employed, for instance, to ensure that the binding portion (TCR or MHC), the multimerization domain and the Igg-Framework of each multimeric fusion polypeptide is juxtaposed to ensure proper folding and formation of a functional multimeric protein complex. Preferably, a polypeptide linker will be relatively non-immunogenic and not inhibit any non-covalent association among monomer subunits of a binding protein.

In some embodiments, the linker is a Gly-Ser polypeptide linker, i.e., a peptide that consists of glycine and serine residues. Non-limiting examples of such Gly-Ser linker include those having an amino acid sequence as shown in SEQ ID NOs: 226-234. One exemplary Gly-Ser polypeptide linker comprises the amino acid sequence (Gly4Ser)n, wherein n=1-6 (SEQ ID NO: 226). In certain embodiments, n=1. In certain embodiments, n=2. In certain embodiments, n=3. In certain embodiments, n=4. In certain embodiments, n=5. In certain embodiments, n=6. Another exemplary Gly-Ser polypeptide linker comprises the amino acid sequence Ser(Gly4Ser)n, wherein n=1-10 (SEQ ID NO: 229). In certain embodiments, n=1. In certain embodiments, n=2. In certain embodiments, n=3, i.e., Ser(Gly4Ser)3. In certain embodiments, n=4, i.e., Ser(Gly4Ser)4. In certain embodiments, n=5. In certain embodiments, n=6. In certain embodiments, n=7. In certain embodiments, n=8. In certain embodiments, n=9. In certain embodiments, n=10.

Other exemplary linkers include GS linkers (i.e., (GS)n), GGSG linkers (i.e., (GGSG)n) (SEQ ID NO: 230), GSAT linkers (SEQ ID NO: 231), SEG linkers, and GGS linkers (i.e., (GGSGGS)n) (SEQ ID NO: 232), wherein n is a positive integer (e.g., 1, 2, 3, 4, or 5), SSSGSSSGSAA linkers (SEQ ID NO: 227), G₅ linkers (GGGGG; SEQ ID NO: 228), (Gly4Ser)4 (GGGGSGGGGSGGGGSGGGGS; SEQ ID NO: 233), and (GS)₂AG₂SGSG₃S linkers (GSGSAGGSGSGGGS; SEQ ID NO: 234).

In various embodiments, an MHC multimer expression construct comprises a GS family linker at one or more of the following locations within the expression construct: between the MHC-binding peptide coding region and the MHC chain-encoding region; between the two MHC chain-encoding regions (e.g., between MHC Class I alpha chain and beta2-microglobulin chain coding regions or between the MHC Class II alpha chain and MHC Class II beta chain coding regions), between the MHC chain-encoding regions and the multimerization domain coding region, and/or between the multimerization domain coding region and a C-terminal tag-encoding region. In certain embodiments, the GS family linker located between the MHC-binding peptide coding region and the MHC chain-encoding region comprises a cleavage site (e.g., a site cleavable by an enzyme, such as a protease). Suitable protease cleavage sites include those cleaved by proteases such as Factor Xa, thrombin, TEV, HRV3C, furin and the like.

In certain embodiments, the GS family linker located between the MHC-binding peptide coding region and the MHC chain-encoding region comprises a Factor Xa cleavable site (e.g., comprises the amino acid sequence shown in SEQ ID NO: 235 or 236).

In certain embodiments, the GS family linker located between the two MHC chain-encoding regions (e.g., between MHC Class I alpha chain and beta2-microglobulin chain coding regions or between the MHC Class II alpha chain and MHC Class II beta chain coding regions) comprises the linker sequence shown in SEQ ID NO: 233.

In certain embodiments, the GS family linker located between the MHC chain-encoding regions and the multimerization domain coding region comprises the linker sequence shown in SEQ ID NO: 234.

Other suitable linkers for use in multimeric fusion proteins can be found using publicly available databases, such as the Linker Database (ibi.vu.nl/programs/linkerdbwww). The Linker Database is a database of inter-domain linkers in multi-functional enzymes which serve as potential linkers in novel multimeric fusion proteins (see, e.g., George et al., Protein Engineering 2002; 15:871-9).

Polypeptide linkers can be introduced into polypeptide sequences using techniques known in the art. Modifications can be confirmed by DNA sequence analysis. Plasmid DNA can be used to transform host cells for stable production of the polypeptides produced.

B. Tags

Additional tags suitable for use in the methods and compositions provided herein include affinity tags, including but not limited to enzymes, protein domains, or small polypeptides which bind with high specificity to a range of substrates, such as carbohydrates, small biomolecules, metal chelates, antibodies, etc. to allow rapid and efficient purification of proteins. Solubility tags enhance proper folding and solubility of a protein and are frequently used in tandem with affinity tags. Sequences encoding such a tag(s) can be incorporated into an expression construct of the disclosure, such as at the C-terminus or N-terminus of the MHC multimer-encoding regions to thereby incorporate a detectable tag into the expressed polypeptide.

Small-size tags which include, but are not limited to, 6×His, FLAG, Strep II and Calmodulin-binding peptide (CBP) tag, have the benefits of minimizing the effect on structure, activity and characteristics of the MHC polypeptide. (Zhao et al. J. Anal. Chem. 2013 581093)

In some embodiments, the tag is a FLAG tag. The FLAG tag is a hydrophilic octapeptide epitope tag that binds to several specific anti-FLAG monoclonal antibodies such as M1, M2, and M5 with different recognition and binding characteristics (Einhauer et al. J. Biochem. Biophys. 49:455-465, 2001: Hopp et al. Mol. Immunol. 33:601-608, 1996). FLAG fusion proteins can be recognized by monoclonal antibody with calcium-dependent (e.g., M2) or calcium-independent manner. In particular, the tag appended to the N-terminus of the fusion protein is necessary for the immunoaffinity purification with M1 monoclonal antibody, while M2 is position-insensitive.

Non-limiting examples of suitable tags include FLAG tags (e.g., having the amino acid sequence shown in SEQ ID NO: 238), 6×His tags (e.g., having the amino acid sequence shown in SEQ ID NO: 239), V5 tags (e.g., having the amino acid sequence shown in SEQ ID NO: 240), Strep-Tags (e.g., having the amino acid sequence shown in SEQ ID NO: 241) and/or a Protein C tags (e.g., having the amino acid sequence shown in SEQ ID NO: 242).

### VI. MHC Peptide Epitopes for Peptide Libraries

A. Peptide Epitope Selection

Various processes have been developed for identifying new MHC binding peptides that may be T cell epitopes and many experimental methods start with constructing an overlapping library of peptide fragments from a given protein sequence, by synthesizing a constant length (n-mer) amino acid sequences which are offset from one another along the protein sequence by fixed number of amino acids. The MHC binding properties and potential for activating T cells of each sequence can then be assessed in a number of assays.

Existing MHC binding peptides that have been identified with the methods outlined above and other methods, such as crystallographic analysis of the conformation of and charge distribution in the MHC binding groove has led to binding motifs being defined for the most common MHC alleles, setting rules for what type of putative MHC binding peptide can actually bind well to MHC molecules of a given allele. These motifs have been translated into predictive computer algorithms for predicting peptide binding to MHC molecules such as the SYFPEITHI algorithm (Rammensee H.-G., et al. (1995), Immunogenetics 41:178-228).

Protein sequences for the desired antigen can analyzed for potential HLA specific antigens by using SYFPEITHI (Rammensee et al. Immungenetics 50:213-219, 1999), and the artificial neural network (ANN) and stabilized matrix method (SMM) algorithms from IEDB (Peters et al. PLoS Biol. 3:e91, 2005). Peptides are selected based on a predicted binding value of either >21 for SYFPEITHI, <6000 for ANN, or <600 for SMM. Selected peptides are synthesized. Other suitable methods for analyzing protein sequences for potential HLA specific antigens also are known in the art and are suitable for use in identifying such HLA specific examples, such as NetMHCpan and NetMHCIIpan.

Binding assays can be performed using a fluorescence polarization (FP) assay as previously described (e.g., Buchi et al. Biochemistry 43:14852-14863, 2004; Sette et al., Mol. Immunol. 31:813-822.). To determine binding capacity of the peptides, percentage inhibition relative to controls can be determined in an FP competition assay with the placeholder peptide.

An epitope library can comprise peptides containing natural amino acids, non-natural amino acids, or a combination of natural and non-natural amino acids. Non-natural amino acids can be included to facilitate post-translational modifications, including but not limited to glycosylation, methylation, deamidation, oxidation, reduction and the like. Methods for preparing epitope libraries including non-natural amino acids are established in the art.

In some embodiments, the peptides bound to the pMHC multimers are from an unbiased library of peptides. In various embodiments, the MHC-binding peptides can be 8mers, 9mers, 10mers, 11mers, 12mers, 13mers, 14mers, 15mers, 16mers, 17mers, 18mers, 19mers, 20mers, 21mers, 22mers, 23mers, 24mers or 25mers. Typically, MHCI-binding peptides are 8mers-10mers, which MHCII-binding peptides are 13mers-25mers. In some embodiments, the MHCI-binding peptides are 9-mers. In some embodiments, the peptides bound to the pMHCI multimers are 9-mers which include an HLA-A2 binding motif with key amino acids at positions 2 and 9 which can include isoleucine (I), valine (V) or leucine (L).

In some embodiments, the library comprises all k-mer peptides produced by transcription and translation of any polynucleotide sequence of interest, for example, in silico production of the transcription and translation products of both the forward and reverse strands of a genome or metagenome in all six reading frames.

In some embodiments, a library of the disclosure comprises all k-mer peptides that can be derived from in silico translation of an exome of interest.

In some embodiments, a library of the disclosure comprises all k-mer peptides that can be derived from in silico translation of a transcriptome of interest.

In some embodiments, a library of the disclosure comprises all k-mer peptides that can be derived from a proteome of interest.

In some embodiments, a library of the disclosure comprises all k-mer peptides that can be derived from in silico translation of an ORFeome of interest.

In some embodiments, an algorithm can be used to select peptides in a peptide library.

For example, an algorithm can be used to predict peptides most likely to fold or dock in an MHC/HLA binding pocket, and peptides above a certain threshold value can be selected for inclusion in the library.

In some embodiments, a library of the disclosure comprises all peptides that can be derived from in silico transcription and translation or translation of a group of genomes, proteomes, transcriptomes, ORFeomes, or any combination thereof.

In some embodiments, the peptides are derived from in silico transcription and translation or translation of polynucleotide sequences from a group of samples, for example, clinical samples from a patient population, or a group of pathogen genomes.

In some embodiments, the peptides are derived from a differential genome, proteome, transcriptome, ORFeome, or any combination thereof, where two or more genomes, proteomes, transcriptomes, ORFeomes, or a combination thereof are compared to identify sequences that are differential sequences (e.g., that differ between them). In some embodiments, the peptide sequences are identified by comparing tissues of interest. In some embodiments, the peptide sequences are identified by comparing cells of interest. In some embodiments, the peptide sequences are identified by comparing diseased versus healthy cells or tissues. In some embodiments, the diseased cells or tissues are cancer cells or tissues. In some embodiments, the diseased cells are derived from an individual with an autoimmune disorder.

In some embodiments, the peptides are derived from homologous sequences of genomes, proteomes, transcriptomes, ORFeomes, or any combination thereof, where two or more genomes, proteomes, transcriptomes, ORFeomes, or a combination thereof are compared to identify sequences that are homologous sequences.

In some embodiments, the peptides are derived from mutations in a sequence of interest, for example, all 9-mer peptides that can be generated from single nucleotide mutations in a polynucleotide sequence encoding an antigen or epitope.

In some embodiments, the peptides an overlapping peptide library, comprising overlapping peptides from a template sequence (e.g., in silico translated genome), wherein overlapping peptides of a set length are offset by a defined number of residues.

In some embodiments, selection of peptides comprises prioritizing peptides based on predicted binding affinity for a certain HLA type.

In some embodiments, selection of peptides for a library of the disclosure prioritizes HLA types or alleles based on prevalence in a population, e.g., a human population.

In some embodiments, the library comprises all k-mer peptides produced by transcription and translation of any polynucleotide sequence of interest, for example, in silico production of the transcription and translation products of both the forward and reverse strands of a genome or metagenome in all six reading frames. In some embodiments, a library of the disclosure comprises all k-mer peptides that can be derived from in silico transcription and translation of a mammalian genome, for example, a mouse genome, a human genome, a patient genome, an autoimmune patient genome, or a cancer genome. In some embodiments, a library of the disclosure comprises all k-mer peptides that can be derived from in silico transcription and translation of a microorganism genome, for example, a bacterial genome, a viral genome, a protozoan genome, a protist genome, a yeast genome, an archaeal genome, or a bacteriophage genome. In some embodiments, a library of the disclosure comprises all k-mer peptides that can be derived from in silico transcription and translation of a pathogen genome, for example, a bacterial pathogen genome, a viral pathogen genome, a fungal pathogen genome, an opportunistic pathogen genome, a conditional pathogen genome, or a eukaryotic parasite genome. In some embodiments, a library of the disclosure can be derived from a plant genome or a fungal genome. In some embodiments, a library of the disclosure comprises k-mer peptides derived from in silico transcription and translation of a genome, wherein the genome is modified during in silico transcription and translation, for example, in silico mutated to produce k-mer peptides comprising mutations (e.g. substitutions, insertions, deletions).

In some embodiments, a library of the disclosure comprises all k-mer peptides that can be derived from in silico translation of an exome of interest, for example, a mammalian exome, a human exome, a mouse exome, a patient exome, an autoimmune patient exome, a cancer exome, a viral exome, a protozoan exome, a protist exome, a yeast exome, a pathogen exome, a eukaryotic parasite exome, a plant exome, or a fungal exome. In some embodiments, a library of the disclosure comprises k-mer peptides derived from in silico translation of a exome, wherein the exome is modified during in silico translation, for example, in silico mutated to produce k-mer peptides comprising mutations (e.g. substitutions, insertions, deletions).

In some embodiments, a library of the disclosure comprises all k-mer peptides that can be derived from in silico translation of a transcriptome of interest, for example, a mammalian transcriptome, a human transcriptome, a mouse transcriptome, a patient transcriptome, an autoimmune patient transcriptome, a cancer transcriptome, a microorganism transcriptome, a bacterial transcriptome, a viral transcriptome, a protozoan transcriptome, a protist transcriptome, a yeast transcriptome, an archaeal transcriptome, a bacteriophage transcriptome, a pathogen transcriptome, a eukaryotic parasite transcriptome, a plant transcriptome, a fungal transcriptome, a transcriptome derived from RNA sequencing, a microbiome transcriptome, or a transcriptome derived from metagenomic RNA-sequencing. In some embodiments, a library of the disclosure comprises k-mer peptides derived from in silico translation of a transcriptome, wherein the transcriptome is modified during in silico translation, for example, in silico mutated to produce k-mer peptides comprising mutations (e.g. substitutions, insertions, deletions).

In some embodiments, a library of the disclosure comprises all k-mer peptides that can be derived from a proteome of interest, for example, a mammalian proteome, a human proteome, a mouse proteome, a patient proteome, an autoimmune patient proteome, a cancer proteome, a microorganism proteome, a bacterial proteome, a viral proteome, a protozoan proteome, a protist proteome, a yeast proteome, an archaeal proteome, a bacteriophage proteome, a pathogen proteome, a eukaryotic parasite proteome, a plant proteome or a fungal proteome. In some embodiments, a library of the disclosure comprises k-mer peptides derived from a proteome wherein the k-mer peptides are modified from the proteome sequence, for example, k-mer peptides comprising mutations (e.g. substitutions, insertions, deletions).

In some embodiments, a library of the disclosure comprises all k-mer peptides that can be derived from in silico translation of an ORFeome of interest, for example, a mammalian ORFeome, a human ORFeome, a mouse ORFeome, a patient ORFeome, an autoimmune patient ORFeome, a cancer ORFeome, a microorganism ORFeome, a bacterial ORFeome, a viral ORFeome, a protozoan ORFeome, a protist ORFeome, a yeast ORFeome, an archaeal ORFeome, a bacteriophage ORFeome, a pathogen ORFeome, a eukaryotic parasite ORFeome, a plant ORFeome or a fungal ORFeome, an ORFeome derived from next-gen sequencing, a microbiome ORFeome, or an ORFeome derived from metagenomic sequencing. In some embodiments, a library of the disclosure comprises k-mer peptides derived from in silico translation of an ORFeome, wherein the ORFeome is modified during in silico translation, for example, in silico mutated to produce k-mer peptides comprising mutations (e.g. substitutions, insertions, deletions).

In some embodiments, a library of the disclosure comprises all k-mer peptides that can be derived from in silico transcription and translation or translation of a group of genomes, proteomes, transcriptomes, ORFeomes, or any combination thereof. In some embodiments, a library of the disclosure comprises all k-mer peptides that can be derived from in silico transcription and translation or translation of polynucleotide sequences from a group of samples, for example, clinical samples from a patient population, or a group of pathogen genomes. In some embodiments, a library of the disclosure comprises all k-mer peptides that can be derived from in silico transcription and translation of a group of viral genomes, for example, the human virome. In some embodiments, a library of the disclosure comprises all k-mer peptides that can be derived from in silico transcription and translation of a group of genomes, proteomes, transcriptomes, ORFeomes, or any combination thereof, wherein the source sequences are modified during in silico translation, for example, in silico mutated to produce k-mer peptides comprising mutations (e.g. substitutions, insertions, deletions).

In some embodiments, a library of the disclosure comprises all k-mer peptides that can be derived from a differential genome, proteome, transcriptome, ORFeome, or any combination thereof, where two or more genomes, proteomes, transcriptomes, ORFeomes, or a combination thereof are compared to identify sequences that are differential sequences (e.g., that differ between them), for example, differing in nucleotide sequence, amino acid sequence, nucleotide abundance, or protein abundance. In some embodiments, differential sequences of a genome, proteome, transcriptome, or ORFeome are generated by comparing tissues of interest. In some embodiments, differential sequences of a genome, proteome, transcriptome, or ORFeome are generated by comparing sequences from cells of interest (e.g., a healthy cell versus a cancer cell). In some embodiments, differential sequences of a genome, proteome, transcriptome, or ORFeome are generated by comparing sequences of organisms of interest. In some embodiments, differential sequences of a genome, proteome, transcriptome, or ORFeome can be generated by comparing subjects of interest (e.g., diseased versus healthy subjects).

In some embodiments, a library of the disclosure comprises all k-mer peptides that can be derived from homologous sequences of genomes, proteomes, transcriptomes, ORFeomes, or any combination thereof, where two or more genomes, proteomes, transcriptomes, ORFeomes, or a combination thereof are compared to identify sequences that are homologous sequences (e.g., that share a degree of homology), for example, homologous nucleotide sequences, homologous amino acid sequences, homologous nucleotide abundance, or homologous protein abundance. In some embodiments, homologous sequences of genomes, proteomes, transcriptomes, or ORFeomes are generated by comparing tissues of interest. In some embodiments, homologous sequences of genomes, proteomes, transcriptomes, or ORFeomes are generated by comparing sequences from cells of interest (e.g., a healthy cell versus a involved in autoimmunity cell (e.g., a cell that induces autoimmunity or a cell that is targeted during autoimmunity). In some embodiments, homologous sequences of genomes, proteomes, transcriptomes, or ORFeomes are generated by comparing sequences of organisms of interest. In some embodiments, homologous sequences of genomes, proteomes, transcriptomes, or ORFeomes are generated by comparing subjects of interest (e.g., diseased versus healthy subjects).

In some embodiments, a library of the disclosure comprises all k-mer peptides that can be derived from a polypeptide sequence of interest, for example, all possible 9-mer peptides covering the complete protein sequence of a viral protein. In some embodiments, a library of the disclosure comprises k-mer peptides that can be generated from a polypeptide sequence of interest, wherein the polypeptide sequence of interest is modified, e.g. in silico mutated to produce k-mer peptides comprising mutations (e.g. substitutions, insertions, deletions).

In some embodiments, a library of the disclosure comprises all k-mer peptides that can be derived from mutations in a sequence of interest, for example, all 9-mer peptides that can be generated from single nucleotide mutations in a polynucleotide sequence encoding an antigen or epitope. For example, a library of the disclosure comprises all 9-mer peptides that can be generated from two, three, four, five, six, seven, eight, or nine nucleotide mutations in a polynucleotide sequence encoding an antigen or epitope. In some embodiments, a library of the disclosure comprises all k-mer peptides that can be derived from alanine substitutions, for example, alanine substitutions at any position in any of the sequences described herein (e.g., a protein, a group of proteins, a proteome, an in silico transcripted and translated genome). In some embodiments, a library of the disclosure comprises a positional scanning library, wherein selected amino acid residues are sequentially substituted with all other natural amino acids. In some embodiments, a library of the disclosure comprises a combinatorial positional scanning library, wherein selected amino acid residues are sequentially substituted with all other natural amino acids, two or more positions at a time. In some embodiments, a library of the disclosure comprises an overlapping peptide library, comprising overlapping peptides from a template sequence (e.g., in silico translated genome), wherein overlapping peptides of a set length are offset by a defined number of residues. In some embodiments, a library of the disclosure comprises a T cell truncated peptide library, wherein each replicate of the library comprises equimolar mixtures of peptides with truncations at one terminus (e.g., 8-mers, 9-mers, 10-mers and 11-mers that can be derived from C-terminal truncations of a nominal 11-mer). In some embodiments, a library of the disclosure comprises a customized set of peptides, wherein the customized set of peptides are provided in a list.

In some embodiments, a genome, exome, transcriptome, proteome, or ORFeome of the disclosure is a viral genome, exome, transcriptome, proteome, or ORFeome. Non-limiting examples of viruses include Adenovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Cytomegalovirus (CMV), Dengue virus, Dhori virus, Dugbe virus, Duvenhage virus, Eastern equine encephalitis virus, Ebolavirus, Echovirus, Encephalomyocarditis virus, Epstein-Barr virus (EBV), European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human endogenous retrovirus (HERV), Human enterovirus, Human herpesvirus (e.g., HHV-1, HHV-2, HHV-6A, HHV-6B, HHV-7, HHV-8, Human immunodeficiency virus (e.g., HIV-1, HIV-2), Human papillomavirus (e.g., HPV-1, HPV-2, HPV-16, HPV-18, Human parainfluenza, Human parvovirus B19, Human respiratory syncytial virus (RSV), Human rhinovirus, Human SARS coronavirus, Human spumaretrovirus, Human T-lymphotropic virus (HTLV, e.g. HTLV-1, HTLV-2, HTLV-3), Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norovirus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus (e.g., rotavirus A, rotavirus B, rotavirus C, rotavirus X), Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

In some embodiments, a genome, exome, transcriptome, proteome, or ORFeome of the disclosure is a cancer genome, exome, transcriptome, proteome, or ORFeome. In some embodiments, a library of the disclosure comprises known cancer neoepitopes. In some embodiments, a library of the disclosure comprises all k-mer peptides that can be derived from known cancer antigenic proteins. In some embodiments, a library of the disclosure comprises all k-mer peptides that can be derived from genes involved in epithelial-mesenchymal transition. In some embodiments, a library of the disclosure comprises all k-mer peptides that can be derived from cancer implicated genes. In some embodiments, a library of the disclosure comprises all k-mer peptides that can be derived from mutational cancer driver genes. In some embodiments, a library of the disclosure comprises all k-mer peptides that can be derived from proto-oncogenes, oncogenes, or tumor suppressor genes. In some embodiments, a library of the disclosure comprises all k-mer peptides that can be derived from proto-oncogenes, oncogenes, or tumor suppressor genes, wherein the k-mers comprise mutations as described herein (e.g., amino acid substitutions, alanine substitutions, positional scanning, combinatorial positional scanning etc.).

Non-limiting examples of cancers include Acute Lymphoblastic Leukemia (ALL), Acute Myeloid Leukemia (AML), Adrenocortical Carcinoma, AIDS-Related Cancers, AIDS-Related Lymphoma, Anal Cancer, Appendix Cancer, Astrocytoma, Atypical Teratoid/Rhabdoid Tumor, Basal Cell Carcinoma, Bile Duct Cancer, Bladder Cancer, Bone Cancer, Brain Tumor, Breast Cancer, Bronchial Tumors, Burkitt Lymphoma, Carcinoid Tumor, Carcinoma of Unknown Primary, Cardiac Tumor, Central Nervous System cancer, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ, Embryonal Tumor, Endometrial Cancer, Epithelial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumors, Gestational Trophoblastic Disease, Hairy Cell Leukemia, Head and Neck Cancer, Hepatocellular Cancer, Histiocytosis, Hodgkin Lymphoma, Hypopharyngeal Cancer, Intraocular Melanoma, Islet Cell Tumors, Kaposi Sarcoma, Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell and Small Cell), Lymphoma, Male Breast Cancer, Malignant Fibrous Histiocytoma of Bone and Osteosarcoma, Melanoma, Merkel Cell Carcinoma, Mesothelioma, Metastatic Cancer, Metastatic Squamous Neck Cancer with Occult Primary, Midline Tract Carcinoma, Mouth Cancer, Multiple Endocrine Neoplasia Syndrome, Multiple Myeloma, Mycosis Fungoides, Myelodysplastic Syndromes, Myelodysplastic/Myeloproliferative Neoplasms, Nasal Cavity Cancer, Nasopharyngeal Cancer, Neuroblastoma, Non-Hodgkin Lymphoma, Non-Small Cell Lung Cancer, Oral Cancer, Lip and Oral Cavity Cancer, Oropharyngeal Cancer, Osteosarcoma, Ovarian Cancer, Pancreatic Cancer, Pancreatic Neuroendocrine Tumors, Papillomatosis, Paraganglioma, Paranasal Sinus Cancer, Parathyroid Cancer, Penile Cancer, Pharyngeal Cancer, Pheochromocytoma, Pituitary Tumor, Plasma Cell Neoplasm, Pleuropulmonary Blastoma, Primary Central Nervous System (CNS) Lymphoma, Primary Peritoneal Cancer, Prostate Cancer, Rectal Cancer, Recurrent Cancer, Retinoblastoma, Rhabdomyosarcoma, Salivary Gland Cancer, Sarcoma, Sézary Syndrome, Skin Cancer, Small Cell Lung Cancer, Small Intestine Cancer, Soft Tissue Sarcoma, Squamous Cell Carcinoma of the Skin, Squamous Neck Cancer with Occult Primary, Stomach Cancer, T-Cell Lymphoma, Testicular Cancer, Throat Cancer, Thymoma and Thymic Carcinoma, Thyroid Cancer, Transitional Cell Cancer, Ureter and Renal Pelvis Cancer, Urethral Cancer, Uterine Cancer, Uterine Sarcoma, Vaginal Cancer, Vascular Tumors, Vulvar Cancer, and Wilms Tumor.

In some embodiments, a genome, exome, transcriptome, proteome, or ORFeome of the disclosure is an inflammatory or autoimmunogenic genome, exome, transcriptome, proteome, or ORFeome. In some embodiments, a library of the disclosure comprises known inflammatory or autoimmunogenic neoepitopes or self-epitopes. In some embodiments, a library of the disclosure comprises all k-mer peptides that can be derived from known inflammatory or autoimmunogenic antigenic proteins. In some embodiments, a library of the disclosure comprises all k-mer peptides that can be derived from inflammatory or autoimmune-implicated genes. In some embodiments, a library of the disclosure comprises all k-mer peptides that can be derived from mutation of inflammatory or autoimmune-related driver genes.

Non-limiting examples of inflammatory or autoimmune diseases or conditions include Acute Disseminated Encephalomyelitis (ADEM); Acute necrotizing hemorrhagic leukoencephalitis; Addison's disease; Adjuvant-induced arthritis; Agammaglobulinemia; Alopecia areata; Amyloidosis; Ankylosing spondylitis; Anti-GBM/Anti-TBM nephritis; Antiphospholipid syndrome (APS); Autoimmune angioedema; Autoimmune aplastic anemia; Autoimmune dysautonomia; Autoimmune gastric atrophy; Autoimmune hemolytic anemia; Autoimmune hepatitis; Autoimmune hyperlipidemia; Autoimmune immunodeficiency; Autoimmune inner ear disease (AIED); Autoimmune myocarditis; Autoimmune oophoritis; Autoimmune pancreatitis; Autoimmune retinopathy; Autoimmune thrombocytopenic purpura (ATP); Autoimmune thyroid disease; Autoimmune urticarial; Axonal & neuronal neuropathies; Balo disease; Behcet's disease; Bullous pemphigoid; Cardiomyopathy; Castleman disease; Celiac disease; Chagas disease; Chronic inflammatory demyelinating polyneuropathy (CIDP); Chronic recurrent multifocal ostomyelitis (CRMO); Churg-Strauss syndrome; Cicatricial pemphigoid/benign mucosal pemphigoid; Crohn's disease; Cogans syndrome; Collagen-induced arthritis; Cold agglutinin disease; Congenital heart block; Coxsackie myocarditis; CREST disease; Essential mixed cryoglobulinemia; Demyelinating neuropathies; Dermatitis herpetiformis; Dermatomyositis; Devic's disease (neuromyelitis optica); Discoid lupus; Dressler's syndrome; Endometriosis; Eosinophilic esophagitis; Eosinophilic fasciitis; Erythema nodosum Experimental allergic encephalomyelitis; Experimental autoimmune encephalomyelitis; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis (GPA) (formerly called Wegener's Granulomatosis); Graves' disease; Guillain-Barre syndrome; Hashimoto's encephalitis; Hashimoto's thyroiditis; Hemolytic anemia; Henoch-Schonlein purpura; Herpes gestationis; Hypogammaglobulinemia; Idiopathic thrombocytopenic purpura (ITP); IgA nephropathy; IgG4-related sclerosing disease; Immunoregulatory lipoproteins; Inclusion body myositis; Interstitial cystitis; Inflammatory bowel disease; Juvenile arthritis; Juvenile oligoarthritis; Juvenile diabetes (Type 1 diabetes); Juvenile myositis; Kawasaki syndrome; Lambert-Eaton syndrome; Leukocytoclastic vasculitis; Lichen planus; Lichen sclerosus; Ligneous conjunctivitis; Linear IgA disease (LAD); Lupus (SLE); Lyme disease, chronic; Meniere's disease; Microscopic polyangiitis; Mixed connective tissue disease (MCTD); Mooren's ulcer; Mucha-Habermann disease; Multiple sclerosis; Myasthenia gravis; Myositis; Narcolepsy; Neuromyelitis optica (Devic's); Neutropenia; Non-obese diabetes; Ocular cicatricial pemphigoid; Optic neuritis; Palindromic rheumatism; PANDAS (Pediatric Autoimmune Neuropsychiatric Disorders Associated with Streptococcus); Paraneoplastic cerebellar degeneration; Paroxysmal nocturnal hemoglobinuria (PNH); Parry Romberg syndrome; Parsonnage-Turner syndrome; Pars planitis (peripheral uveitis); Pemphigus; Pemphigus vulgaris; Peripheral neuropathy; Perivenous encephalomyelitis; Pernicious anemia; POEMS syndrome; Polyarteritis nodosa; Type I, II, & III autoimmune polyglandular syndromes; Polymyalgia rheumatic; Polymyositis; Postmyocardial infarction syndrome; Postpericardiotomy syndrome; Progesterone dermatitis; Primary biliary cirrhosis; Primary sclerosing cholangitis; Psoriasis; Plaque Psoriasis; Psoriatic arthritis; Idiopathic pulmonary fibrosis; Pyoderma gangrenosum; Pure red cell aplasia; Raynauds phenomenon; Reactive Arthritis; Reflex sympathetic dystrophy; Reiter's syndrome; Relapsing polychondritis; Restless legs syndrome; Retroperitoneal fibrosis; Rheumatic fever; Rheumatoid arthritis; Sarcoidosis; Schmidt syndrome; Scleritis; Scleroderma; Sclerosing cholangitis; Sclerosing sialadenitis; Sjogren's syndrome; Sperm & testicular autoimmunity; Stiff person syndrome; Subacute bacterial endocarditis (SBE); Susac's syndrome; Sympathetic ophthalmia; Systemic lupus erythematosus (SLE); Systemic sclerosis; Takayasu's arteritis; Temporal arteritis/Giant cell arteritis; Thrombocytopenic purpura (TTP); Tolosa-Hunt syndrome; Transverse myelitis; Type 1 diabetes; Ulcerative colitis; Undifferentiated connective tissue disease (UCTD); Uveitis; Vasculitis; Vesiculobullous dermatosis; Vitiligo; Wegener's granulomatosis (now termed Granulomatosis with Polyangiitis (GPA). Non-limiting examples of inflammatory or autoimmune diseases or conditions include infection, such as a chronic infection, latent infection, slow infection, persistent viral infection, bacterial infection, fungal infection, mycoplasma infection or parasitic infection.

As described, for example, in U.S. Provisional Application No. 62/791,601, hereby incorporated by reference in its entirety.

B. Peptide Production

While the placeholder peptide loaded onto the MHC multimer is prepared recombinantly through expression of the MHC multimer expression construct in a host cell, additional peptides for use in peptide exchange can be prepared either recombinantly or chemically. Peptides suitable for use in the pMHC multimers can be generated according to methods known in the art, or synthetically produced by a commercial vendor or using a peptide synthesizer according to manufacturer's instructions. For example, in some embodiments, peptides suitable for use in the pMHC multimers can be made by in silico production methods.

In other embodiments, peptides can be synthesized via chemical methods, for example, tea bag synthesis, digital photolithography, pin synthesis, and SPOT synthesis. For example, an array of peptides can be generated via SPOT synthesis, where amino acid chains are built on a cellulose membrane by repeated cycles of adding amino acids, and cleaving side-chain protection groups.

In other embodiments, peptides can be expressed using recombinant DNA technology, for example, introducing an expression construct into bacterial cells, insect cells, or mammalian cells, and purifying the recombinant protein from cell extracts.

In some embodiments, peptides can be synthesized by in vitro transcription and translation, where synthesis utilizes the biological principles of transcription and translation in a cell-free context, for example, by providing a nucleic acid template, relevant building blocks (e.g., RNAs, amino acids), enzymes (e.g., RNA polymerase, ribosomes), and conditions.

In some embodiments, in vitro transcription and translation can include cell-free protein synthesis (CFPS). Obtaining a high yield by CFPS requires the usage of bacterial systems, in which the first amino acid of the translated sequence is N-formylmethionine (fMet). This residue differs from methionine by containing a neutral formyl group (HCO) instead of a positively charged amino-terminus (NH₃⁺). Constructs are engineered to include genes encoding an enzymatic cleavage domain and a library polypeptide as described in U.S. Provisional Application No. 62/791,601, hereby incorporated by reference in its entirety. [0078]. Removal of at least the initial methionine amino acid allows successful peptide folding and loading onto MHC protein. In addition, removal of the initial methionine amino acid provides a greater upper limit of peptide library diversity, e.g., 20^{x}, where x is the length of the peptide, while inclusion of this residue will restrict the library diversity to 20^{(x-1)}.

In some embodiments, the peptides are synthesized utilizing an in vitro transcription/translation (IVTT) system that can both transcribe, for example, a DNA construct into RNA, and then translate the RNA into a protein. For example, the methods of the present disclosure comprise a method for performing in vitro transcription/translation (IVTT) to produce a high diversity peptide library and allow for correct folding of proteins. IVTT can allow for protein production in a cell-free environment directly from a DNA or RNA template.

An IVTT method used herein can be performed using, for example, a PCR product, a linear DNA plasmid, a circular DNA plasmid, or an mRNA template with a ribosome-binding site (RBS) sequence. After the appropriate template has been isolated, transcription components can be added to the template including, for example, ribonucleotide triphosphates, and RNA polymerase. After transcription has been completed, translation components can be added, which can be found in, for example, rabbit reticulocyte lysate, or wheat germ extract. In some methods, the transcription and translation can occur during a single step, in which purified translation components found in, for example, rabbit reticulocyte lysate or wheat germ extract are added at the same time as adding the transcription components to the nucleic acid template.

In some embodiments, nucleotide sequence encoding a methionine residue at the N-terminus of the peptide and a cleavable moiety can be encoded in the DNA construct or RNA construct. The cleavable moiety is situated such that at least one N-terminus amino acid residue of the peptide is before or within the cleavable moiety. In some embodiments, the method comprises encoding a cleavable moiety that is situated such that one N-terminus amino acid residue of the peptide is before or within the cleavable moiety. In some embodiments, the one N-terminus amino acid residue is a methionine residue. The cleavable moiety can be cleaved using an enzyme, e.g., a protease, specific to the cleavable moiety, which can also cleave off the cleavable moiety from the remainder of the peptide.

An example of a cleavable moiety that can be encoded in a DNA or RNA construct as described herein includes any cleavable moiety cleaved by an enzyme. In some embodiments, a cleavable moiety can be cleaved by a protease. The cleavage moiety can be cleaved off of the peptide using an enzyme specific for the cleavage moiety. The enzyme can be, for example, Factor Xa, human rhinovirus 3C protease, AcTEV™ Protease, WELQut Protease, Genenase™ small ubiquitin-like modifier (SUMO) protein, Ulp1 protease, furin, caspase 1-10, collagenase, or enterokinase. The Ulp1 protease can cleave off a cleavage moiety in a specific manner by recognizing the tertiary structure, rather than an amino acid sequence. Enterokinase (enteropeptidase) can also be used to cleave the cleavage moiety from the candidate peptide. Enterokinase can cleave after lysine at the following cleavage site: DDDDK (SEQ ID NO.: 237). Enterokinase can also cleave at other basic residues, depending on the sequence and conformation of the protein substrate.

In some embodiments, the cleavable moiety can be a small ubiquitin-like modifier (SUMO) protein. The SUMO domain can be cleaved off of the peptide using a protease specific to SUMO. In some embodiments, the cleavable moiety can be an enterokinase cleavage site: DDDDK (SEQ ID NO.: 237). The protease can be, for example, Ulp1 protease or enterokinase. The Ulp1 protease can cleave off SUMO in a specific manner by recognizing the tertiary structure of SUMO, rather than an amino acid sequence. Enterokinase (enteropeptidase) can also be used to cleave after lysine at the following cleavage site: DDDDK (SEQ ID NO.: 237). Enterokinase can also cleave at other basic residues, depending on the sequence of the protein substrate.

During or after translation of the construct encoding the peptide, the N-terminus amino acid residue(s) (e.g., a SUMO domain) can be efficiently cleaved to produce the properly folded peptide. In some embodiments, at least one N-terminus amino acid residue is cleaved to produce the peptide. In some embodiments, one, two, three, four, five six, seven, eight, nine, ten or more N-terminus amino acid residues are cleaved to produce the peptide. The N-terminus amino acid can be any amino acid residue. The N-terminus amino acid residue can be a methionine amino acid residue. This properly folded peptide is thus not constrained to have an N-terminus methionine, and can be part of a high diversity peptide library produce by cell-free in vitro methods.

After translation of the construct encoding the peptide, an N-terminus amino acid residue can be cleaved to produce the peptide for the high diversity peptide library. In some embodiments, at least one N-terminus amino acid residue is cleaved to produce the peptide. In some embodiments, one or more N-terminus amino acids are cleaved, such as 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 140, 150, 160, 170, 180, 190, 200, 250 or more, N-terminus amino acid residues are cleaved to produce the peptide. The N-terminus amino acid can be any amino acid residue. The N-terminus amino acid residue can be a methionine amino acid residue.

In some embodiments, a DNA or RNA construct comprises a spacer sequence lacking a stop codon. In some embodiments, the peptides are purified by affinity tag purification (e.g., with a FLAG-tag). In some embodiments, the peptides comprise a HaloTag enzymatic sequence. In some embodiments, peptides comprise an avidin or streptavidin.

Peptides can be purified from cell culture supernatants with anti-Flag affinity chromatography (Genscript) or by Ni-affinity chromatography. Size exclusion chromatography (SEC) was performed on a hydrophilic resin (GE Life Sciences) pre-equilibrated in 20 mM HEPES, 150 mM NaCl, pH 7.2.

Alternatively, peptides were purified by Ni-affinity chromatography without SEC purification, using a column buffer of 23 mM sodium phosphate, 500 mM sodium chloride, 500 mM imidazole, pH 7.4.

Peptides produced in mammalian cells were quantitated by UV at 280 nm, whereas CFPS-produced peptides were quantitated by a sandwich ELISA relative to a standard protein.

### VII. Peptide Exchange and MHC Multimer Library Preparation

Recombinantly-expressed p*MHC multimers, loaded with a placeholder peptide (p*), prepared using the expression constructs of the disclosure can be used to generate a library of or microarray of pMHC multimers loaded with a diversity of unique peptide epitopes by in situ or in vitro peptide exchange reactions as described herein. In some embodiments, the peptide exchange reactions are performed in multiwell formats and under native conditions. Peptide binding, and thus peptide exchange, can be determined by a number of techniques, such as ELISA or Differential scanning fluorimetry (DSF), which monitors the stability of the MHC structure, or by biophysical techniques that monitor peptide binding, such as fluorescence polarization. Non-limiting exemplifications of peptide exchange is described in detail in Examples 4-6. In Example 4, cleavage of the placeholder peptide from the MHC multimer is performed (using Factor Xa) and peptide exchange with four different rescue peptides is carried out through a temperature shift. Example 5 confirms peptide exchange by specific T cell staining. Example 6 confirms peptide exchange by Differential Scanning Fluorescence (DSF).

In some embodiments, to measure the dissociation efficiency of placeholder peptides or peptide fragments a fluorescently labeled placeholder peptide is used in exchange reactions in the presence of unlabeled exchange peptides. Aliquots of fluorescently labeled p*MHC multimers are either left untreated or exposed to peptide exchange conditions (e.g., UV exposure) for different time periods. The amount of remaining p*MHC-containing the placeholder peptide is monitored by fluorescence analysis to monitor the reduction in p*MHC complexes.

In some embodiments, the placeholder peptide has a lower affinity for the MHC peptide binding groove than the exchanged peptide epitope, and wherein step (d) comprises contacting the p*MHC monomer with an excess of peptide epitope in a competition assay. In some embodiments, the placeholder peptide has a KD that is about 10-fold lower than the exchanged peptide epitope.

Peptides that bind to the peptide binding groove of the MHC molecule can be a naturally occurring peptide but can also be synthetically created using the knowledge of the binding specificity of the B and F pocket of the particular MHC molecule or the supertype family it belongs to. Suitable ligands can be generated using the available 3D structures of MHC complexes and the knowledge on the binding pocket specificity of the respective MHC molecules.

Peptide binding specificity of MHC I polypeptides is primarily governed by the physiochemical properties of the B and F binding pockets in a coupled fashion. The B and F binding pockets typically bind to “anchor residues” in the peptide that define the binding of the peptide in the peptide binding groove of the MHC. The observed diversity in the amino acid residues of the peptide binding groove of the MHC molecules defines the peptide-binding and the presentation repertoire of the individual MHC molecule (Chang et al. 2011; Frontiers in Bioscience, Landmark Edition, Vol. 16:3014-3035). The specificity of the pockets for anchor residues has been elucidated for a large number MHC molecules, for example, as described in Sidney et al. (BMC Immunology Vol. 9:1, 2008)

The disclosure further provides a method of producing a p*MHC multimer comprising: producing an p*MHC multimer in which the peptide in the binding groove is a placeholder peptide; reacting the p*MHC multimer under conditions suitable to remove the placeholder peptide (e.g., proteolytic cleavage, temperature shift, UV cleavage, contact with a reducing agent); and contacting the p*MHC multimer with an MHC peptide epitope (e.g., rescue peptide) under conditions sufficient for binding of the peptide epitope in the MHC peptide binding groove.

In one embodiment, the two contacting steps are performed by providing a sample comprising the MHC molecule with the MHC peptide epitope and a reducing agent. It is preferred that the MHC peptide epitope is present when the reducing agent is added. In some embodiments, one MHC peptide epitope is added per reaction. In some embodiments, two or more peptide epitopes are added to the reaction.

In some embodiments, peptide exchange is induced by elevating the temperature of the mixture to between about 30°-37° C. In some embodiments, the mixture is elevated to 31°, 32°, 33°, 34°, 35°, 36° or 37°.

In some embodiments, peptide exchange is induced by reducing the pH of the mixture to between about pH 2.5-5.5. In some embodiments, peptide exchange is induced by increasing the pH of the mixture to about pH 9-11.

In some embodiments, the placeholder peptide is an HLA-A*02:01-restricted peptide. In one embodiment, the HLA-A*02:01-restricted peptide is a CMV pp65 peptide epitope. In one embodiment, the CMV pp65 peptide epitope comprises the amino acid sequence NLVPMVATV (SEQ ID NO: 4). In some embodiments, the CMV pp65 peptide epitope consists of the amino acid sequence NLVPMVATV (SEQ ID NO: 4). Other HLA-A*02:01-restricted peptide sequences include the MART-1 sequence EAAGIGILTV (SEQ ID NO: 6) or its heteroclitic variant ELAGIGILTV (SEQ ID NO: 322), the HPV sequence YMLDLQPETT (SEQ ID NO: 7), the HSV sequence SLPITVYYA (SEQ ID NO: 8) and the WT-1 sequence RMFPNAPYL (SEQ ID NO: 9).

In some embodiments, the placeholder peptide is an HLA-A1, A2, A3, All, A23, A24, A26, A30, A31, A32, A33, A68, A74, B7, B8, B13, B14, B15, B18, B27, B35, B37, B38, B39, B40, B42, B44, B45, B50, B52, B53, B55, B57, B58, C1, C3, C4, C5, C7, C8, C14 or C15, non-limiting examples of which include p*A1:01, VTEHDTLLY (SEQ ID NO: 212); p*A3:01, TVRSHCVSK (SEQ ID NO:213); p*A11:01, TTFLQTMLR (SEQ ID NO: 214); p*A24:02, RYPLTFGWCF (SEQ ID NO: 207); p*B7:02, RPHERNGFTVL (SEQ ID NO: 210); p*B35:01, IPSINVHHY (SEQ ID NO: 215); p*C3:04, FVYGGSKTSL (SEQ ID NO: 216), p*B8:01, FLRGRAYGL (SEQ ID NO: 217); p*C7:02, RYRPGTVAL (SEQ ID NO: 218); p*C4:01, QYDPVAALF (SEQ ID NO: 219); p*B15:01, GQFLTPNSH (SEQ ID NO: 220); p*B40:01, KEVNSQLSL (SEQ ID NO: 221); p*B58:01, VSFIEFVGW (SEQ ID NO: 222); and p*C8:02, IAPWYAFAL (SEQ ID NO: 223). Additional peptide/HLA allele combinations are shown in FIG. 10A-D and in SEQ ID NOs: 204-223 and 267-320.

In one embodiment, the MHCII placeholder peptide is a CLIP peptide, such as having the amino acid sequence KPVSKMRMATPLLMQA (SEQ ID NO: 224). Additional suitable CLIP peptides (or CLIP peptide variants) include those having the amino acid sequence RMATPLLMQALPMGAL (SEQ ID NO: 323) or the amino acid sequence LMQALPMGALPQGP (SEQ ID NO: 324).

In some embodiments, the placeholder peptide further comprises a fluorescent label. In so embodiments, the fluorescent label is attached to a cysteine residue in the placeholder peptide.

Upon initiation of exchange as described above, the placeholder peptide dissociates from the MHC complex in the presence of one or more exchangeable peptides (also referred to herein as rescue peptides) to facilitate the formation of stable pMHC monomers or multimers in which the placeholder peptide has been replaced with the exchangeable peptides. Typically, MHC peptide exchange is performed in multiwell format for high-throughput screening of peptide ligands as described herein. Only peptide candidates that can effectively bind and stabilize the peptide-receptive MHC molecules prevent dissociation of the MHC complexes. Peptide exchange can be monitored by a number of techniques such as ELISA or fluorescence polarization, for example, as generally described in Rodenko et al. (Nat. Protocol. 1:1120-1132, 2006).

The resulting pMHC multimers are subsequently analyzed by gel-filtration HPLC, DSF and MHC ELISA to determine the efficiency of exchange and the stability of the new pMHC complex. Certain di-peptides can assist folding and peptide exchange of MHC class I molecules. Di-peptides bind specifically to the F pocket of MHC class I molecules to facilitate peptide exchange and have so far been described and validated for peptide exchange in HLA-A*02:01, HLA-B*27:05, and H-2Kb molecules (Saini et al. Proc Natl Acad Sci USA. 2013 Sep. 17; 110(38):15383-8).

Accordingly, in some embodiments, peptide exchange of the placeholder peptide with a peptide or peptides of interest are catalyzed by dipeptides which catalyze rapid peptide exchange on MHC class I molecules (see, e.g., Saini et al., Proc Natl Acad Sci USA. 2015 Jan. 6; 112(1):202). Suitable dipeptides are those with a hydrophobic second residue. In some embodiments, the dipeptide is glycyl-leucine (GL), glycyl-valine (GV), glycyl-methione (GM), glycyl-cyclohexylalanine (GCha), glycyl-homoleucine (GHle) or glycyl-phenylalanine (GF).

In another embodiment, chaperone-mediated exchange, as described in Overall et al. (2020) Nat. Comm. 11:1909, can be used as the approach for peptide exchange.

In another aspect, the disclosure pertains to methods of producing a library of pMHC multimers comprising a diversity of loaded peptide epitopes. Various steps in the preparation of peptide-exchanged, barcoded pMHC libraries have been described in the art. These steps use standard methods known in the art for preparing barcoded libraries, including use of single-cell sequencing, use of porous hydrogels, use of single template PCR to generate peptide-encoding amplicons (barcodes) and use of in-drop in vitro transcription/translation (IVTT).

### VIII. Labeling

pMHC multimers can be conjugated with a fluorescent label, allowing for identification of T cells that bind the peptide-MHC multimer, for example, via flow cytometry or microscopy. T cells can also be selected based on a fluorescence label through, e.g., fluorescence or magnetic activated cell sorting.

In some embodiments, one or more detectable labels are conjugated to a linker. According to this invention, a “detectable label” is any molecule or functional group that allows for the detection of a biological or chemical characteristic or change in a system, such as the presence of a target substance in the sample.

Examples of detectable labels which may be used include fluorophores, chromophores, electro chemiluminescent labels, bioluminescent labels, polymers, polymer particles, bead or other solid surfaces, gold or other metal particles or heavy atoms, spin labels, radioisotopes, enzyme substrates, haptens, antigens, Quantum Dots, aminohexyl, pyrene, nucleic acids or nucleic acid analogs, or proteins, such as receptors, peptide ligands or substrates, enzymes, and antibodies (including antibody fragments).

Examples of polymer particles labels which may be used include micro particles, beads, or latex particles of polystyrene, PMMA or silica, which can be embedded with fluorescent dyes, or polymer micelles or capsules which contain dyes, enzymes or substrates. Examples of metal particles which may be used include gold particles and coated gold particles, which can be converted by silver stains. Examples of haptens that may be conjugated in some embodiments are fluorophores, myc, nitrotyrosine, biotin, avidin, streptavidin, 2,4-dinitrophenyl, digoxigenin, bromodeoxy uridine, sulfonate, acetylaminoflurene, mercury trintrophonol, and estradiol.

Examples of enzymes which may be used comprise horse radish peroxidase (HRP), alkaline phosphatase (AP),beta-galactosidase (GAL), glucose-6-phosphate dehydrogenase, beta-N-acetylglucosaminidase, Oglucuronidase, invertase, Xanthine Oxidase, firefly luciferase and glucose oxidase (GO). Examples of commonly used substrates for horse radish peroxidase (HRP) include3,3′-diaminobenzidine (DAB), diaminobenzidine with nickel enhancement, 3-amino-9-ethylcarbazole (AEC), Benzidine dihydrochloride (BDHC),Hanker-Yates reagent (HYR), Indophane blue (IB), tetramethylbenzidine (TMB), 4-chloro-1-naphtol (CN), alpha-naphtol pyronin (.alpha.-NP),o-dianisidine (OD), 5-bromo-4-chloro-3-indolylphosphate (BCIP), Nitroblue tetrazolium (NBT), 2-(p-iodophenyl)-3-p-nitrophenyl-5-phenyltetrazolium chloride (INT), tetranitro blue tetrazolium (TNBT), .delta.-bromo-chloro-S-indoxyl-beta-D-galactoside/ferro-ferricyanide (BCIG/FF). Examples of commonly used substrates for Alkaline Phosphatase include Naphthol-AS-B1-phosphate/fast red TR (NABP/FR),Naphthol-AS-MX-phosphate/fast red TR (NAMP/FR),Naphthol-AS-B1-phosphate/fast red TR (NABP/FR),Naphthol-AS-MX-phosphate/fast red TR (NAMP/FR),Naphthol-AS-B1-phosphate/new fuschin (NABP/NF), bromochloroindolylphosphate/nitroblue tetrazolium (BCIP/NBT), b-Bromo-chloro-S-indolyl-beta-delta-galactopyranoside (BCIG).

Examples of luminescent labels which may be used include luminol, isoluminol, acridinium esters, 1,2-dioxetanes and pyridopyridazines. Examples of electrochemiluminescent labels include ruthenium derivatives. Examples of radioactive labels which may be used include radioactive isotopes of iodide, cobalt, selenium, hydrogen, carbon, sulfur, and phosphorous.

Some “detectable labels” also include “colour labels,” in which the biological change or event in the system may be assayed by the presence of a colour, or a change in colour. Examples of “colour labels” are chromophores, fluorophores, chemiluminescent compounds, electrochemiluminescent labels, bioluminescent labels, and enzymes that catalyze a colour change in a substrate.

“Fluorophores” as described herein are molecules that emit detectable electro-magnetic radiation upon excitation with electro-magnetic radiation at one or more wavelengths. A large variety of fluorophores are known in the art and are developed by chemists for use as detectable molecular labels and can be conjugated to the pMHC multimers provided herein. Examples include FLUORESCEIN™ or its derivatives, such as FLUORESCEIN®-5-isothiocyanate (FITC), 5-(and6)-carboxyFLUORESCEIN®, 5- or 6-carboxyFLUORESCEIN®, 6-(FLUORESCEIN®)-5-(and 6)-carboxamido hexanoic acid, FLUORESCEIN® isothiocyanate, rhodamine or its derivatives such as tetramethyl rhodamine and tetramethylrhodamine-5-(and-6) isothiocyanate (TRITC). Other fluorophores include: coumarin dyes such as (diethyl-amino)coumarin or7-amino-4-methylcoumarin-3-acetic acid, succinimidyl ester (AMCA); sulforhodamine 101 sulfonyl chloride (TexasRed® or TexasRed® sulfonyl chloride; 5-(and-6)-carboxyrhodamine 101, succinimidyl ester, also known as 5-(and-6)-carboxy-X-rhodamine, succinimidyl ester (CXR); lissamine or lissamine derivatives such as lissamine rhodamine B sulfonyl Chloride (LisR); 5-(and-6)-carboxyFLUORESCEIN®, succinimidyl ester (CFI); FLUORESCEIN®5-isothiocyanate (FITC); 7-diethylaminocoumarin-3-carboxylic acid, succinimidyl ester (DECCA); 5-(and-6)-carboxytetramethyl-rhodamine, succinimidyl ester (CTMR); 7-hydroxycoumarin-3-carboxylic acid, succinimidyl ester (HCCA); 6->FLUORESCEIN®.-5-(and-6)-carboxamidolhexanoic acid (FCHA); N-(4,4-difluoro-5,7-dimethyl-4-bora-3a,4a-diaza-3-indacenepropionic acid, succinimidyl ester; also known as 5,7-dimethylBODIPY® propionic acid, succinimidyl ester (DMBP); “activated FLUORESCEIN® derivative” (FAP), available from Probes, Inc.; eosin-5-isothiocyanate (EITC); erythrosin-5-isothiocyanate (ErlTC); and Cascade® Blue acetylazide (CBAA) (the O-acetylazide derivative of 1-hydroxy-3,6,8-pyrene-trisulfonic acid). Yet other potential fluorophores useful in this invention include fluorescent proteins such as green fluorescent protein and its analogs or derivatives, fluorescent amino acids such as tyrosine and tryptophan and their analogs, fluorescent nucleosides, and other fluorescent molecules such as Cy2,Cy3, Cy 3.5, CY5™, CY5™5, Cy 7, IR dyes, Dyomics dyes, phycoerythrine, Oregon green 488, pacific blue, rhodamine green, and Alexa dyes. Yet other examples of fluorescent labels include conjugates of R-phycoerythrin orallophycoerythrin, inorganic fluorescent labels such as particles based on semiconductor material like coated CdSe nanocrystallites.

A number of the fluorophores above, as well as others, are available commercially, from companies such as Probes, Inc. (Eugene, Oreg.), Pierce Chemical Co. (Rockford, Ill.), or Sigma-Aldrich Co. (St. Louis, Mo.).

The detectable label can be detected by numerous methods, including, for example, reflectance, transmittance, light scatter, optical rotation, and fluorescence or combinations hereof in the case of optical labels or by film, scintillation counting, or phosphorimaging in the case of radioactive labels. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.). In some embodiments, more than one detectable labels employed.

### IX. Identifiers and Barcoding

In certain embodiments, an MHC multimer of the disclosure comprises an identifier tag or label, such as an oligonucleotide barcode, that facilitates identification of the MHC multimer. Typically, the identifier tag, e.g., oligonucleotide barcode, is attached to the multimerization domain of the MHC multimer, such as through a binding moiety on the identifier tag, e.g., oligonucleotide barcode, that binds to a binding site on the multimerization domain. For example, when the multimerization domain is streptavidin or avidin, since the pMHC monomers are conjugated to the multimerization domain at a site other than the biotin-binding site, the MHC multimer can be labeled with an identifier tag, e.g., oligonucleotide barcode, using a biotinylated form of the identifier tag, e.g., a biotinylated oligonucleotide barcode. Labeling of the MHC multimer is then easily achieved by incubation of the MHC multimer with the biotinylated identifier tag, e.g., biotinylated oligonucleotide barcode. A non-limiting exemplification of barcoding of recombinantly expressed MHC multimers using biotinylated oligonucleotides is described in detail in Example 3.

In another embodiment, the MHC multimer is labeled with an identifier tag, e.g., oligonucleotide barcode, in the peptide portion of the multimer. That is, barcode-labeled MHC-binding peptides can be used in an exchange reaction as described herein to the load the MHC multimers with barcode-labeled peptides.

Typically, an oligonucleotide barcode is a unique oligonucleotide sequence ranging for 10 to more than 50 nucleotides. The barcode has shared amplification sequences in the 3′ and 5′ ends, and a unique sequence in the middle. This sequence can be revealed by sequencing and can serve as a specific barcode for a given molecule.

In one embodiment, the nucleic acid component of the barcode (typically DNA) has a special structure. Thus, in one embodiment, the at least one nucleic acid molecule is composed of at least a 5′ first primer region, a central region (barcode region), and a 3′ second primer region. In this way the central region (the barcode region) can be amplified by a primer set. The length of the nucleic acid molecule may also vary. Thus, in other embodiments, the at least one nucleic acid molecule has a length in the range 20-100 nucleotides, such as 30-100, such as 30-80, such as 30-50 nucleotides. In one embodiment, the nucleic acid identifier is from 40 nucleotides to 120 nucleotides in length. The coupling of the oligonucleotide barcode to the MHC multimer may also vary. Thus, in one embodiment, the at least one oligonucleotide barcode is linked to said MHC multimer via a biotin binding domain interacting with streptavidin or avidin within the MHC multimer. Other coupling moieties may also be used, depending on the availability of an appropriate binding site with the MHC multimer (e.g., within the multimerization domain of the MHC multimer) and an appropriate corresponding binding domain that can be attached to the oligonucleotide barcodes molecules to facilitate attachment.

In a further embodiment, the at least oligonucleotide barcode molecule comprises or consists of DNA, RNA, and/or artificial nucleotides such as PLA or LNA. Preferably DNA, but other nucleotides may be included to e.g. increase stability.

The use of barcode technology is well known in the art, see for example Shiroguchi et al., Proc. Natl. Acad. Sci. USA., 2012 Jan. 24; 109(4):1347-52; and Smith et al., Nucleic Acids Research, 2010 July; 38(13)11:e142. Further methods and compositions for using barcode technology include those described in U.S. 2016/0060621. Use of barcode technology specifically to label MHC multimers also has been described, see for example Bentzen et al., Nature Biotech. 34:10: 1037-1045, 2016; Bentzen and Hadrup, Cancer Immunol. Immunotherap. 66:657-666, 2017. Standard methods for preparing barcode oligonucleotides, including conjugating them with a suitable binding moiety (e.g., biotinylation) that can bind the MHC multimer, are known in the art and can be applied to preparing barcode oligonucleotides for labeling the MHC multimers.

Methods for generating customizable DNA barcode libraries are publicly available. Programs include Generator and nxCode, consisting of 96-587 barcodes, respectively, as well as The DNA Barcodes Package and TagD software (reporting generating libraries consisting of 100,000 barcodes).

Preparation of a variety of large-scale barcode libraries have been described in the art, which approaches can be used to obtain barcode libraries for labeling pMHC multimer libraries. For example, Xu et al. describe a set of 240,000 unique 25-mer oligonucleotides with sequences that have similar amplifications properties while maintaining maximum diversity of their identification motifs (Xu et al. PNAS 106:2289-2294, 2008). Wang et al. describe construction of barcode sets using particle swarm optimization (Wang et al. IEEE/ACM Trans. Comput. Biol. Bioinform. 15:999-1002). Lyons describes generation of large-scale libraries of DNA barcodes of up to one million members. (Lyons, Sci. Reports 7:13899, 2017).

In some cases, the unique molecular identifier barcode is encoded by a contiguous sequence of nucleotides tagged to one end of a target nucleic acid. In other cases, the unique molecular identifier (UMI) barcode is encoded by a non-contiguous sequence. Non-contiguous UMIs can have a portion of the barcode at a first end of the target nucleic acid and a portion of the barcode at a second end of the target nucleic acid. In some cases, the UMI is a non-contiguous barcode containing a variable length barcode sequence at a first end and a second identifier sequence at a second end of the target nucleic acid. In some cases, the UMI is a non-contiguous barcode having a variable length barcode sequence at a first end and a second identifier sequence at a second end of the target nucleic acid, wherein the second identifier sequence is determined by a position of a transposase fragmentation event, e.g., a transposase fragmentation site and transposon end insertion event.

In some cases, the barcode is a “variable length barcode.” As used herein, a variable length barcode is an oligonucleotide that differs from other variable length barcode oligonucleotides in a population, by length, which can be identified by the number of contiguous nucleotides in the barcode. In some cases, additional barcode complexity for the variable length barcode can be provided by the use of variable nucleotide sequence, as described in the paragraphs above, in addition to the variable length.

In an exemplary embodiment, a variable length barcode can have a length of from 0 to no more than 5 nucleotides. Such a variable length barcode can be denoted by the term “[0-5].” In such an embodiment, it is understood that a population of target nucleic acids that are attached to such a variable length barcode is expected to include at least one target nucleic acid attached to a variable length barcode that has at least 1 nucleotide (e.g., attached to a variable length barcode having only 1, only 2, only 3, only 4, or only 5 nucleotides). In such an embodiment, it is further understood that a population of target nucleic acids that are attached to such a variable length barcode can include at least one target nucleic acid that contains no variable length barcode (i.e., a variable length barcode having a length of 0), and/or at least one target nucleic acid that contains a variable length barcode having only 1 nucleotide, and/or at least one target nucleic acid that contains a variable length barcode having only 2 nucleotides, and/or at least one target nucleic acid that contains a variable length barcode having only 3 nucleotides, and/or at least one target nucleic acid that contains a variable length barcode having only 4 nucleotides, and/or and at least one target nucleic acid that contains a variable length barcode having only 5 nucleotides. In such an embodiment, the [0-5] variable length barcode can uniquely identify (differentiate), by itself, 5 different target nucleic acid molecules of the same sequence. Further, in such an embodiment, the [0-5] variable length barcode can uniquely identify (differentiate) 5 different target nucleic molecules of a first sequence, 5 different target nucleic acid molecules of a second sequence, etc. for each different target nucleic acid sequence. Furthermore, barcode labelled MHC-multimers can be used in combination with single-cell sorting and TCR sequencing, where the specificity of the TCR can be determined by the co-attached barcode. This will enable us to identify TCR specificity for potentially 1000+ different antigen responsive T-cells in parallel from the same sample, and match the TCR sequence to the antigen specificity. The future potential of this technology relates to the ability to predict antigen responsiveness based on the TCR sequence.

The complexity of the barcode labeled MHC multimer libraries will allow for personalized selection of relevant TCRs in a given individual.

The barcode is co-attached to the multimer and serves as a specific label for a particular peptide-MHC complex. In this way at least 1000 to 10,000 or more different peptide-MHC multimers can be mixed, allow specific interaction with T-cells from blood or other biological specimens, wash-out unbound MHC-multimers and determine the sequence of the DNA-barcodes. When selecting a cell population of interest, the sequence of barcodes present above background level, will provide a fingerprint for identification of the antigen responsive cells present in the given cell-population. The number of sequence-reads for each specific barcode will correlate with the frequency of specific T-cells, and the frequency can be estimated by comparing the frequency of reads to the input-frequency of T-cells.

The DNA-barcode serves as a specific label for the antigen specific T-cells and can be used to determine the specificity of a T-cell after e.g. single-cell sorting, functional analyses or phenotypical assessments. In this way antigen specificity can be linked to both the T-cell receptor sequence (that can be revealed by single-cell sequencing methods) and functional and phenotypical characteristics of the antigen specific cells.

Barcode labeled MHC multimer libraries can be used for the quantitative assessment of MHC multimer binding to a given T-cell clone or TCR transduced/transfected cells. Since sequencing of the barcode label allow several different labels to be determined simultaneously on the same cell population, this strategy can be used to determine the avidity of a given TCR relative to a library of related peptide-MHC multimers. The relative contribution of the different DNA-barcode sequences in the final readout is determined based on the quantitative contribution of the TCR binding for each of the different peptide-MHC multimers in the library. Via titration based analyses it is possible to determine the quantitative binding properties of a TCR in relation to a large library of peptide-MHC multimers, all merged into a single sample. For this particular purpose the MHC multimer library may specifically hold related peptide sequences or alanine-substitution peptide libraries.

In some embodiments, unique identifiers can be used for each sample of a plurality of samples. In some embodiments, identifiers can be shared between two or more samples. In some embodiments, identifiers can comprise some sequences that are shared between all samples, and other sequences that are unique to one sample. In some embodiments, an identifier can comprise a sequence shared between all samples, and a sequence unique to one sample. In some embodiments, a sequence shared between samples can be used for identifier amplification (e.g., PCR amplification with suitable primers). In some embodiments, a sequence unique to one sample or shared between a subset of samples can be used for detection or quantification via qPCR (e.g., sequences for hydrolysis probes, such as TaqMan probes). In some embodiments, a sequence unique to one sample or shared between a subset of samples can be used for detection or quantification via sequencing.

In some embodiments, an identifier can comprise a unique, in silico-generated sequence; each identifier sequence can be assigned to a sample of a plurality of samples and the identifier-sample assignment can be stored in a database. In some embodiments, an identifier can comprise a nucleotide sequence that codes for all or part of a peptide or protein. In some embodiments, an identifier can comprise a nucleotide sequence that codes for an open reading frame. In some embodiments, an identifier can comprise a nucleotide sequence that includes a promoter sequence. In some embodiments, an identifier can comprise a nucleotide sequence that includes a binding site for a DNA-binding protein, e.g. a transcription factor or polymerase enzyme. In some embodiments, an identifier can comprise one or more sequences targeted by a nuclease, e.g. a restriction enzyme. In some embodiments, an identifier can comprise all sequence elements necessary for in vitro transcription and translation of a sequence. In some embodiments, an identifier does not comprise all sequence elements necessary for in vitro transcription and translation of a sequence.

In some embodiments, an identifier can comprise a biotinylated nucleotide sequence. In some embodiments, an identifier can be biotinylated by PCR amplification with a biotinylated primer(s). In some embodiments, an identifier can be biotinylated by enzymatic incorporation of a biotinylated label, e.g. a biotin dUTP label, by use of Klenow DNA polymerase enzyme, nick translation or mixed primer labeling RNA polymerases, including T7, T3, and SP6 RNA polymerases. In some embodiments, an identifier can be biotinylated by photobiotinylation, e.g. photoactivatable biotin can be added to the sample, and the sample irradiated with UV light.

In some embodiments, an identifier can be generated from a template polynucleotide, e.g. via PCR amplification of a template DNA. In some embodiments, a template polynucleotide can comprise a nucleotide sequence that codes for an open reading frame. In some embodiments, a template polynucleotide can comprise a nucleotide sequence that includes a promoter sequence. In some embodiments, a template polynucleotide can comprise a nucleotide sequence that includes a binding site for a DNA-binding protein, e.g. a transcription factor or polymerase enzyme. In some embodiments, a template polynucleotide can comprise one or more sequences targeted by a nuclease, e.g. a restriction enzyme. In some embodiments, a template polynucleotide can comprise all sequence elements necessary for in vitro transcription and translation of a sequence. In some embodiments, a template polynucleotide does not comprise all sequence elements necessary for in vitro transcription and translation of a sequence.

pMHC multimers with attached identifiers (e.g., oligonucleotide barcodes) can be incubated with a plurality of T cells, followed by sorting of T cells into single-cell compartments. T cells are lysed, and nucleic acids from lysed T cells comprising identifiers are produced. Nucleic acids are pooled and sequenced. Identifiers allow matching of peptide identifiers to T cell sequences from the same compartment. TCR-antigen specificity profiles are determined by identifying a TCR sequence (e.g., variable region, hypervariable region, or CDR) from a compartment, and quantifying peptide identifier reads from the same compartment.

Multiple TCRs can be identified that exhibit binding affinity for peptides of the peptide library, and multiple peptides can be identified that exhibit binding affinity for specific TCRs.

Epitope mutations in an antigen of an identified TCR-antigen pair can be identified that result in increased TCR binding affinity.

Peptides and TCR sequences can be identified that are associated with control of disease associated protein, and can be used to design vaccines and cell therapies.

For assessing response to therapy, for each peptide identifier sequenced, corresponding TCR sequences are identified. Multiple TCRs are identified that exhibit binding affinity for some peptides of the peptide library, and multiple peptides are identified that exhibit binding affinity for some TCRs. Subjects are followed longitudinally and results of assays are compared to identify peptides and TCR sequences that are associated with successful response to immunotherapy.

### X. Compositions and Kits

In another aspect, the disclosure comprises compositions and kits for use in the methods described herein. In one embodiment, the disclosure provides a pMHC multimer expression construct composition. In one embodiment, the pMHC multimer expression construct composition is a pMHC tetramer expression construct composition. In one embodiment, the multimerization domain of the tetramer is streptavidin or avidin. In one embodiment, the pMHC expression construct tetramer comprises four MHC monomers covalently conjugated to the streptavidin or avidin molecule at sites other than the biotin-binding site of streptavidin or avidin.

In one embodiment, the four MHC monomers each comprise (i.e., are loaded with) an MHC-binding peptide, wherein each monomer comprises the same MHC-binding peptide. In one embodiment, the pMHC tetramer further comprises a biotinylated oligonucleotide barcode bound to the biotin-binding site of streptavidin or avidin. In one embodiment, the pMHC multimer (e.g., tetramer) is a pMHC Class I multimer (e.g., tetramer). In another embodiment, the pMHC multimer (e.g., tetramer) is a pMHC Class II multimer (e.g., tetramer).

In one embodiment, the disclosure comprises a kit comprising at least one MHC multimer expression construct and host cells for expression of the construct. The kit can further comprise means for purifying the MHC multimers from the host cells (e.g., from the supernatant of the host cells). In another embodiment, the disclosure comprises a kit comprising a plurality of pMHC multimer compositions. In one embodiment, each pMHC multimer in the plurality is a pMHC tetramer. In one embodiment, the multimerization domain of each tetramer is streptavidin or avidin. In one embodiment, each MHC tetramer comprises four MHC monomers covalently conjugated to the streptavidin or avidin molecule at sites other than the biotin-binding site of streptavidin or avidin. In one embodiment, the four MHC monomers each comprise an MHC-binding peptide, wherein each MHC monomer within each single tetramer comprises (i.e., is loaded with) the same MHC-binding peptide and wherein each MHC tetramer within the plurality comprises (i.e., is loaded with) a different MHC-binding peptide, thereby forming a library of MHC-binding peptides. In one embodiment, each MHC tetramer within the plurality further comprises a biotinylated oligonucleotide barcode bound to the biotin-binding site of streptavidin or avidin. In one embodiment, each pMHC multimer (e.g., tetramer) of the plurality is a pMHC Class I multimer (e.g., tetramer). In another embodiment, each pMHC multimer (e.g., tetramer) of the plurality is a pMHC Class II multimer (e.g., tetramer).

### XI. Methods of Use

Another aspect of the invention relates to methods for detecting antigen responsive T cells, for example in a sample. Generally, the methods comprise providing a plurality of pMHC multimers of the disclosure; contacting the pMHC multimers with said sample; and detecting binding of the pMHC multimers to antigen responsive T cells within the sample, thereby detecting T cells responsive to an antigenic peptide present in the plurality of pMHC multimers. In one embodiment, binding is detected by amplifying the barcode region of the oligonucleotide barcode linked to the pMHC multimer. Typically, for pMHCI multimers, the antigen responsive T cell is a CD8+ T cell, whose TCRs recognize peptide-bound MHC Class I molecules, whereas for pMHCII multimers, the antigen responsive T cell is a CD4+ T cell, whose TCRs recognize peptide-bound MHC Class II molecules.

This pMHC multimer technology allows for detection of multiple (potentially >1000) different antigen-specific T cells in a single sample. The technology can be used, for example, for T-cell epitope mapping, immune-recognition discovery, diagnostics tests and measuring immune reactivity after vaccination or immune-related therapies. For therapeutic use, the pMHC multimers allow for identification and selection of antigen-specific T cells to be administered for therapy, such as for adoptive T cell transfer therapy.

A. Assays

In one embodiment of the present invention MHC multimers can be used for detection of individual T-cells in fluid samples using flowcytometry or flow cytometry-like analysis.

Liquid cell samples can be analyzed using a flow cytometer, able to detect and count individual cells passing in a stream through a laser beam. For identification of specific T-cells using MHC multimers, cells are stained with fluorescently labeled MHC multimer by incubating cells with MHC multimer and then forcing the cells with a large volume of liquid through a nozzle creating a stream of spaced cells. Each cell passes through a laser beam and any fluorochrome bound to the cell is excited and thereby fluoresces. Sensitive photomultipliers detect emitted fluorescence, providing information about the amount of MHC multimer bound to the cell. By this method MHC multimers can be used to identify individual T-cells and/or specific T-cell populations in liquid samples.

Cell samples capable of being analyzed by MHC multimers in flowcytometry analysis include, but is not limited to, blood samples or fractions thereof, T-cell lines (hybridomas, transfected cells) and homogenized tissues like spleen, lymph nodes, tumors, brain or any other tissue comprising T-cells.

When analyzing blood samples whole blood can be used with or without lysis of red blood cells prior to analysis on flow cytometer. Lysing reagent can be added before or after staining with MHC multimers. When analyzing blood samples without lysis of red blood cells one or more gating reagents may be included to distinguish lymphocytes from red blood cells.

Preferred gating reagent are marker molecules specific for surface proteins on red blood cells, enabling subtraction of this cell population from the remaining cells of the sample. As an example, a fluorochrome labelled CD45 specific marker molecule e.g. an antibody can be used to set the trigger discriminator to allow the flow cytometer to distinguish between red blood corpuscles and stained white blood cells.

Alternative to analysis of whole blood, lymphocytes can be purified before flow cytometry analysis e.g. using standard procedures like a FICOLL®-Hypaque gradient. Another possibility is to isolate T-cells from the blood sample, for example, by adding the sample to antibodies or other T-cell specific markers immobilized on solid support. Marker specific T-cells are then attached to the solid support and following washing specific T-cells can be eluted. This purified T-cell population can then be used for flow cytometry analysis together with MHC multimers.

T-cells may also be purified from other lymphocytes or blood cells by rosetting. Human T-cells form spontaneous rosettes with sheep erythrocytes, also called E-rossette formation. E-rossette formation can be carried out by incubating lymphocytes with sheep red erythrocytes followed by purification over a density gradient e.g. a FICOLL® Hypaque gradient.

Instead of actively isolating T-cells, unwanted cells like B-cells, NK cells or other cell populations can be removed prior to the analysis. A preferred method for removal of unwanted cells is to incubate the sample with marker molecules specific or one or more surface proteins on the unwanted cells immobilized unto solid support. An example includes use of beads coated with antibodies or other marker molecule specific for surface receptors on the unwanted cells e.g. markers directed against CD19, CD56, CD14, CD15 or others. Briefly beads coated with the specific surface marker(s) are added to the cell sample. Cells different from the wanted T-cells with appropriate surface receptors will bind the beads. Beads are removed by e.g. centrifugation or magnetic withdrawal (when using magnetic beads) and remaining cell are enriched for T-cells.

Another example is affinity chromatography using columns with material coated with antibodies or other markers specific for the unwanted cells.

Alternatively, specific antibodies or markers can be added to the blood sample together with complement, thereby killing cells recognized by the antibodies or markers.

Various gating reagents can be included in the analysis. Gating reagents here means labeled antibodies or other labelled marker molecules identifying subsets of cells by binding to unique surface proteins or intracellular components or intracellular secreted components. Preferred gating reagents when using MHC multimers are antibodies and marker molecules directed against CD2, CD3, CD4, and CD8 identifying major subsets of T-cells. Other preferred gating reagents are antibodies and markers against CD11a, CD14, CD15, CD19, CD25, CD30, CD37, CD49a, CD49e, CD56, CD27, CD28, CD45, CD45RA, CD45RO, CD45RB, CCR7, CCR5, CD62L, CD75, CD94, CD99, CD107b, CD109, CD152, CD153, CD154, CD160, CD161, CD178, CDw197, CDw217, Cd229, CD245, CD247, Foxp3, or other antibodies or marker molecules recognizing specific proteins unique for different lymphocytes, lymphocyte populations or other cell populations. Also included are antibodies and markers directed against interleukins e.g. IL-2, IL-4, IL-6, IL-10, IL-12, IL-21; Interferons e.g., INFγ, TNFα, TNFβ or other cytokine or chemokines.

Gating reagents can be added before, after or simultaneous with addition of MHC multimer to the sample. Following labelling with MHC multimers and before analysis on a flow cytometer stained cells can be treated with a fixation reagent (e.g., formaldehyde, ethanol or methanol) to cross-link bound MHC multimer to the cell surface. Stained cells can also be analyzed directly without fixation.

The flow cytometer can in one embodiment be equipped to separate and collect particular types of cells. This is called cell sorting. MHC multimers in combination with sorting on a flow cytometer can be used to isolate antigen specific T-cell populations. Gating reagents as described above can be including further specifying the T-cell population to be isolated. Isolated and collected specific T-cell populations can then be further manipulated as described elsewhere herein, e.g. expanded in vitro.

Direct determination of the concentration of MHC-peptide specific T-cells in a sample can be obtained by staining blood cells or other cell samples with MHC multimers and relevant gating reagents followed by addition of an exact amount of counting beads of known concentration. In general, the counting beads are microparticles with scatter properties that put them in the context of the cells of interest when registered by a flow cytometer. They can be either labelled with antibodies, fluorochromes or other marker molecules or they may be unlabelled. In some embodiments, the beads are polystyrene beads with molecules embedded in the polymer that are fluorescent in most channels of the flow-cytometer. Inhere the terms “counting bead” and “microparticle” are used interchangeably.

Beads or microparticles suitable for use include those which are used for gel chromatography, for example, gel filtration media such as SEPHADEX®. Suitable microbeads of this sort include, but is not limited to, SEPHADEX® G-10 having a bead size of 40-120 μm (SigmaAldrich catalogue number 27, 103-9), SEPHADEX®. G-15 having a bead size of 40-120 μm (Sigma Aldrich catalogue number 27, 104-7), SEPHADEX®. G-25 having a bead size of 20-50 μm (Sigma Aldrich catalogue number 27, 106-3), SEPHADEX®. G-25 having a bead size of 20-80 μm (Sigma Aldrich catalogue number 27, 107-1), SEPHADEX®. G-25 having a bead size of 50-150 μm (Sigma Aldrich catalogue number 27, 109-8), SEPHADEX.®. G-25 having a bead size of 100-300 μm (Sigma Aldrich catalogue number 27, 110-1), SEPHADEX® G-50 having a bead size of 20-50 μm (Sigma Aldrich catalogue number 27, 112-8), SEPHADEX® G-50 having a bead size of 20-80 μm (Sigma Aldrich catalogue number 27, 113-6), SEPHADEX® G-50 having a bead size of 50-150 μm (Sigma Aldrich catalogue number 27, 114-4), SEPHADEX®G-50 having a bead size of 100-300 μm (SigmaAldrich catalogue number 27, 115-2), SEPHADEX® G-75 having a bead size of 20-50 μm (Sigma Aldrich catalogue number 27, 116-0), SEPHADEX®G-75 having a bead size of 40-120 μm (Sigma Aldrich catalogue number 27, 117-9), SEPHADEX® G-100 having a bead size of 20-50 μm (SigmaAldrich catalogue number 27, 118-7), SEPHADEX® G-100 having a bead size of 40-120 μm (Sigma Aldrich catalogue number 27, 119-5),SEPHADEX®G-150 having a bead size of 40-120 μm (Sigma Aldrich catalogue number 27, 121-7), and SEPHADEX® G-200 having a bead size of 40-120 μm (Sigma Aldrich catalogue number 27, 123-3).

Other preferred particles for use in the methods and compositions described here comprise plastic microbeads. While plastic microbeads are usually solid, they may also be hollow inside and could be vesicles and other microcarriers. They do not have to be perfect spheres in order to function in the methods described here. Plastic materials such as polystyrene, polyacrylamide and other latex materials may be employed for fabricating the beads, but other plastic materials such as polyvinylchloride, polypropylene and the like may also be used.

The counting beads are used as reference population to measure the exact volume of analyzed sample. The sample(s) are analyzed on a flow cytometer and the amount of MHC-specific T-cell is determined using e.g. a predefined gating strategy and then correlating this number to the number of counted counting beads in the same sample

Detection of specific T-cells in a sample combined with simultaneous detection of activation status of T-cells can also be measured using marker molecules specific for up- or down-regulated surface exposed receptors together with MHC multimers. The marker molecule and MHC multimer can be labelled with the same label or different labelling molecules and added to the sample simultaneously or sequentially or separately.

1. Detection of Individual T-Cells in Fluid Samples Using Microscopy

Another preferred method for detection of individual T-cells in fluid samples is using microscopy. Microscopy comprises any type of microscopy including optical, electron and scanning probe microscopy, Bright field microscopy, Dark field microscopy, Phase contrast microscopy, Differential interference contrast microscopy, Fluorescence microscopy, Confocal laser scanning microscopy, X-ray microscopy, Transmission electron microscopy, Scanning electron microscopy, atomic force microscope, Scanning tunneling microscope and photonic force microscope. This can be done as follows: A suspension of T-cells are added to MHC multimers, the sample washed and then the amount of MHC multimer bound to each cell is measured. Bound MHC multimers may be labelled directly or measured through addition of labelled marker molecules. The sample is then spread out on a slide or similar in a thin layer able to distinguish individual cells and labelled cells identified using a microscope. Depending on the type of label different types of microscopes may be used, e.g. if fluorescent labels are used a fluorescent microscope is used for the analysis. For example, MHC multimers can be labeled with a flourochrome or bound MHC multimer detected with a fluorescent antibody. Cells with bound fluorescent MHC multimers can then be visualized using e.g. an immunofluorescence microscope or a confocal fluorescence microscope.

2. Immunohistochemistry (IHC)

IHC is a method where MHC multimers can be used to directly detect specific T-cells e.g. in sections of solid tissue. In some embodiments, sections of fixed or frozen tissue sample are incubated with MHC multimer allowing MHC multimer to bind specific T-cells in the tissue. The MHC multimer may be labelled with a fluorochrome, chromophore, or any other labelling molecule that can be detected. The labeling of the MHC multimer may be directly or through a second marker molecule. As an example, the MHC multimer can be labelled with a tag that can be recognized by e.g. a secondary antibody, optionally labelled with HRP or another label. The bound MHC multimer is then detected by its fluorescence or absorbance (for fluorophore or chromophore), or by addition of an enzyme-labelled antibody directed against this tag, or another component of the MHC multimer (e.g. one of the protein chains, a label on the one or more multimerization domain). The enzyme can e.g. be Horseradish Peroxidase (HRP) or Alkaline Phosphatase (AP), both of which convert a colorless substrate into a colored reaction product in situ. This colored deposit identifies the binding site of the MHC multimer and can be visualized under e.g. alight microscope. The MHC multimer can also be directly labelled with e.g. HRP or AP, and used in IHC without an additional antibody.

In some embodiments, the detection of T-cells in solid tissue includes use of tissue embedded in paraffin, from which tissue sections are made and fixed in formalin before staining. Antibodies are standard reagents used for staining of formalin-fixed tissue sections; these antibodies often recognize linear epitopes. In contrast, most MHC multimers are expected to recognize a conformational epitope on the TCR. In this case, the native structure of TCR needs to be at least partly preserved in the fixed tissue.

In other embodiments, staining performed tissue sections from frozen tissue blocks. In this type of staining fixation is done after MHC multimer staining.

3. Immunofluorescence Microscopy

In some embodiments, MHC multimers can be used to identify specific T-cells in sections of solid tissue. Instead of visualization of bound MHC multimer by an enzymatic reaction, MHC multimers are labelled with a fluorochrome or bound MHC multimer are detected by a fluorescent antibody. Cells with bound fluorescent MHC multimers can be visualized in an immunofluorescence microscope or in a confocal fluorescence microscope. This method can also be used for detection of T-cells in fluid samples using the principles described for detection of T-cells in fluid sample described elsewhere herein.

4. Detection of T-Cells in Solid Tissue In Vivo

MHC multimers may also be used for detection of T-cells in solid tissue in vivo. For in vivo detection of T-cells labeled MHC multimers are injected into the body of the individual to be investigated. The MHC multimers may be labeled with e.g. a paramagnetic isotope. Using a magnetic resonance imaging (MRI) scanner or electron spin resonance (ESR) scanner MHC multimer binding T-cells can then be measured and localized. In general, any conventional method for diagnostic imaging visualization can be utilized. Usually gamma and positron emitting radioisotopes are used for camera and paramagnetic isotopes for MRI.

5. Detection of T-Cells Immobilized on Solid Support.

In a number of applications, it may be advantageous immobilize the T-cell onto a solid or semi-solid support. Such support may be any which is suited for immobilization, separation etc. Non-limiting examples include particles, beads, biodegradable particles, sheets, gels, filters, membranes (e. g. nylon membranes), fibres, capillaries, needles, microtitre strips, tubes, plates or wells, combs, pipette tips, microarrays, chips, slides, or indeed any solid surface material. The solid or semi-solid support may be labelled, if this is desired. The support may also have scattering properties or sizes, which enable discrimination among supports of the same nature, e.g. particles of different sizes or scattering properties, color or intensities.

An example of a method where MHC multimers can be used for detection of immobilized T-cells is ELISA (Enzyme-Linked ImmunosorbentAssay). ELISA is a binding assay originally used for detection of antibody-antigen interaction. Detection is based on an enzymatic reaction, and commonly used enzymes are e.g. HRP and AP. MHC multimers can be used in ELISA-based assays for analysis of purified TCR's and T-cells immobilized in wells of a microtiter plate. The bound MHC multimers can be labelled either by direct chemical coupling of e.g. HRP or AP to the MHC multimer (e.g. the one or more multimerization domain or the MHC proteins), or e.g. by an HRP- or AP-coupled antibody or other marker molecule that binds to the MHC multimer. Detection of the enzyme-label is then by addition of a substrate (e.g. colorless) that is turned into a detectable product (e.g. colored) by the HRP or AP enzyme.

The solid support may be made of e.g. glass, silica, latex, plastic or any polymeric material. The support may also be made from a biodegradable material. Generally speaking, the nature of the support is not critical and a variety of materials may be used. The surface of support may be hydrophobic or hydrophilic. Non-magnetic polymer beads may also be applicable. Such are available from a wide range of manufactures, e.g. Dynal Particles AS, Qiagen, Amersham Biosciences, Serotec, Seradyne, Merck, Nippon Paint, Chemagen, Promega, Prolabo, Polysciences, Agowa, and Bangs Laboratories.

Another example of a suitable support is magnetic beads or particles. The term “magnetic” as used everywhere herein is intended to mean that the support is capable of having a magnetic moment imparted to it when placed in a magnetic field, and thus is displaceable under the action of that magnetic field. In other words, a support comprising magnetic beads or particles may readily be removed by magnetic aggregation, which provides a quick, simple and efficient way of separating out the beads or particles from a solution. Magnetic beads and particles may suitably be paramagnetic or superparamagnetic. Superparamagnetic beads and particles are e.g. described in EP 0 106 873. Magnetic beads and particles are available from several manufacturers, e.g. Dynal Biotech ASA (Oslo, Norway, previously Dynal AS, e.g. DYNABEADS®).

6. Microchip MHC Multimer Technology

A microarray of MHC multimers can be formed, by immobilization of different MHC multimers on solid support, to form a spatial array where the position specifies the identity of the MHC-peptide complex or specific empty MHC immobilized at this position. When labelled cells are passed over the microarray (e.g. blood cells), the cells carrying TCRs specific for MHC multimers in the microarray will become immobilized. The label will thus be located at specific regions of the microarray, which will allow identification of the MHC multimers that bind the cells, and thus, allows the identification of e.g. T-cells with recognition specificity for the immobilized MHC multimers. Alternatively, the cells can be labelled after they have been bound to the MHC multimers. The label can be specific for the type of cell that is expected to bind the MHC multimer, or the label can stain cells in general (e.g. a label that binds DNA). Alternatively, cytokine capture antibodies can be co-spotted together with MHC on the solid support and the cytokine secretion from bound antigen specific T-cells analyzed. This is possible because T-cells are stimulated to secrete cytokines when recognizing and binding specific MHC-peptide complexes.

7. Indirect Detection of T-Cell Using pMHC Multimers

T-cells in a sample may also be detected indirectly using MHC multimers. In indirect detection, the number or activity of T-cells are measured, by detection of events that are the result of TCR-MHC-peptide complex interaction. Interaction between MHC multimer and T-cell may stimulate the T-cell resulting in activation of T-cells, in cell division and proliferation of T-cell populations or alternatively result in inactivation of T-cells. All these mechanisms can be measured using detection methods able to detect these events.

Example measurement of activation include measurement of secretion of specific soluble factor e.g. cytokine that can be measured using flowcytometry as described in the section with flow cytometry, measurement of expression of activation markers e.g. measurement of expression of CD27 and CD28 and/or other receptors by e.g. flow cytometry and/or ELISA-like methods and measurement of T-cell effector function e.g. CD8 T-cell cytotoxicity that can be measured in cytotoxicity assays like chromium release assay's know by persons skilled in the art.

Example measurement of proliferation include but is not limited to measurement of mRNA, measurement of incorporation of thymidine or incorporation of other molecules like bromo-2′-deoxyuridine (BrdU).

Example measurements of inactivation of T-cells include but is not limited to measurement of effect of blockade of specific TCR and measurement of apoptosis.

When contacted with a diverse population of T cells, such as is contained in a sample of the peripheral blood lymphocytes (PBLs) of a subject, those tetramers containing pMHCs that are recognized by a T cell in the sample will bind to the matched T cell. Contents of the reaction is analyzed using fluorescence flow cytometry, to determine, quantify and/or isolate those T-cells having an MHC tetramer bound thereto.

B. Screening

The pMHC multimers of the disclosure can be used in a variety of different screening assays. For example, in on embodiment, a library of fluorescently-labeled peptides derived from one or more antigens is applied to pMHC multimers comprising a placeholder peptide under conditions to induce release of the placeholder peptide and binding of the antigen-derived peptides. Peptide exchange is monitored by fluorescence polarization assay. The use of placeholder peptides permits the generation of empty, peptide-receptive MHC multimers under physiological conditions. This screening approach can be used to identify peptide ligands that bind to an MHC molecule. Peptide exchange reactions can be performed in multiwell formats and under native conditions. Binding can be determined by a number of techniques, such as ELISA, which monitors the stability of the MHC structure, or by biophysical techniques that monitor peptide binding, such as fluorescence polarization. This screening approach can also be used to scan peptide sets (such as those derived from pathogen genomes, tumor-associated antigens or autoimmune antigens) for MHC ligands.

The pMHC multimers, and libraries thereof, disclosed herein can be used in a number of screening methods that allow for the convenient detection and quantification of antigen-specific binding to immune cell receptors. Such pMHC multimer libraries can allow, for example, detection of T cells specific for a given antigen, multiplex detection of T cell specificities in a given sample, matching of TCR sequence with specificity (e.g., via single cell sequencing), comparative TCR affinity determination, determination of a consensus specificity sequence of a given TCR, or mapping of antigen responsiveness of T cells against sequences of interest. The pMHC multimers can also be used in detecting natural killer (NK) cells that bear receptors specific for particular MHC I polypeptides.

The resulting pMHC multimer libraries may be used in T cell screens to determine antigen-reactive T cells as described, for example, in Simon et al, Cancer Immunol Res, 2014, 2(12):1230-1244.

In some embodiments, the disclosure provides a method for isolating a TCR-expressing cell-pMHC pairs comprises contacting a plurality of TCR-expressing cells with a pMHC multimer library as described herein; generating a plurality of compartments, wherein a compartment of the plurality comprises a TCR-expressing cell of the plurality of TCR-expressing cells bound to a pMHC of the library, thereby isolating the TCR-expressing cell-pMHC pair in the compartment. In some embodiments, the TCR-expressing cell is a T cell, e.g., a CD8+ T cell when using a pMHCI multimer library or a CD4+ T cell when using a pMHCII multimer library. In some embodiments, a cell can be transfected or transduced to express a TCR. In some embodiments, a non-lymphocyte cell can be transfected or transduced to express TCR.

C. Methods of Identifying

The pMHC multimers of the disclosure can be used to identify antigen-specific T cells of interest, for example by screening a plurality of T cells with a library of pMHCI multimers. In various embodiments, the library comprises pMHC Conjugated Multimers loaded with a diversity of more than 10, more than 100, more than 500, 1000, more than 2,000, more than 5,000, more than 10,000, more than 10⁶, more than 10⁷, more than 10⁸, more than 10⁹, or more than 10¹⁰ unique peptides. The identification approach can comprise compartmentalizing a cell of the plurality of cells bound to a pMHC multimer of the library in a single compartment, wherein the pMHC multimer comprises a unique identifier; and determining the unique identifier for each pMHC multimer bound to the compartmentalized cell. A compartment can be a separate space, e.g., a well, a plate, a divided boundary, a phase shift, a vessel, a vesicle, a cell, etc.

In some embodiments, the compositions and methods disclosed herein can be used to identify a plurality of peptides that bind to a TCR. In some embodiments, the compositions and methods disclosed herein can be used to identify a plurality of TCRs that bind a pMHC. In some embodiments, the compositions and methods disclosed herein can be used to identify a plurality of TCRs that bind a plurality of pMHCs (for example, a plurality of TCRs that bind to pMHC multimers derived from a pathogen library, cancer library, or autoimmune library).

In some embodiments, the compositions and methods disclosed herein are used for identifying TCR-antigen specificity.

In some embodiments, the identity of a TCR on a selected T cell is determined by sequencing (e.g., sequencing a variable, hypervariable region or complementarity determining region (CDR) of a TCR). In some embodiments, the identity of the peptide of the pMHC bound which binds to a TCR is determined by sequencing (e.g., using an identifier as disclosed herein).

In one embodiment, pMHC multimers of the disclosure can be used for the detection of antigen-specific T cells by flow cytometry or for can be used for T-cell purification. The compositions and methods of the disclosure allow for the production of very large collections of peptide-loaded MHC multimers that are well suited for rapid identification of cytotoxic T-cell (i.e., CD8+ T cell) antigens when using pMHCI multimers and helper T cell (i.e., CD4+ T cell) antigens when using pMHCII multimers.

In one embodiment, pMHC multimers that are attached to solid surfaces can be used to probe T cell function. The peptide-MHC antigenic complexes fixed to the solid surface can function to stimulate T cell activity through the TCR, thereby allowing for study of downstream T cell functions subsequent to TCR stimulation.

In some embodiments, the compositions and methods disclosed herein are used to determine how mutations in an identified MHC-binding peptide affect TCR binding. In some embodiments, the compositions and methods disclosed herein are used to identify mutations in an identified MHC-binding peptide that result in enhanced or reduced TCR binding affinity. In some embodiments, the compositions and methods disclosed herein are used to identify mutations in an identified MHC-binding peptide that retain TCR binding affinity. In some embodiments, the compositions and methods disclosed herein are used to identify mutations in an identified MHC-binding peptide that result in loss of TCR binding affinity.

In some embodiments, the compositions and methods disclosed herein are used to determine how mutations in a TCR identified using the methods described herein alter the binding of a peptide epitope. In some embodiments, the compositions and methods disclosed herein are used to identify mutations in a TCR that result in decreased or increased binding affinity for a peptide epitope. In some embodiments, the compositions and methods disclosed herein can be used to identify mutations in a TCR that retain binding of a peptide epitope. In some embodiments, the compositions and methods disclosed herein can be used to identify mutations in a TCR that result in loss of binding of a peptide epitope.

In some embodiments, the methods disclosed herein are performed on T cells from a plurality of subjects. In some embodiments, analysis of data from multiple subjects allows identification of MHC-binding peptide epitopes recognized by multiple subjects. In some embodiments, analysis of data from multiple subjects allows identification of MHC-binding peptide epitopes recognized by multiple TCR clonotypes. In some embodiments, analysis of data from multiple subjects allows identification of MHC-binding peptide epitopes recognized by multiple patients, e.g., multiple cancer patients, multiple patients with an autoimmune condition, or multiple patients with protective immunity against a pathogen. In some embodiments, analysis of data from multiple subjects allows identification of MHC-binding peptide epitopes recognized in subjects comprising different HLA types or alleles. In some embodiments, analysis of data from multiple subjects allows identification of distinct hypervariable or complementarity determining region sequences of TCRs that exhibit convergent antigen binding.

In some embodiments, the methods disclosed herein are performed using a plurality of libraries. In some embodiments, analysis of data from multiple libraries allows identification of shared reactive MHC-binding peptide epitopes between libraries, e.g., antigens exhibiting TCR affinity that are present in multiple strains of a pathogen, multiple cancer types, multiple cancer patients, multiple autoimmune diseases, or multiple autoimmune conditions. In some embodiments, analysis of data from multiple libraries allows identification of distinct reactive MHCI-binding peptide epitopes among libraries, e.g., antigens present in a subset of pathogen strains, cancers, conditions, or patients.

In some embodiments, T cells identified using a pMHC multimer library of the disclosure are subjected to gene expression analysis (e.g., RNA-seq, qPCR). In some embodiments, gene expression analysis is conducted on cells identified as possessing a receptor exhibiting specificity for a peptide in a library of the disclosure. For example, cells determined to express TCRs that bind to a pMHC multimer derived from a pathogen library, cancer library, or autoimmune library are subjected to gene expression analysis. Gene expression analysis can be global or targeted. Genes analyzed for expression include, but are not limited to, genes with known functions, genes coding for immune effector molecules (e.g., perforin, granzyme, cytokines, chemokines), immune checkpoint molecules, pro-inflammatory molecules, anti-inflammatory molecules, lineage markers, integrins, selectins, lymphocyte memory markers, death receptors, caspases, cell cycle checkpoint molecules, enzymes, phosphatases, kinases, lipases, and metabolic genes.

In some embodiments, gene expression analysis can be conducted concurrently with pMHC multimer library screening. In some embodiments, gene expression analysis can be conducted after analysis of pMHC multimer library screening results. In some embodiments, gene expression analysis can be conducted before analysis of pMHC multimer library screening results. In some embodiments, gene expression analysis allows for immunotyping of cells identified as of interest from pMHC-T cell receptor pairings produced using the methods described herein.

The methods and compositions described herein can be used for screening assays. For example, a library comprising a plurality of pMHC multimers as described herein is contacted with a T cell sample, and one or more T cell functions are determined including, but not limited to, T cell proliferation, T cell cytotoxicity, suppression of T cell proliferation, suppression by a T cell, and cytokine production of a T cell.

In some embodiments, pMHC multimers that can induce the functional property can then be made into a peptide library subset. For example, a library subset can comprise pMHC multimers that induce proliferation of a T cell upon binding to TCR, cytotoxicity upon binding to TCR, T cell suppression upon binding to TCR, suppression by a T cell upon binding to TCR, cytokine production upon binding to TCR, or any combination thereof. Proliferation can be determined by, for example, a dye-dilution assay (e.g., CFSE dilution assay), or quantification of DNA replication (e.g., BrdU incorporation assay). Cytotoxicity can be determined by, for example, assays that are based on release of an intracellular enzyme by dead cells (e.g., lactate dehydrogenase), dye exclusion assays (e.g., propidium iodide), or expression of cytolytic markers (e.g., granzyme, CD107a) by flow cytometry or qPCR. Cytokine production can be determined by, for example, ELISA, multiplex immunoassay, intracellular cytokine staining, ELISPOT, Western Blot, or qPCR. T cell suppression can be determined by, for example, co-incubating a T cell clone with effector cells and target antigen, and measuring proliferation, cytotoxicity, cytokine production, expression of activation markers, etc.

In some embodiments, the compositions and methods disclosed herein are used to identify antigen-specific T cell effector clones associated with protective immunity, non-protective immunity, or autoimmunity. In some embodiments, compositions and methods disclosed herein are used to identify antigen-specific T cell effector clones that exhibit anergy, exhaustion, tolerogenic properties, autoimmune properties, inflammatory properties, or anti-inflammatory properties (e.g., Tregs). In some embodiments, compositions and methods disclosed herein are used to identify antigen-specific T cell effector clones that exhibit certain effector or memory properties (e.g., naïve, terminal effector, effector memory, central memory, resident memory, T_{H}1, T_{H}2, T_{H}17, T_{H}9, T_{C}1, T_{C}2, T_{C}17, production of certain cytokines).

In some embodiments, a TCR identified using compositions and methods disclosed herein are used as part of a therapeutic intervention. For example, a TCR sequence, TCR variable region sequence, or CDR sequence can be transfected or transduced into T cells to generate modified T cells of the same antigenic specificity. The modified T cells can be expanded, polarized to a desired effector phenotype (e.g., T_{H}1, T_{C}1, Treg), and infused into a subject. In some embodiments, multiple TCRs identified using compositions and methods disclosed herein are used in an oligoclonal therapy.

In some embodiments, a peptide, ligand, agonist, antagonist, antigen, or epitope identified using methods disclosed herein is used as part of a therapeutic intervention. In some embodiments, a peptide, antigen, or epitope is used to expand a population of cells ex vivo, e.g. using antigen presenting cells, artificial antigen presenting cells, immobilized peptide, or soluble peptide. In some embodiments, expanded cells are infused into a patient. In some embodiments, peripheral blood lymphocytes are expanded. In some embodiments, tumor-infiltrating lymphocytes (TILs) are expanded. In some embodiments, T_{H}1 cells are expanded. In some embodiments, cytotoxic T lymphocytes are expanded. In some embodiments, T regulatory cells are expanded.

In some embodiments, the compositions and methods disclosed herein are used to identify MHC-binding antigenic peptides for use in development of a vaccine, e.g. a subunit vaccine, a vaccine eliciting coverage against a range of protective antigens, or a universal vaccine.

In some embodiments, the compositions and methods disclosed herein can be used for diagnosis of a medical condition. In some embodiments, the compositions and methods disclosed herein are used to guide clinical decision making, e.g. treatment selection, identification of prognostic factors, monitoring of treatment response or disease progression, or implementation of preventative measures.

In some embodiments, the compositions and methods disclosed herein can be used in the selection and/or design of treatments for medical conditions, in particular in the selection of antigen-specific T cells (e.g., CD8+ cytotoxic T cells and/or CD4+ helper T cells), or TCRs derived therefrom, for use in adoptive transfer T cell therapy. For example, the pMHC Conjugated Multimers can be used to identify T cells within a patient sample the react to an antigen(s) of interest, such as a cancer antigen(s) or pathogen antigen(s) to thereby select those cells for expansion in vitro followed by reintroduction into the patient. Moreover, TCRs identified from such antigen-specific T cells can be sequences and recombinantly introduced into T cells to increase the population of cells expressing TCRs that bind to an antigen(s) of therapeutic interest in a patient.

### EXAMPLES

Below are examples of specific embodiments for carrying out the present invention. The examples are offered for illustrative purposes only and are not intended to limit the scope of the present invention.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al, Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

Unless otherwise stated, all reagents and chemicals were obtained from commercial sources and used without further purification.

### Example 1—Design of a Peptide-Loadable Barcode-Exchangeable MHCI Tetramer Expression Construct

In this example, an expression construct was prepared that encodes an exchangeable HLA-A*02:01-binding peptide, an MHC Class I alpha (heavy) chain (HLA-A*02:01), a β2-microglobulin (β2m) chain and a tetramerization domain (streptavidin). A schematic diagram of the construct is shown in FIG. 1. The nucleotide sequence of the coding region of the expression construct is shown in SEQ ID NO: 1. The complete amino acid sequence of the encoded MHCI multimer polypeptide, including signal sequence and tags is shown in SEQ ID NO: 2. The amino acid sequence of the encoded MHCI multimer polypeptide without signal sequence and tags is shown in SEQ ID NO: 3. The From 5′ to 3′, the nucleic acid construct encodes: (i) the Ig Kappa chain V-III region CLL signal peptide, which facilitates the secretion of the tetramer in human cells; (ii) the HLA-A*02:01 restricted CMV pp65 epitope NLVPMVATV (SEQ ID NO: 4); (iii) human beta-2-microglobulin; (iv) the soluble domain of HLA-A*02:01 (residues 25-302; SEQ ID NO: 5); and (v) streptavidin.

The CMV pp65 peptide epitope is operatively linked to the N terminus of the human beta-2-microglobulin via a linker containing a Factor Xa cleavage site in the center of the linker. Cleavage of the expression product by Factor X results in the native CMV pp65 peptide epitope with a portion of the linker upstream of the Factor Xa site attached to its C terminus, which promotes dissociation of the CMV pp65 peptide epitope from the HLA peptide groove. The C terminus of the human beta-2-microglobulin is connected to the N terminus of the soluble domain of HLA-A*02:01 via a standard (G₄S)₄ linker. The C terminus of the soluble HLA-A*02:01 domain is linked to streptavidin, which facilitates the tetramerization of the protein, with a (GS)₂AG₂SGSG₃S linker in between the two polypeptides. The C terminus of streptavidin is followed by a 6×His tag and FLAG tag for purification and detection.

### Example 2—Screening of Candidate MHCI Tetramer Constructs

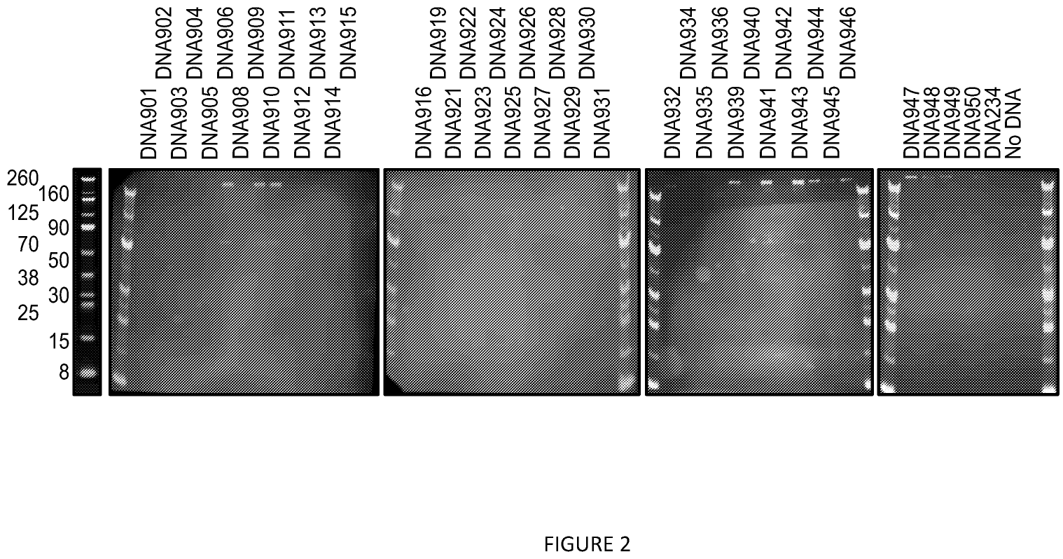

Expression plasmids encoding various pMHCI tetramers were transfected individually into Expi293™ human embryonic kidney (HEK) cells using the Expi293™ Transfection Kit (ThermoFisher Scientific) according to kit protocols. Six days post-transfection, supernatant from individual cultures were reduced and boiled and resolved on a 4-12% Bis-Tris polyacrylamide gel. Proteins were subsequently transferred onto a nitrocellulose membrane and the membrane was blocked using Intercept® Blocking Buffer (Licor) on a rocker. The membrane was then blotted with Dylight-800™ conjugated anti-FLAG antibody, diluted 1:1000 in the blocking buffer, for 1 hour at room temperature on a rocker. The membrane was washed three times in 1×PBS for 5 min each on a rocker and imaged using the Licor Odyssey® Fc instrument. As shown in FIG. 2, a band corresponding to the apparent size of the tetramer was seen for candidate constructs, demonstrating successful expression of the construct. Despite reducing conditions and boiling, the tetrameric structure was maintained due to the stabilization of the streptavidin by biotin-binding, which biotin was present in the culture medium during expression.

### Example 3—Biotin-Mediated Barcode-Labeling of MHCI Multimers

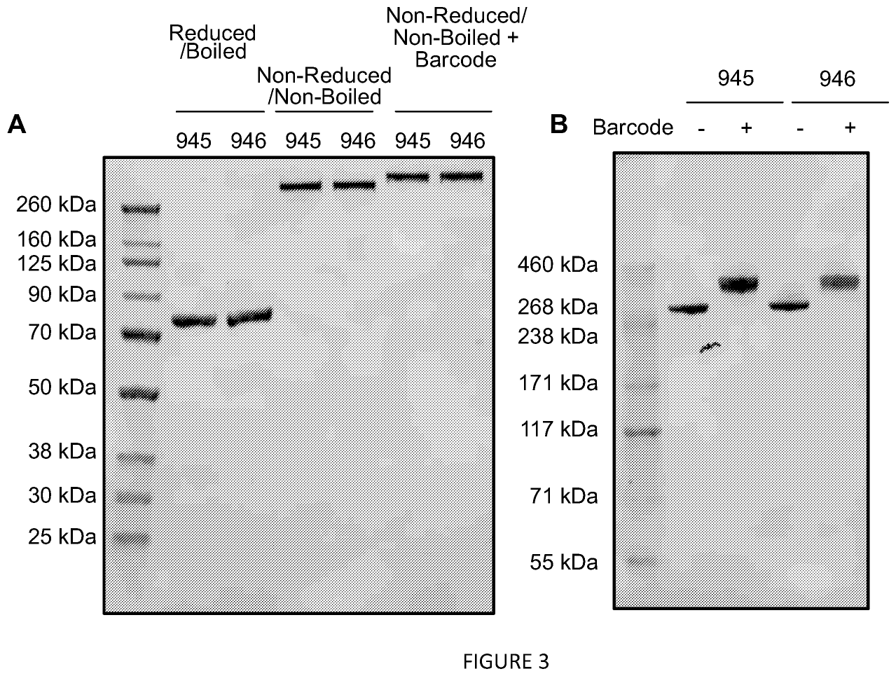

Candidate expression construct plasmids (as described in Example 1) were transfected individually into Expi293™ cells using the Expi293™ Transfection Kit according to kit protocols. Immediately prior to adding the transfection complexes to cells, avidin was added to 2.5 uM in the culture medium to quench free biotin present in the media during expression, allowing for the production of tetramers with free biotin-binding sites in the streptavidin. Six days post-transfection, the supernatant from the cultures were harvested by centrifugation followed by 0.45 um filtration. Standard IMAC purification was performed on the supernatants followed by a size-exclusion polishing step. The purification of the tetramers was confirmed by SDS-PAGE followed by Coomassie staining. As shown in FIG. 3A, on a 4-12% Bis-Tris polyacrylamide gel, when non-reduced and non-boiled, a band corresponding to the apparent size of the tetramer was observed, whereas when boiled and reduced, a band corresponding to the size of the monomer was observed. Furthermore, the ability to reduce the tetramer into its monomeric species suggested that the biotin-binding pocket of the streptavidin was unoccupied. To confirm that the biotin-binding pockets were indeed empty (and thus available for use in barcoding), tetramers were incubated with a single-stranded DNA barcode with a biotin molecule conjugated to its 5′ end. The tetramer and the barcode were combined in a 1:2 tetramer: barcode molar ratio and incubated on ice for 1 hour. As shown in FIG. 3A and FIG. 3B, when resolved by SDS-PAGE, the barcoded tetramer had an increased apparent molecular weight as compared to the unbarcoded tetramer. This molecular weight difference was more prominent when the proteins were run on a low percentage gel such as a 3-8% Tris-Acetate polyacrylamide gel (FIG. 3B).

### Example 4—Factor Xa Cleavage and Peptide Exchange of pMHCI Tetramers

500 nM of pMHCI tetramers, prepared as described in Examples 1-3, were incubated with 2 ug of Factor Xa in the presence of 2 mM CaCl₂ and 60 uM of individual HLA-A*02:01-restricted peptide epitopes, including MART-1 (EAAGIGILTV; SEQ ID NO: 6), HPV (YMLDLQPETT; SEQ ID NO: 7), HSV (SLPITVYYA; SEQ ID NO: 8), and WT-1 (RMFPNAPYL; SEQ ID NO: 9). This panel of peptides span a range of binding affinities for HLA-A*02:01 according to netMHC, from 5.9 nM to 8.5 uM. The mixture was incubated for 3 hours at room temperature, overnight at 4° C., 3 hours at room temperature, and 90 minutes at 30° C. Overnight incubation ensured complete Factor Xa cleavage and subsequent incubation at 30° C. promoted the exchange of the native CMV peptide for peptides of interest. As additional controls, untreated tetramers and digested tetramers in the absence of peptide were included.

### Example 5—Confirmation of Digestion and Peptide Exchange of pMHCI Multimers by Specific Cell Staining

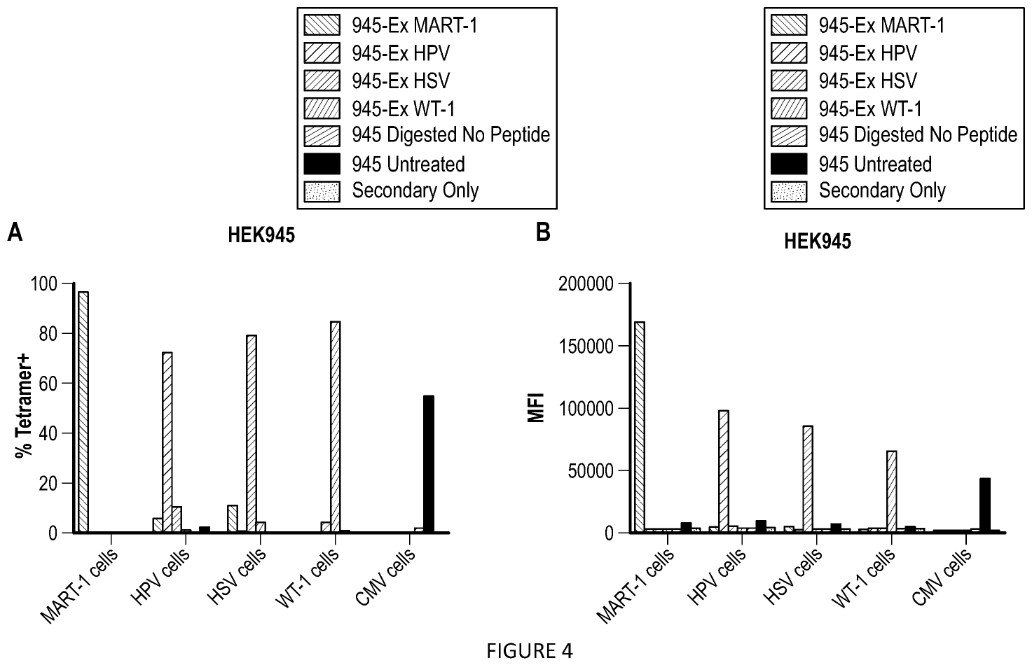

Specific cell staining was performed to confirm the Factor Xa digestion and peptide exchange that was carried out as described in Example 4. Exchanged tetramers were used to stain antigen-specific CD8+ T cells corresponding to each of the peptides. Antigen-specific CD8+ T cells were seeded in a 96-well plate at 100K cells/well and washed once in FACS buffer (1×PBS+2% FBS). Cells were resuspended in 5 nM of tetramers, diluted in FACS buffer, for 20 min at 4° C. Cells were washed twice in FACS buffer and resuspended in PE-conjugated anti-streptavidin antibody, used 1:50 diluted in FACS buffer. Cells were incubated for 20 min at 4° C. then washed once in FACS buffer and once in 1×PBS. Cells were then resuspended in Fixable Viability Dye eFluor780, used 1:8000 diluted in PBS, and incubated at 4° C. for 10 min. Cells were washed twice in FACS buffer and fixed in Fixation buffer (FACS buffer+4% Paraformaldehyde). Cells were read on a flow cytometer (Sartorius Intellicyte iQue Screener Plus). As shown in FIG. 4A (expressed as % tetramer binding) and FIG. 4B (expressed as mean fluorescence intensity; MFI), antigen-specific T cells demonstrated robust binding only to the tetramers that have been exchanged with its cognate peptide suggesting successful exchange. More importantly, all exchanged tetramers lost reactivity towards CMV-specific cells suggesting that the Factor Xa digestion and dissociation of the native peptide was complete.

### Example 6—Confirmation of Digestion and Peptide Exchange of pMHCI Multimers by Differential Scanning Fluorimetry

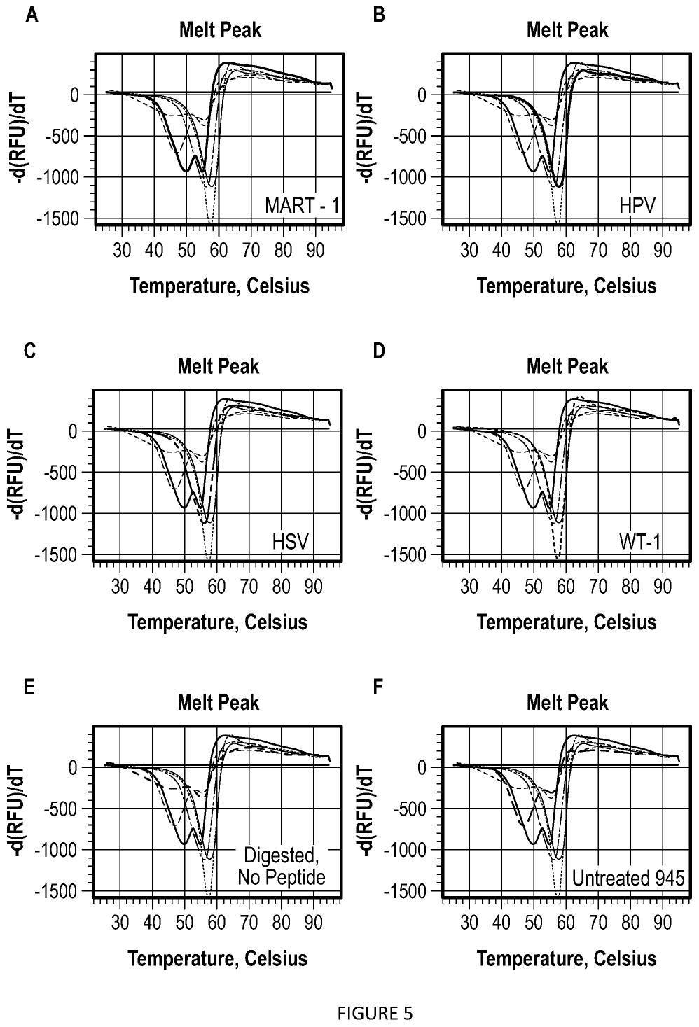

Factor Xa digestion and peptide exchange was carried out with pMHC multimers prepared as described in Examples 1-3, and differential scanning fluorimetry (DSF) was performed to confirm digestion and peptide exchange. 2 uM of tetramers were incubated with 2 ug of Factor Xa in the presence of 2 mM CaCl₂) and 240 uM of individual peptides of interest, including MART-1 (EAAGIGILTV; SEQ ID NO: 6), HPV (YMLDLQPETT; SEQ ID NO: 7), HSV (SLPITVYYA; SEQ ID NO: 8), and WT-1 (RMFPNAPYL; SEQ ID NO: 9). This mixture was incubated for 3 hours at room temperature, overnight at 4° C., 3 hours at room temperature, and 90 minutes at 30° C. As additional controls, untreated tetramers and digested tetramers in the absence of peptide were included. 18 ul of the exchanged tetramers were mixed with 2 ul of 100×Sypro orange dye, resulting in a final concentration of 10× dye. The mixture was then subjected to a 0.05° C./s ramp from 25° C. to 99° C. in a qPCR instrument. A peak in the first derivative of the melt curve indicates the Tm of the tetramer. As shown in FIG. 5A-5F, when digestion and exchange occurred in the presence of another peptide, stabilization of the tetramer was observed, demonstrated by a single defined peak with an increase in the Tm suggesting successful exchange of the native NLV peptide for the peptides of interest. In contrast, digestion and exchange of the tetramer in the absence of peptide or the untreated tetramer alone lacked a well-defined and characteristic peak. Exchange with the MART-1 peptide EAAGIGILTV (SEQ ID NO: 6) showed two distinct peaks, an observation previously seen with this peptide on the HLA-A*02:01 allele.

### Example 7—Stability of Exchangeable HLA-A*02:01 Tetramers

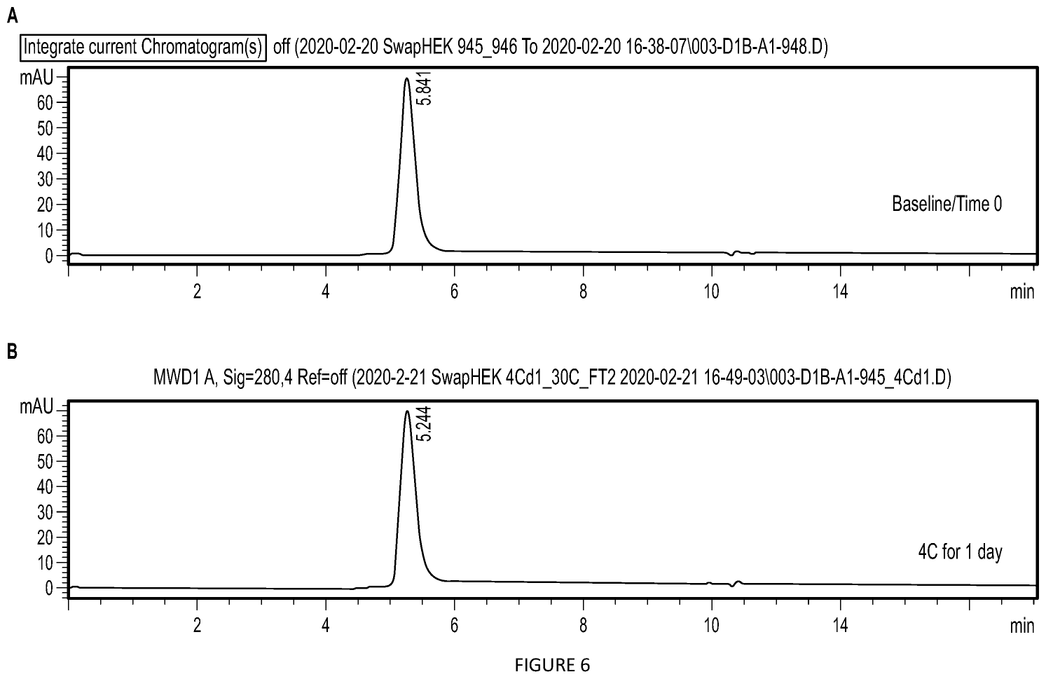

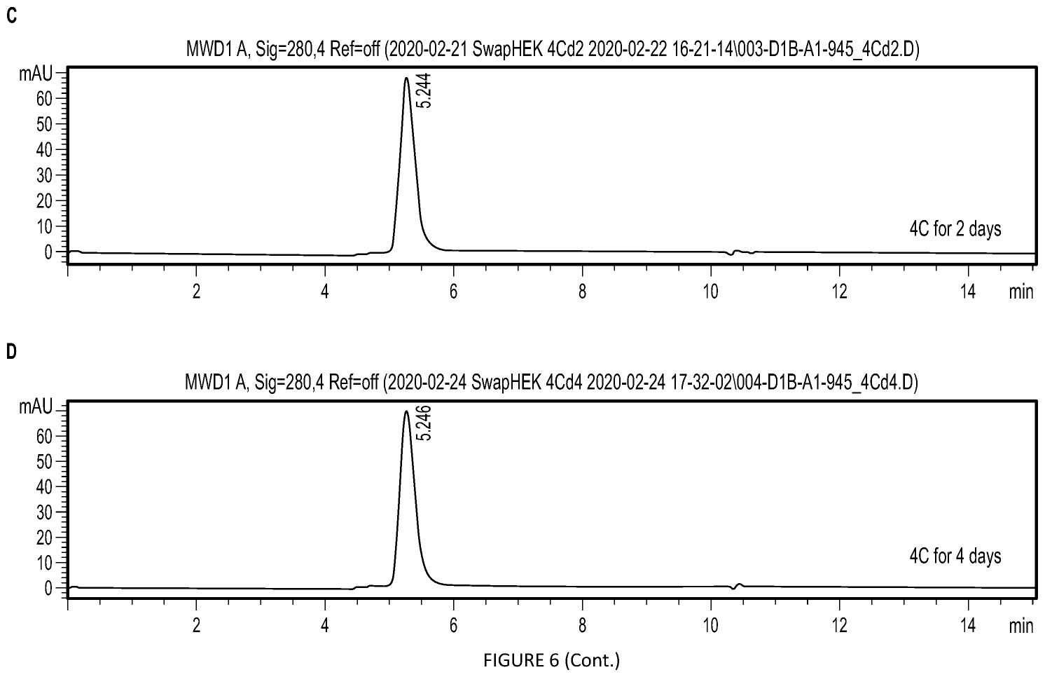

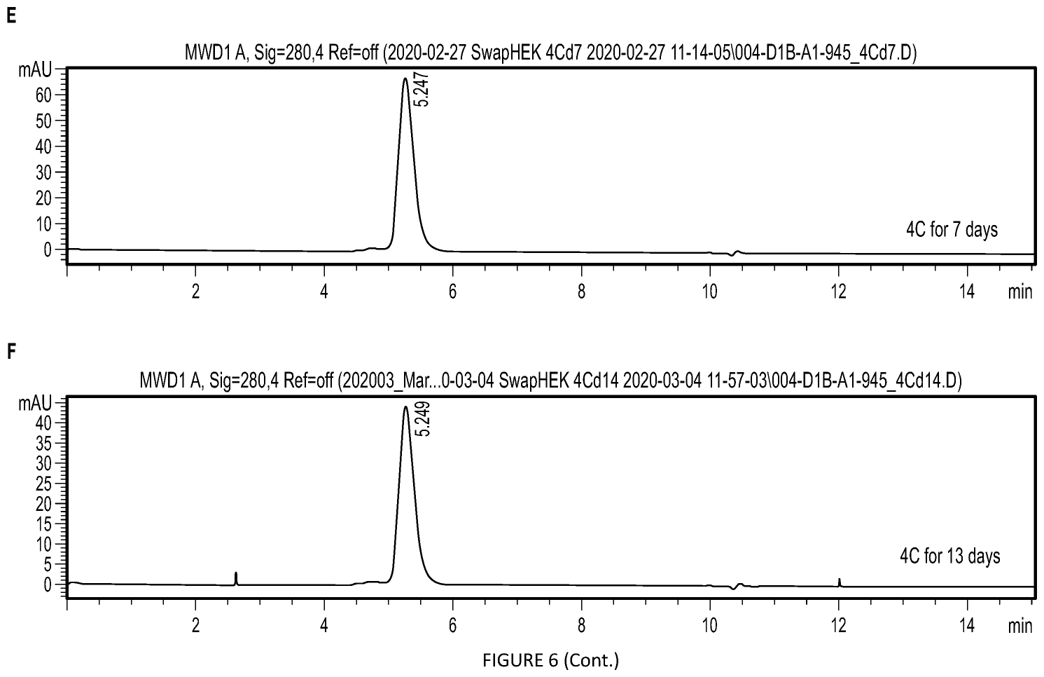

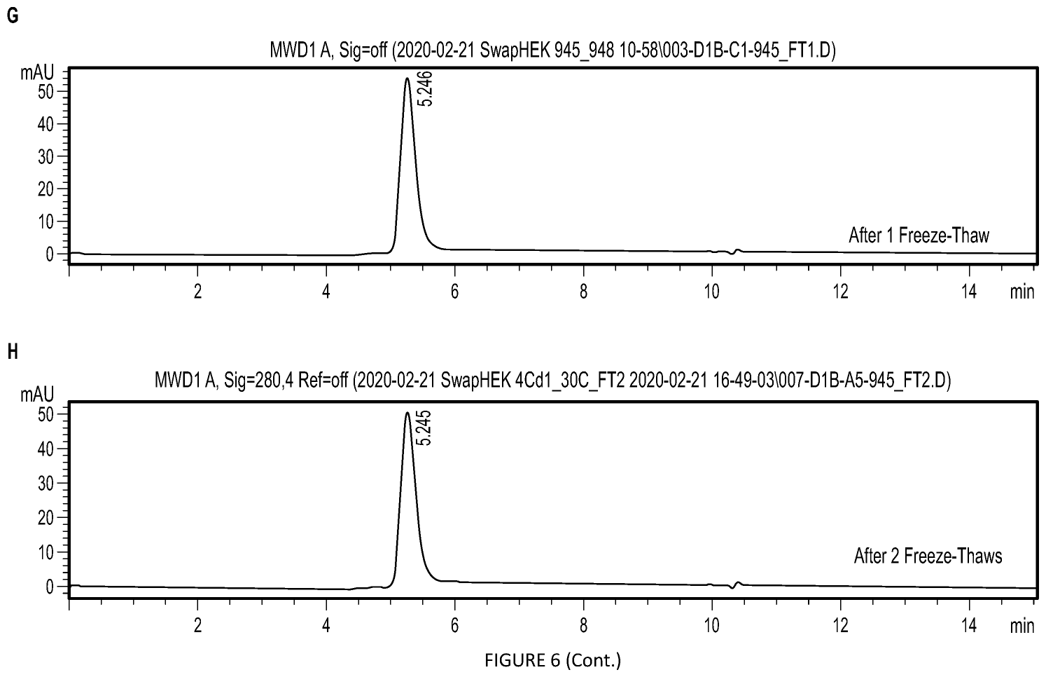

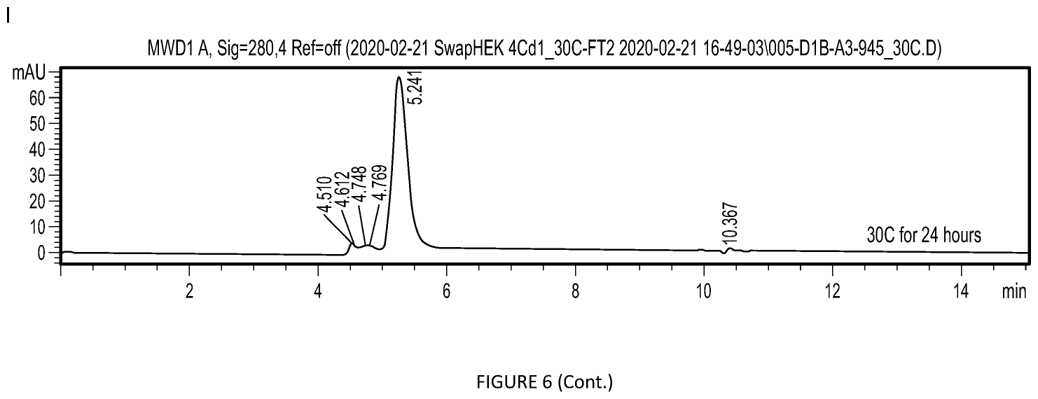

Exchangeable HLA-A*02:01 tetramers at 2.328 mg/mL in PBS were treated under various conditions and analyzed for the change in the percentage of tetrameric species by analytical size-exclusion chromatography. As shown in FIG. 6A-6I, no change in the percentage of tetrameric species was observed during storage at 4° C. for up to 13 days, nor after two freeze-thaw cycles. A very marginal decrease in the percentage of tetrameric species was observed when incubated at 30 C for 24 hours. The results in FIG. 6A-6I are summarized below in Table 1.

TABLE 1
Stability of pMHCI Tetramers Under Various Conditions

| Condition | % Tetramer |
|---|---|
| Baseline/Time 0 | 100% |
| 4° C. for 1 day | 100% |
| 4° C. for 2 days | 100% |
| 4° C. for 4 days | 100% |
| 4° C. for 7 days | 100% |
| 4° C. for 13 days | 100% |
| After 1 round of freeze/thaw | 100% |
| After 2 rounds of freeze/thaw | 100% |
| 30° C. for 24 hours | 93.4% |

Taken together, the data demonstrate that the exchangeable HLA-A*02:01 tetramers are highly stable.

### Example 8—Stability of HLA-A*02:01 Tetramers after Peptide Exchange

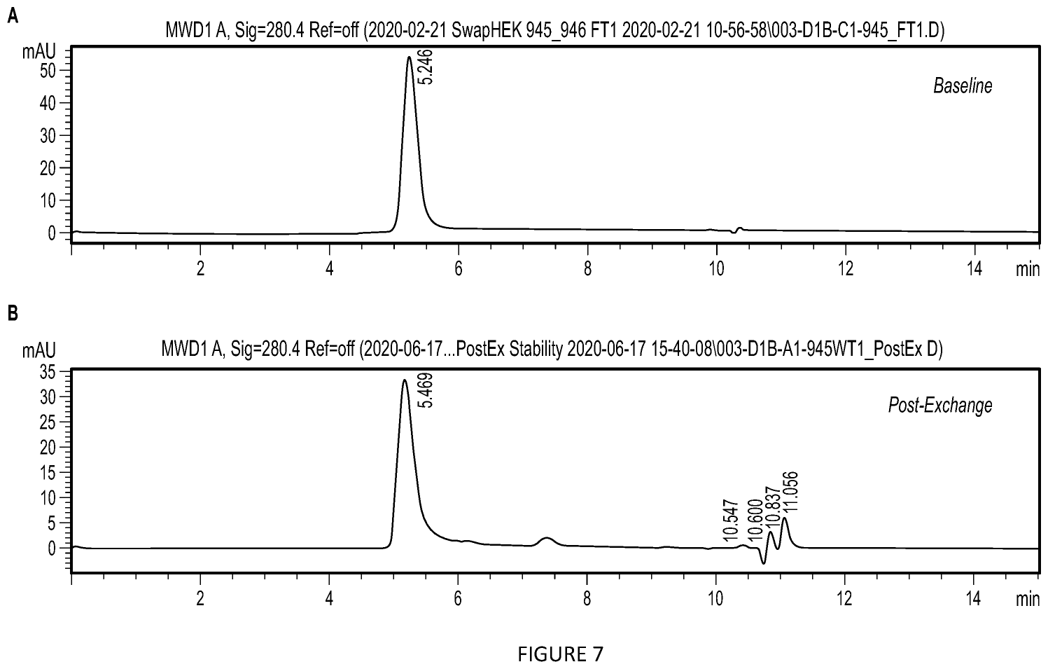

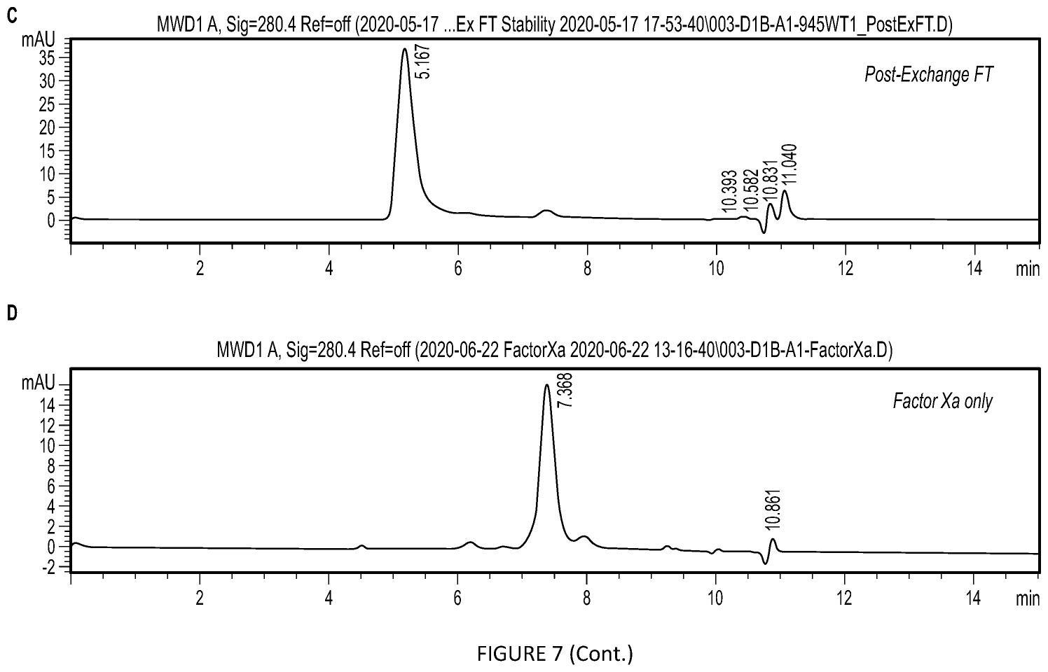

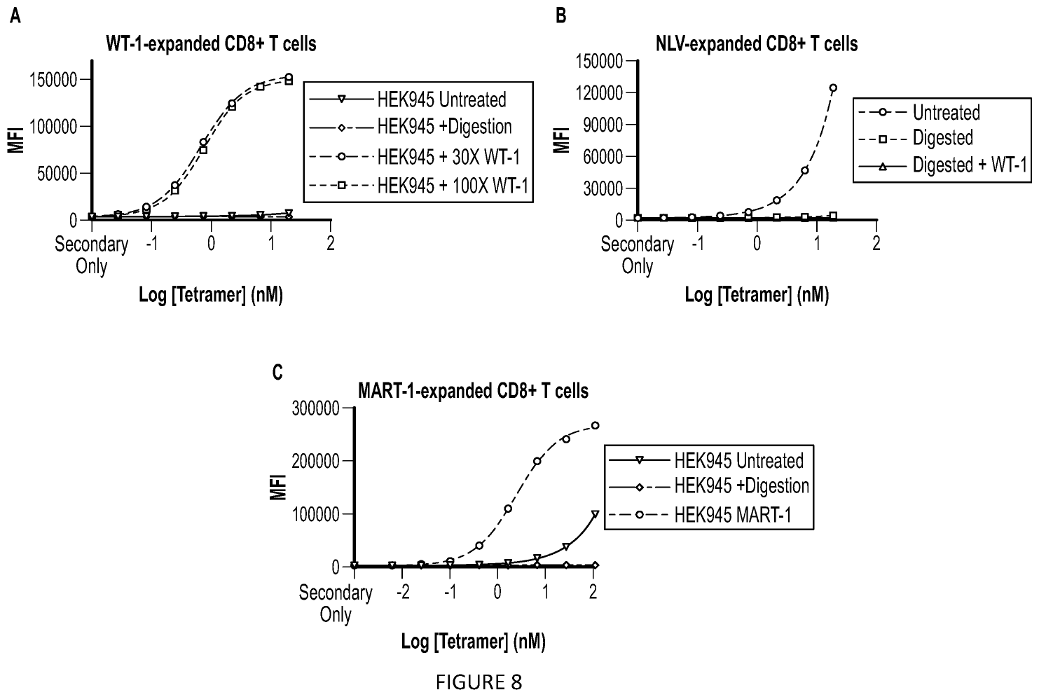

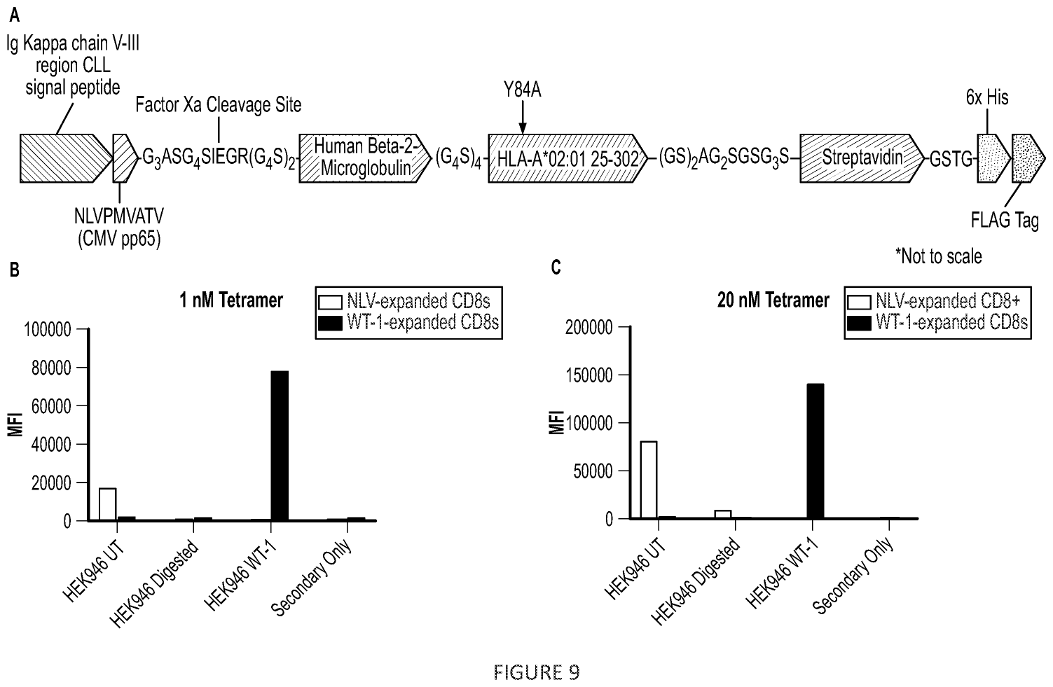

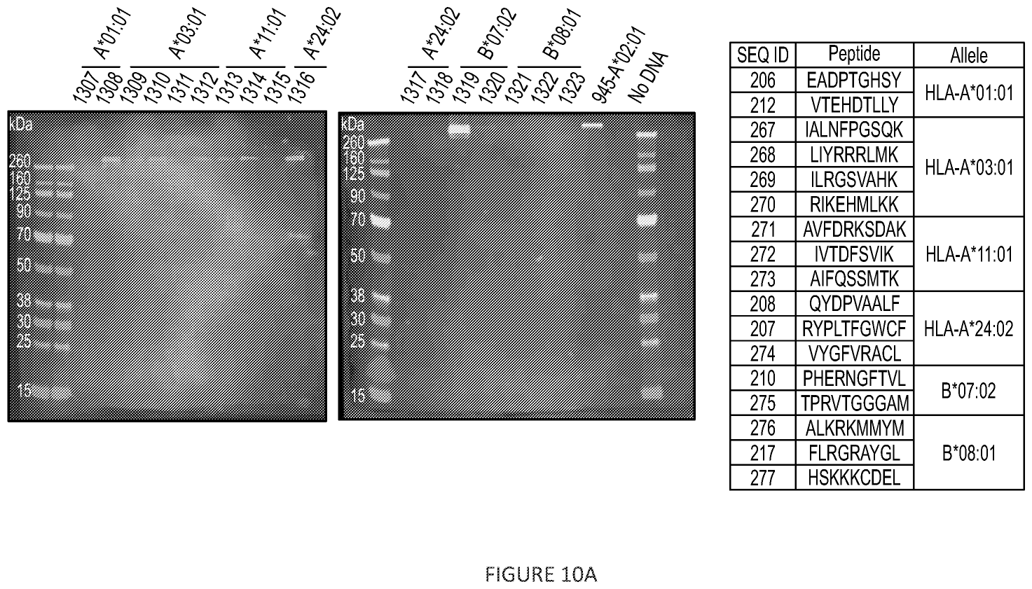

Exchangeable HLA-A*02:01 tetramers were digested and peptide-exchanged as described in Example 4, and analyzed for the change in the percentage of tetrameric species by analytical size-exclusion chromatography. As shown in FIG. 7A-7C, no change in the percentage of tetrameric species was observed after the exchange protocol, nor after one freeze-thaw cycle. A smaller species that arises upon digestion is consistent with the presence of Factor Xa, as seen in the chromatogram in FIG. 7D.

### Example 9—Confirmation of Digestion and Peptide Exchange of pMHCI Multimers by Titration on Antigen-Specific T Cells

Activity of exchanged tetramers was further confirmed by specific cell staining using titrations of untreated, digested and exchanged tetramers, following the procedure outlined in Examples 4 and 5. As shown in FIG. 9A and FIG. 9B, upon digestion with Factor Xa, binding to NLV-specific T cells is lost. Incubation with either 30× or 100× excess of WT1 peptide rescued strong and specific binding to WT-1-specific T cells. Similarly, in FIG. 9C, Factor Xa-digested tetramers show no binding to MART-1-specific T cells. Strong binding to these cells occurs upon further exchange with 30× excess of the MART-1 peptide.

### Example 10—Confirmation of Digestion and Peptide Exchange of pMHCI Multimers Featuring a Single-Chain-Stabilizing Mutation by Specific Cell Staining

A Y84A variant of the A*02:01 tetramer (shown schematically in FIG. 9A) was produced, digested and subjected to WT-1 peptide exchange as shown in Examples 4 and 5. The amino acid sequence of the Y84A HLA alpha chain is shown in SEQ ID NO: 321. This mutation is known in the art to stabilize binding of the tethered peptide by reducing steric conflict with the peptide linker. Staining of untreated (UT), digested, and WT-1-exchanged tetramers on NLV- and WT-1-specific CD8+ T cells at 1 nM (FIG. 9B) and 20 nM (FIG. 9C) confirmed that efficient peptide exchange occurred, converting NLV reactivity to WT-1 reactivity.

### Example 11—Screening of HLA-A, -B and -C Alleles as Candidate pMHCI Tetramers

56 additional common MHCI alleles were generated using designs similar to FIG. 1, but with unique peptides and swapped heavy chains. These were transiently expressed in HEK cells, and supernatants were visualized by anti-Flag Western blots as in Example 2. The results are shown in FIG. 10A-D, which also show the HLA allele/peptide sequence combinations. Bands of the correct size were observed for roughly half of the alleles, indicating successful expression of tetramers with the indicated peptides using the standard format.

### Example 12—Screening Functionality of HLA-A, -B and -C Alleles as Candidate pMHCI Tetramers Using Conformation-Dependent ELISA

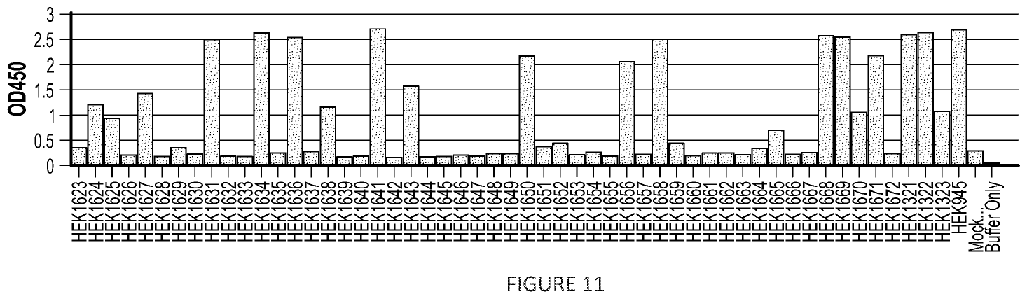

The transient expression supernatants produced as in Example 11 were screened using an ELISA format. Maxisorp plates were coated with W6/32 antibody at 100 ng/well during an overnight incubation at 4° C. Plates were blocked with 200 ul of Blocking Buffer (PBST+2% BSA) for 2 hours at room temperature. Transient HEK supernatant samples were added to the wells and incubated at room temperature for 1 hour, followed by detection with HRP-conjugated Anti-human B2M (Biolegend #280303). Because W6/32 is a conformationally-sensitive antibody that only recognizes peptide-loaded MHCI, signal in this ELISA format indicates tetramers are correctly folded. As seen in FIG. 11, nearly half of the supernatants detected gave positive signal, corroborating the Western but also providing evidence of proper folding and peptide presentation.

### INCORPORATION BY REFERENCE

Each patent, publication, and non-patent literature cited in the application is hereby incorporated by reference in its entirety as if each was incorporated by reference individually.

SEQUENCE LISTING SUMMARY

| SEQ | |
|---|---|
| ID | |
| NO: | DESCRIPTION |
| 1 | ATGGAGGCTCCGGCTCAGCTGCTGTTCCTTCTGCTGCTGTGGCTGCCCGACACCACCGGA |
| | AATTTGGTCCCGATGGTTGCAACGGTTGGCGGAGGGGCGTCTGGGGGCGGTGGTAGTATA |
| | GAAGGACGAGGCGGTGGCGGAAGTGGTGGTGGAGGCTCTATCCAACGCACCCCTAAAAT |
| | CCAGGTCTACTCGAGACACCCGGCTGAGAACGGGAAGTCCAACTTCCTGAACTGCTACGT |
| | GTCCGGTTTTCACCCGTCCGACATTGAGGTGGACCTCCTGAAGAACGGAGAGCGCATCGA |
| | GAAGGTGGAACACTCCGACCTTAGCTTCTCCAAGGATTGGTCATTCTACCTGTTGTACTAC |
| | ACCGAGTTCACTCCGACCGAAAAGGACGAATACGCATGCAGGGTGAACCACGTGACCCT |
| | GTCCCAGCCGAAGATCGTGAAGTGGGACCGGGACATGGGAGGCGGCGGATCAGGAGGC |
| | GGAGGATCTGGGGGTGGAGGAAGCGGTGGTGGCGGATCCGGAAGCCACTCCATGCGGTA |
| | CTTCTTCACCTCCGTGTCACGCCCTGGTCGGGGAGAGCCTCGATTCATCGCCGTCGGCTAC |
| | GTGGACGACACTCAGTTCGTCCGCTTTGATTCGGACGCTGCAAGCCAGCGGATGGAACCA |
| | AGGGCGCCTTGGATCGAACAGGAGGGCCCCGAGTACTGGGACGGGGAAACTCGGAAAGT |
| | GAAGGCCCACTCTCAGACTCACCGGGTGGATCTCGGGACGCTCAGAGGCTACTACAACC |
| | AGTCAGAGGCCGGCAGCCATACTGTCCAACGGATGTACGGATGCGACGTGGGCTCCGAT |
| | TGGAGGTTCCTGAGAGGATACCATCAGTACGCGTACGACGGAAAGGACTATATCGCGCT |
| | CAAGGAGGACCTGAGATCCTGGACTGCGGCCGATATGGCCGCTCAGACGACTAAACACA |
| | AGTGGGAAGCAGCTCACGTGGCCGAGCAGCTGAGGGCCTACCTGGAGGGAACTTGCGTC |
| | GAGTGGCTGCGGAGATATCTGGAGAATGGGAAGGAAACCCTCCAGAGGACAGATGCACC |
| | CAAGACCCATATGACTCACCATGCCGTGAGCGACCACGAAGCCACCCTGCGGTGTTGGGC |
| | CCTGTCCTTCTACCCGGCCGAAATCACGCTGACCTGGCAACGCGATGGAGAGGACCAGAC |
| | CCAAGACACTGAACTCGTGGAAACCAGACCCGCGGGAGATGGCACCTTCCAAAAGTGGG |
| | CCGCTGTGGTGGTCCCGTCGGGACAGGAGCAGCGGTACACTTGCCACGTCCAGCACGAG |
| | GGACTCCCCAAGCCTCTGACCCTGCGCTGGGAACCTAGCTCCGGAAGCGGATCCGCAGGC |
| | GGATCGGGATCAGGCGGTGGCTCTGACCCCTCCAAGGACAGCAAGGCTCAGGTGTCAGC |
| | CGCCGAAGCAGGCATCACCGGCACCTGGTACAACCAGCTTGGGTCCACCTTTATCGTGAC |
| | CGCGGGAGCAGATGGCGCCCTGACTGGCACCTACGAATCCGCCGTCGGAAACGCCGAGT |
| | CCAGATACGTGCTGACCGGGCGCTACGACTCCGCGCCTGCAACCGATGGCTCGGGTACAG |
| | CCCTTGGATGGACTGTCGCCTGGAAGAACAACTACAGGAACGCCCACTCCGCCACCACTT |
| | GGAGCGGGCAGTATGTCGGAGGAGCTGAGGCGCGGATTAACACTCAATGGCTGCTGACC |
| | TCCGGTACCACCGAAGCCAATGCATGGAAGTCGACCCTGGTCGGCCATGACACCTTCACC |
| | AAGGTGAAACCTTCGGCCGCCTCCATTGACGCCGCCAAGAAGGCGGGGGTGAACAACGG |
| | CAACCCGCTGGATGCCGTGCAGCAGGGCTCCACTGGCCACCACCACCATCACCACGACTA |
| | TAAGGACGACGATGACAAGTGA |
| | (nucleotide sequence of the coding region of the expression |
| | construct shown in FIG. 1) |
| 2 | MEAPAQLLFLLLLWLPDTTGNLVPMVATVGGGASGGGGSIEGRGGGGSGGGGSIQRTPKIQV |
| | YSRHPAENGKSNFLNCYVSGFHPSDIEVDLLKNGERIEKVEHSDLSFSKDWSFYLLYYTEFTPT |
| | EKDEYACRVNHVTLSQPKIVKWDRDMGGGGSGGGGSGGGGSGGGGSGSHSMRYFFTSVSR |
| | PGRGEPRFIAVGYVDDTQFVRFDSDAASQRMEPRAPWIEQEGPEYWDGETRKVKAHSQTHR |
| | VDLGTLRGYYNQSEAGSHTVQRMYGCDVGSDWRFLRGYHQYAYDGKDYIALKEDLRSWT |
| | AADMAAQTTKHKWEAAHVAEQLRAYLEGTCVEWLRRYLENGKETLQRTDAPKTHMTHHA |
| | QRYTCHVQHEGLPKPLTLRWEPSSGSGSAGGSGSGGGSDPSKDSKAQVSAAEAGITGTWYN |
| | QLGSTFIVTAGADGALTGTYESAVGNAESRYVLTGRYDSAPATDGSGTALGWTVAWKNNY |
| | RNAHSATTWSGQYVGGAEARINTQWLLTSGTTEANAWKSTLVGHDTFTKVKPSAASIDAAK |
| | KAGVNNGNPLDAVQQGSTGHHHHHHDYKDDDDK |
| | (amino acid sequence of the coding region of the expression |
| | construct shown in FIG. 1, including signal sequence and tags) |
| 3 | NLVPMVATVGGGASGGGGSIEGRGGGGSGGGGSIQRTPKIQVYSRHPAENGKSNFLNCYVSG |
| | FHPSDIEVDLLKNGERIEKVEHSDLSFSKDWSFYLLYYTEFTPTEKDEYACRVNHVTLSQPKIV |
| | KWDRDMGGGGSGGGGSGGGGSGGGGSGSHSMRYFFTSVSRPGRGEPRFIAVGYVDDTQFV |
| | RFDSDAASQRMEPRAPWIEQEGPEYWDGETRKVKAHSQTHRVDLGTLRGYYNQSEAGSHTV |
| | QRMYGCDVGSDWRFLRGYHQYAYDGKDYIALKEDLRSWTAADMAAQTTKHKWEAAHVA |
| | EQLRAYLEGTCVEWLRRYLENGKETLQRTDAPKTHMTHHAVSDHEATLRCWALSFYPAEIT |
| | LTWQRDGEDQTQDTELVETRPAGDGTFQKWAAVVVPSGQEQRYTCHVQHEGLPKPLTLRW |
| | EPSSGSGSAGGSGSGGGSDPSKDSKAQVSAAEAGITGTWYNQLGSTFIVTAGADGALTGTYE |
| | SAVGNAESRYVLTGRYDSAPATDGSGTALGWTVAWKNNYRNAHSATTWSGQYVGGAEARI |
| | NTQWLLTSGTTEANAWKSTLVGHDTFTKVKPSAASIDAAKKAGVNNGNPLDAVQQGSTG |
| | (amino acid sequence of the coding region of the expression |
| | construct shown in FIG. 1, without signal sequence and tags) |
| 4 | NLVPMVATV |
| | (HLA-A*02:01 restricted CMV pp65 epitope) |
| 5 | GSHSMRYFFTSVSRPGRGEPRFIAVGYVDDTQFVRFDSDAASQRMEPRAPWIEQEGPEYWDG |
| | ETRKVKAHSQTHRVDLGTLRGYYNQSEAGSHTVQRMYGCDVGSDWRFLRGYHQYAYDGK |
| | DYIALKEDLRSWTAADMAAQTTKHKWEAAHVAEQLRAYLEGTCVEWLRRYLENGKETLQR |
| | TDAPKTHMTHHAVSDHEATLRCWALSFYPAEITLTWQRDGEDQTQDTELVETRPAGDGTFQ |
| | KWAAVVVPSGQEQRYTCHVQHEGLPKPLTLRWEPSS |
| | (the soluble domain of HLA-A*02:01; residues 25-302) |
| 6 | EAAGIGILTV |
| | (HLA-A*02:01 restricted MART-1 epitope) |
| 7 | YMLDLQPETT |
| | (HLA-A*02:01 restricted HPV epitope) |
| 8 | SLPITVYYA |
| | (HLA-A*02:01 restricted HSV epitope) |
| 9 | RMFPNAPYL |
| | (HLA-A*02:01 restricted WT-1 epitope) |
| 10 | MAVMAPRTLLLLLSGALALTQTWAGSHSMRYFFTSVSRPGRGEPRFIAVGYVDDTQFVRFDS |
| | DAASQKMEPRAPWIEQEGPEYWDQETRNMKAHSQTDRANLGTLRGYYNQSEDGSHTIQIMY |
| | GCDVGPDGRFLRGYRQDAYDGKDYIALNEDLRSWTAADMAAQITKRKWEAVHAAEQRRV |
| | YLEGRCVDGLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWELSSQPTI |
| | PIVGIIAGLVLLGAVITGAVVAAVMWRRKSSDRKGGSYTQAASSDSAQGSDVSLTACKV |
| | (HLA-A*01:01 full-length) |
| 11 | MAVMAPRTLLLLLSGALALTQTWAGSHSMRYFFTSVSRPGRGEPRFIAVGYVDDTQFVRFDS |
| | DAASQRMEPRAPWIEQEGPEYWDQETRNVKAQSQTDRVDLGTLRGYYNQSEAGSHTIQIMY |
| | GCDVGSDGRFLRGYRQDAYDGKDYIALNEDLRSWTAADMAAQITKRKWEAAHEAEQLRAY |
| | LDGTCVEWLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWELSSQPTI |
| | PIVGIIAGLVLLGAVITGAVVAAVMWRRKSSDRKGGSYTQAASSDSAQGSDVSLTACKV |
| | (HLA-A*03:01 full-length) |
| 12 | MAVMAPRTLLLLLSGALALTQTWAGSHSMRYFYTSVSRPGRGEPRFIAVGYVDDTQFVRFDS |
| | DAASQRMEPRAPWIEQEGPEYWDQETRNVKAQSQTDRVDLGTLRGYYNQSEDGSHTIQIMY |
| | GCDVGPDGRFLRGYRQDAYDGKDYIALNEDLRSWTAADMAAQITKRKWEAAHAAEQQRA |
| | YLEGRCVEWLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWELSSQPTI |
| | PIVGIIAGLVLLGAVITGAVVAAVMWRRKSSDRKGGSYTQAASSDSAQGSDVSLTACKV |
| | (HLA-A*11:01 full-length) |
| 13 | MAVMAPRTLVLLLSGALALTQTWAGSHSMRYFSTSVSRPGRGEPRFIAVGYVDDTQFVRFDS |
| | DAASQRMEPRAPWIEQEGPEYWDEETGKVKAHSQTDRENLRIALRYYNQSEAGSHTLQMMF |
| | GCDVGSDGRFLRGYHQYAYDGKDYIALKEDLRSWTAADMAAQITKRKWEAAHVAEQQRA |
| | YLEGTCVDGLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWEPSSQPT |
| | VPIVGIIAGLVLLGAVITGAVVAAVMWRRNSSDRKGGSYSQAASSDSAQGSDVSLTACKV |
| | (HLA-A*24:02 full-length) |
| 14 | MLVMAPRTVLLLLSAALALTETWAGSHSMRYFYTSVSRPGRGEPRFISVGYVDDTQFVRFDS |
| | DAASPREEPRAPWIEQEGPEYWDRNTQIYKAQAQTDRESLRNLRGYYNQSEAGSHTLQSMY |
| | GCDVGPDGRLLRGHDQYAYDGKDYIALNEDLRSWTAADTAAQITQRKWEAAREAEQRRAY |
| | LEGECVEWLRRYLENGKDKLERADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAACSDSAQGSDVSLTA |
| | (HLA-B*07:02 full-length) |
| 15 | MRVMAPRTLILLLSGALALTETWAGSHSMRYFSTSVSWPGRGEPRFIAVGYVDDTQFVRFDS |
| | DAASPRGEPREPWVEQEGPEYWDRETQKYKRQAQADRVNLRKLRGYYNQSEDGSHTLQRM |
| | FGCDLGPDGRLLRGYNQFAYDGKDYIALNEDLRSWTAADTAAQITQRKWEAAREAEQRRA |
| | YLEGTCVEWLRRYLENGKETLQRAEHPKTHVTHHPVSDHEATLRCWALGFYPAEITLTWQW |
| | DGEDQTQDTELVETRPAGDGTFQKWAAVVVPSGEEQRYTCHVQHEGLPEPLTLRWKPSSQP |
| | TIPIVGIVAGLAVLAVLAVLGAMVAVVMCRRKSSGGKGGSCSQAASSNSAQGSDESLIACKA |
| | (HLA-C*04:01 full-length) |
| 16 | MRVMAPRALLLLLSGGLALTETWACSHSMRYFDTAVSRPGRGEPRFISVGYVDDTQFVRFDS |
| | DAASPRGEPRAPWVEQEGPEYWDRETQKYKRQAQADRVSLRNLRGYYNQSEDGSHTLQRM |
| | SGCDLGPDGRLLRGYDQSAYDGKDYIALNEDLRSWTAADTAAQITQRKLEAARAAEQLRAY |
| | LEGTCVEWLRRYLENGKETLQRAEPPKTHVTHHPLSDHEATLRCWALGFYPAEITLTWQRD |
| | GEDQTQDTELVETRPAGDGTFQKWAAVVVPSGQEQRYTCHMQHEGLQEPLTLSWEPSSQPTI |
| | PIMGIVAGLAVLVVLAVLGAVVTAMMCRRKSSGGKGGSCSQAACSNSAQGSDESLITCKA |
| | (HLA-C*07:02 full-length) |
| 17 | MLVMAPRTVLLLLSAALALTETWAGSHSMRYFDTAMSRPGRGEPRFISVGYVDDTQFVRFD |
| | SDAASPREEPRAPWIEQEGPEYWDRNTQIFKTNTQTDRESLRNLRGYYNQSEAGSHTLQSMY |
| | GCDVGPDGRLLRGHNQYAYDGKDYIALNEDLRSWTAADTAAQITQRKWEAARVAEQDRAY |
| | LEGTCVEWLRRYLENGKDTLERADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAACSDSAQGSDVSLTA |
| | (HLA-B*08:01 full-length) |
| 18 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRESLRNLRGYYNQSEAGSHIIQRMYG |
| | CDLGPDGRLLRGHDQSAYDGKDYIALNEDLSSWTAADTAAQITQRKWEAARVAEQLRAYLE |
| | GLCVEWLRRYLENGKETLQRADPPKTHVTHHPVSDHEATLRCWALGFYPAEITLTWQRDGE |
| | DQTQDTELVETRPAGDRTFQKWAAVVVPSGEEQRYTCHVQHEGLPKPLTLRWEPSSQSTIPIV |
| | GIVAGLAVLAVVVIGAVVATVMCRRKSSGGKGGSYSQAASSDSAQGSDVSLTA |
| | (HLA-B*35:01 full-length) |
| 19 | MRVTAPRTVLLLLWGAVALTETWAGSHSMRYFYTAMSRPGRGEPRFIAVGYVDDTQFVRFD |
| | SDAASPRMAPRAPWIEQEGPEYWDGETRNMKASAQTYRENLRIALRYYNQSEAGSHIIQVM |
| | YGCDVGPDGRLLRGHDQSAYDGKDYIALNEDLSSWTAADTAAQITQRKWEAARVAEQLRA |
| | YLEGLCVEWLRRYLENGKETLQRADPPKTHVTHHPISDHEATLRCWALGFYPAEITLTWQRD |
| | GEDQTQDTELVETRPAGDRTFQKWAAVVVPSGEEQRYTCHVQHEGLPKPLTLRWEPSSQST |
| | VPIVGIVAGLAVLAVVVIGAVVAAVMCRRKSSGGKGGSYSQAACSDSAQGSDVSLTA |
| | (HLA-B*57:01 full-length) |
| 20 | MRVTAPRTVLLLLWGAVALTETWAGSHSMRYFYTAMSRPGRGEPRFIAVGYVDDTQFVRFD |
| | SDAASPRMAPRAPWIEQEGPEYWDGETRNMKASAQTYRENLRIALRYYNQSEAGSHIIQVM |
| | YGCDVGPDGRLLRGHNQYAYDGKDYIALNEDLSSWTAADTAAQITQRKWEAARVAEQLRA |
| | YLEGLCVEWLRRYLENGKETLQRADPPKTHVTHHPISDHEATLRCWALGFYPAEITLTWQRD |
| | GEDQTQDTELVETRPAGDRTFQKWAAVVVPSGEEQRYTCHVQHEGLPKPLTLRWEPSSQST |
| | VPIVGIVAGLAVLAVVVIGAVVAAVMCRRKSSGGKGGSYSQAACSDSAQGSDVSLTA |
| | (HLA-B*57:03 full-length) |
| 21 | MVDGTLLLLLSEALALTQTWAGSHSLKYFHTSVSRPGRGEPRFISVGYVDDTQFVRFDNDAA |
| | SPRMVPRAPWMEQEGSEYWDRETRSARDTAQIFRVNLRTLRGYYNQSEAGSHTLQWMHGC |
| | ELGPDGRFLRGYEQFAYDGKDYLTLNEDLRSWTAVDTAAQISEQKSNDASEAEHQRAYLED |
| | TCVEWLHKYLEKGKETLLHLEPPKTHVTHHPISDHEATLRCWALGFYPAEITLTWQQDGEGH |
| | TQDTELVETRPAGDGTFQKWAAVVVPSGEEQRYTCHVQHEGLPEPVTLRWKPASQPTIPIVGI |
| | IAGLVLLGSVVSGAVVAAVIWRKKSSGGKGGSYSKAEWSDSAQGSESHSL |
| | (HLA-E full-length) |
| 22 | MRVMAPRTLILLLSGALALTETWACSHSMRYFYTAVSRPGRGEPRFIAVGYVDDTQFVRFDS |
| | DAASPRGEPRAPWVEQEGPEYWDRETQKYKRQAQTDRVSLRNLRGYYNQSEAGSHTLQWM |
| | YGCDLGPDGRLLRGYDQSAYDGKDYIALNEDLRSWTAADTAAQITQRKWEAARAAEQQRA |
| | YLEGTCVEWLRRYLENGKETLQRAEHPKTHVTHHLVSDHEATLRCWALGFYPAEITLTWQR |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVVAVVMCRRKSSGGKGGSCSQAASSNSAQGSDESLIACKA |
| | (HLA-C*16:01 full-length) |
| 23 | MRVMAPRTLILLLSGALALTETWACSHSMRYFYTAVSRPGRGEPRFIA VGYVDDTQFVQFDS |
| | DAASPRGEPRAPWVEQEGPEYWDRETQKYKRQAQTDRVSLRNLRGYYNQSEAGSHTLQRM |
| | YGCDLGPDGRLLRGYNQFAYDGKDYIALNEDLRSWTAADKAAQITQRKWEAAREAEQRRA |
| | YLEGTCVEWLRRYLENGKKTLQRAEHPKTHVTHHPVSDHEATLRCWALGFYPAEITLTWQR |
| | DGEDQTQDTELVETRPAGDGTFQKWAAVVVPSGEEQRYTCHVQHEGLPEPLTLRWGPSSQP |
| | TIPIVGIVAGLAVLAVLAVLGAVMAVVMCRRKSSGGKGGSCSQAASSNSAQGSDESLIACKA |
| | (HLA-C*08:02 full-length) |
| 24 | MRVMAPRALLLLLSGGLALTETWACSHSMRYFDTAVSRPGRGEPRFISVGYVDDTQFVRFDS |
| | DAASPRGEPRAPWVEQEGPEYWDRETQNYKRQAQADRVSLRNLRGYYNQSEDGSHTLQRM |
| | YGCDLGPDGRLLRGYDQSAYDGKDYIALNEDLRSWTAADTAAQITQRKLEAARAAEQLRAY |
| | LEGTCVEWLRRYLENGKETLQRAEPPKTHVTHHPLSDHEATLRCWALGFYPAEITLTWQRD |
| | GEDQTQDTELVETRPAGDGTFQKWAAVVVPSGQEQRYTCHMQHEGLQEPLTLSWEPSSQPTI |
| | PIMGIVAGLAVLVVLAVLGAVVTAMMCRRKSSGGKGGSCSQAACSNSAQGSDESLITCKA |
| | (HLA-C*07:01 full-length) |
| 25 | MRVMAPRTLILLLSGALALTETWACSHSMRYFYTAVSRPGRGEPRFIA VGYVDDTQFVQFDS |
| | DAASPRGEPRAPWVEQEGPEYWDRETQKYKRQAQTDRVNLRKLRGYYNQSEAGSHTLQRM |
| | YGCDLGPDGRLLRGYNQFAYDGKDYIALNEDLRSWTAADKAAQITQRKWEAAREAEQRRA |
| | YLEGTCVEWLRRYLENGKKTLQRAEHPKTHVTHHPVSDHEATLRCWALGFYPAEITLTWQR |
| | DGEDQTQDTELVETRPAGDGTFQKWAAVVVPSGEEQRYTCHVQHEGLPEPLTLRWGPSSQP |
| | TIPIVGIVAGLAVLAVLAVLGAVMAVVMCRRKSSGGKGGSCSQAASSNSAQGSDESLIACKA |
| | (HLA-C*05:01 full-length) |
| 26 | MRVTAPRTLLLLLWGAVALTETWAGSHSMRYFYTAMSRPGRGEPRFITVGYVDDTLFVRFD |
| | SDATSPRKEPRAPWIEQEGPEYWDRETQISKTNTQTYRENLRTALRYYNQSEAGSHIIQRMYG |
| | CDVGPDGRLLRGYDQDAYDGKDYIALNEDLSSWTAADTAAQITQRKWEAARVAEQDRAYL |
| | EGLCVESLRRYLENGKETLQRADPPKTHVTHHPISDHEVTLRCWALGFYPAEITLTWQRDGE |
| | DQTQDTELVETRPAGDRTFQKWAAVVVPSGEEQRYTCHVQHEGLPKPLTLRWEPSSQSTVPI |
| | VGIVAGLAVLAVVVIGAVVAAVMCRRKSSGGKGGSYSQAACSDSAQGSDVSLTA |
| | (HLA-B*44:02 full-length) |
| 27 | MAVMAPRTLLLLLLGALALTQTWAGSHSMRYFTTSVSRPGRGEPRFIAVGYVDDTQFVRFDS |
| | DAASQRMEPRAPWIEQEGPEYWDLQTRNVKAQSQTDRANLGTLRGYYNQSEAGSHTIQMM |
| | YGCDVGSDGRFLRGYRQDAYDGKDYIALNEDLRSWTAADMAAQITQRKWEAARVAEQLR |
| | AYLEGTCVEWLRRYLENGKETLQRTDAPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVFAGAVVAAVRWRRKSSDRKGGSYSQAASSDSAQGSDMSLTACKV |
| | (HLA-A*29:02 full-length) |
| 28 | MRVTAPRTLLLLLWGAVALTETWAGSHSMRYFYTAMSRPGRGEPRFITVGYVDDTLFVRFD |
| | SDATSPRKEPRAPWIEQEGPEYWDRETQISKTNTQTYRENLRTALRYYNQSEAGSHIIQRMYG |
| | CDVGPDGRLLRGYDQDAYDGKDYIALNEDLSSWTAADTAAQITQRKWEAARVAEQLRAYL |
| | EGLCVESLRRYLENGKETLQRADPPKTHVTHHPISDHEVTLRCWALGFYPAEITLTWQRDGE |
| | DQTQDTELVETRPAGDRTFQKWAAVVVPSGEEQRYTCHVQHEGLPKPLTLRWEPSSQSTVPI |
| | VGIVAGLAVLAVVVIGAVVAAVMCRRKSSGGKGGSYSQAACSDSAQGSDVSLTA |
| | (HLA-B*44:03 full-length) |
| 29 | MRVMAPRTLILLLSGALALTETWAGSHSMRYFYTAVSRPGRGEPHFIAVGYVDDTQFVRFDS |
| | DAASPRGEPRAPWVEQEGPEYWDRETQKYKRQAQTDRVSLRNLRGYYNQSEAGSHIIQRMY |
| | GCDVGPDGRLLRGYDQYAYDGKDYIALNEDLRSWTAADTAAQITQRKWEAAREAEQLRAY |
| | LEGLCVEWLRRYLKNGKETLQRAEHPKTHVTHHPVSDHEATLRCWALGFYPAEITLTWQWD |
| | GEDQTQDTELVETRPAGDGTFQKWAAVVVPSGEEQRYTCHVQHEGLPEPLTLRWEPSSQPTI |
| | PIVGIVAGLAVLAVLAVLGAVVAVVMCRRKSSGGKGGSCSQAASSNSAQGSDESLIACKA |
| | (HLA-C*03:04 full-length) |
| 30 | MRVTAPRTVLLLLSAALALTETWAGSHSMRYFHTAMSRPGRGEPRFITVGYVDDTLFVRFDS |
| | DATSPRKEPRAPWIEQEGPEYWDRETQISKTNTQTYRESLRNLRGYYNQSEAGSHTLQRMYG |
| | CDVGPDGRLLRGHNQYAYDGKDYIALNEDLRSWTAADTAAQISQRKLEAARVAEQLRAYLE |
| | GECVEWLRRYLENGKDKLERADPPKTHVTHHPISDHEATLRCWALGFYPAEITLTWQRDGE |
| | DQTQDTELVETRPAGDRTFQKWAAVVVPSGEEQRYTCHVQHEGLPKPLTLRWEPSSQSTVPI |
| | VGIVAGLAVLAVVVIGAVVAAVMCRRKSSGGKGGSYSQAACSDSAQGSDVSLTA |
| | (HLA-B*40:01 full-length) |
| 31 | MRVMAPRTLILLLSGALALTETWACSHSMRYFDTAVSRPGRGEPRFISVGYVDDTQFVRFDS |
| | DAASPRGEPRAPWVEQEGPEYWDRETQKYKRQAQADRVNLRKLRGYYNQSEDGSHTLQW |
| | MYGCDLGPDGRLLRGYDQSAYDGKDYIALNEDLRSWTAADTAAQITQRKWEAAREAEQWR |
| | AYLEGTCVEWLRRYLENGKETLQRAEHPKTHVTHHPVSDHEATLRCWALGFYPAEITLTWQ |
| | RDGEDQTQDTELVETRPAGDGTFQKWAAVVVPSGEEQRYTCHVQHEGLPEPLTLRWEPSSQ |
| | PTIPIVGIVAGLAVLAVLAVLGAVMAVVMCRRKSSGGKGGSCSQAASSNSAQGSDESLIACKA |
| | (HLA-C*06:02 full-length) |
| 32 | MRVTAPRTVLLLLSGALALTETWAGSHSMRYFYTAMSRPGRGEPRFIAVGYVDDTQFVRFDS |
| | DAASPRMAPRAPWIEQEGPEYWDRETQISKTNTQTYRESLRNLRGYYNQSEAGSHTLQRMY |
| | GCDVGPDGRLLRGHDQSAYDGKDYIALNEDLSSWTAADTAAQITQRKWEAAREAEQWRAY |
| | LEGLCVEWLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTIPI |
| | VGIVAGLAVLAVVVIGAVVATVMCRRKSSGGKGGSYSQAASSDSAQGSDVSLTA |
| | (HLA-B*15:01 full-length) |
| 33 | MRVMAPRTLILLLSGALALTETWAGSHSMRYFYTAVSRPGRGEPHFIAVGYVDDTQFVRFDS |
| | DAASPRGEPRAPWVEQEGPEYWDRETQKYKRQAQTDRVSLRNLRGYYNQSEARSHIIQRMY |
| | GCDVGPDGRLLRGYDQYAYDGKDYIALNEDLRSWTAADTAAQITQRKWEAAREAEQLRAY |
| | LEGLCVEWLRRYLKNGKETLQRAEHPKTHVTHHPVSDHEATLRCWALGFYPAEITLTWQWD |
| | GEDQTQDTELVETRPAGDGTFQKWAAVVVPSGEEQRYTCHVQHEGLPEPLTLRWEPSSQPTI |
| | PIVGIVAGLAVLAVLAVLGAVVAVVMCRRKSSGGKGGSCSQAASSNSAQGSDESLIACKA |
| | (HLA-C*03:03 full-length) |
| 34 | MAVMAPRTLLLLLSGALALTQTWAGSHSMRYFSTSVSRPGSGEPRFIAVGYVDDTQFVRFDS |
| | DAASQRMEPRAPWIEQERPEYWDQETRNVKAQSQTDRVDLGTLRGYYNQSEAGSHTIQIMY |
| | GCDVGSDGRFLRGYEQHAYDGKDYIALNEDLRSWTAADMAAQITQRKWEAARWAEQLRA |
| | YLEGTCVEWLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWELSSQPTI |
| | PIVGIIAGLVLLGAVITGAVVAAVMWRRKSSDRKGGSYTQAASSDSAQGSDVSLTACKV |
| | (HLA-A*30:01 full-length) |
| 35 | MRVTAPRTLLLLLWGAVALTETWAGSHSMRYFYTAMSRPGRGEPRFITVGYVDDTQFVRFD |
| | SDATSPRMAPRAPWIEQEGPEYWDRETQISKTNTQTYRENLRTALRYYNQSEAGSHTWQTM |
| | YGCDLGPDGRLLRGHNQLAYDGKDYIALNEDLSSWTAADTAAQITQLKWEAARVAEQLRA |
| | YLEGECVEWLRRYLENGKETLQRADPPKTHVTHHPISDHEATLRCWALGFYPAEITLTWQRD |
| | GEDQTQDTELVETRPAGDRTFQKWAAVVVPSGEEQRYTCHVQHEGLPKPLTLRWEPSSQST |
| | VPIVGIVAGLAVLAVVVIGAVVAAVMCRRKSSGGKGGSYSQAACSDSAQGSDVSLTA |
| | (HLA-B*13:02 full-length) |
| 36 | MRVMAPRTLILLLSGALALTETWACSHSMRYFYTAVSRPGRGEPRFIAVGYVDDTQFVRFDS |
| | DAASPRGEPRAPWVEQEGPEYWDRETQKYKRQAQADRVSLRNLRGYYNQSEAGSHTLQW |
| | MYGCDLGPDGRLLRGYDQSAYDGKDYIALNEDLRSWTAADTAAQITQRKWEAAREAEQWR |
| | AYLEGTCVEWLRRYLENGKETLQRAEHPKTHVTHHPVSDHEATLRCWALGFYPAEITLTWQ |
| | RDGEDQTQDTELVETRPAGDGTFQKWAAVVVPSGEEQRYTCHVQHEGLPEPLTLRWEPSSQ |
| | PTIPIVGIVAGLAVLAVLAVLGAVMAVVMCRRKSSGGKGGSCSQAASSNSAQGSDESLIACKA |
| | (HLA-C*12:03 full-length) |
| 37 | MAVMAPRTLVLLLSGALALTQTWAGSHSMRYFYTSVSRPGRGEPRFIAVGYVDDTQFVRFD |
| | SDAASQRMEPRAPWIEQEGPEYWDRNTRNVKAHSQTDRANLGTLRGYYNQSEDGSHTIQRM |
| | YGCDVGPDGRFLRGYQQDAYDGKDYIALNEDLRSWTAADMAAQITQRKWETAHEAEQWR |
| | AYLEGRCVEWLRRYLENGKETLQRTDAPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVIAGAVVAAVMWRRKSSDRKGGSYSQAASSDSAQGSDMSLTACKV |
| | (HLA-A*26:01 full-length) |
| 38 | MLVMAPRTVLLLLSAALALTETWAGSHSMRYFYTSVSRPGRGEPRFISVGYVDDTQFVRFDS |
| | DAASPREEPRAPWIEQEGPEYWDRNTQICKTNTQTYRENLRIALRYYNQSEAGSHTLQRMYG |
| | CDVGPDGRLLRGHNQFAYDGKDYIALNEDLSSWTAADTAAQITQRKWEAARVAEQLRTYLE |
| | GTCVEWLRRYLENGKETLQRADPPKTHVTHHPISDHEATLRCWALGFYPAEITLTWQRDGED |
| | QTQDTELVETRPAGDRTFQKWAAVVVPSGEEQRYTCHVQHEGLPKPLTLRWEPSSQSTVPIV |
| | GIVAGLAVLAVVVIGAVVAAVMCRRKSSGGKGGSYSQAASSDSAQGSDVSLTA |
| | (HLA-B*38:01 full-length) |
| 39 | MLVMAPRTVLLLLSAALALTETWAGSHSMRYFYTAVSRPGRGEPRFISVGYVDDTQFVRFDS |
| | DAASPREEPRAPWIEQEGPEYWDRNTQICKTNTQTDRESLRNLRGYYNQSEAGSHTLQWMY |
| | GCDVGPDGRLLRGYNQFAYDGKDYIALNEDLSSWTAADTAAQITQRKWEAAREAEQLRAY |
| | LEGTCVEWLRRHLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAASSDSAQGSDVSLTA |
| | (HLA-B*14:02 full-length) |
| 40 | MAVMAPRTLLLLLLGALALTQTWAGSHSMRYFTTSVSRPGRGEPRFIAVGYVDDTQFVRFDS |
| | DAASQRMEPRAPWIEQEGPEYWDRNTRNVKAHSQIDRVDLGTLRGYYNQSEAGSHTIQMM |
| | YGCDVGSDGRFLRGYQQDAYDGKDYIALNEDLRSWTAADMAAQITQRKWEAARVAEQLR |
| | AYLEGTCVEWLRRHLENGKETLQRTDPPRTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVFAGAVVAAVRWRRKSSDRKGGSYSQAASSDSAQGSDMSLTACKV |
| | (HLA-A*33:01 full-length) |
| 41 | MAVMAPRTLVLLLSGALALTQTWAGSHSMRYFSTSVSRPGRGEPRFIAVGYVDDTQFVRFDS |
| | DAASQRMEPRAPWIEQEGPEYWDEETGKVKAHSQTDRENLRIALRYYNQSEAGSHTLQMMF |
| | GCDVGSDGRFLRGYHQYAYDGKDYIALKEDLRSWTAADMAAQITQRKWEAARVAEQLRA |
| | YLEGTCVDGLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWEPSSQPT |
| | VHIVGIIAGLVLLGAVITGAVVAAVMWRRNSSDRKGGSYSQAASSDSAQGSDVSLTACKV |
| | (HLA-A*23:01 full-length) |
| 42 | MAVMAPRTLVLLLSGALALTQTWAGSHSMRYFYTSVSRPGRGEPRFIAVGYVDDTQFVRFD |
| | SDAASQRMEPRAPWIEQEGPEYWDRNTRNVKAHSQTDRESLRIALRYYNQSEDGSHTIQRM |
| | YGCDVGPDGRFLRGYQQDAYDGKDYIALNEDLRSWTAADMAAQITQRKWETAHEAEQWR |
| | AYLEGRCVEWLRRYLENGKETLQRTDAPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVIAGAVVAAVMWRRKSSDRKGGSYSQAASSDSAQGSDMSLTACKV |
| | (HLA-A*25:01 full-length) |
| 43 | MRVTAPRTLLLLLWGAVALTETWAGSHSMRYFHTSVSRPGRGEPRFISVGYVDGTQFVRFDS |
| | DAASPRTEPRAPWIEQEGPEYWDRNTQISKTNTQTYRESLRNLRGYYNQSEAGSHTLQRMYG |
| | CDVGPDGRLLRGHDQSAYDGKDYIALNEDLSSWTAADTAAQITQRKWEAARVAEQLRAYL |
| | EGTCVEWLRRHLENGKETLQRADPPKTHVTHHPISDHEATLRCWALGFYPAEITLTWQRDGE |
| | DQTQDTELVETRPAGDRTFQKWAAVVVPSGEEQRYTCHVQHEGLPKPLTLRWEPSSQSTIPIV |
| | GIVAGLAVLAVVVIGAVVATVMCRRKSSGGKGGSYSQAASSDSAQGSDVSLTA |
| | (HLA-B*18:01 full-length) |
| 44 | MRVTAPRTLLLLLWGAVALTETWAGSHSMRYFHTSVSRPGRGEPRFISVGYVDDTQFVRFDS |
| | DAASPRTEPRAPWIEQEGPEYWDRETQISKTNTQTYREDLRTLLRYYNQSEAGSHTIQRMSGC |
| | DVGPDGRLLRGYNQFAYDGKDYIALNEDLSSWTAADTAAQITQRKWEAARVAEQDRAYLE |
| | GTCVEWLRRYLENGKETLQRADPPKTHVTHHPISDHEATLRCWALGFYPAEITLTWQRDGED |
| | QTQDTELVETRPAGDRTFQKWAAVVVPSGEEQRYTCHVQHEGLPKPLTLRWEPSSQSTIPIVG |
| | IVAGLAVLAVVVIGAVVATVMCRRKSSGGKGGSYSQAASSDSAQGSDVSLTA |
| | (HLA-B*37:01 full length) |
| 45 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRENLRIALRYYNQSEAGSHTWQTMY |
| | GCDVGPDGRLLRGHNQYAYDGKDYIALNEDLSSWTAADTAAQITQRKWEAAREAEQLRAY |
| | LEGLCVEWLRRHLENGKETLQRADPPKTHVTHHPVSDHEATLRCWALGFYPAEITLTWQRD |
| | GEDQTQDTELVETRPAGDRTFQKWAAVVVPSGEEQRYTCHVQHEGLPKPLTLRWEPSSQSTI |
| | PIVGIVAGLAVLAVVVIGAVVATVMCRRKSSGGKGGSYSQAASSDSAQGSDVSLTA |
| | (HLA-B*51:01 full-length) |
| 46 | MRVMAPRTLILLLSGALALTETWACSHSMRYFSTSVSRPGRGEPRFIAVGYVDDTQFVRFDS |
| | DAASPRGEPRAPWVEQEGPEYWDRETQKYKRQAQTDRVSLRNLRGYYNQSEAGSHTLQWM |
| | FGCDLGPDGRLLRGYDQSAYDGKDYIALNEDLRSWTAADTAAQITQRKWEAAREAEQRRA |
| | YLEGTCVEWLRRYLENGKETLQRAEHPKTHVTHHPVSDHEATLRCWALGFYPAEITLTWQW |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVVAVVMCRRKSSGGKGGSCSQAASSNSAQGSDESLIACKA |
| | (HLA-C*14:02 full-length) |
| 47 | MRVMAPRTLLLLLSGALALTETWACSHSMRYFYTAVSRPGRGEPHFIAVGYVDDTQFVRFDS |
| | DAASPRGEPRAPWVEQEGPEYWDRETQNYKRQAQTDRVNLRKLRGYYNQSEAGSHIIQRMY |
| | GCDLGPDGRLLRGHDQLAYDGKDYIALNEDLRSWTAADTAAQITQRKWEAAREAEQLRAY |
| | LEGTCVEWLRRYLENGKETLQRAEHPKTHVTHHPVSDHEATLRCWALGFYPAEITLTWQRD |
| | GEDQTQDTELVETRPAGDGTFQKWAAVVVPSGEEQRYTCHVQHEGLPEPLTLRWEPSSQPTI |
| | PIVGIVAGLAVLAVLAVLGAVMAVVMCRRKSSGGKGGSCSQAASSNSAQGSDESLIACKA |
| | (HLA-C*15:02 full-length) |
| 48 | MRVMAPRTLLLLLSGALALTETWACSHSMRYFYTAVSRPSRGEPHFIAVGYVDDTQFVRFDS |
| | DAASPRGEPRAPWVEQEGPEYWDRETQKYKRQAQTDRVNLRKLRGYYNQSEAGSHTLQRM |
| | YGCDLGPDGRLLRGYDQSAYDGKDYIALNEDLRSWTAADTAAQITQRKWEAAREAEQWRA |
| | YLEGECVEWLRRYLENGKETLQRAEHPKTHVTHHPVSDHEATLRCWALGFYPTEITLTWQR |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVVAVVMCRRKSSGGKGGSCSQAASSNSAQGSDESLIACKA |
| | (HLA-C*02:02 full-length) |
| 49 | MRVTAPRTLLLLLWGAVALTETWAGSHSMRYFHTSVSRPGRGEPRFITVGYVDDTLFVRFDS |
| | DAASPREEPRAPWIEQEGPEYWDRETQICKAKAQTDREDLRTLLRYYNQSEAGSHTLQNMY |
| | GCDVGPDGRLLRGYHQDAYDGKDYIALNEDLSSWTAADTAAQITQRKWEAARVAEQLRAY |
| | LEGECVEWLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAACSDSAQGSDVSLTA |
| | (HLA-B*27:05 full-length) |
| 50 | MAVMAPRTLLLLLLGALALTQTWAGSHSMRYFTTSVSRPGRGEPRFIAVGYVDDTQFVRFDS |
| | DAASQRMEPRAPWIEQERPEYWDQETRNVKAHSQIDRVDLGTLRGYYNQSEAGSHTIQMMY |
| | GCDVGSDGRFLRGYQQDAYDGKDYIALNEDLRSWTAADMAAQITQRKWEAARVAEQLRA |
| | YLEGTCVEWLRRYLENGKETLQRTDPPKTHMTHHAVSDHEATLRCWALSFYPAEITLTWQR |
| | DGEDQTQDTELVETRPAGDGTFQKWASVVVPSGQEQRYTCHVQHEGLPKPLTLRWEPSSQP |
| | TIPIVGIIAGLVLFGAVFAGAVVAAVRWRRKSSDRKGGSYSQAASSDSAQGSDMSLTACKV |
| | (HLA-A*31:01 full-length) |
| 51 | MAVMAPRTLLLLLSGALALTQTWAGSHSMRYFSTSVSRPGSGEPRFIAVGYVDDTQFVRFDS |
| | DAASQRMEPRAPWIEQERPEYWDQETRNVKAHSQTDRENLGTLRGYYNQSEAGSHTIQIMY |
| | GCDVGSDGRFLRGYEQHAYDGKDYIALNEDLRSWTAADMAAQITQRKWEAARRAEQLRAY |
| | LEGTCVEWLRRYLENGKETLQRTDPPKTHMTHHPISDHEATLRCWALGFYPAEITLTWQRDG |
| | EDQTQDTELVETRPAGDGTFQKWAAVVVPSGEEQRYTCHVQHEGLPKPLTLRWELSSQPTIPI |
| | VGIIAGLVLLGAVITGAVVAAVMWRRKSSDRKGGSYTQAASSDSAQGSDVSLTACKV |
| | (HLA-A*30:02 full-length) |
| 52 | MLVMAPRTVLLLLSAALALTETWAGSHSMRYFYTSVSRPGRGEPRFISVGYVDDTQFVRFDS |
| | DAASPREEPRAPWIEQEGPEYWDRNTQIYKAQAQTDRESLRNLRGYYNQSEAGSHTLQSMY |
| | GCDVGPDGRLLRGHNQYAYDGKDYIALNEDLRSWTAADTAAQITQRKWEAARVAEQDRAY |
| | LEGTCVEWLRRYLENGKDTLERADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAACSDSAQGSDVSLTA |
| | (HLA-B*42:01 full-length) |
| 53 | MRVMAPQALLLLLSGALALIETWAGSHSMRYFYTAVSRPGRGEPRFIAVGYVDDTQFVRFDS |
| | DAASPRGEPRAPWVEQEGPEYWDRETQKYKRQAQADRVNLRKLRGYYNQSEAGSHTIQRMY |
| | GCDLGPDGRLLRGYNQFAYDGKDYIALNEDLRSWTAADTAAQISQRKLEAAREAEQLRAYLE |
| | GECVEWLRGYLENGKETLQRAERPKTHVTHHPVSDHEATLRCWALGFYPAEITLTWQRDGED |
| | QTQDTELVETRPAGDGTFQKWAAVVVPSGQEQRYTCHVQHEGLQEPCTLRWKPSSQPTIPNLGI |
| | VSGPAVLAVLAVLAVLAVLGAVVAAVIHRRKSSGGKGGSCSQAASSNSAQGSDESLIACKA |
| | (HLA-C*17:01 full-length) |
| 54 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRESLRNLRGYYNQSEAGSHIIQRMYG |
| | CDLGPDGRFLRGHNQYAYDGKDYIALNEDLSSWTAADTAAQITQRKWEAARVAEQLRAYL |
| | EGLCVEWLRRYLENGKETLQRADPPKTHVTHHPVSDHEATLRCWALGFYPAEITLTWQRDG |
| | EDQTQDTELVETRPAGDRTFQKWAAVVVPSGEEQRYTCHVQHEGLPKPLTLRWEPSSQSTIPI |
| | VGIVAGLAVLAVVVIGAVVATVMCRRKSSGGKGGSYSQAASSDSAQGSDVSLTA |
| | (HLA-B*35:02 full-length) |
| 55 | MLVMAPRTVLLLLSAALALTETWAGSHSMRYFYTSVSRPGRGEPRFISVGYVDDTQFVRFDS |
| | DAASPREEPRAPWIEQEGPEYWDRNTQICKTNTQTDRESLRNLRGYYNQSEAGSHTWQTMY |
| | GCDVGPDGRLLRGHNQFAYDGKDYIALNEDLSSWTAADTAAQITQRKWEAARVAEQLRTY |
| | LEGTCVEWLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAASSDSAQGSDVSLTA |
| | (HLA-B*39:06 full-length) |
| 56 | MRVMAPRTLILLLSGALALTETWAGSHSMRYFYTAVSRPGRGEPHFIAVGYVDDTQFVRFDS |
| | DAASPRGEPRAPWVEQEGPEYWDRETQKYKRQAQTDRVSLRNLRGYYNQSEAGSHILQRM |
| | YGCDVGPDGRLLRGYDQSAYDGKDYIALNEDLRSWTAADTAAQITQRKWEAAREAEQLRA |
| | YLEGLCVEWLRRYLKNGKETLQRAEHPKTHVTHHPVSDHEATLRCWALGFYPAEITLTWQW |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVVAVVMCRRKSSGGKGGSCSQAASSNSAQGSDESLIACKA |
| | (HLA-C*03:02 full-length) |
| 57 | MRVTAPRTVLLLLWGAVALTETWAGSHSMRYFYTAMSRPGRGEPRFIAVGYVDDTQFVRFD |
| | SDAASPRTEPRAPWIEQEGPEYWDGETRNMKASAQTYRENLRIALRYYNQSEAGSHIIQRMY |
| | GCDLGPDGRLLRGHDQSAYDGKDYIALNEDLSSWTAADTAAQITQRKWEAARVAEQLRAY |
| | LEGLCVEWLRRYLENGKETLQRADPPKTHVTHHPVSDHEATLRCWALGFYPAEITLTWQRD |
| | GEDQTQDTELVETRPAGDRTFQKWAAVVVPSGEEQRYTCHVQHEGLPKPLTLRWEPSSQSTI |
| | PIVGIVAGLAVLAVVVIGAVVATVMCRRKSSGGKGGSYSQAASSDSAQGSDVSLTA |
| | (HLA-B*58:01 full-length) |
| 58 | MAVMAPRTLLLLLLGALALTQTWAGSHSMRYFTTSVSRPGRGEPRFIAVGYVDDTQFVRFDS |
| | DAASQRMEPRAPWIEQEGPEYWDRNTRNVKAHSQIDRVDLGTLRGYYNQSEAGSHTIQMM |
| | YGCDVGSDGRFLRGYQQDAYDGKDYIALNEDLRSWTAADMAAQITQRKWEAARVAEQLR |
| | AYLEGTCVEWLRRYLENGKETLQRTDPPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVFAGAVVAAVRWRRKSSDRKGGSYSQAASSDSAQGSDMSLTACKV |
| | (HLA-A*33:03 full-length) |
| 59 | MAVMAPRTLVLLLSGALALTQTWAGSHSMRYFYTSMSRPGRGEPRFIAVGYVDDTQFVRFD |
| | SDAASQRMEPRAPWIEQEGPEYWDRNTRNVKAQSQTDRVDLGTLRGYYNQSEAGSHTIQRM |
| | YGCDVGPDGRFLRGYHQYAYDGKDYIALKEDLRSWTAADMAAQTTKHKWEAAHVAEQW |
| | RAYLEGTCVEWLRRYLENGKETLQRTDAPKTHMTHHAVSDHEATLRCWALSFYPAEITLTW |
| | QRDGEDQTQDTELVETRPAGDGTFQKWVAVVVPSGQEQRYTCHVQHEGLPKPLTLRWEPSS |
| | QPTIPIVGIIAGLVLFGAVITGAVVAAVMWRRKSSDRKGGSYSQAASSDSAQGSDVSLTACKV |
| | (HLA-A*68:02 full-length) |
| 60 | MRVMAPRTLILLLSGALALTETWACSHSMKYFFTSVSRPGRGEPRFISVGYVDDTQFVRFDSD |
| | AASPRGEPRAPWVEQEGPEYWDRETQKYKRQAQTDRVSLRNLRGYYNQSEAGSHTLQWMC |
| | GCDLGPDGRLLRGYDQYAYDGKDYIALNEDLRSWTAADTAAQITQRKWEAAREAEQRRAY |
| | LEGTCVEWLRRYLENGKETLQRAEHPKTHVTHHPVSDHEATLRCWALGFYPAEITLTWQWD |
| | GEDQTQDTELVETRPAGDGTFQKWAAVMVPSGEEQRYTCHVQHEGLPEPLTLRWEPSSQPTI |
| | PIVGIVAGLAVLAVLAVLGAVVAVVMCRRKSSGGKGGSCSQAASSNSAQGSDESLIACKA |
| | (HLA-C*01:02 full-length) |
| 61 | MRVMAPRALLLLLSGGLALTETWACSHSMRYFDTAVSRPGRGEPRFISVGYVDDTQFVRFDS |
| | DAASPRGEPRAPWVEQEGPEYWDRETQKYKRQAQADRVSLRNLRGYYNQSEDGSHTFQRM |
| | YGCDLGPDGRLLRGYDQFAYDGKDYIALNEDLRSWTAADTAAQITQRKLEAARAAEQDRA |
| | YLEGTCVEWLRRYLENGKKTLQRAEPPKTHVTHHPLSDHEATLRCWALGFYPAEITLTWQR |
| | DGEDQTQDTELVETRPAGDGTFQKWAAVVVPSGQEQRYTCHMQHEGLQEPLTLSWEPSSQP |
| | TIPIMGIVAGLAVLVVLAVLGAVVTAMMCRRKSSGGKGGSCSQAACSNSAQGSDESLITCKA |
| | (HLA-C*07:04 full-length) |
| 62 | MAVMAPRTLVLLLSGALALTQTWAGSHSMRYFYTSVSRPGRGEPRFIAVGYVDDTQFVRFD |
| | SDAASQRMEPRAPWIEQEGPEYWDRNTRNVKAQSQTDRVDLGTLRGYYNQSEAGSHTIQM |
| | MYGCDVGSDGRFLRGYRQDAYDGKDYIALKEDLRSWTAADMAAQTTKHKWEAAHVAEQ |
| | WRAYLEGTCVEWLRRYLENGKETLQRTDAPKTHMTHHAVSDHEATLRCWALSFYPAEITLT |
| | WQRDGEDQTQDTELVETRPAGDGTFQKWVAVVVPSGQEQRYTCHVQHEGLPKPLTLRWEPSSQ |
| | PTIPIVGIIAGLVLFGAVITGAVVAAVMWRRKSSDRKGGSYSQAASSDSAQGSDVSLTACKV |
| | (HLA-A*68:01 full-length) |
| 63 | MAVMAPRTLLLLLLGALALTQTWAGSHSMRYFFTSVSRPGRGEPRFIAVGYVDDTQFVRFDS |
| | DAASQRMEPRAPWIEQEGPEYWDQETRNVKAHSQTDRESLRIALRYYNQSEAGSHTIQMMY |
| | GCDVGPDGRLLRGYQQDAYDGKDYIALNEDLRSWTAADMAAQITQRKWEAARVAEQLRA |
| | YLEGTCVEWLRRYLENGKETLQRTDAPKTHMTHHAVSDHEATLRCWALSFYPAEITLTWQR |
| | DGEDQTQDTELVETRPAGDGTFQKWASVVVPSGQEQRYTCHVQHEGLPKPLTLRWEPSSQP |
| | TIPIVGIIAGLVLFGAMFAGAVVAAVRWRRKSSDRKGGSYSQAASSDSAQGSDMSLTACKV |
| | (HLA-A*32:01 full-length) |
| 64 | MRVTAPRTVLLLLSAALALTETWAGSHSMRYFHTAMSRPGRGEPRFITVGYVDDTLFVRFDS |
| | DATSPRKEPRAPWIEQEGPEYWDRETQISKTNTQTYRENLRIALRYYNQSEAGSHTWQRMYG |
| | CDLGPDGRLLRGYNQLAYDGKDYIALNEDLSSWTAADTAAQITQRKWEAAREAEQLRAYLE |
| | GLCVEWLRRYLENGKETLQRADPPKTHVTHHPISDHEATLRCWALGFYPAEITLTWQRDGED |
| | QTQDTELVETRPAGDRTFQKWAAVVVPSGEEQRYTCHVQHEGLPKPLTLRWEPSSQSTIPIVG |
| | IVAGLAVLAVVVIGAVVATVMCRRKSSGGKGGSYSQAASSDSAQGSDVSLTA |
| | (HLA-B*49:01 full-length) |
| 65 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRENLRIALRYYNQSEAGSHIIQRMYG |
| | CDLGPDGRLLRGHDQSAYDGKDYIALNEDLSSWTAADTAAQITQRKWEAARVAEQLRAYLE |
| | GLCVEWLRRYLENGKETLQRADPPKTHVTHHPVSDHEATLRCWALGFYPAEITLTWQRDGE |
| | DQTQDTELVETRPAGDRTFQKWAAVVVPSGEEQRYTCHVQHEGLPKPLTLRWEPSSQSTIPIV |
| | GIVAGLAVLAVVVIGAVVATVMCRRKSSGGKGGSYSQAASSDSAQGSDVSLTA |
| | (HLA-B*53:01 full-length) |
| 66 | MRVTAPRTVLLLLSAALALTETWAGSHSMRYFHTAMSRPGRGEPRFITVGYVDDTLFVRFDS |
| | DATSPRKEPRAPWIEQEGPEYWDRETQISKTNTQTYRESLRNLRGYYNQSEAGSHTWQRMY |
| | GCDLGPDGRLLRGYNQLAYDGKDYIALNEDLSSWTAADTAAQITQRKWEAAREAEQLRAY |
| | LEGLCVEWLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTIPI |
| | VGIVAGLAVLAVVVIGAVVATVMCRRKSSGGKGGSYSQAASSDSAQGSDVSLTA |
| | (HLA-B*50:01 full-length) |
| 67 | MAVMAPRTLVLLLSGALALTQTWAGSHSMRYFYTSVSRPGRGEPRFIAVGYVDDTQFVRFD |
| | SDAASRRMEPRAPWIEQEGPEYWDGETRKVKAHSQTHRVDLGTLRGYYNQSEAGSHTLQR |
| | MYGCDVGSDWRFLRGYHQYAYDGKDYIALKEDLRSWTAADMAAQTTKHKWEAAHVAEQWR |
| | AYLEGTCVEWLRRYLENGKETLQRTDAPKTHMTHHAVSDHEATLRCWALSFYPAEITLTWQR |
| | DGEDQTQDTELVETRPAGDGTFQKWAAVVVPSGQEQRYTCHVQHEGLPKPLTLRWEPSSQ |
| | PTIPIVGIIAGLVLFGAVITGAVVAAVMWRRKSSDRKGGSYSQAASSDSAQGSDVSLTACKV |
| | (HLA-A*02:05 full-length) |
| 68 | MRVTAPRTLLLLLWGALALTETWAGSHSMRYFYTAMSRPGRGEPRFIAVGYVDDTQFVRFD |
| | SDAASPREEPRAPWIEQEGPEYWDRNTQIYKAQAQTDRESLRNLRGYYNQSEAGSHTWQTM |
| | YGCDLGPDGRLLRGHNQLAYDGKDYIALNEDLSSWTAADTAAQITQRKWEAAREAEQLRA |
| | YLEGTCVEWLRRYLENGKETLQRADPPKTHVTHHPISDHEATLRCWALGFYPAEITLTWQRD |
| | GEDQTQDTELVETRPAGDRTFQKWAAVVVPSGEEQRYTCHVQHEGLPKPLTLRWEPSSQSTI |
| | PIVGIVAGLAVLAVVVIGAVVATVMCRRKSSGGKGGSYSQAASSDSAQGSDVSLTA |
| | (HLA-B*55:01 full-length) |
| 69 | MRVTAPRTVLLLLSAALALTETWAGSHSMRYFHTAMSRPGRGEPRFITVGYVDDTLFVRFDS |
| | DATSPRKEPRAPWIEQEGPEYWDRETQISKTNTQTYRESLRNLRGYYNQSEAGSHTWQRMY |
| | GCDLGPDGRLLRGYNQLAYDGKDYIALNEDLSSWTAADTAAQITQRKWEAARVAEQDRAY |
| | LEGLCVESLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTIPI |
| | VGIVAGLAVLAVVVIGAVVATVMCRRKSSGGKGGSYSQAASSDSAQGSDVSLTA |
| | (HLA-B*45:01 full-length) |
| 70 | MRVTAPRTVLLLLWGAVALTETWAGSHSMRYFYTAMSRPGRGEPRFIAVGYVDDTQFVRFD |
| | SDAASPRTEPRAPWIEQEGPEYWDRETQISKTNTQTYRENLRIALRYYNQSEAGSHTWQTMY |
| | GCDVGPDGRLLRGHNQYAYDGKDYIALNEDLSSWTAADTAAQITQRKWEAAREAEQLRAY |
| | LEGLCVEWLRRHLENGKETLQRADPPKTHVTHHPVSDHEATLRCWALGFYPAEITLTWQRD |
| | GEDQTQDTELVETRPAGDRTFQKWAAVVVPSGEEQRYTCHVQHEGLPKPLTLRWEPSSQSTI |
| | PIVGIVAGLAVLAVVVIGAVVATVMCRRKSSGGKGGSYSQAASSDSAQGSDVSLTA |
| | (HLA-B*52:01 full-length) |
| 71 | MRVMAPRTLILLLSGALALTETWACSHSMRYFYTAVSRPGRGEPRFIA VGYVDDTQFVRFDS |
| | DAASPRGEPRAPWVEQEGPEYWDRETQKYKRQAQADRVSLRNLRGYYNQSEAGSHTLQRM |
| | YGCDLGPDGRLLRGYDQSAYDGKDYIALNEDLRSWTAADTAAQITQRKWEAAREAEQWRA |
| | YLEGTCVEWLRRYLENGKETLQRAEHPKTHVTHHPVSDHEATLRCWALGFYPAEITLTWQR |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVMAVVMCRRKSSGGKGGSCSQAASSNSAQGSDESLIACKA |
| | (HLA-C*12:02 full-length) |
| 72 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRESLRNLRGYYNQSEAGSHIIQRMYG |
| | CDLGPDGRLLRGHDQFAYDGKDYIALNEDLSSWTAADTAAQITQRKWEAARVAEQLRAYLE |
| | GLCVEWLRRYLENGKETLQRADPPKTHVTHHPVSDHEATLRCWALGFYPAEITLTWQRDGE |
| | DQTQDTELVETRPAGDRTFQKWAAVVVPSGEEQRYTCHVQHEGLPKPLTLRWEPSSQSTIPIV |
| | GIVAGLAVLAVVVIGAVVATVMCRRKSSGGKGGSYSQAASSDSAQGSDVSLTA |
| | (HLA-B*35:03 full-length) |
| 73 | MRVTAPRTLLLLLWGAVALTETWAGSHSMRYFHTSVSRPGRGEPRFITVGYVDDTLFVRFDS |
| | DATSPRKEPRAPWIEQEGPEYWDRETQISKTNTQTYRESLRNLRGYYNQSEAGSHTLQSMYG |
| | CDVGPDGRLLRGHNQYAYDGKDYIALNEDLRSWTAADTAAQITQRKWEAARVAEQLRAYL |
| | EGECVEWLRRYLENGKETLQRADPPKTHVTHHPISDHEATLRCWALGFYPAEITLTWQRDGE |
| | DQTQDTELVETRPAGDRTFQKWAAVVVPSGEEQRYTCHVQHEGLPKPLTLRWEPSSQSTVPI |
| | VGIVAGLAVLAVVVIGAVVAAVMCRRKSSGGKGGSYSQAACSDSAQGSDVSLTA |
| | (HLA-B*40:02 full-length) |
| 74 | MRVTAPRTVLLLLSGALALTETWAGSHSMRYFYTAMSRPGRGEPRFISVGYVDDTQFVRFDS |
| | DAASPREEPRAPWIEQEGPEYWDRETQISKTNTQTYRESLRNLRGYYNQSEAGSHTLQRMYG |
| | CDVGPDGRLLRGHDQSAYDGKDYIALNEDLSSWTAADTAAQITQRKWEAAREAEQLRAYLE |
| | GLCVEWLRRYLENGKETLQRADPPKTHVTHHPISDHEATLRCWALGFYPAEITLTWQRDGED |
| | QTQDTELVETRPAGDRTFQKWAAVVVPSGEEQRYTCHVQHEGLPKPLTLRWEPSSQSTIPIVG |
| | IVAGLAVLAVVVIGAVVATVMCRRKSSGGKGGSYSQAASSDSAQGSDVSLTA |
| | (HLA-B*15:03 full-length) |
| 75 | MAVMAPRTLLLLLLGALALTQTRAGSHSMRYFFTSVSRPGRGEPRFIAVGYVDDTQFVRFDS |
| | DAASQRMEPRAPWIEQEGPEYWDQETRNVKAHSQTDRVDLGTLRGYYNQSEAGSHTIQMM |
| | YGCDVGPDGRLLRGYQQDAYDGKDYIALNEDLRSWTAADMAAQITQRKWEAARVAEQLR |
| | AYLEGTCVEWLRRYLENGKETLQRTDAPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAMFAGAVVAAVRWRRKSSDRKGGSYSQAASSDSAQGSDMSLTACKV |
| | (HLA-A*74:01 full-length) |
| 76 | GSHSMRYFFTSVSRPGRGEPRFIAVGYVDDTQFVRFDSDAASQKMEPRAPWIEQEGPEYWDQ |
| | ETRNMKAHSQTDRANLGTLRGYYNQSEDGSHTIQIMYGCDVGPDGRFLRGYRQDAYDGKD |
| | YIALNEDLRSWTAADMAAQITKRKWEAVHAAEQRRVYLEGRCVDGLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWELSS |
| | (HLA-A*01:01 soluble) |
| 77 | GSHSMRYFFTSVSRPGRGEPRFIAVGYVDDTQFVRFDSDAASQRMEPRAPWIEQEGPEYWDQ |
| | ETRNVKAQSQTDRVDLGTLRGYYNQSEAGSHTIQIMYGCDVGSDGRFLRGYRQDAYDGKD |
| | YIALNEDLRSWTAADMAAQITKRKWEAAHEAEQLRAYLDGTCVEWLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWELSS |
| | (HLA-A*03:01 soluble) |
| 78 | GSHSMRYFYTSVSRPGRGEPRFIAVGYVDDTQFVRFDSDAASQRMEPRAPWIEQEGPEYWDQ |
| | ETRNVKAQSQTDRVDLGTLRGYYNQSEDGSHTIQIMYGCDVGPDGRFLRGYRQDAYDGKD |
| | YIALNEDLRSWTAADMAAQITKRKWEAAHAAEQQRAYLEGRCVEWLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWELSS |
| | (HLA-A*11:01 soluble) |
| 79 | GSHSMRYFSTSVSRPGRGEPRFIAVGYVDDTQFVRFDSDAASQRMEPRAPWIEQEGPEYWDE |
| | ETGKVKAHSQTDRENLRIALRYYNQSEAGSHTLQMMFGCDVGSDGRFLRGYHQYAYDGKD |
| | YIALKEDLRSWTAADMAAQITKRKWEAAHVAEQQRAYLEGTCVDGLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWEPSS |
| | (HLA-A*24:02 soluble) |
| 80 | GSHSMRYFYTSVSRPGRGEPRFISVGYVDDTQFVRFDSDAASPREEPRAPWIEQEGPEYWDRN |
| | TQIYKAQAQTDRESLRNLRGYYNQSEAGSHTLQSMYGCDVGPDGRLLRGHDQYAYDGKDYI |
| | ALNEDLRSWTAADTAAQITQRKWEAAREAEQRRAYLEGECVEWLRRYLENGKDKLERADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*07:02 soluble) |
| 81 | GSHSMRYFSTSVSWPGRGEPRFIAVGYVDDTQFVRFDSDAASPRGEPREPWVEQEGPEYWDR |
| | ETQKYKRQAQADRVNLRKLRGYYNQSEDGSHTLQRMFGCDLGPDGRLLRGYNQFAYDGKD |
| | YIALNEDLRSWTAADTAAQITQRKWEAAREAEQRRAYLEGTCVEWLRRYLENGKETLQRAE |
| | HPKTHVTHHPVSDHEATLRCWALGFYPAEITLTWQWDGEDQTQDTELVETRPAGDGTFQK |
| | WAAVVVPSGEEQRYTCHVQHEGLPEPLTLRWKPSS |
| | (HLA-C*04:01 soluble) |
| 82 | CSHSMRYFDTAVSRPGRGEPRFISVGYVDDTQFVRFDSDAASPRGEPRAPWVEQEGPEYWDR |
| | ETQKYKRQAQADRVSLRNLRGYYNQSEDGSHTLQRMSGCDLGPDGRLLRGYDQSAYDGKD |
| | YIALNEDLRSWTAADTAAQITQRKLEAARAAEQLRAYLEGTCVEWLRRYLENGKETLQRAE |
| | PPKTHVTHHPLSDHEATLRCWALGFYPAEITLTWQRDGEDQTQDTELVETRPAGDGTFQKW |
| | AAVVVPSGQEQRYTCHMQHEGLQEPLTLSWEPSS |
| | (HLA-C*07:02 soluble) |
| 83 | GSHSMRYFDTAMSRPGRGEPRFISVGYVDDTQFVRFDSDAASPREEPRAPWIEQEGPEYWDR |
| | NTQIFKTNTQTDRESLRNLRGYYNQSEAGSHTLQSMYGCDVGPDGRLLRGHNQYAYDGKDY |
| | IALNEDLRSWTAADTAAQITQRKWEAARVAEQDRAYLEGTCVEWLRRYLENGKDTLERADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*08:01 soluble) |
| 84 | GSHSMRYFYTAMSRPGRGEPRFIAVGYVDDTQFVRFDSDAASPRTEPRAPWIEQEGPEYWDR |
| | NTQIFKTNTQTYRESLRNLRGYYNQSEAGSHIIQRMYGCDLGPDGRLLRGHDQSAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*35:01 soluble) |
| 85 | GSHSMRYFYTAMSRPGRGEPRFIAVGYVDDTQFVRFDSDAASPRMAPRAPWIEQEGPEYWD |
| | GETRNMKASAQTYRENLRIALRYYNQSEAGSHIIQVMYGCDVGPDGRLLRGHDQSAYDGKD |
| | YIALNEDLSSWTAADTAAQITQRKWEAARVAEQLRAYLEGLCVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*57:01 soluble) |
| 86 | GSHSMRYFYTAMSRPGRGEPRFIAVGYVDDTQFVRFDSDAASPRMAPRAPWIEQEGPEYWD |
| | GETRNMKASAQTYRENLRIALRYYNQSEAGSHIIQVMYGCDVGPDGRLLRGHNQYAYDGKD |
| | YIALNEDLSSWTAADTAAQITQRKWEAARVAEQLRAYLEGLCVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*57:03 soluble) |
| 87 | GSHSLKYFHTSVSRPGRGEPRFISVGYVDDTQFVRFDNDAASPRMVPRAPWMEQEGSEYWD |
| | RETRSARDTAQIFRVNLRTLRGYYNQSEAGSHTLQWMHGCELGPDGRFLRGYEQFAYDGKD |
| | YLTLNEDLRSWTAVDTAAQISEQKSNDASEAEHQRAYLEDTCVEWLHKYLEKGKETLLHLE |
| | PPKTHVTHHPISDHEATLRCWALGFYPAEITLTWQQDGEGHTQDTELVETRPAGDGTFQKWA |
| | AVVVPSGEEQRYTCHVQHEGLPEPVTLRWKPAS |
| | (HLA-E soluble) |
| 88 | CSHSMRYFYTAVSRPGRGEPRFIAVGYVDDTQFVRFDSDAASPRGEPRAPWVEQEGPEYWD |
| | RETQKYKRQAQTDRVSLRNLRGYYNQSEAGSHTLQWMYGCDLGPDGRLLRGYDQSAYDG |
| | KDYIALNEDLRSWTAADTAAQITQRKWEAARAAEQQRAYLEGTCVEWLRRYLENGKETLQ |
| | RAEHPKTHVTHHLVSDHEATLRCWALGFYPAEITLTWQRDGEDQTQDTELVETRPAGDGTF |
| | QKWAAVVVPSGEEQRYTCHVQHEGLPEPLTLRWEPSS |
| | (HLA-C*16:01 soluble) |
| 89 | CSHSMRYFYTAVSRPGRGEPRFIAVGYVDDTQFVQFDSDAASPRGEPRAPWVEQEGPEYWD |
| | RETQKYKRQAQTDRVSLRNLRGYYNQSEAGSHTLQRMYGCDLGPDGRLLRGYNQFAYDGK |
| | DYIALNEDLRSWTAADKAAQITQRKWEAAREAEQRRAYLEGTCVEWLRRYLENGKKTLQR |
| | AEHPKTHVTHHPVSDHEATLRCWALGFYPAEITLTWQRDGEDQTQDTELVETRPAGDGTFQ |
| | KWAAVVVPSGEEQRYTCHVQHEGLPEPLTLRWGPSS |
| | (HLA-C*08:02 soluble) |
| 90 | CSHSMRYFDTAVSRPGRGEPRFISVGYVDDTQFVRFDSDAASPRGEPRAPWVEQEGPEYWDR |
| | ETQNYKRQAQADRVSLRNLRGYYNQSEDGSHTLQRMYGCDLGPDGRLLRGYDQSAYDGKD |
| | YIALNEDLRSWTAADTAAQITQRKLEAARAAEQLRAYLEGTCVEWLRRYLENGKETLQRAE |
| | PPKTHVTHHPLSDHEATLRCWALGFYPAEITLTWQRDGEDQTQDTELVETRPAGDGTFQKW |
| | AAVVVPSGQEQRYTCHMQHEGLQEPLTLSWEPSS |
| | (HLA-C*07:01 soluble) |
| 91 | CSHSMRYFYTAVSRPGRGEPRFIAVGYVDDTQFVQFDSDAASPRGEPRAPWVEQEGPEYWD |
| | RETQKYKRQAQTDRVNLRKLRGYYNQSEAGSHTLQRMYGCDLGPDGRLLRGYNQFAYDGK |
| | DYIALNEDLRSWTAADKAAQITQRKWEAAREAEQRRAYLEGTCVEWLRRYLENGKKTLQR |
| | AEHPKTHVTHHPVSDHEATLRCWALGFYPAEITLTWQRDGEDQTQDTELVETRPAGDGTFQ |
| | KWAAVVVPSGEEQRYTCHVQHEGLPEPLTLRWGPSS |
| | (HLA-C*05:01 soluble) |
| 92 | GSHSMRYFYTAMSRPGRGEPRFITVGYVDDTLFVRFDSDATSPRKEPRAPWIEQEGPEYWDR |
| | ETQISKTNTQTYRENLRTALRYYNQSEAGSHIIQRMYGCDVGPDGRLLRGYDQDAYDGKDYI |
| | ALNEDLSSWTAADTAAQITQRKWEAARVAEQDRAYLEGLCVESLRRYLENGKETLQRADPP |
| | KTHVTHHPISDHEVTLRCWALGFYPAEITLTWQRDGEDQTQDTELVETRPAGDRTFQKWAA |
| | VVVPSGEEQRYTCHVQHEGLPKPLTLRWEPSS |
| | (HLA-B*44:02 soluble) |
| 93 | GSHSMRYFTTSVSRPGRGEPRFIAVGYVDDTQFVRFDSDAASQRMEPRAPWIEQEGPEYWDL |
| | QTRNVKAQSQTDRANLGTLRGYYNQSEAGSHTIQMMYGCDVGSDGRFLRGYRQDAYDGKD |
| | YIALNEDLRSWTAADMAAQITQRKWEAARVAEQLRAYLEGTCVEWLRRYLENGKETLQRT |
| | DAPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*29:02 soluble) |
| 94 | GSHSMRYFYTAMSRPGRGEPRFITVGYVDDTLFVRFDSDATSPRKEPRAPWIEQEGPEYWDR |
| | ETQISKTNTQTYRENLRTALRYYNQSEAGSHIIQRMYGCDVGPDGRLLRGYDQDAYDGKDYI |
| | ALNEDLSSWTAADTAAQITQRKWEAARVAEQLRAYLEGLCVESLRRYLENGKETLQRADPP |
| | KTHVTHHPISDHEVTLRCWALGFYPAEITLTWQRDGEDQTQDTELVETRPAGDRTFQKWAA |
| | VVVPSGEEQRYTCHVQHEGLPKPLTLRWEPSS |
| | (HLA-B*44:03 soluble) |
| 95 | GSHSMRYFYTAVSRPGRGEPHFIAVGYVDDTQFVRFDSDAASPRGEPRAPWVEQEGPEYWD |
| | RETQKYKRQAQTDRVSLRNLRGYYNQSEAGSHIIQRMYGCDVGPDGRLLRGYDQYAYDGK |
| | DYIALNEDLRSWTAADTAAQITQRKWEAAREAEQLRAYLEGLCVEWLRRYLKNGKETLQR |
| | AEHPKTHVTHHPVSDHEATLRCWALGFYPAEITLTWQWDGEDQTQDTELVETRPAGDGTFQ |
| | KWAAVVVPSGEEQRYTCHVQHEGLPEPLTLRWEPSS |
| | (HLA-C*03:04 soluble) |
| 96 | GSHSMRYFHTAMSRPGRGEPRFITVGYVDDTLFVRFDSDATSPRKEPRAPWIEQEGPEYWDR |
| | ETQISKTNTQTYRESLRNLRGYYNQSEAGSHTLQRMYGCDVGPDGRLLRGHNQYAYDGKDY |
| | IALNEDLRSWTAADTAAQISQRKLEAARVAEQLRAYLEGECVEWLRRYLENGKDKLERADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*40:01 soluble) |
| 97 | CSHSMRYFDTAVSRPGRGEPRFISVGYVDDTQFVRFDSDAASPRGEPRAPWVEQEGPEYWDR |
| | ETQKYKRQAQADRVNLRKLRGYYNQSEDGSHTLQWMYGCDLGPDGRLLRGYDQSAYDGK |
| | DYIALNEDLRSWTAADTAAQITQRKWEAAREAEQWRAYLEGTCVEWLRRYLENGKETLQR |
| | AEHPKTHVTHHPVSDHEATLRCWALGFYPAEITLTWQRDGEDQTQDTELVETRPAGDGTFQ |
| | KWAAVVVPSGEEQRYTCHVQHEGLPEPLTLRWEPSS |
| | (HLA-C*06:02 soluble) |
| 98 | GSHSMRYFYTAMSRPGRGEPRFIAVGYVDDTQFVRFDSDAASPRMAPRAPWIEQEGPEYWD |
| | RETQISKTNTQTYRESLRNLRGYYNQSEAGSHTLQRMYGCDVGPDGRLLRGHDQSAYDGKD |
| | YIALNEDLSSWTAADTAAQITQRKWEAAREAEQWRAYLEGLCVEWLRRYLENGKETLQRA |
| | DPPKTHVTHHPISDHEATLRCWALGFYPAEITLTWQRDGEDQTQDTELVETRPAGDRTFQKW |
| | AAVVVPSGEEQRYTCHVQHEGLPKPLTLRWEPSS |
| | (HLA-B*15:01 soluble) |
| 99 | GSHSMRYFYTAVSRPGRGEPHFIAVGYVDDTQFVRFDSDAASPRGEPRAPWVEQEGPEYWD |
| | RETQKYKRQAQTDRVSLRNLRGYYNQSEARSHIIQRMYGCDVGPDGRLLRGYDQYAYDGK |
| | DYIALNEDLRSWTAADTAAQITQRKWEAAREAEQLRAYLEGLCVEWLRRYLKNGKETLQR |
| | AEHPKTHVTHHPVSDHEATLRCWALGFYPAEITLTWQWDGEDQTQDTELVETRPAGDGTFQ |
| | KWAAVVVPSGEEQRYTCHVQHEGLPEPLTLRWEPSS |
| | (HLA-C*03:03 soluble) |
| 100 | GSHSMRYFSTSVSRPGSGEPRFIAVGYVDDTQFVRFDSDAASQRMEPRAPWIEQERPEYWDQ |
| | ETRNVKAQSQTDRVDLGTLRGYYNQSEAGSHTIQIMYGCDVGSDGRFLRGYEQHAYDGKDY |
| | IALNEDLRSWTAADMAAQITQRKWEAARWAEQLRAYLEGTCVEWLRRYLENGKETLQRTD |
| | PPKTHMTHHPISDHEATLRCWALGFYPAEITLTWQRDGEDQTQDTELVETRPAGDGTFQKW |
| | AAVVVPSGEEQRYTCHVQHEGLPKPLTLRWELSS |
| | (HLA-A*30:01 soluble) |
| 101 | GSHSMRYFYTAMSRPGRGEPRFITVGYVDDTQFVRFDSDATSPRMAPRAPWIEQEGPEYWDR |
| | ETQISKTNTQTYRENLRTALRYYNQSEAGSHTWQTMYGCDLGPDGRLLRGHNQLAYDGKD |
| | YIALNEDLSSWTAADTAAQITQLKWEAARVAEQLRAYLEGECVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*13:02 soluble) |
| 102 | CSHSMRYFYTAVSRPGRGEPRFIAVGYVDDTQFVRFDSDAASPRGEPRAPWVEQEGPEYWD |
| | RETQKYKRQAQADRVSLRNLRGYYNQSEAGSHTLQWMYGCDLGPDGRLLRGYDQSAYDG |
| | KDYIALNEDLRSWTAADTAAQITQRKWEAAREAEQWRAYLEGTCVEWLRRYLENGKETLQ |
| | RAEHPKTHVTHHPVSDHEATLRCWALGFYPAEITLTWQRDGEDQTQDTELVETRPAGDGTF |
| | QKWAAVVVPSGEEQRYTCHVQHEGLPEPLTLRWEPSS |
| | (HLA-C*12:03 soluble) |
| 103 | GSHSMRYFYTSVSRPGRGEPRFIAVGYVDDTQFVRFDSDAASQRMEPRAPWIEQEGPEYWDR |
| | NTRNVKAHSQTDRANLGTLRGYYNQSEDGSHTIQRMYGCDVGPDGRFLRGYQQDAYDGKD |
| | YIALNEDLRSWTAADMAAQITQRKWETAHEAEQWRAYLEGRCVEWLRRYLENGKETLQRT |
| | DAPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*26:01 soluble) |
| 104 | GSHSMRYFYTSVSRPGRGEPRFISVGYVDDTQFVRFDSDAASPREEPRAPWIEQEGPEYWDRN |
| | TQICKTNTQTYRENLRIALRYYNQSEAGSHTLQRMYGCDVGPDGRLLRGHNQFAYDGKDYI |
| | ALNEDLSSWTAADTAAQITQRKWEAARVAEQLRTYLEGTCVEWLRRYLENGKETLQRADPP |
| | KTHVTHHPISDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*38:01 soluble) |
| 105 | GSHSMRYFYTAVSRPGRGEPRFISVGYVDDTQFVRFDSDAASPREEPRAPWIEQEGPEYWDR |
| | NTQICKTNTQTDRESLRNLRGYYNQSEAGSHTLQWMYGCDVGPDGRLLRGYNQFAYDGKD |
| | YIALNEDLSSWTAADTAAQITQRKWEAAREAEQLRAYLEGTCVEWLRRHLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*14:02 soluble) |
| 106 | GSHSMRYFTTSVSRPGRGEPRFIAVGYVDDTQFVRFDSDAASQRMEPRAPWIEQEGPEYWDR |
| | NTRNVKAHSQIDRVDLGTLRGYYNQSEAGSHTIQMMYGCDVGSDGRFLRGYQQDAYDGKD |
| | YIALNEDLRSWTAADMAAQITQRKWEAARVAEQLRAYLEGTCVEWLRRHLENGKETLQRT |
| | DPPRTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*33:01 soluble) |
| 107 | GSHSMRYFSTSVSRPGRGEPRFIAVGYVDDTQFVRFDSDAASQRMEPRAPWIEQEGPEYWDE |
| | ETGKVKAHSQTDRENLRIALRYYNQSEAGSHTLQMMFGCDVGSDGRFLRGYHQYAYDGKD |
| | YIALKEDLRSWTAADMAAQITQRKWEAARVAEQLRAYLEGTCVDGLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWEPSS |
| | (HLA-A*23:01 soluble) |
| 108 | GSHSMRYFYTSVSRPGRGEPRFIAVGYVDDTQFVRFDSDAASQRMEPRAPWIEQEGPEYWDR |
| | NTRNVKAHSQTDRESLRIALRYYNQSEDGSHTIQRMYGCDVGPDGRFLRGYQQDAYDGKDY |
| | IALNEDLRSWTAADMAAQITQRKWETAHEAEQWRAYLEGRCVEWLRRYLENGKETLQRTD |
| | APKTHMTHHAVSDHEATLRCWALSFYPAEITLTWQRDGEDQTQDTELVETRPAGDGTFQKW |
| | ASVVVPSGQEQRYTCHVQHEGLPKPLTLRWEPSS |
| | (HLA-A*25:01 soluble) |
| 109 | GSHSMRYFHTSVSRPGRGEPRFISVGYVDGTQFVRFDSDAASPRTEPRAPWIEQEGPEYWDRN |
| | TQISKTNTQTYRESLRNLRGYYNQSEAGSHTLQRMYGCDVGPDGRLLRGHDQSAYDGKDYI |
| | ALNEDLSSWTAADTAAQITQRKWEAARVAEQLRAYLEGTCVEWLRRHLENGKETLQRADPP |
| | KTHVTHHPISDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*18:01 soluble) |
| 110 | GSHSMRYFHTSVSRPGRGEPRFISVGYVDDTQFVRFDSDAASPRTEPRAPWIEQEGPEYWDRE |
| | TQISKTNTQTYREDLRTLLRYYNQSEAGSHTIQRMSGCDVGPDGRLLRGYNQFAYDGKDYIA |
| | LNEDLSSWTAADTAAQITQRKWEAARVAEQDRAYLEGTCVEWLRRYLENGKETLQRADPP |
| | KTHVTHHPISDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*37:01 soluble) |
| 111 | GSHSMRYFYTAMSRPGRGEPRFIAVGYVDDTQFVRFDSDAASPRTEPRAPWIEQEGPEYWDR |
| | NTQIFKTNTQTYRENLRIALRYYNQSEAGSHTWQTMYGCDVGPDGRLLRGHNQYAYDGKD |
| | YIALNEDLSSWTAADTAAQITQRKWEAAREAEQLRAYLEGLCVEWLRRHLENGKETLQRAD |
| | PPKTHVTHHPVSDHEATLRCWALGFYPAEITLTWQRDGEDQTQDTELVETRPAGDRTFQKW |
| | AAVVVPSGEEQRYTCHVQHEGLPKPLTLRWEPSS |
| | (HLA-B*51:01 soluble) |
| 112 | CSHSMRYFSTSVSRPGRGEPRFIAVGYVDDTQFVRFDSDAASPRGEPRAPWVEQEGPEYWDR |
| | ETQKYKRQAQTDRVSLRNLRGYYNQSEAGSHTLQWMFGCDLGPDGRLLRGYDQSAYDGKD |
| | YIALNEDLRSWTAADTAAQITQRKWEAAREAEQRRAYLEGTCVEWLRRYLENGKETLQRAE |
| | HPKTHVTHHPVSDHEATLRCWALGFYPAEITLTWQWDGEDQTQDTELVETRPAGDGTFQK |
| | WAAVVVPSGEEQRYTCHVQHEGLPEPLTLRWEPSS |
| | (HLA-C*14:02 soluble) |
| 113 | CSHSMRYFYTAVSRPGRGEPHFIAVGYVDDTQFVRFDSDAASPRGEPRAPWVEQEGPEYWD |
| | RETQNYKRQAQTDRVNLRKLRGYYNQSEAGSHIIQRMYGCDLGPDGRLLRGHDQLAYDGK |
| | DYIALNEDLRSWTAADTAAQITQRKWEAAREAEQLRAYLEGTCVEWLRRYLENGKETLQRA |
| | EHPKTHVTHHPVSDHEATLRCWALGFYPAEITLTWQRDGEDQTQDTELVETRPAGDGTFQK |
| | WAAVVVPSGEEQRYTCHVQHEGLPEPLTLRWEPSS |
| | (HLA-C*15:02 soluble) |
| 114 | CSHSMRYFYTAVSRPSRGEPHFIAVGYVDDTQFVRFDSDAASPRGEPRAPWVEQEGPEYWDR |
| | ETQKYKRQAQTDRVNLRKLRGYYNQSEAGSHTLQRMYGCDLGPDGRLLRGYDQSAYDGKD |
| | YIALNEDLRSWTAADTAAQITQRKWEAAREAEQWRAYLEGECVEWLRRYLENGKETLQRA |
| | EHPKTHVTHHPVSDHEATLRCWALGFYPTEITLTWQRDGEDQTQDTELVETRPAGDGTFQK |
| | WAAVVVPSGEEQRYTCHVQHEGLPEPLTLRWEPSS |
| | (HLA-C*02:02 soluble) |
| 115 | GSHSMRYFHTSVSRPGRGEPRFITVGYVDDTLFVRFDSDAASPREEPRAPWIEQEGPEYWDRE |
| | TQICKAKAQTDREDLRTLLRYYNQSEAGSHTLQNMYGCDVGPDGRLLRGYHQDAYDGKDY |
| | IALNEDLSSWTAADTAAQITQRKWEAARVAEQLRAYLEGECVEWLRRYLENGKETLQRADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*27:05 soluble) |
| 116 | GSHSMRYFTTSVSRPGRGEPRFIAVGYVDDTQFVRFDSDAASQRMEPRAPWIEQERPEYWDQ |
| | ETRNVKAHSQIDRVDLGTLRGYYNQSEAGSHTIQMMYGCDVGSDGRFLRGYQQDAYDGKD |
| | YIALNEDLRSWTAADMAAQITQRKWEAARVAEQLRAYLEGTCVEWLRRYLENGKETLQRT |
| | DPPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*31:01 soluble) |
| 117 | GSHSMRYFSTSVSRPGSGEPRFIAVGYVDDTQFVRFDSDAASQRMEPRAPWIEQERPEYWDQ |
| | ETRNVKAHSQTDRENLGTLRGYYNQSEAGSHTIQIMYGCDVGSDGRFLRGYEQHAYDGKDY |
| | IALNEDLRSWTAADMAAQITQRKWEAARRAEQLRAYLEGTCVEWLRRYLENGKETLQRTDP |
| | PKTHMTHHPISDHEATLRCWALGFYPAEITLTWQRDGEDQTQDTELVETRPAGDGTFQKWA |
| | AVVVPSGEEQRYTCHVQHEGLPKPLTLRWELSS |
| | (HLA-A*30:02 soluble) |
| 118 | GSHSMRYFYTSVSRPGRGEPRFISVGYVDDTQFVRFDSDAASPREEPRAPWIEQEGPEYWDRN |
| | TQIYKAQAQTDRESLRNLRGYYNQSEAGSHTLQSMYGCDVGPDGRLLRGHNQYAYDGKDYI |
| | ALNEDLRSWTAADTAAQITQRKWEAARVAEQDRAYLEGTCVEWLRRYLENGKDTLERADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*42:01 soluble) |
| 119 | GSHSMRYFYTAVSRPGRGEPRFIAVGYVDDTQFVRFDSDAASPRGEPRAPWVEQEGPEYWD |
| | RETQKYKRQAQADRVNLRKLRGYYNQSEAGSHTIQRMYGCDLGPDGRLLRGYNQFAYDGK |
| | DYIALNEDLRSWTAADTAAQISQRKLEAAREAEQLRAYLEGECVEWLRGYLENGKETLQRA |
| | ERPKTHVTHHPVSDHEATLRCWALGFYPAEITLTWQRDGEDQTQDTELVETRPAGDGTFQK |
| | WAAVVVPSGQEQRYTCHVQHEGLQEPCTLRWKPSS |
| | (HLA-C*17:01 soluble) |
| 120 | GSHSMRYFYTAMSRPGRGEPRFIAVGYVDDTQFVRFDSDAASPRTEPRAPWIEQEGPEYWDR |
| | NTQIFKTNTQTYRESLRNLRGYYNQSEAGSHIIQRMYGCDLGPDGRFLRGHNQYAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*35:02 soluble) |
| 121 | GSHSMRYFYTSVSRPGRGEPRFISVGYVDDTQFVRFDSDAASPREEPRAPWIEQEGPEYWDRN |
| | TQICKTNTQTDRESLRNLRGYYNQSEAGSHTWQTMYGCDVGPDGRLLRGHNQFAYDGKDYI |
| | ALNEDLSSWTAADTAAQITQRKWEAARVAEQLRTYLEGTCVEWLRRYLENGKETLQRADPP |
| | KTHVTHHPISDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*39:06 soluble) |
| 122 | GSHSMRYFYTAVSRPGRGEPHFIAVGYVDDTQFVRFDSDAASPRGEPRAPWVEQEGPEYWD |
| | RETQKYKRQAQTDRVSLRNLRGYYNQSEAGSHILQRMYGCDVGPDGRLLRGYDQSAYDGK |
| | DYIALNEDLRSWTAADTAAQITQRKWEAAREAEQLRAYLEGLCVEWLRRYLKNGKETLQR |
| | AEHPKTHVTHHPVSDHEATLRCWALGFYPAEITLTWQWDGEDQTQDTELVETRPAGDGTFQ |
| | KWAAVVVPSGEEQRYTCHVQHEGLPEPLTLRWEPSS |
| | (HLA-C*03:02 soluble) |
| 123 | GSHSMRYFYTAMSRPGRGEPRFIAVGYVDDTQFVRFDSDAASPRTEPRAPWIEQEGPEYWDG |
| | ETRNMKASAQTYRENLRIALRYYNQSEAGSHIIQRMYGCDLGPDGRLLRGHDQSAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*58:01 soluble) |
| 124 | GSHSMRYFTTSVSRPGRGEPRFIAVGYVDDTQFVRFDSDAASQRMEPRAPWIEQEGPEYWDR |
| | NTRNVKAHSQIDRVDLGTLRGYYNQSEAGSHTIQMMYGCDVGSDGRFLRGYQQDAYDGKD |
| | YIALNEDLRSWTAADMAAQITQRKWEAARVAEQLRAYLEGTCVEWLRRYLENGKETLQRT |
| | DPPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*33:03 soluble) |
| 125 | GSHSMRYFYTSMSRPGRGEPRFIAVGYVDDTQFVRFDSDAASQRMEPRAPWIEQEGPEYWD |
| | RNTRNVKAQSQTDRVDLGTLRGYYNQSEAGSHTIQRMYGCDVGPDGRFLRGYHQYAYDGK |
| | DYIALKEDLRSWTAADMAAQTTKHKWEAAHVAEQWRAYLEGTCVEWLRRYLENGKETLQ |
| | RTDAPKTHMTHHAVSDHEATLRCWALSFYPAEITLTWQRDGEDQTQDTELVETRPAGDGTF |
| | QKWVAVVVPSGQEQRYTCHVQHEGLPKPLTLRWEPSS |
| | (HLA-A*68:02 soluble) |
| 126 | CSHSMKYFFTSVSRPGRGEPRFISVGYVDDTQFVRFDSDAASPRGEPRAPWVEQEGPEYWDR |
| | ETQKYKRQAQTDRVSLRNLRGYYNQSEAGSHTLQWMCGCDLGPDGRLLRGYDQYAYDGK |
| | DYIALNEDLRSWTAADTAAQITQRKWEAAREAEQRRAYLEGTCVEWLRRYLENGKETLQRA |
| | EHPKTHVTHHPVSDHEATLRCWALGFYPAEITLTWQWDGEDQTQDTELVETRPAGDGTFQK |
| | WAAVMVPSGEEQRYTCHVQHEGLPEPLTLRWEPSS |
| | (HLA-C*01:02 soluble) |
| 127 | CSHSMRYFDTAVSRPGRGEPRFISVGYVDDTQFVRFDSDAASPRGEPRAPWVEQEGPEYWDR |
| | ETQKYKRQAQADRVSLRNLRGYYNQSEDGSHTFQRMYGCDLGPDGRLLRGYDQFAYDGKD |
| | YIALNEDLRSWTAADTAAQITQRKLEAARAAEQDRAYLEGTCVEWLRRYLENGKKTLQRAE |
| | PPKTHVTHHPLSDHEATLRCWALGFYPAEITLTWQRDGEDQTQDTELVETRPAGDGTFQKW |
| | AAVVVPSGQEQRYTCHMQHEGLQEPLTLSWEPSS |
| | (HLA-C*07:04 soluble) |
| 128 | GSHSMRYFYTSVSRPGRGEPRFIAVGYVDDTQFVRFDSDAASQRMEPRAPWIEQEGPEYWDR |
| | NTRNVKAQSQTDRVDLGTLRGYYNQSEAGSHTIQMMYGCDVGSDGRFLRGYRQDAYDGKD |
| | YIALKEDLRSWTAADMAAQTTKHKWEAAHVAEQWRAYLEGTCVEWLRRYLENGKETLQR |
| | TDAPKTHMTHHAVSDHEATLRCWWALSFYPAEITLTWQRDGEDQTQDTELVETRPAGDGTFQ |
| | KWVAVVVPSGQEQRYTCHVQHEGLPKPLTLRWEPSS |
| | (HLA-A*68:01 soluble) |
| 129 | GSHSMRYFFTSVSRPGRGEPRFIAVGYVDDTQFVRFDSDAASQRMEPRAPWIEQEGPEYWDQ |
| | ETRNVKAHSQTDRESLRIALRYYNQSEAGSHTIQMMYGCDVGPDGRLLRGYQQDAYDGKD |
| | YIALNEDLRSWTAADMAAQITQRKWEAARVAEQLRAYLEGTCVEWLRRYLENGKETLQRT |
| | DAPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*32:01 soluble) |
| 130 | GSHSMRYFHTAMSRPGRGEPRFITVGYVDDTLFVRFDSDATSPRKEPRAPWIEQEGPEYWDR |
| | ETQISKTNTQTYRENLRIALRYYNQSEAGSHTWQRMYGCDLGPDGRLLRGYNQLAYDGKDY |
| | IALNEDLSSWTAADTAAQITQRKWEAAREAEQLRAYLEGLCVEWLRRYLENGKETLQRADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*49:01 soluble) |
| 131 | GSHSMRYFYTAMSRPGRGEPRFIAVGYVDDTQFVRFDSDAASPRTEPRAPWIEQEGPEYWDR |
| | NTQIFKTNTQTYRENLRIALRYYNQSEAGSHIIQRMYGCDLGPDGRLLRGHDQSAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*53:01 soluble) |
| 132 | GSHSMRYFHTAMSRPGRGEPRFITVGYVDDTLFVRFDSDATSPRKEPRAPWIEQEGPEYWDR |
| | ETQISKTNTQTYRESLRNLRGYYNQSEAGSHTWQRMYGCDLGPDGRLLRGYNQLAYDGKD |
| | YIALNEDLSSWTAADTAAQITQRKWEAAREAEQLRAYLEGLCVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*50:01 soluble) |
| 133 | GSHSMRYFYTSVSRPGRGEPRFIAVGYVDDTQFVRFDSDAASRRMEPRAPWIEQEGPEYWDG |
| | ETRKVKAHSQTHRVDLGTLRGYYNQSEAGSHTLQRMYGCDVGSDWRFLRGYHQYAYDGK |
| | DYIALKEDLRSWTAADMAAQTTKHKWEAAHVAEQWRAYLEGTCVEWLRRYLENGKETLQ |
| | RTDAPKTHMTHHAVSDHEATLRCWALSFYPAEITLTWQRDGEDQTQDTELVETRPAGDGTF |
| | QKWAAVVVPSGQEQRYTCHVQHEGLPKPLTLRWEPSS |
| | (HLA-A*02:05 soluble) |
| 134 | GSHSMRYFYTAMSRPGRGEPRFIAVGYVDDTQFVRFDSDAASPREEPRAPWIEQEGPEYWDR |
| | NTQIYKAQAQTDRESLRNLRGYYNQSEAGSHTWQTMYGCDLGPDGRLLRGHNQLAYDGKD |
| | YIALNEDLSSWTAADTAAQITQRKWEAAREAEQLRAYLEGTCVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*55:01 soluble) |
| 135 | GSHSMRYFHTAMSRPGRGEPRFITVGYVDDTLFVRFDSDATSPRKEPRAPWIEQEGPEYWDR |
| | ETQISKTNTQTYRESLRNLRGYYNQSEAGSHTWQRMYGCDLGPDGRLLRGYNQLAYDGKD |
| | YIALNEDLSSWTAADTAAQITQRKWEAARVAEQDRAYLEGLCVESLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*45:01 soluble) |
| 136 | GSHSMRYFYTAMSRPGRGEPRFIAVGYVDDTQFVRFDSDAASPRTEPRAPWIEQEGPEYWDR |
| | ETQISKTNTQTYRENLRIALRYYNQSEAGSHTWQTMYGCDVGPDGRLLRGHNQYAYDGKD |
| | YIALNEDLSSWTAADTAAQITQRKWEAAREAEQLRAYLEGLCVEWLRRHLENGKETLQRAD |
| | PPKTHVTHHPVSDHEATLRCWALGFYPAEITLTWQRDGEDQTQDTELVETRPAGDRTFQKW |
| | AAVVVPSGEEQRYTCHVQHEGLPKPLTLRWEPSS |
| | (HLA-B*52:01 soluble) |
| 137 | CSHSMRYFYTAVSRPGRGEPRFIAVGYVDDTQFVRFDSDAASPRGEPRAPWVEQEGPEYWD |
| | RETQKYKRQAQADRVSLRNLRGYYNQSEAGSHTLQRMYGCDLGPDGRLLRGYDQSAYDGK |
| | DYIALNEDLRSWTAADTAAQITQRKWEAAREAEQWRAYLEGTCVEWLRRYLENGKETLQR |
| | AEHPKTHVTHHPVSDHEATLRCWALGFYPAEITLTWQRDGEDQTQDTELVETRPAGDGTFQ |
| | KWAAVVVPSGEEQRYTCHVQHEGLPEPLTLRWEPSS |
| | (HLA-C*12:02 soluble) |
| 138 | GSHSMRYFYTAMSRPGRGEPRFIAVGYVDDTQFVRFDSDAASPRTEPRAPWIEQEGPEYWDR |
| | NTQIFKTNTQTYRESLRNLRGYYNQSEAGSHIIQRMYGCDLGPDGRLLRGHDQFAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*35:03 soluble) |
| 139 | GSHSMRYFHTSVSRPGRGEPRFITVGYVDDTLFVRFDSDATSPRKEPRAPWIEQEGPEYWDRE |
| | TQISKTNTQTYRESLRNLRGYYNQSEAGSHTLQSMYGCDVGPDGRLLRGHNQYAYDGKDYI |
| | ALNEDLRSWTAADTAAQITQRKWEAARVAEQLRAYLEGECVEWLRRYLENGKETLQRADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*40:02 soluble) |
| 140 | GSHSMRYFYTAMSRPGRGEPRFISVGYVDDTQFVRFDSDAASPREEPRAPWIEQEGPEYWDR |
| | ETQISKTNTQTYRESLRNLRGYYNQSEAGSHTLQRMYGCDVGPDGRLLRGHDQSAYDGKDY |
| | IALNEDLSSWTAADTAAQITQRKWEAAREAEQLRAYLEGLCVEWLRRYLENGKETLQRADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*15:03 soluble) |
| 141 | GSHSMRYFFTSVSRPGRGEPRFIAVGYVDDTQFVRFDSDAASQRMEPRAPWIEQEGPEYWDQ |
| | ETRNVKAHSQTDRVDLGTLRGYYNQSEAGSHTIQMMYGCDVGPDGRLLRGYQQDAYDGKD |
| | YIALNEDLRSWTAADMAAQITQRKWEAARVAEQLRAYLEGTCVEWLRRYLENGKETLQRT |
| | DAPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*74:01 soluble) |
| 142 | MSRSVALAVLALLSLSGLEAIQRTPKIQVYSRHPAENGKSNFLNCYVSGFHPSDIEVDLL |
| | KNGERIEKVEHSDLSFSKDWSFYLLYYTEFTPTEKDEYACRVNHVTLSQPKIVKWDRDM |
| | (full length human beta-2-microglobulin) |
| 143 | IQRTPKIQVYSRHPAENGKSNFLNCYVSGFHPSDIEVDLLKNGERIEKVEHSDLSFSKDWSFYL |
| | LYYTEFTPTEKDEYACRVNHVTLSQPKIVKWDRDM |
| | (human β-2 microglobulin, without signal sequence) |
| 144 | MAISGVPVLGFFIIAVLMSAQESWAIKEEHVIIQAEFYLNPDQSGEFMFDFDGDEIFHVDMAK |
| | KETVWRLEEFGRFASFEAQGALANIAVDKANLEIMTKRSNYTPITNVPPEVTVLTNSPVELRE |
| | PNVLICFIDKFTPPVVNVTWLRNGKPVTTGVSETVFLPREDHLFRKFHYLPFLPSTEDVYDCRV |
| | EHWGLDEPLLKHWEFDAPSPLPETTENVVCALGLTVGLVGIIIGTIFIIKGVRKSNAAERRGPL |
| | (HLA-DRA*01:01 full-length) |
| 145 | MVCLKLPGGSCMTALTVTLMVLSSPLALAGDTRPRFLWQLKFECHFFNGTERVRLLERCIYN |
| | QEESVRFDSDVGEYRAVTELGRPDAEYWNSQKDLLEQRRAAVDTYCRHNYGVGESFTVQRR |
| | VEPKVTVYPSKTQPLQHHNLLVCSVSGFYPGSIEVRWFRNGQEEKAGVVSTGLIQNGDWTFQ |
| | TLVMLETVPRSGEVYTCQVEHPSVTSPLTVEWRARSESAQSKMLSGVGGFVLGLLFLGAGLFI |
| | YFRNQKGHSGLQPTGFLS |
| | (HLA-DRB1*01:01 full-length) |
| 146 | MVCLKLPGGSCMTALTVTLMVLSSPLALAGDTRPRFLWQLKFECHFFNGTERVRLLERCIYN |
| | QEESVRFDSDVGEYRAVTELGRPDAEYWNSQKDLLEQRRAAVDTYCRHNYGAVESFTVQRR |
| | VEPKVTVYPSKTQPLQHHNLLVCSVSGFYPGSIEVRWFRNGQEEKAGVVSTGLIQNGDWTFQ |
| | TLVMLETVPRSGEVYTCQVEHPSVTSPLTVEWRARSESAQSKMLSGVGGFVLGLLFLGAGLFI |
| | YFRNQKGHSGLQPTGFLS |
| | (HLA-DRB1*01:02 full-length) |
| 147 | MVCLRLPGGSCMAVLTVTLMVLSSPLALAGDTRPRFLEYSTSECHFFNGTERVRYLDRYFHN |
| | QEENVRFDSDVGEFRAVTELGRPDAEYWNSQKDLLEQKRGRVDNYCRHNYGVVESFTVQR |
| | RVHPKVTVYPSKTQPLQHHNLLVCSVSGFYPGSIEVRWFRNGQEEKTGVVSTGLIHNGDWTF |
| | QTLVMLETVPRSGEVYTCQVEHPSVTSPLTVEWRARSESAQSKMLSGVGGFVLGLLFLGAGL |
| | FIYFRNQKGHSGLQPRGFLS |
| | (HLA-DRB1*03:01 full-length) |
| 148 | MVCLKFPGGSCMAALTVTLMVLSSPLALAGDTRPRFLEQVKHECHFFNGTERVRFLDRYFYH |
| | QEEYVRFDSDVGEYRAVTELGRPDAEYWNSQKDLLEQKRAAVDTYCRHNYGVGESFTVQR |
| | RVYPEVTVYPAKTQPLQHHNLLVCSVNGFYPGSIEVRWFRNGQEEKTGVVSTGLIQNGDWTF |
| | QTLVMLETVPRSGEVYTCQVEHPSLTSPLTVEWRARSESAQSKMLSGVGGFVLGLLFLGAGL |
| | FIYFRNQKGHSGLQPTGFLS |
| | (HLA-DRB1*04:01 full-length) |
| 149 | MVCLKFPGGSCMAALTVTLMVLSSPLALAGDTRPRFLEQVKHECHFFNGTERVRFLDRYFYH |
| | QEEYVRFDSDVGEYRAVTELGRPDAEYWNSQKDLLEQRRAAVDTYCRHNYGVVESFTVQR |
| | RVYPEVTVYPAKTQPLQHHNLLVCSVNGFYPGSIEVRWFRNGQEEKTGVVSTGLIQNGDWTF |
| | QTLVMLETVPRSGEVYTCQVEHPSLTSPLTVEWRARSESAQSKMLSGVGGFVLGLLFLGAGL |
| | FIYFRNQKGHSGLQPTGFLS |
| | (HLA-DRB1*04:04 full-length) |
| 150 | MVCLKLPGGSCMAALTVTLMVLSSPLALAGDTQPRFLWQGKYKCHFFNGTERVQFLERLFY |
| | NQEEFVRFDSDVGEYRAVTELGRPVAESWNSQKDILEDRRGQVDTVCRHNYGVGESFTVQR |
| | RVHPEVTVYPAKTQPLQHHNLLVCSVSGFYPGSIEVRWFRNGQEEKAGVVSTGLIQNGDWTF |
| | QTLVMLETVPRSGEVYTCQVEHPSVMSPLTVEWRARSESAQSKMLSGVGGFVLGLLFLGAG |
| | LFIYFRNQKGHSGLQPTGFLS |
| | (HLA-DRB1*07:01 full-length) |
| 151 | MVCLRLPGGSCMAVLTVTLMVLSSPLALAGDTRPRFLEYSTGECYFFNGTERVRFLDRYFYN |
| | QEEYVRFDSDVGEYRAVTELGRPSAEYWNSQKDFLEDRRALVDTYCRHNYGVGESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWSARSESAQSKMLSGVGGFVLGLLFLGAGLFI |
| | YFRNQKGHSGLQPTGFLS |
| | (HLA-DRB1*08:01 full-length) |
| 152 | MVCLRLPGGSCMAVLTVTLMVLSSPLALAGDTRPRFLEEVKFECHFFNGTERVRLLERRVHN |
| | QEEYARYDSDVGEYRAVTELGRPDAEYWNSQKDLLERRRAAVDTYCRHNYGVGESFTVQR |
| | RVQPKVTVYPSKTQPLQHHNLLVCSVNGFYPGSIEVRWFRNGQEEKTGVVSTGLIQNGDWTF |
| | QTLVMLETVPQSGEVYTCQVEHPSVMSPLTVEWRARSESAQSKMLSGVGGFVLGLLFLGAG |
| | LFIYFRNQKGHSGLPPTGFLS |
| | (HLA-DRB1*10:01 full-length) |
| 153 | MVCLRLPGGSCMAVLTVTLMVLSSPLALAGDTRPRFLEYSTSECHFFNGTERVRFLDRYFYN |
| | QEEYVRFDSDVGEFRAVTELGRPDEEYWNSQKDFLEDRRAAVDTYCRHNYGVGESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*11:01 full-length) |
| 154 | MVCLRLPGGSCMAVLTVTLMVLSSPLALAGDTRPRFLEYSTSECHFFNGTERVRFLDRYFYN |
| | QEEYVRFDSDVGEFRAVTELGRPDEEYWNSQKDFLEDRRAAVDTYCRHNYGVVESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*11:04 full-length) |
| 155 | MVCLRLPGGSCMAVLTVTLMVLSSPLALAGDTRPRFLEYSTSECHFFNGTERVRFLDRYFHN |
| | QEENVRFDSDVGEFRAVTELGRPDAEYWNSQKDILEDERAAVDTYCRHNYGVVESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*13:01 full-length) |
| 156 | MVCLRLPGGSCMAVLTVTLMVLSSPLALAGDTRPRFLEYSTSECHFFNGTERVRFLDRYFHN |
| | QEENVRFDSDVGEFRAVTELGRPDAEYWNSQKDILEDERAAVDTYCRHNYGVGESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*13:02 full-length) |
| 157 | MVCLRLPGGSCMAVLTVTLMVLSSPLALAGDTRPRFLEYSTSECHFFNGTERVRFLDRYFHN |
| | QEEFVRFDSDVGEYRAVTELGRPAAEHWNSQKDLLERRRAEVDTYCRHNYGVVESFTVQRR |
| | VHPKVTVYPSKTQPLQHYNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*14:01 full-length) |
| 158 | MVCLKLPGGSCMTALTVTLMVLSSPLALSGDTRPRFLWQPKRECHFFNGTERVRFLDRYFYN |
| | QEESVRFDSDVGEFRAVTELGRPDAEYWNSQKDILEQARAAVDTYCRHNYGVVESFTVQRR |
| | VQPKVTVYPSKTQPLQHHNLLVCSVSGFYPGSIEVRWFLNGQEEKAGMVSTGLIQNGDWTFQ |
| | TLVMLETVPRSGEVYTCQVEHPSVTSPLTVEWRARSESAQSKMLSGVGGFVLGLLFLGAGLFI |
| | YFRNQKGHSGLQPTGFLS |
| | (HLA-DRB1*15:01 full-length) |
| 159 | MVCLKLPGGSCMTALTVTLMVLSSPLALSGDTRPRFLWQPKRECHFFNGTERVRFLDRHFYN |
| | QEESVRFDSDVGEFRAVTELGRPDAEYWNSQKDILEQARAAVDTYCRHNYGVVESFTVQRR |
| | VQPKVTVYPSKTQPLQHHNLLVCSVSGFYPGSIEVRWFLNGQEEKAGMVSTGLIQNGDWTFQ |
| | TLVMLETVPRSGEVYTCQVEHPSVTSPLTVEWRARSESAQSKMLSGVGGFVLGLLFLGAGLFI |
| | YFRNQKGHSGLQPTGFLS |
| | (HLA-DRB1*15:03 full-length) |
| 160 | MILNKALLLGALALTTVMSPCGGEDIVADHVASCGVNLYQFYGPSGQYTHEFDGDEEFYVD |
| | LERKETAWRWPEFSKFGGFDPQGALRNMAVAKHNLNIMIKRYNSTAATNEVPEVTVFSKSP |
| | VTLGQPNTLICLVDNIFPPVVNITWLSNGQSVTEGVSETSFLSKSDHSFFKISYLTFLPSADEIYD |
| | CKVEHWGLDQPLLKHWEPEIPAPMSELTETVVCALGLSVGLVGIVVGTVFIIQGLRSVGASRH |
| | QGPL |
| | (HLA-DQA1*01:01 full-length) |
| 161 | MSWKKSLRIPGDLRVATVTLMLAILSSSLAEGRDSPEDFVYQFKGLCYFTNGTERVRGVTRHI |
| | YNREEYVRFDSDVGVYRAVTPQGRPVAEYWNSQKEVLEGARASVDRVCRHNYEVAYRGIL |
| | QRRVEPTVTISPSRTEALNHHNLLICSVTDFYPSQIKVRWFRNDQEETAGVVSTPLIRNGDWTF |
| | QILVMLEMTPQRGDVYTCHVEHPSLQSPITVEWRAQSESAQSKMLSGVGGFVLGLIFLGLGLI |
| | IRQRSRKGLLH |
| | (DQB1*05:01 full-length) |
| 162 | MILNKALLLGALALTTVMSPCGGEDIVADHVASCGVNLYQFYGPSGQYTHEFDGDEQFYVD |
| | LERKETAWRWPEFSKFGGFDPQGALRNMAVAKHNLNIMIKRYNSTAATNEVPEVTVFSKSP |
| | VTLGQPNTLICLVDNIFPPVVNITWLSNGQSVTEGVSETSFLSKSDHSFFKISYLTFLPSADEIYD |
| | CKVEHWGLDQPLLKHWEPEIPAPMSELTETVVCALGLSVGLMGIVVGTVFIIQGLRSVGASR |
| | HQGPL |
| | (HLA-DQA1*01:02 full-length) |
| 163 | MSWKKALRIPGDLRVATVTLMLAMLSSLLAEGRDSPEDFVFQFKGMCYFTNGTERVRLVTR |
| | YIYNREEYARFDSDVGVYRAVTPQGRPDAEYWNSQKEVLEGTRAELDTVCRHNYEVAFRGI |
| | LQRRVEPTVTISPSRTEALNHHNLLVCSVTDFYPGQIKVRWFRNDQEETAGVVSTPLIRNGDW |
| | TFQILVMLEMTPQRGDVYTCHVEHPSLQSPITVEWRAQSESAQSKMLSGVGGFVLGLIFLGLG |
| | LIIRQRSQKGLLH |
| | (HLA-DQB1*06:02 full-length) |
| 164 | MILNKALMLGALALTTVMSPCGGEDIVADHVASYGVNLYQSYGPSGQYSHEFDGDEEFYVD |
| | LERKETVWQLPLFRRFRRFDPQFALTNIAVLKHNLNIVIKRSNSTAATNEVPEVTVFSKSPVTL |
| | GQPNTLICLVDNIFPPVVNITWLSNGHSVTEGVSETSFLSKSDHSFFKISYLTFLPSADEIYDCK |
| | VEHWGLDEPLLKHWEPEIPTPMSELTETVVCALGLSVGLVGIVVGTVLIIRGLRSVGASRHQG |
| | PL |
| | (HLA-DQA1*03:01 full-length) |
| 165 | MSWKKALRIPGGLRVATVTLMLAMLSTPVAEGRDSPEDFVYQFKGMCYFTNGTERVRLVTR |
| | YIYNREEYARFDSDVGVYRAVTPLGPPAAEYWNSQKEVLERTRAELDTVCRHNYQLELRTTL |
| | QRRVEPTVTISPSRTEALNHHNLLVCSVTDFYPAQIKVRWFRNDQEETTGVVSTPLIRNGDWT |
| | FQILVMLEMTPQRGDVYTCHVEHPSLQNPIIVEWRAQSESAQSKMLSGIGGFVLGLIFLGLGLI |
| | IHHRSQKGLLH |
| | (HLA-DQB1*03:02 full-length) |
| 166 | MILNKALMLGALALTTVMSPCGGEDIVADHVASYGVNLYQSYGPSGQYTHEFDGDEQFYVD |
| | LGRKETVWCLPVLRQFRFDPQFALTNIAVLKHNLNSLIKRSNSTAATNEVPEVTVFSKSPVTL |
| | GQPNILICLVDNIFPPVVNITWLSNGHSVTEGVSETSFLSKSDHSFFKISYLTLLPSAEESYDCKV |
| | EHWGLDKPLLKHWEPEIPAPMSELTETVVCALGLSVGLVGIVVGTVFIIRGLRSVGASRHQGPL |
| | (HLA-DQA1*05:01 full-length) |
| 167 | MSWKKALRIPGGLRAATVTLMLSMLSTPVAEGRDSPEDFVYQFKGMCYFTNGTERVRLVSR |
| | SIYNREEIVRFDSDVGEFRAVTLLGLPAAEYWNSQKDILERKRAAVDRVCRHNYQLELRTTL |
| | QRRVEPTVTISPSRTEALNHHNLLVCSVTDFYPAQIKVRWFRNDQEETAGVVSTPLIRNGDWT |
| | FQILVMLEMTPQRGDVYTCHVEHPSLQSPITVEWRAQSESAQSKMLSGIGGFVLGLIFLGLGLI |
| | IHHRSQKGLLH |
| | (HLA-DQB1*02:01 full-length) |
| 168 | MSWKKALRIPGGLRAATVTLMLAMLSTPVAEGRDSPEDFVYQFKAMCYFTNGTERVRYVTR |
| | YIYNREEYARFDSDVEVYRAVTPLGPPDAEYWNSQKEVLERTRAELDTVCRHNYQLELRTTL |
| | QRRVEPTVTISPSRTEALNHHNLLVCSVTDFYPAQIKVRWFRNDQEETTGVVSTPLIRNGDWT |
| | FQILVMLEMTPQHGDVYTCHVEHPSLQNPITVEWRAQSESAQSKMLSGIGGFVLGLIFLGLGL |
| | IIHHRSQKGLLH |
| | (HLA-DQB1*03:01 full-length) |
| 169 | MSWKKALRIPGGLRVATVTLMLAMLSTPVAEGRDSPEDFVYQFKGMCYFTNGTERVRLVTR |
| | YIYNREEYARFDSDVGVYRAVTPLGPPDAEYWNSQKEVLERTRAELDTVCRHNYQLELRTTL |
| | QRRVEPTVTISPSRTEALNHHNLLVCSVTDFYPAQIKVRWFRNDQEETTGVVSTPLIRNGDWT |
| | FQILVMLEMTPQRGDVYTCHVEHPSLQNPIIVEWRAQSESAQSKMLSGIGGFVLGLIFLGLGLI |
| | IHHRSQKGLLH |
| | (HLA-DQB1*03:03 full-length) |
| 170 | MSWKKALRIPGGLRVATVTLMLAMLSTPVAEGRDSPEDFVFQFKGMCYFTNGTERVRGVTR |
| | YIYNREEYARFDSDVGVYRAVTPLGRLDAEYWNSQKDILEEDRASVDTVCRHNYQLELRTTL |
| | QRRVEPTVTISPSRTEALNHHNLLVCSVTDFYPAQIKVRWFRNDQEETTGVVSTPLIRNGDWT |
| | FQILVMLEMTPQRGDVYTCHVEHPSLQNPIIVEWRAQSESAQSKMLSGIGGFVLGLIFLGLGLI |
| | IHHRSQKGLLH |
| | (HLA-DQB1*04:02 full-length) |
| 171 | MSWKKSLRIPGDLRVATVTLMLAILSSSLAEGRDSPEDFVYQFKGLCYFTNGTERVRGVTRHI |
| | YNREEYVRFDSDVGVYRAVTPQGRPDAEYWNSQKEVLEGARASVDRVCRHNYEVAYRGIL |
| | QRRVEPTVTISPSRTEALNHHNLLICSVTDFYPSQIKVRWFRNDQEETAGVVSTPLIRNGDWTF |
| | QILVMLEMTPQRGDVYTCHVEHPSLQSPITVEWRAQSESAQSKMLSGVGGFVLGLIFLGLGLI |
| | IRQRSRKGPQGPPPAGLLH |
| | (HLA-DQB1*05:03 full-length) |
| 172 | MSWKKALRIPGDLRVATVTLMLAMLSSLLAEGRDSPEDFVYQFKGMCYFTNGTERVRLVTR |
| | HIYNREEYARFDSDVGVYRAVTPQGRPDAEYWNSQKEVLEGTRAELDTVCRHNYEVAFRGI |
| | LQRRVEPTVTISPSRTEALNHHNLLVCSVTDFYPGQIKVRWFRNDQEETAGVVSTPLIRNGDW |
| | TFQILVMLEMTPQRGDVYTCHVEHPSLQSPITVEWRAQSESAQSKMLSGVGGFVLGLIFLGLG |
| | LIIRQRSQKGLLH |
| | (HLA-DQB1*06:03 full-length) |
| 173 | MSWKKALRIPGDLRVATVTLMLAMLSSLLAEGRDSPEDFVYQFKGMCYFTNGTERVRLVTR |
| | HIYNREEYARFDSDVGVYRAVTPQGRPVAEYWNSQKEVLERTRAELDTVCRHNYEVGYRGI |
| | LQRRVEPTVTISPSRTEALNHHNLLVCSVTDFYPGQIKVQWFRNDQEETAGVVSTPLIRNGDW |
| | TFQILVMLEMTPQRGDVYTCHVEHPSLQSPITVEWRAQSESAQSKMLSGVGGFVLGLIFLGLG |
| | LIIRQRSQKGLLH |
| | (HLA-DQB1*06:04 full-length) |
| 174 | IKEEHVIIQAEFYLNPDQSGEFMFDFDGDEIFHVDMAKKETVWRLEEFGRFASFEAQGALANI |
| | AVDKANLEIMTKRSNYTPITNVPPEVTVLTNSPVELREPNVLICFIDKFTPPVVNVTWLRNGKP |
| | VTTGVSETVFLPREDHLFRKFHYLPFLPSTEDVYDCRVEHWGLDEPLLKHWEFDAPSPLPETTE |
| | (HLA-DRA*01:01 soluble) |
| 175 | GDTRPRFLWQLKFECHFFNGTERVRLLERCIYNQEESVRFDSDVGEYRAVTELGRPDAEYWN |
| | SQKDLLEQRRAAVDTYCRHNYGVGESFTVQRRVEPKVTVYPSKTQPLQHHNLLVCSVSGFY |
| | PGSIEVRWFRNGQEEKAGVVSTGLIQNGDWTFQTLVMLETVPRSGEVYTCQVEHPSVTSPLT |
| | VEWRARSESAQSK |
| | (HLA-DRB1*01:01 soluble) |
| 176 | GDTRPRFLWQLKFECHFFNGTERVRLLERCIYNQEESVRFDSDVGEYRAVTELGRPDAEYWN |
| | SQKDLLEQRRAAVDTYCRHNYGAVESFTVQRRVEPKVTVYPSKTQPLQHHNLLVCSVSGFY |
| | PGSIEVRWFRNGQEEKAGVVSTGLIQNGDWTFQTLVMLETVPRSGEVYTCQVEHPSVTSPLT |
| | VEWRARSESAQSK |
| | (HLA-DRB1*01:02 soluble) |
| 177 | GDTRPRFLEYSTSECHFFNGTERVRYLDRYFHNQEENVRFDSDVGEFRAVTELGRPDAEYWN |
| | SQKDLLEQKRGRVDNYCRHNYGVVESFTVQRRVHPKVTVYPSKTQPLQHHNLLVCSVSGFY |
| | PGSIEVRWFRNGQEEKTGVVSTGLIHNGDWTFQTLVMLETVPRSGEVYTCQVEHPSVTSPLT |
| | VEWRARSESAQSK |
| | (HLA-DRB1*03:01 soluble) |
| 178 | GDTRPRFLEQVKHECHFFNGTERVRFLDRYFYHQEEYVRFDSDVGEYRAVTELGRPDAEYW |
| | NSQKDLLEQKRAAVDTYCRHNYGVGESFTVQRRVYPEVTVYPAKTQPLQHHNLLVCSVNGF |
| | YPGSIEVRWFRNGQEEKTGVVSTGLIQNGDWTFQTLVMLETVPRSGEVYTCQVEHPSLTSPLT |
| | VEWRARSESAQSK |
| | (HLA-DRB1*04:01 soluble) |
| 179 | GDTRPRFLEQVKHECHFFNGTERVRFLDRYFYHQEEYVRFDSDVGEYRAVTELGRPDAEYW |
| | NSQKDLLEQRRAAVDTYCRHNYGVVESFTVQRRVYPEVTVYPAKTQPLQHHNLLVCSVNGF |
| | YPGSIEVRWFRNGQEEKTGVVSTGLIQNGDWTFQTLVMLETVPRSGEVYTCQVEHPSLTSPLT |
| | VEWRARSESAQSK |
| | (HLA-DRB1*04:04 soluble) |
| 180 | GDTQPRFLWQGKYKCHFFNGTERVQFLERLFYNQEEFVRFDSDVGEYRAVTELGRPVAESW |
| | NSQKDILEDRRGQVDTVCRHNYGVGESFTVQRRVHPEVTVYPAKTQPLQHHNLLVCSVSGF |
| | YPGSIEVRWFRNGQEEKAGVVSTGLIQNGDWTFQTLVMLETVPRSGEVYTCQVEHPSVMSPL |
| | TVEWRARSESAQSK |
| | (HLA-DRB1*07:01 soluble) |
| 181 | GDTRPRFLEYSTGECYFFNGTERVRFLDRYFYNQEEYVRFDSDVGEYRAVTELGRPSAEYWN |
| | SQKDFLEDRRALVDTYCRHNYGVGESFTVQRRVHPKVTVYPSKTQPLQHHNLLVCSVSGFYP |
| | GSIEVRWFRNGQEEKTGVVSTGLIHNGDWTFQTLVMLETVPRSGEVYTCQVEHPSVTSPLTV |
| | EWSARSESAQSK |
| | (HLA-DRB1*08:01 soluble) |
| 182 | GDTRPRFLEEVKFECHFFNGTERVRLLERRVHNQEEYARYDSDVGEYRAVTELGRPDAEYW |
| | NSQKDLLERRRAAVDTYCRHNYGVGESFTVQRRVQPKVTVYPSKTQPLQHHNLLVCSVNGF |
| | YPGSIEVRWFRNGQEEKTGVVSTGLIQNGDWTFQTLVMLETVPQSGEVYTCQVEHPSVMSPL |
| | TVEWRARSESAQSK |
| | (HLA-DRB1*10:01 soluble) |
| 183 | GDTRPRFLEYSTSECHFFNGTERVRFLDRYFYNQEEYVRFDSDVGEFRAVTELGRPDEEYWN |
| | SQKDFLEDRRAAVDTYCRHNYGVGESFTVQRRVHPKVTVYPSKTQPLQHHNLLVCSVSGFY |
| | PGSIEVRWFRNGQEEKTGVVSTGLIHNGDWTFQTLVMLETVPRSGEVYTCQVEHPSVTSPLT |
| | VEWRARSESAQSK |
| | (HLA-DRB1*11:01 soluble) |
| 184 | GDTRPRFLEYSTSECHFFNGTERVRFLDRYFYNQEEYVRFDSDVGEFRAVTELGRPDEEYWN |
| | SQKDFLEDRRAAVDTYCRHNYGVVESFTVQRRVHPKVTVYPSKTQPLQHHNLLVCSVSGFY |
| | PGSIEVRWFRNGQEEKTGVVSTGLIHNGDWTFQTLVMLETVPRSGEVYTCQVEHPSVTSPLT |
| | VEWRARSESAQSK |
| | (HLA-DRB1*11:04 soluble) |
| 185 | GDTRPRFLEYSTSECHFFNGTERVRFLDRYFHNQEENVRFDSDVGEFRAVTELGRPDAEYWN |
| | SQKDILEDERAAVDTYCRHNYGVVESFTVQRRVHPKVTVYPSKTQPLQHHNLLVCSVSGFYP |
| | GSIEVRWFRNGQEEKTGVVSTGLIHNGDWTFQTLVMLETVPRSGEVYTCQVEHPSVTSPLTV |
| | EWRARSESAQSK |
| | (HLA-DRB1*13:01 soluble) |
| 186 | GDTRPRFLEYSTSECHFFNGTERVRFLDRYFHNQEENVRFDSDVGEFRAVTELGRPDAEYWN |
| | SQKDILEDERAAVDTYCRHNYGVGESFTVQRRVHPKVTVYPSKTQPLQHHNLLVCSVSGFYP |
| | GSIEVRWFRNGQEEKTGVVSTGLIHNGDWTFQTLVMLETVPRSGEVYTCQVEHPSVTSPLTV |
| | EWRARSESAQSK |
| | (HLA-DRB1*13:02 soluble) |
| 187 | GDTRPRFLEYSTSECHFFNGTERVRFLDRYFHNQEEFVRFDSDVGEYRAVTELGRPAAEHWN |
| | SQKDLLERRRAEVDTYCRHNYGVVESFTVQRRVHPKVTVYPSKTQPLQHYNLLVCSVSGFYP |
| | GSIEVRWFRNGQEEKTGVVSTGLIHNGDWTFQTLVMLETVPRSGEVYTCQVEHPSVTSPLTV |
| | EWRARSESAQSK |
| | (HLA-DRB1*14:01 soluble) |
| 188 | GDTRPRFLWQPKRECHFFNGTERVRFLDRYFYNQEESVRFDSDVGEFRAVTELGRPDAEYW |
| | NSQKDILEQARAAVDTYCRHNYGVVESFTVQRRVQPKVTVYPSKTQPLQHHNLLVCSVSGF |
| | YPGSIEVRWFLNGQEEKAGMVSTGLIQNGDWTFQTLVMLETVPRSGEVYTCQVEHPSVTSPL |
| | TVEWRARSESAQSK |
| | (HLA-DRB1*15:01 soluble) |
| 189 | GDTRPRFLWQPKRECHFFNGTERVRFLDRHFYNQEESVRFDSDVGEFRAVTELGRPDAEYW |
| | NSQKDILEQARAAVDTYCRHNYGVVESFTVQRRVQPKVTVYPSKTQPLQHHNLLVCSVSGF |
| | YPGSIEVRWFLNGQEEKAGMVSTGLIQNGDWTFQTLVMLETVPRSGEVYTCQVEHPSVTSPL |
| | TVEWRARSESAQSK |
| | (HLA-DRB1*15:03 soluble) |
| 190 | EDIVADHVASCGVNLYQFYGPSGQYTHEFDGDEEFYVDLERKETAWRWPEFSKFGGFDPQG |
| | ALRNMAVAKHNLNIMIKRYNSTAATNEVPEVTVFSKSPVTLGQPNTLICLVDNIFPPVVNITW |
| | LSNGQSVTEGVSETSFLSKSDHSFFKISYLTFLPSADEIYDCKVEHWGLDQPLLKHWEPEIPAP |
| | MSELTET |
| | (HLA-DQA1*01:01 soluble) |
| 191 | GRDSPEDFVYQFKGLCYFTNGTERVRGVTRHIYNREEYVRFDSDVGVYRAVTPQGRPVAEY |
| | WNSQKEVLEGARASVDRVCRHNYEVAYRGILQRRVEPTVTISPSRTEALNHHNLLICSVTDFY |
| | PSQIKVRWFRNDQEETAGVVSTPLIRNGDWTFQILVMLEMTPQRGDVYTCHVEHPSLQSPITV |
| | EWRAQSESAQSK |
| | (HLA-DQB1*05:01 soluble) |
| 192 | EDIVADHVASCGVNLYQFYGPSGQYTHEFDGDEQFYVDLERKETAWRWPEFSKFGGFDPQG |
| | ALRNMAVAKHNLNIMIKRYNSTAATNEVPEVTVFSKSPVTLGQPNTLICLVDNIFPPVVNITW |
| | LSNGQSVTEGVSETSFLSKSDHSFFKISYLTFLPSADEIYDCKVEHWGLDQPLLKHWEPEIPAP |
| | MSELTET |
| | (HLA-DQA1*01:02 soluble) |
| 193 | GRDSPEDFVFQFKGMCYFTNGTERVRLVTRYIYNREEYARFDSDVGVYRAVTPQGRPDAEY |
| | WNSQKEVLEGTRAELDTVCRHNYEVAFRGILQRRVEPTVTISPSRTEALNHHNLLVCSVTDFY |
| | PGQIKVRWFRNDQEETAGVVSTPLIRNGDWTFQILVMLEMTPQRGDVYTCHVEHPSLQSPIT |
| | VEWRAQSESAQSK |
| | (HLA-DQB1*06:02 soluble) |
| 194 | EDIVADHVASYGVNLYQSYGPSGQYSHEFDGDEEFYVDLERKETVWQLPLFRRFRRFDPQFA |
| | LTNIAVLKHNLNIVIKRSNSTAATNEVPEVTVFSKSPVTLGQPNTLICLVDNIFPPVVNITWLSN |
| | GHSVTEGVSETSFLSKSDHSFFKISYLTFLPSADEIYDCKVEHWGLDEPLLKHWEPEIPTPMSE |
| | LTET |
| | (HLA-DQA1*03:01 soluble) |
| 195 | GRDSPEDFVYQFKGMCYFTNGTERVRLVTRYIYNREEYARFDSDVGVYRAVTPLGPPAAEY |
| | WNSQKEVLERTRAELDTVCRHNYQLELRTTLQRRVEPTVTISPSRTEALNHHNLLVCSVTDF |
| | YPAQIKVRWFRNDQEETTGVVSTPLIRNGDWTFQILVMLEMTPQRGDVYTCHVEHPSLQNPII |
| | VEWRAQSESAQSK |
| | (HLA-DQB1*03:02 soluble) |
| 196 | EDIVADHVASYGVNLYQSYGPSGQYTHEFDGDEQFYVDLGRKETVWCLPVLRQFRFDPQFA |
| | LTNIAVLKHNLNSLIKRSNSTAATNEVPEVTVFSKSPVTLGQPNILICLVDNIFPPVVNITWLSN |
| | GHSVTEGVSETSFLSKSDHSFFKISYLTLLPSAEESYDCKVEHWGLDKPLLKHWEPEIPAPMSE |
| | LTET |
| | (HLA-DQA1*05:01 soluble) |
| 197 | GRDSPEDFVYQFKGMCYFTNGTERVRLVSRSIYNREEIVRFDSDVGEFRAVTLLGLPAAEYW |
| | NSQKDILERKRAAVDRVCRHNYQLELRTTLQRRVEPTVTISPSRTEALNHHNLLVCSVTDFYP |
| | AQIKVRWFRNDQEETAGVVSTPLIRNGDWTFQILVMLEMTPQRGDVYTCHVEHPSLQSPITV |
| | EWRAQSESAQSK |
| | (HLA-DQB1*02:01 soluble) |
| 198 | GRDSPEDFVYQFKAMCYFTNGTERVRYVTRYIYNREEYARFDSDVEVYRAVTPLGPPDAEY |
| | WNSQKEVLERTRAELDTVCRHNYQLELRTTLQRRVEPTVTISPSRTEALNHHNLLVCSVTDF |
| | YPAQIKVRWFRNDQEETTGVVSTPLIRNGDWTFQILVMLEMTPQHGDVYTCHVEHPSLQNPI |
| | TVEWRAQSESAQSK |
| | (HLA-DQB1*03:01 soluble) |
| 199 | GRDSPEDFVYQFKGMCYFTNGTERVRLVTRYIYNREEYARFDSDVGVYRAVTPLGPPDAEY |
| | WNSQKEVLERTRAELDTVCRHNYQLELRTTLQRRVEPTVTISPSRTEALNHHNLLVCSVTDF |
| | YPAQIKVRWFRNDQEETTGVVSTPLIRNGDWTFQILVMLEMTPQRGDVYTCHVEHPSLQNPII |
| | VEWRAQSESAQSK |
| | (HLA-DQB1*03:03 soluble) |
| 200 | GRDSPEDFVFQFKGMCYFTNGTERVRGVTRYIYNREEYARFDSDVGVYRAVTPLGRLDAEY |
| | WNSQKDILEEDRASVDTVCRHNYQLELRTTLQRRVEPTVTISPSRTEALNHHNLLVCSVTDFY |
| | PAQIKVRWFRNDQEETTGVVSTPLIRNGDWTFQILVMLEMTPQRGDVYTCHVEHPSLQNPIIV |
| | EWRAQSESAQSK |
| | (HLA-DQB1*04:02 soluble) |
| 201 | GRDSPEDFVYQFKGLCYFTNGTERVRGVTRHIYNREEYVRFDSDVGVYRAVTPQGRPDAEY |
| | WNSQKEVLEGARASVDRVCRHNYEVAYRGILQRRVEPTVTISPSRTEALNHHNLLICSVTDFY |
| | PSQIKVRWFRNDQEETAGVVSTPLIRNGDWTFQILVMLEMTPQRGDVYTCHVEHPSLQSPITV |
| | EWRAQSESAQSK |
| | (HLA-DQB1*05:03 soluble) |
| 202 | GRDSPEDFVYQFKGMCYFTNGTERVRLVTRHIYNREEYARFDSDVGVYRAVTPQGRPDAEY |
| | WNSQKEVLEGTRAELDTVCRHNYEVAFRGILQRRVEPTVTISPSRTEALNHHNLLVCSVTDFY |
| | PGQIKVRWFRNDQEETAGVVSTPLIRNGDWTFQILVMLEMTPQRGDVYTCHVEHPSLQSPIT |
| | VEWRAQSESAQSK |
| | (HLA-DQB1*06:03 soluble) |
| 203 | GRDSPEDFVYQFKGMCYFTNGTERVRLVTRHIYNREEYARFDSDVGVYRAVTPQGRPVAEY |
| | WNSQKEVLERTRAELDTVCRHNYEVGYRGILQRRVEPTVTISPSRTEALNHHNLLVCSVTDF |
| | YPGQIKVQWFRNDQEETAGVVSTPLIRNGDWTFQILVMLEMTPQRGDVYTCHVEHPSLQSPI |
| | TVEWRAQSESAQSK |
| | (HLA-DQB1*06:04 soluble) |
| 204 | GILGFVFTL |
| | (A02:01 binding peptide) |
| 205 | NLVPMVGTV |
| | (A02:01 binding peptide) |
| 206 | EADPTGHSY |
| | (A01:01 binding peptide) |
| 207 | RYPLTFGWCF |
| | (A24:02 placeholder peptide) |
| 208 | QYDPVAALF |
| | (A24:02 binding peptide) |
| 209 | RPHERNGFTVL |
| | (B7:02 binding peptide) |
| 210 | PHERNGFTVL |
| | (B7:02 placeholder peptide) |
| 211 | KILGFVFTV |
| | (A2:01 placeholder peptide) |
| 212 | VTEHDTLLY |
| | (A1:01 placeholder peptide) |
| 213 | TVRSHCVSK |
| | (A3:01 placeholder peptide) |
| 214 | TTFLQTMLR |
| | (A11:01 placeholder peptide) |
| 215 | IPSINVHHY |
| | (B35:01 placeholder peptide) |
| 216 | FVYGGSKTSL |
| | (C3:04 placeholder peptide) |
| 217 | FLRGRAYGL |
| | (B8:01 placeholder peptide) |
| 218 | RYRPGTVAL |
| | (C7:02 placeholder peptide) |
| 219 | QYDPVAALF |
| | (C4:01 placeholder peptide) |
| 220 | GQFLTPNSH |
| | (B15:01 placeholder peptide) |
| 221 | KEVNSQLSL |
| | (B40:01 placeholder peptide) |
| 222 | VSFIEFVGW |
| | (B58:01 placeholder peptide) |
| 223 | IAPWYAFAL |
| | (C8:01 placeholder peptide) |
| 224 | KPVSKMRMATPLLMQA |
| | (CLIP peptide) |
| 225 | QIYKANSKFIGITEL |
| | (TT p2 peptide) |
| 226 | (GGGGS)n, wherein n = 1-6 |
| | (linker) |
| 227 | SSSSGSSSSGSAA |
| | (linker) |
| 228 | GGGGG |
| | (linker) |
| 229 | S(GGGGS)n, wherein n = 1-10 |
| | (linker) |
| 230 | (GGSG)n, wherein n = 1-5 |
| | (linker) |
| 231 | GSAT |
| | (linker) |
| 232 | (GGSGGS)n, wherein n = 1-5 |
| | (linker) |
| 233 | GGGGSGGGGSGGGGSGGGGS |
| | ((G₄S)₄ linker) |
| 234 | GSGSAGGSGSGGGS |
| | ((GS)₂AG₂SGSG₃S linker) |
| 235 | IEGR |
| | (Factor Xa cleavage site) |
| 236 | GGGASGGGGSIEGRGGGGSGGGGS |
| | (GS linker including Factor Xa cleavage site) |
| 237 | DDDDK |
| | (enterokinase cleavage site) |
| 238 | DYKDDDDK |
| | (FLAG Tag) |
| 239 | HHHHHH |
| | (6x His Tag) |
| 240 | GKPIPNPLLGLDST |
| | (V5 Tag) |
| 241 | WSHPQFEK |
| | (strep-tag) |
| 242 | EDQVDPRLIDGK |
| | (protein C tag) |
| 243 | EQKLISEEDL |
| | (Myc tag) |
| 244 | GLNDIFEAQKIEWHEGSGEQKLISEEDL |
| | (avitag-Myc (biotin-mediated)) |
| 245 | GLNDIFEAQKIEWHEGSGEQKLISEEDLHHHHHH |
| | (avitag-Myc-His (biotin-mediated)) |
| 246 | GSGSAGGGLNDIFEAQKIEWHEGSTGHHHHHHDYKDDDDK |
| | (Avitag sequence with His6 tag and Flag tag) |
| 247 | LTDTLQAETDQLEDEKSALQTEIANLLKEKEKLEFILAA |
| | (Fos) |
| 248 | RIARLEEKVKTLKAQNSELASTANMLREQVAQLKQKVMNH |
| | (Jun) |
| 249 | TTAPSAQLEKELQALQKENAQLEWELQALEKELAQ |
| | (acidic leucine zipper) |
| 250 | TTAPSAQLKKKLQALKKKNAQLKWKLQALKKKLAQ |
| | (basic leucine zipper) |
| 251 | EPKSADKTHTCPPCPAPEAAGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALGAPIEKTISKA |
| | KGQPREPQVYTLPPCRDELTKNQVSLWCLVKGFYPSDIAVEWESNGQPENNYKTTPPVLDSD |
| | GSFFLYSKLTVDKSRWQQGNVFSCSVMHEALHNHYTQKSLSLSPGK |
| | (knob (knob-in-hole)) |
| 252 | EPKSADKTHTCPPCPAPEAAGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALGAPIEKTISKA |
| | KGQPREPQVCTLPPSRDELTKNQVSLSCAVKGFYPSDIAVEWESNGQPENNYKTTPPVLDSDG |
| | SFFLVSKLTVDKSRWQQGNVFSCSVMHEALHNHYTQKSLSLSPGK |
| | (hole (knob-in-hole) |
| 253 | RGVPHIVMVDAYKRYK |
| | (spytag) |
| 254 | VTTLSGLSGEQGPSGDMTTEEDSATHIKFSKRDEDGRELAGATMELRDSSGKTISTWISDGHV |
| | KDFYLYPGKYTFVETAAPDGYEVATPIEFTVNEDGQVTVDGEATEGDAHT |
| | (spycatcher) |
| 255 | MEAPAQLLFLLLLWLPDTTG |
| | (Ig Kappa chain V-III region CLL signal peptide) |
| 256 | MNRGVPFRHLLLVLQLALLPAAT |
| | (signal peptide of human CD4 signal peptide) |
| 257 | METDTLLLWVLLLWVPGSTG |
| | (mouse Ig kappa chain V-III region signal peptide) |
| 258 | MVPCTLLLLLAAALAPTQTRA |
| | (mouse H-2Kb signal peptide) |
| 259 | MKWVTFISLLFLFSSAYS |
| | (human serum albumin signal peptide) |
| 260 | MYRMQLLSCIALSLALVTNS |
| | (human IL-2 signal peptide) |
| 261 | MAVMAPRTLLLLLSGALALTQTWA |
| | (human HLA-A*02:01 signal peptide) |
| 262 | MSRSVALAVLALLSLSGLEA |
| | (Human b2m signal peptide) |
| 263 | DPSKDSKAQVSAAEAGITGTWYNQLGSTFIVTAGADGALTGTYESAVGNAESRYVLTGRYDS |
| | APATDGSGTALGWTVAWKNNYRNAHSATTWSGQYVGGAEARINTQWLLTSGTTEANAWK |
| | STLVGHDTFTKVKPSAASIDAAKKAGVNNGNPLDAVQQ |
| | (full-length steptavadin) |
| 264 | AEAGITGTWYNQLGSTFIVTAGADGALTGTYESAVGNAESRYVLTGRYDSAPATDGSGTALG |
| | WTVAWKNNYRNAHSATTWSGQYVGGAEARINTQWLLTSGTTEANAWKSTLVGHDTFTKV |
| | KPSAAS |
| | (natural core streptavidin) |
| 265 | MEAGITGTWYNQLGSTFIVTAGADGALTGTYESAVGNAESRYVLTGRYDSAPATDGSGTAL |
| | GWTVAWKNNYRNAHSATTWSGQYVGGAEARINTQWLLTSGTTEANAWKSTLVGHDTFTK |
| | VKPSAA |
| | (recombinant core streptavidin STV25) |
| 266 | MGITGTWYNQLGSTFIVTAGADGALTGTYESAVGNAESRYVLTGRYDSAPATDGSGTALGW |
| | TVAWKNNYRNAHSATTWSGQYVGGAEARINTQWLLTSGTTEANAWKSTLVGHDTFTKV |
| | (recombinant core streptavidin STV13) |
| 267 | IALNFPGSQK |
| | (A03:01 binding peptide) |
| 268 | LIYRRRLMK |
| | (A03:01 binding peptide) |
| 269 | ILRGSVAHK |
| | (A03:01 binding peptide) |
| 270 | RIKEHMLKK |
| | (A03:01 binding peptide) |
| 271 | AVFDRKSDAK |
| | (A11:01 binding peptide) |
| 272 | IVTDFSVIK |
| | (A11:01 binding peptide) |
| 273 | AIFQSSMTK |
| | (A11:01 binding peptide) |
| 274 | VYGFVRACL |
| | (A24:02 binding peptide) |
| 275 | TPRVTGGGAM |
| | (B07:02 binding peptide) |
| 276 | ALKRKMMYM |
| | (B08:01 binding peptide) |
| 277 | HSKKKCDEL |
| | (B08:01 binding peptide) |
| 278 | WLSLLVPFV |
| | (A02:05 binding peptide) |
| 279 | VMAPRTLIL |
| | (HLA-E binding peptide) |
| 280 | TRATKMQVI |
| | (C06:02 binding peptide) |
| 281 | RQYDPVAAL |
| | (A30:01 binding peptide) |
| 282 | FVYGGSKTSL |
| | (C03:03 binding peptide) |
| 283 | VSDGGPNLY |
| | (C08:02 binding peptide) |
| 284 | YILGADPLRV |
| | (B13:02 binding peptide) |
| 285 | YHSIEWAI |
| | (B38:01 binding peptide) |
| 286 | RRRWRRLTV |
| | (B14:01 binding peptide) |
| 287 | ALFFFDIDL |
| | (A32:01 binding peptide) |
| 288 | FPTKDVAL |
| | (B35:02 binding peptide) |
| 289 | YRSGIIAVV |
| | (B39:06 binding peptide) |
| 290 | SYMIMEIEL |
| | (C14:01 binding peptide) |
| 291 | TAFTIPSI |
| | (B51:01 binding peptide) |
| 292 | TVCGGIMFL |
| | (C15:02 binding peptide) |
| 293 | SFSFGGFTFK |
| | (A31:01 binding peptide) |
| 294 | FEDLRVSSF |
| | (B37:01 binding peptide) |
| 295 | SFSFGGFTFK |
| | (A33:01 binding peptide) |
| 296 | SELEIKRY |
| | (B18:01 binding peptide) |
| 297 | CVIGGAGNNT |
| | (B50:01 binding peptide) |
| 298 | YLLEMLWR |
| | (A68:02 binding peptide) |
| 299 | KWMRELVLY |
| | (B15:03 binding peptide) |
| 300 | VAFTSHEHF |
| | (C01:02 binding peptide) |
| 301 | AIMESGVAL |
| | (C07:04 binding peptide) |
| 302 | KRWIILGLNK |
| | (B27:05 binding peptide) |
| 303 | HSNLNDATY |
| | (A26:01 binding peptide) |
| 304 | VSDGGPNLY |
| | (C08:02 binding peptide) |
| 305 | EENLLDFVRF |
| | (B44:02 binding peptide) |
| 306 | KAFSPEVIPMF |
| | (B57:01 binding peptide) |
| 307 | FLDKGTYTL |
| | (C05:01 binding peptide) |
| 308 | FCRVLCCYV |
| | (C07:01 binding peptide) |
| 309 | TAFTIPSI |
| | (B52:01 binding peptide) |
| 310 | LQAKARAKKDELRRK |
| | (A74:01 binding peptide) |
| 311 | AENAGNDAC |
| | (B45:01 binding peptide) |
| 312 | HPVGEADYF |
| | (B53:01 binding peptide) |
| 313 | PHGPVQLSYYD |
| | (C12:02 binding peptide) |
| 314 | YPLHEQHGM |
| | (B35:03 binding peptide) |
| 315 | HERNGFTVL |
| | (B40:02 binding peptide) |
| 316 | KAFSPEVIPMF |
| | (B57:03 binding peptide) |
| 317 | FPKTTNGCSQA |
| | (B55:01 binding peptide) |
| 318 | EENLLDFVRF |
| | (B44:03 binding peptide) |
| 319 | ELKRKMMYM |
| | (B08:01 binding peptide) |
| 320 | HSKKKCDEL |
| | (B08:01 binding peptide) |
| 321 | GSHSMRYFFTSVSRPGRGEPRFIAVGYVDDTQFVRFDSDAASQRMEPRAPWIEQEGPEYWDG |
| | ETRKVKAHSQTHRVDLGTLRGAYNQSEAGSHTVQRMYGCDVGSDWRFLRGYHQYAYDGK |
| | DYIALKEDLRSWTAADMAAQTTKHKWEAAHVAEQLRAYLEGTCVEWLRRYLENGKETLQR |
| | TDAPKTHMTHHAVSDHEATLRCWALSFYPAEITLTWQRDGEDQTQDTELVETRPAGDGTFQ |
| | KWAAVVVPSGQEQRYTCHVQHEGLPKPLTLRWEPSS |
| | (the soluble domain of HLA-A*02:01 with Y84A mutation) |
| 322 | ELAGIGILTV |
| | (HLA-A*02:01 restricted MART-1 epitope) |
| 323 | RMATPLLMQALPMGAL |
| | (CLIP peptide) |
| 324 | LMQALPMGALPQGP |
| | (CLIP peptide) |

1. A method of producing a Major Histocompatibility Complex (MHC) multimer, the method comprising:
(a) providing an MHC multimer expression construct comprising a nucleic acid encoding (i) an MHC-binding peptide operatively linked to a cleavage site; (ii) a first MHC subunit; (iii) a second MHC subunit; and (iv) a multimerization domain;
(b) introducing the MHC multimer expression construct into a host cell; and
(c) expressing the MHC multimer in the host cell.

2. The method of claim 1, wherein the first MHC subunit is a beta2-microglobulin chain, the second MHC subunit is an MHC Class I alpha chain and the MHC-binding peptide is an MHC Class I binding peptide.

3. The method of claim 1, wherein the first MHC subunit is an MHC Class I alpha chain, the second MHC subunit is a beta2-microglobulin chain and the MHC-binding peptide is an MHC Class I binding peptide.

4. The method of claim 2 or claim 3, wherein the MHC Class I binding peptide is a CMV pp65 peptide comprising the amino acid sequence NLVPMVATV (SEQ ID NO: 4).

5. The method of claim 2 or claim 3, wherein the MHC Class I binding peptide is a peptide comprising an amino acid sequence selected from the group consisting of SEQ ID NOs: 204-223 and 267-320.

6. The method of any one of claims 2-5, wherein the MHC Class I alpha chain is an HLA-A*02:01 polypeptide comprising the amino acid sequence shown in SEQ ID NO: 5 or 321.

7. The method of any one of claims 2-5, wherein the MHC Class I alpha chain comprises an amino acid sequence shown selected from the group of sequences shown in SEQ ID NOs: 76-141.

8. The method of any one of claims 2-7, wherein the beta2-microglobulin chain comprises an amino acid sequence shown in SEQ ID NO: 143.

9. The method of claim 1, wherein the first MHC subunit is an MHC Class II alpha chain, the second MHC subunit is an MHC Class II beta chain and the MHC-binding peptide is an MHC Class II binding peptide.

10. The method of claim 1, wherein the first MHC subunit is an MHC Class II beta chain, the second MHC subunit is an MHC Class II alpha chain and the MHC-binding peptide is an MHC Class II binding peptide.

11. The method of claim 9 or claim 10, wherein the MHC Class II binding peptide is a CLIP peptide comprising the amino acid sequence KPVSKMRMATPLLMQA (SEQ ID NO: 224).

12. The method of claim 9 or claim 10, wherein the MHC Class II alpha chain comprises an amino acid sequence shown selected from the group of sequences shown in SEQ ID NOs: 174, 190, 192, 194 and 196.

13. The method of claim 9 or claim 10, wherein the MHC Class II beta chain comprises an amino acid sequence shown selected from the group of sequences shown in SEQ ID NOs: 174-189, 191, 193, 195 and 197-203.

14. The method of any one of claims 1-13, wherein the MHC multimer expression construct encodes a linker between the first MHC subunit and the second MHC subunit.

15. The method of claim 14, wherein the linker is a (G₄S)₄ linker.

16. The method of any one of claims 1-15, wherein the MHC multimer expression construct encodes a linker between (i) the first and second MHC subunits and (ii) the multimerization domain.

17. The method of claim 16, wherein the linker is a (GS)₂AG₂SGSG₃S linker.

18. The method of any one of claims 1-17, wherein the cleavage site comprises a Factor Xa cleavage site (SEQ ID NO: 235).

19. The method of any one of claims 1-18, wherein the multimerization domain comprises streptavidin.

20. The method of any one of claims 1-19, wherein the MHC multimer expression construct further encodes a signal peptide.

21. The method of claim 20, wherein the signal peptide is an Ig Kappa chain V-III region CLL signal peptide.

22. The method of any one of claims 1-21, wherein the MHC multimer expression construct further encodes an expression tag.

23. The method of claim 22, wherein the expression tag is selected from the group consisting of 6×His tag, FLAG tag, V5 tag, Myc tag, protein C tag and combinations thereof.

24. The method of any one of claims 1-23, wherein the MHC multimer expression construct comprises a nucleic acid encoding, from 5′ to 3′: an optional signal peptide-an MHC-binding peptide-a cleavage site-a first MHC subunit-a linker-a second MHC subunit-a linker-and a multimerization domain.

25. The method of claim 24, wherein the MHC multimer expression construct comprises a nucleic acid encoding from 5′ to 3′: a signal peptide-an MHC Class I binding peptide-a Factor Xa cleavage site-beta2-microglobulin-a linker-an MHC Class I alpha chain-a linker-and streptavidin.

26. The method of claim 25, wherein the MHC multimer expression construct encodes an amino acid sequence shown in SEQ ID NO: 3.

27. The method of claim 25, wherein the MHC multimer expression construct comprises the nucleotide sequence shown in SEQ ID NO: 1.

28. The method of any one of claims 1-27, wherein the MHC multimer further comprises an oligonucleotide barcode.

29. The method of any one of claims 1-28, wherein the host cell is a mammalian host cell.

30. The method of claim 29, wherein the host cell is a human embryonic kidney (HEK) cell line.

31. The method of any one of claims 1-30, wherein the MHC multimer is secreted from the host cell into cell culture medium.

32. The method of claim 31, wherein the cell culture medium lacks biotin and the method further comprises incubating the MHC multimer with a biotin-conjugated oligonucleotide barcode.

33. The method of any one of claims 1-32, which further comprises incubating the MHC multimer with an agent that cleaves the cleavage site.

34. The method of claim 33, which further comprises incubating the MHC multimer with at least one MHC-binding rescue peptide such that peptide exchange occurs between the MHC-binding peptide and the MHC-binding rescue peptide.

35. The method of claim 34, which comprises incubating the MHC multimer with a plurality of MHC-binding rescue peptides thereby to produce a library of peptide-bound MHC multimers.

36. An isolated Major Histocompatibility Complex (MHC) multimer expression construct, the construct comprising a nucleic acid encoding (i) an MHC-binding peptide operatively linked to a cleavage site; (ii) a first MHC subunit; (iii) a second MHC subunit; and (iv) a multimerization domain.

37. The construct of claim 36, wherein the first MHC subunit is a beta2-microglobulin chain, the second MHC subunit is an MHC Class I alpha chain and the MHC-binding peptide is an MHC Class I binding peptide.

38. The construct of claim 36, wherein the first MHC subunit is an MHC Class I alpha chain, the second MHC subunit is a beta2-microglobulin chain and the MHC-binding peptide is an MHC Class I binding peptide.

39. The construct of claim 37 or claim 38, wherein the MHC Class I binding peptide is a CMV pp65 peptide comprising the amino acid sequence NLVPMVATV (SEQ ID NO: 4).

40. The construct of claim 37 or claim 38, wherein the MHC Class I alpha chain is an HLA-A*02:01 polypeptide comprising the amino acid sequence shown in SEQ ID NO: 3.

41. The construct of claim 37 or claim 38, wherein the MHC Class I alpha chain comprises an amino acid sequence shown selected from the group of sequences shown in SEQ ID NOs: 76-141.

42. The construct of claim 37 or claim 38, wherein the beta2-microglobulin chain comprises an amino acid sequence shown in SEQ ID NO: 143.

43. The construct of claim 36, wherein the first MHC subunit is an MHC Class II alpha chain, the second MHC subunit is an MHC Class II beta chain and the MHC-binding peptide is an MHC Class II binding peptide.

44. The construct of claim 36, wherein the first MHC subunit is an MHC Class II beta chain, the second MHC subunit is an MHC Class II alpha chain and the MHC-binding peptide is an MHC Class II binding peptide.

45. The construct of claim 43 or claim 44, wherein the MHC Class II binding peptide is a CLIP peptide comprising the amino acid sequence KPVSKMRMATPLLMQA (SEQ ID NO: 224).

46. The construct of claim 43 or claim 44, wherein the MHC Class II alpha chain comprises an amino acid sequence shown selected from the group of sequences shown in SEQ ID NOs: 174, 190, 192, 194 and 196.

47. The construct of claim 43 or claim 44, wherein the MHC Class II beta chain comprises an amino acid sequence shown selected from the group of sequences shown in SEQ ID NOs: 174-189, 191, 193, 195 and 197-203.

48. The construct of any one of claims 36-47, wherein the MHC multimer expression construct encodes a linker between the first MHC subunit and the second MHC subunit.

49. The construct of claim 48, wherein the linker is a (G₄S)₄ linker.

50. The construct of any one of claims 36-49, wherein the MHC multimer expression construct encodes a linker between (i) the first and second MHC subunits and (ii) the multimerization domain.

51. The construct of claim 50, wherein the linker is a (GS)₂AG₂SGSG₃S linker.

52. The construct of any one of claims 36-51, wherein the cleavage site comprises a Factor Xa cleavage site (SEQ ID NO: 235).

53. The construct of any one of claims 36-52, wherein the multimerization domain comprises streptavidin.

54. The construct of any one of claims 36-53, wherein the MHC multimer expression construct further encodes a signal peptide.

55. The construct of claim 54, wherein the signal peptide is an Ig Kappa chain V-III region CLL signal peptide.

56. The construct of any one of claims 36-55, wherein the MHC multimer expression construct further encodes an expression tag.

57. The construct of claim 56, wherein the expression tag is selected from the group consisting of 6×His tag, FLAG tag, V5 tag, Myc tag, protein C tag and combinations thereof.

58. The construct of any one of claims 36-57, wherein the MHC multimer expression construct comprises a nucleic acid encoding, from 5′ to 3′: an optional signal peptide-an MHC-binding peptide-a cleavage site-a first MHC subunit-a linker-a second MHC subunit-a linker-and a multimerization domain.

59. The construct of claim 58, wherein the MHC multimer expression construct comprises a nucleic acid encoding from 5′ to 3′: a signal peptide-an MHC Class I binding peptide-a Factor Xa cleavage site-beta2-microglobulin-a linker-an MHC Class I alpha chain-a linker-and streptavidin.

60. The construct of claim 59, wherein the MHC multimer expression construct encodes an amino acid sequence shown in SEQ ID NO: 3.

61. The construct of claim 59, wherein the MHC multimer expression construct comprises the nucleotide sequence shown in SEQ ID NO: 1.

62. The construct of any one of claims 36-61, which is a plasmid.

63. A host cell transfected with the construct of any one of claims 36-62.

64. The host cell of claim 63, which is a mammalian host cell.

65. The host cell of claim 64, which is a human embryonic kidney (HEK) cell line.

66. An isolated supernatant comprising a recombinant MHC multimer, wherein the supernatant is isolated from culture medium of the host cell of any one of claims 63-65.

67. The supernatant of claim 66, wherein the culture medium lacks biotin and the supernatant further comprises a biotin-conjugated oligonucleotide barcode.

68. The supernatant of claim 66 or claim 67, wherein the supernatant further comprises an agent that cleaves the cleavage site.

69. The supernatant of claim 68, which further comprises at least one MHC-binding rescue peptide such that peptide exchange occurs between the MHC-binding peptide and the MHC-binding rescue peptide.

70. The supernatant of claim 69, which comprises a plurality of MHC-binding rescue peptides such that following peptide exchange a library of peptide-bound MHC multimers is contained in the supernatant.

71. A polypeptide library comprising a plurality of peptide loaded MHC (pMHC) multimers, wherein each of the pMHC multimers comprises two or more pMHC monomers conjugated to a multimerization domain, wherein the polypeptide library is prepared according to the method of claim 35.

72. The polypeptide library of claim 71, which comprises pMHCI multimers.

73. The polypeptide library of claim 71, which comprises pMHCII multimers.

74. A method of isolating pMHC-multimer bound lymphocytes comprising:
(a) contacting a plurality of lymphocytes with the library of pMHC multimers of claim 71, thereby to produce a corresponding plurality of lymphocytes each bound to a pMHC-multimer; and
(b) isolating a pMHC-multimer bound lymphocyte.

75. A method of identifying a lymphocyte bound to an pMHC multimer comprising:
(a) contacting a plurality of lymphocytes with the library of pMHC multimers of claim 71;
(b) compartmentalizing a lymphocyte of the plurality of lymphocytes bound to a pMHC multimer of the library in a single compartment, wherein the pMHC multimer comprises a unique identifier; and
(c) determining the unique identifier for the pMHC bound to the compartmentalized lymphocyte.

76. The method of claim 74 or claim 75, wherein the pMHC multimers are pMHCI multimers.

77. The method of claim 74 or claim 75, wherein the pMHC multimers are pMHCII multimers.

78. The method of any one of claims 74-77, wherein the lymphocyte is a T cell, B cell, or NK cell.


SEQUENCE LISTING (327 sequences)

SEQ ID NO: 1 - DNA, 1926 bp, Artificial Sequence
  Description of Artificial Sequence Synthetic polynucleotide
atggaggctccggctcagctgctgttccttctgctgctgtggctgcccgacaccaccggaaatttggtcccgatggttgcaacggttggcggaggggcgtctgggggcggtggtagtatagaaggacgaggcggtggcggaagtggtggtggaggctctatccaacgcacccctaaaatccaggtctactcgagacacccggctgagaacgggaagtccaacttcctgaactgctacgtgtccggttttcacccgtccgacattgaggtggacctcctgaagaacggagagcgcatcgagaaggtggaacactccgaccttagcttctccaaggattggtcattctacctgttgtactacaccgagttcactccgaccgaaaaggacgaatacgcatgcagggtgaaccacgtgaccctgtcccagccgaagatcgtgaagtgggaccgggacatgggaggcggcggatcaggaggcggaggatctgggggtggaggaagcggtggtggcggatccggaagccactccatgcggtacttcttcacctccgtgtcacgccctggtcggggagagcctcgattcatcgccgtcggctacgtggacgacactcagttcgtccgctttgattcggacgctgcaagccagcggatggaaccaagggcgccttggatcgaacaggagggccccgagtactgggacggggaaactcggaaagtgaaggcccactctcagactcaccgggtggatctcgggacgctcagaggctactacaaccagtcagaggccggcagccatactgtccaacggatgtacggatgcgacgtgggctccgattggaggttcctgagaggataccatcagtacgcgtacgacggaaaggactatatcgcgctcaaggaggacctgagatcctggactgcggccgatatggccgctcagacgactaaacacaagtgggaagcagctcacgtggccgagcagctgagggcctacctggagggaacttgcgtcgagtggctgcggagatatctggagaatgggaaggaaaccctccagaggacagatgcacccaagacccatatgactcaccatgccgtgagcgaccacgaagccaccctgcggtgttgggccctgtccttctacccggccgaaatcacgctgacctggcaacgcgatggagaggaccagacccaagacactgaactcgtggaaaccagacccgcgggagatggcaccttccaaaagtgggccgctgtggtggtcccgtcgggacaggagcagcggtacacttgccacgtccagcacgagggactccccaagcctctgaccctgcgctgggaacctagctccggaagcggatccgcaggcggatcgggatcaggcggtggctctgacccctccaaggacagcaaggctcaggtgtcagccgccgaagcaggcatcaccggcacctggtacaaccagcttgggtccacctttatcgtgaccgcgggagcagatggcgccctgactggcacctacgaatccgccgtcggaaacgccgagtccagatacgtgctgaccgggcgctacgactccgcgcctgcaaccgatggctcgggtacagcccttggatggactgtcgcctggaagaacaactacaggaacgcccactccgccaccacttggagcgggcagtatgtcggaggagctgaggcgcggattaacactcaatggctgctgacctccggtaccaccgaagccaatgcatggaagtcgaccctggtcggccatgacaccttcaccaaggtgaaaccttcggccgcctccattgacgccgccaagaaggcgggggtgaacaacggcaacccgctggatgccgtgcagcagggctccactggccaccaccaccatcaccacgactataaggacgacgatgacaagtga

SEQ ID NO: 2 - protein, 641 aa, Artificial Sequence
  Description of Artificial Sequence Synthetic polypeptide
MEAPAQLLFLLLLWLPDTTGNLVPMVATVGGGASGGGGSIEGRGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSGSGSAGGSGSGGGSDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGSTGHHHHHHDYKDDDDK

SEQ ID NO: 3 - protein, 607 aa, Artificial Sequence
  Description of Artificial Sequence Synthetic polypeptide
NLVPMVATVGGGASGGGGSIEGRGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSGSGSAGGSGSGGGSDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGSTG

SEQ ID NO: 4 - protein, 9 aa, Human betaherpesvirus 5
NLVPMVATV

SEQ ID NO: 5 - protein, 278 aa, Homo sapiens [sequence omitted]

SEQ ID NO: 6 - protein, 10 aa, Homo sapiens
EAAGIGILTV

SEQ ID NO: 7 - protein, 10 aa, Alphapapillomavirus 1
YMLDLQPETT

SEQ ID NO: 8 - protein, 9 aa, Human alphaherpesvirus 1
SLPITVYYA

SEQ ID NO: 9 - protein, 9 aa, Homo sapiens
RMFPNAPYL

SEQ ID NO: 10 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 11 - protein, 365 aa, Homo sapiens
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 12 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 13 - protein, 365 aa, Homo sapiens [sequence omitted]

SEQ ID NO: 14 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 15 - protein, 366 aa, Homo sapiens
MRVMAPRTLILLLSGALALTETWAGSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKPSSQPTIPIVGIVAGLAVLAVLAVLGAMVAVVMCRRKSSGGKGGSCSQAASSNSAQGSDESLIACKA

SEQ ID NO: 16 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 17 - protein, 362 aa, Homo sapiens
MLVMAPRTVLLLLSAALALTETWAGSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 18 - protein, 362 aa, Homo sapiens [sequence omitted]

SEQ ID NO: 19 - protein, 362 aa, Homo sapiens [sequence omitted]

SEQ ID NO: 20 - protein, 362 aa, Homo sapiens [sequence omitted]

SEQ ID NO: 21 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 22 - protein, 366 aa, Homo sapiens
MRVMAPRTLILLLSGALALTETWACSHSMRYFYTAVSRPGRGEPRFIAVGYVDDTQFVRFDSDAASPRGEPRAPWVEQEGPEYWDRETQKYKRQAQTDRVSLRNLRGYYNQSEAGSHTLQWMYGCDLGPDGRLLRGYDQSAYDGKDYIALNEDLRSWTAADTAAQITQRKWEAARAAEQQRAYLEGTCVEWLRRYLENGKETLQRAEHPKTHVTHHLVSDHEATLRCWALGFYPAEITLTWQRDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 23 - protein, 366 aa, Homo sapiens [sequence omitted]

SEQ ID NO: 24 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 25 - protein, 366 aa, Homo sapiens
MRVMAPRTLILLLSGALALTETWACSHSMRYFYTAVSRPGRGEPRFIAVGYVDDTQFVQFDSDAASPRGEPRAPWVEQEGPEYWDRETQKYKRQAQTDRVNLRKLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSSQPTIPIVGIVAGLAVLAVLAVLGAVMAVVMCRRKSSGGKGGSCSQAASSNSAQGSDESLIACKA

SEQ ID NO: 26 - protein, 362 aa, Homo sapiens
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 27 - protein, 365 aa, Homo sapiens
MAVMAPRTLLLLLLGALALTQTWAGSHSMRYFTTSVSRPGRGEPRFIAVGYVDDTQFVRFDSDAASQRMEPRAPWIEQEGPEYWDLQTRNVKAQSQTDRANLGTLRGYYNQSEAGSHTIQMMYGCDVGSDGRFLRGYRQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVFAGAVVAAVRWRRKSSDRKGGSYSQAASSDSAQGSDMSLTACKV

SEQ ID NO: 28 - protein, 362 aa, Homo sapiens
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQLRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 29 - protein, 366 aa, Homo sapiens
MRVMAPRTLILLLSGALALTETWAGSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 30 - protein, 362 aa, Homo sapiens [sequence omitted]

SEQ ID NO: 31 - protein, 366 aa, Homo sapiens
MRVMAPRTLILLLSGALALTETWACSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSSQPTIPIVGIVAGLAVLAVLAVLGAVMAVVMCRRKSSGGKGGSCSQAASSNSAQGSDESLIACKA

SEQ ID NO: 32 - protein, 362 aa, Homo sapiens
MRVTAPRTVLLLLSGALALTETWAGSHSMRYFYTAMSRPGRGEPRFIAVGYVDDTQFVRFDSDAASPRMAPRAPWIEQEGPEYWDRETQISKTNTQTYRESLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTIPIVGIVAGLAVLAVVVIGAVVATVMCRRKSSGGKGGSYSQAASSDSAQGSDVSLTA

SEQ ID NO: 33 - protein, 366 aa, Homo sapiens [sequence omitted]

SEQ ID NO: 34 - protein, 365 aa, Homo sapiens [sequence omitted]

SEQ ID NO: 35 - protein, 362 aa, Homo sapiens
MRVTAPRTLLLLLWGAVALTETWAGSHSMRYFYTAMSRPGRGEPRFITVGYVDDTQFVRFDSDATSPRMAPRAPWIEQEGPEYWDRETQISKTNTQTYRENLRTALRYYNQSEAGSHTWQTMYGCDLGPDGRLLRGHNQLAYDGKDYIALNEDLSSWTAADTAAQITQLKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 36 - protein, 366 aa, Homo sapiens [sequence omitted]

SEQ ID NO: 37 - protein, 365 aa, Homo sapiens
MAVMAPRTLVLLLSGALALTQTWAGSHSMRYFYTSVSRPGRGEPRFIAVGYVDDTQFVRFDSDAASQRMEPRAPWIEQEGPEYWDRNTRNVKAHSQTDRANLGTLRGYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVIAGAVVAAVMWRRKSSDRKGGSYSQAASSDSAQGSDMSLTACKV

SEQ ID NO: 38 - protein, 362 aa, Homo sapiens [sequence omitted]

SEQ ID NO: 39 - protein, 362 aa, Homo sapiens
MLVMAPRTVLLLLSAALALTETWAGSHSMRYFYTAVSRPGRGEPRFISVGYVDDTQFVRFDSDAASPREEPRAPWIEQEGPEYWDRNTQICKTNTQTDRESLRNLRGYYNQSEAGSHTLQWMYGCDVGPDGRLLRGYNQFAYDGKDYIALNEDLSSWTAADTAAQITQRKWEAAREAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAASSDSAQGSDVSLTA

SEQ ID NO: 40 - protein, 365 aa, Homo sapiens [sequence omitted]

SEQ ID NO: 41 - protein, 365 aa, Homo sapiens [sequence omitted]

SEQ ID NO: 42 - protein, 365 aa, Homo sapiens
MAVMAPRTLVLLLSGALALTQTWAGSHSMRYFYTSVSRPGRGEPRFIAVGYVDDTQFVRFDSDAASQRMEPRAPWIEQEGPEYWDRNTRNVKAHSQTDRESLRIALRYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVIAGAVVAAVMWRRKSSDRKGGSYSQAASSDSAQGSDMSLTACKV

SEQ ID NO: 43 - protein, 362 aa, Homo sapiens
MRVTAPRTLLLLLWGAVALTETWAGSHSMRYFHTSVSRPGRGEPRFISVGYVDGTQFVRFDSDAASPRTEPRAPWIEQEGPEYWDRNTQISKTNTQTYRESLRNLRGYYNQSEAGSHTLQRMYGCDVGPDGRLLRGHDQSAYDGKDYIALNEDLSSWTAADTAAQITQRKWEAARVAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTIPIVGIVAGLAVLAVVVIGAVVATVMCRRKSSGGKGGSYSQAASSDSAQGSDVSLTA

SEQ ID NO: 44 - protein, 362 aa, Homo sapiens [sequence omitted]

SEQ ID NO: 45 - protein, 362 aa, Homo sapiens [sequence omitted]

SEQ ID NO: 46 - protein, 366 aa, Homo sapiens [sequence omitted]

SEQ ID NO: 47 - protein, 366 aa, Homo sapiens [sequence omitted]

SEQ ID NO: 48 - protein, 366 aa, Homo sapiens [sequence omitted]

SEQ ID NO: 49 - protein, 362 aa, Homo sapiens [sequence omitted]

SEQ ID NO: 50 - protein, 365 aa, Homo sapiens [sequence omitted]

SEQ ID NO: 51 - protein, 365 aa, Homo sapiens [sequence omitted]

SEQ ID NO: 52 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 53 - protein, 372 aa, Homo sapiens
MRVMAPQALLLLLSGALALIETWAGSHSMRYFYTAVSRPGRGEPRFIAVGYVDDTQFVRFDSDAASPRGEPRAPWVEQEGPEYWDRETQKYKRQAQADRVNLRKLRGYYNQSEAGSHTIQRMYGCDLGPDGRLLRGYNQFAYDGKDYIALNEDLRSWTAADTAAQISQRKLEAAREAEQLRAYLEGECVEWLRGYLENGKETLQRAERPKTHVTHHPVSDHEATLRCWALGFYPAEITLTWQRDGEDQTQDTELVETRPAGDGTFQKWAAVVVPSGQEQRYTCHVQHEGLQEPCTLRWKPSSQPTIPNLGIVSGPAVLAVLAVLAVLAVLGAVVAAVIHRRKSSGGKGGSCSQAASSNSAQGSDESLIACKA

SEQ ID NO: 54 - protein, 362 aa, Homo sapiens [sequence omitted]

SEQ ID NO: 55 - protein, 362 aa, Homo sapiens [sequence omitted]

SEQ ID NO: 56 - protein, 366 aa, Homo sapiens [sequence omitted]

SEQ ID NO: 57 - protein, 362 aa, Homo sapiens [sequence omitted]

SEQ ID NO: 58 - protein, 365 aa, Homo sapiens [sequence omitted]

SEQ ID NO: 59 - protein, 365 aa, Homo sapiens [sequence omitted]

SEQ ID NO: 60 - protein, 366 aa, Homo sapiens
MRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 61 - protein, 366 aa, Homo sapiens [sequence omitted]

SEQ ID NO: 62 - protein, 365 aa, Homo sapiens [sequence omitted]

SEQ ID NO: 63 - protein, 365 aa, Homo sapiens
MAVMAPRTLLLLLLGALALTQTWAGSHSMRYFFTSVSRPGRGEPRFIAVGYVDDTQFVRFDSDAASQRMEPRAPWIEQEGPEYWDQETRNVKAHSQTDRESLRIALRYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAMFAGAVVAAVRWRRKSSDRKGGSYSQAASSDSAQGSDMSLTACKV

SEQ ID NO: 64 - protein, 362 aa, Homo sapiens [sequence omitted]

SEQ ID NO: 65 - protein, 362 aa, Homo sapiens [sequence omitted]

SEQ ID NO: 66 - protein, 362 aa, Homo sapiens [sequence omitted]

SEQ ID NO: 67 - protein, 365 aa, Homo sapiens [sequence omitted]

SEQ ID NO: 68 - protein, 362 aa, Homo sapiens [sequence omitted]

SEQ ID NO: 69 - protein, 362 aa, Homo sapiens [sequence omitted]

SEQ ID NO: 70 - protein, 362 aa, Homo sapiens [sequence omitted]

SEQ ID NO: 71 - protein, 366 aa, Homo sapiens
MRVMAPRTLILLLSGALALTETWACSHSMRYFYTAVSRPGRGEPRFIAVGYVDDTQFVRFDSDAASPRGEPRAPWVEQEGPEYWDRETQKYKRQAQADRVSLRNLRGYYNQSEAGSHTLQRMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSSQPTIPIVGIVAGLAVLAVLAVLGAVMAVVMCRRKSSGGKGGSCSQAASSNSAQGSDESLIACKA

SEQ ID NO: 72 - protein, 362 aa, Homo sapiens [sequence omitted]

SEQ ID NO: 73 - protein, 362 aa, Homo sapiens [sequence omitted]

SEQ ID NO: 74 - protein, 362 aa, Homo sapiens [sequence omitted]

SEQ ID NO: 75 - protein, 365 aa, Homo sapiens [sequence omitted]

SEQ ID NO: 76 - protein, 278 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 77 - protein, 278 aa, Homo sapiens
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 78 - protein, 278 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 79 - protein, 278 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSS

SEQ ID NO: 80 - protein, 278 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 81 - protein, 278 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKPSS

SEQ ID NO: 82 - protein, 278 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSS

SEQ ID NO: 83 - protein, 278 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 84 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 85 - protein, 278 aa, Homo sapiens
GSHSMRYFYTAMSRPGRGEPRFIAVGYVDDTQFVRFDSDAASPRMAPRAPWIEQEGPEYWDGETRNMKASAQTYRENLRIALRYYNQSEAGSHIIQVMYGCDVGPDGRLLRGHDQSAYDGKDYIALNEDLSSWTAADTAAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 86 - protein, 278 aa, Homo sapiens
GSHSMRYFYTAMSRPGRGEPRFIAVGYVDDTQFVRFDSDAASPRMAPRAPWIEQEGPEYWDGETRNMKASAQTYRENLRIALRYYNQSEAGSHIIQVMYGCDVGPDGRLLRGHNQYAYDGKDYIALNEDLSSWTAADTAAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 87 - protein, 278 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPAS

SEQ ID NO: 88 - protein, 278 aa, Homo sapiens
CSHSMRYFYTAVSRPGRGEPRFIAVGYVDDTQFVRFDSDAASPRGEPRAPWVEQEGPEYWDRETQKYKRQAQTDRVSLRNLRGYYNQSEAGSHTLQWMYGCDLGPDGRLLRGYDQSAYDGKDYIALNEDLRSWTAADTAAQITQRKWEAARAAEQQRAYLEGTCVEWLRRYLENGKETLQRAEHPKTHVTHHLVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 89 - protein, 278 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSS

SEQ ID NO: 90 - protein, 278 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSS

SEQ ID NO: 91 - protein, 278 aa, Homo sapiens
CSHSMRYFYTAVSRPGRGEPRFIAVGYVDDTQFVQFDSDAASPRGEPRAPWVEQEGPEYWDRETQKYKRQAQTDRVNLRKLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSS

SEQ ID NO: 92 - protein, 278 aa, Homo sapiens
GSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSS

SEQ ID NO: 93 - protein, 278 aa, Homo sapiens
GSHSMRYFTTSVSRPGRGEPRFIAVGYVDDTQFVRFDSDAASQRMEPRAPWIEQEGPEYWDLQTRNVKAQSQTDRANLGTLRGYYNQSEAGSHTIQMMYGCDVGSDGRFLRGYRQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 94 - protein, 278 aa, Homo sapiens
GSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQLRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSS

SEQ ID NO: 95 - protein, 278 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSS

SEQ ID NO: 96 - protein, 278 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 97 - protein, 278 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 98 - protein, 278 aa, Homo sapiens
GSHSMRYFYTAMSRPGRGEPRFIAVGYVDDTQFVRFDSDAASPRMAPRAPWIEQEGPEYWDRETQISKTNTQTYRESLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 99 - protein, 278 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSS

SEQ ID NO: 100 - protein, 278 aa, Homo sapiens
GSHSMRYFSTSVSRPGSGEPRFIAVGYVDDTQFVRFDSDAASQRMEPRAPWIEQERPEYWDQETRNVKAQSQTDRVDLGTLRGYYNQSEAGSHTIQIMYGCDVGSDGRFLRGYEQHAYDGKDYIALNEDLRSWTAADMAAQITQRKWEAARWAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 101 - protein, 278 aa, Homo sapiens
GSHSMRYFYTAMSRPGRGEPRFITVGYVDDTQFVRFDSDATSPRMAPRAPWIEQEGPEYWDRETQISKTNTQTYRENLRTALRYYNQSEAGSHTWQTMYGCDLGPDGRLLRGHNQLAYDGKDYIALNEDLSSWTAADTAAQITQLKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 102 - protein, 278 aa, Homo sapiens
CSHSMRYFYTAVSRPGRGEPRFIAVGYVDDTQFVRFDSDAASPRGEPRAPWVEQEGPEYWDRETQKYKRQAQADRVSLRNLRGYYNQSEAGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 103 - protein, 278 aa, Homo sapiens
GSHSMRYFYTSVSRPGRGEPRFIAVGYVDDTQFVRFDSDAASQRMEPRAPWIEQEGPEYWDRNTRNVKAHSQTDRANLGTLRGYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 104 - protein, 278 aa, Homo sapiens
GSHSMRYFYTSVSRPGRGEPRFISVGYVDDTQFVRFDSDAASPREEPRAPWIEQEGPEYWDRNTQICKTNTQTYRENLRIALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 105 - protein, 278 aa, Homo sapiens
GSHSMRYFYTAVSRPGRGEPRFISVGYVDDTQFVRFDSDAASPREEPRAPWIEQEGPEYWDRNTQICKTNTQTDRESLRNLRGYYNQSEAGSHTLQWMYGCDVGPDGRLLRGYNQFAYDGKDYIALNEDLSSWTAADTAAQITQRKWEAAREAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 106 - protein, 278 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRHLENGKETLQRTDPPRTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 107 - protein, 278 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSS

SEQ ID NO: 108 - protein, 278 aa, Homo sapiens
GSHSMRYFYTSVSRPGRGEPRFIAVGYVDDTQFVRFDSDAASQRMEPRAPWIEQEGPEYWDRNTRNVKAHSQTDRESLRIALRYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 109 - protein, 278 aa, Homo sapiens
GSHSMRYFHTSVSRPGRGEPRFISVGYVDGTQFVRFDSDAASPRTEPRAPWIEQEGPEYWDRNTQISKTNTQTYRESLRNLRGYYNQSEAGSHTLQRMYGCDVGPDGRLLRGHDQSAYDGKDYIALNEDLSSWTAADTAAQITQRKWEAARVAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 110 - protein, 278 aa, Homo sapiens
GSHSMRYFHTSVSRPGRGEPRFISVGYVDDTQFVRFDSDAASPRTEPRAPWIEQEGPEYWDRETQISKTNTQTYREDLRTLLRYYNQSEAGSHTIQRMSGCDVGPDGRLLRGYNQFAYDGKDYIALNEDLSSWTAADTAAQITQRKWEAARVAEQDRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 111 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHTWQTMYGCDVGPDGRLLRGHNQYAYDGKDYIALNEDLSSWTAADTAAQITQRKWEAAREAEQLRAYLEGLCVEWLRRHLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 112 - protein, 278 aa, Homo sapiens
CSHSMRYFSTSVSRPGRGEPRFIAVGYVDDTQFVRFDSDAASPRGEPRAPWVEQEGPEYWDRETQKYKRQAQTDRVSLRNLRGYYNQSEAGSHTLQWMFGCDLGPDGRLLRGYDQSAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSS

SEQ ID NO: 113 - protein, 278 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 114 - protein, 278 aa, Homo sapiens
CSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEPSS

SEQ ID NO: 115 - protein, 278 aa, Homo sapiens
GSHSMRYFHTSVSRPGRGEPRFITVGYVDDTLFVRFDSDAASPREEPRAPWIEQEGPEYWDRETQICKAKAQTDREDLRTLLRYYNQSEAGSHTLQNMYGCDVGPDGRLLRGYHQDAYDGKDYIALNEDLSSWTAADTAAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 116 - protein, 278 aa, Homo sapiens
GSHSMRYFTTSVSRPGRGEPRFIAVGYVDDTQFVRFDSDAASQRMEPRAPWIEQERPEYWDQETRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 117 - protein, 278 aa, Homo sapiens
GSHSMRYFSTSVSRPGSGEPRFIAVGYVDDTQFVRFDSDAASQRMEPRAPWIEQERPEYWDQETRNVKAHSQTDRENLGTLRGYYNQSEAGSHTIQIMYGCDVGSDGRFLRGYEQHAYDGKDYIALNEDLRSWTAADMAAQITQRKWEAARRAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 118 - protein, 278 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 119 - protein, 278 aa, Homo sapiens
GSHSMRYFYTAVSRPGRGEPRFIAVGYVDDTQFVRFDSDAASPRGEPRAPWVEQEGPEYWDRETQKYKRQAQADRVNLRKLRGYYNQSEAGSHTIQRMYGCDLGPDGRLLRGYNQFAYDGKDYIALNEDLRSWTAADTAAQISQRKLEAAREAEQLRAYLEGECVEWLRGYLENGKETLQRAERPKTHVTHHPVSDHEATLRCWALGFYPAEITLTWQRDGEDQTQDTELVETRPAGDGTFQKWAAVVVPSGQEQRYTCHVQHEGLQEPCTLRWKPSS

SEQ ID NO: 120 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRFLRGHNQYAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 121 - protein, 278 aa, Homo sapiens
GSHSMRYFYTSVSRPGRGEPRFISVGYVDDTQFVRFDSDAASPREEPRAPWIEQEGPEYWDRNTQICKTNTQTDRESLRNLRGYYNQSEAGSHTWQTMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 122 - protein, 278 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHILQRMYGCDVGPDGRLLRGYDQSAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSS

SEQ ID NO: 123 - protein, 278 aa, Homo sapiens
GSHSMRYFYTAMSRPGRGEPRFIAVGYVDDTQFVRFDSDAASPRTEPRAPWIEQEGPEYWDGETRNMKASAQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 124 - protein, 278 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 125 - protein, 278 aa, Homo sapiens
GSHSMRYFYTSMSRPGRGEPRFIAVGYVDDTQFVRFDSDAASQRMEPRAPWIEQEGPEYWDRNTRNVKAQSQTDRVDLGTLRGYYNQSEAGSHTIQRMYGCDVGPDGRFLRGYHQYAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEPSS

SEQ ID NO: 126 - protein, 278 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSS

SEQ ID NO: 127 - protein, 278 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTFQRMYGCDLGPDGRLLRGYDQFAYDGKDYIALNEDLRSWTAADTAAQITQRKLEAARAAEQDRAYLEGTCVEWLRRYLENGKKTLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSS

SEQ ID NO: 128 - protein, 278 aa, Homo sapiens
GSHSMRYFYTSVSRPGRGEPRFIAVGYVDDTQFVRFDSDAASQRMEPRAPWIEQEGPEYWDRNTRNVKAQSQTDRVDLGTLRGYYNQSEAGSHTIQMMYGCDVGSDGRFLRGYRQDAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEPSS

SEQ ID NO: 129 - protein, 278 aa, Homo sapiens
GSHSMRYFFTSVSRPGRGEPRFIAVGYVDDTQFVRFDSDAASQRMEPRAPWIEQEGPEYWDQETRNVKAHSQTDRESLRIALRYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 130 - protein, 278 aa, Homo sapiens
GSHSMRYFHTAMSRPGRGEPRFITVGYVDDTLFVRFDSDATSPRKEPRAPWIEQEGPEYWDRETQISKTNTQTYRENLRIALRYYNQSEAGSHTWQRMYGCDLGPDGRLLRGYNQLAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 131 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 132 - protein, 278 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTWQRMYGCDLGPDGRLLRGYNQLAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 133 - protein, 278 aa, Homo sapiens
GSHSMRYFYTSVSRPGRGEPRFIAVGYVDDTQFVRFDSDAASRRMEPRAPWIEQEGPEYWDGETRKVKAHSQTHRVDLGTLRGYYNQSEAGSHTLQRMYGCDVGSDWRFLRGYHQYAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSS

SEQ ID NO: 134 - protein, 278 aa, Homo sapiens
GSHSMRYFYTAMSRPGRGEPRFIAVGYVDDTQFVRFDSDAASPREEPRAPWIEQEGPEYWDRNTQIYKAQAQTDRESLRNLRGYYNQSEAGSHTWQTMYGCDLGPDGRLLRGHNQLAYDGKDYIALNEDLSSWTAADTAAQITQRKWEAAREAEQLRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 135 - protein, 278 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTWQRMYGCDLGPDGRLLRGYNQLAYDGKDYIALNEDLSSWTAADTAAQITQRKWEAARVAEQDRAYLEGLCVESLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 136 - protein, 278 aa, Homo sapiens
GSHSMRYFYTAMSRPGRGEPRFIAVGYVDDTQFVRFDSDAASPRTEPRAPWIEQEGPEYWDRETQISKTNTQTYRENLRIALRYYNQSEAGSHTWQTMYGCDVGPDGRLLRGHNQYAYDGKDYIALNEDLSSWTAADTAAQITQRKWEAAREAEQLRAYLEGLCVEWLRRHLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 137 - protein, 278 aa, Homo sapiens
CSHSMRYFYTAVSRPGRGEPRFIAVGYVDDTQFVRFDSDAASPRGEPRAPWVEQEGPEYWDRETQKYKRQAQADRVSLRNLRGYYNQSEAGSHTLQRMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 138 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRLLRGHDQFAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 139 - protein, 278 aa, Homo sapiens
GSHSMRYFHTSVSRPGRGEPRFITVGYVDDTLFVRFDSDATSPRKEPRAPWIEQEGPEYWDRETQISKTNTQTYRESLRNLRGYYNQSEAGSHTLQSMYGCDVGPDGRLLRGHNQYAYDGKDYIALNEDLRSWTAADTAAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 140 - protein, 278 aa, Homo sapiens
GSHSMRYFYTAMSRPGRGEPRFISVGYVDDTQFVRFDSDAASPREEPRAPWIEQEGPEYWDRETQISKTNTQTYRESLRNLRGYYNQSEAGSHTLQRMYGCDVGPDGRLLRGHDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 141 - protein, 278 aa, Homo sapiens
GSHSMRYFFTSVSRPGRGEPRFIAVGYVDDTQFVRFDSDAASQRMEPRAPWIEQEGPEYWDQETRNVKAHSQTDRVDLGTLRGYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 142 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 143 - protein, 99 aa, Homo sapiens [sequence omitted]

SEQ ID NO: 144 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 145 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 146 - protein, 266 aa, Homo sapiens [sequence omitted]

SEQ ID NO: 147 - protein, 266 aa, Homo sapiens
MVCLRLPGGSCMAVLTVTLMVLSSPLALAGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 148 - protein, 266 aa, Homo sapiens [sequence omitted]

SEQ ID NO: 149 - protein, 266 aa, Homo sapiens [sequence omitted]

SEQ ID NO: 150 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 151 - protein, 266 aa, Homo sapiens [sequence omitted]

SEQ ID NO: 152 - protein, 266 aa, Homo sapiens [sequence omitted]

SEQ ID NO: 153 - protein, 266 aa, Homo sapiens [sequence omitted]

SEQ ID NO: 154 - protein, 266 aa, Homo sapiens [sequence omitted]

SEQ ID NO: 155 - protein, 266 aa, Homo sapiens [sequence omitted]

SEQ ID NO: 156 - protein, 266 aa, Homo sapiens [sequence omitted]

SEQ ID NO: 157 - protein, 266 aa, Homo sapiens [sequence omitted]

SEQ ID NO: 158 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 159 - protein, 266 aa, Homo sapiens [sequence omitted]

SEQ ID NO: 160 - protein, 255 aa, Homo sapiens [sequence omitted]

SEQ ID NO: 161 - protein, 261 aa, Homo sapiens [sequence omitted]

SEQ ID NO: 162 - protein, 255 aa, Homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 163 - protein, 261 aa, Homo sapiens
MSWKKALRIPGDLRVATVTLMLAMLSSLLAEGRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKMLSGVGGFVLGLIFLGLGLIIRQRSQKGLLH

SEQ ID NO: 164 - protein, 255 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTETVVCALGLSVGLVGIVVGTVLIIRGLRSVGASRHQGPL

SEQ ID NO: 165 - protein, 261 aa, Homo sapiens [sequence omitted]

SEQ ID NO: 166 - protein, 254 aa, Homo sapiens
MILNKALMLGALALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 167 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 168 - protein, 261 aa, Homo sapiens
MSWKKALRIPGGLRAATVTLMLAMLSTPVAEGRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSKMLSGIGGFVLGLIFLGLGLIIHHRSQKGLLH

SEQ ID NO: 169 - protein, 261 aa, Homo sapiens [sequence omitted]

SEQ ID NO: 170 - protein, 261 aa, Homo sapiens [sequence omitted]

SEQ ID NO: 171 - protein, 269 aa, Homo sapiens [sequence omitted]

SEQ ID NO: 172 - protein, 261 aa, Homo sapiens [sequence omitted]

SEQ ID NO: 173 - protein, 261 aa, Homo sapiens [sequence omitted]

SEQ ID NO: 174 - protein, 191 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTE

SEQ ID NO: 175 - protein, 198 aa, Homo sapiens
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 176 - protein, 198 aa, Homo sapiens
GDTRPRFLWQLKFECHFFNGTERVRLLERCIYNQEESVRFDSDVGEYRAVTELGRPDAEYWNSQKDLLEQRRAAVDTYCRHNYGAVESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 177 - protein, 198 aa, Homo sapiens
GDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 178 - protein, 198 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 179 - protein, 198 aa, Homo sapiens
GDTRPRFLEQVKHECHFFNGTERVRFLDRYFYHQEEYVRFDSDVGEYRAVTELGRPDAEYWNSQKDLLEQRRAAVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 180 - protein, 198 aa, Homo sapiens
GDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSK

SEQ ID NO: 181 - protein, 198 aa, Homo sapiens
GDTRPRFLEYSTGECYFFNGTERVRFLDRYFYNQEEYVRFDSDVGEYRAVTELGRPSAEYWNSQKDFLEDRRALVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWSARSESAQSK

SEQ ID NO: 182 - protein, 198 aa, Homo sapiens
GDTRPRFLEEVKFECHFFNGTERVRLLERRVHNQEEYARYDSDVGEYRAVTELGRPDAEYWNSQKDLLERRRAAVDTYCRHNYGVGESFTVQRRVQPKVTVYPSKTQPLQHHNLLVCSVNGFYPGSIEVRWFRNGQEEKTGVVSTGLIQNGDWTFQTLVMLETVPQSGEVYTCQVEHPSVMSPLTVEWRARSESAQSK

SEQ ID NO: 183 - protein, 198 aa, Homo sapiens
GDTRPRFLEYSTSECHFFNGTERVRFLDRYFYNQEEYVRFDSDVGEFRAVTELGRPDEEYWNSQKDFLEDRRAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 184 - protein, 198 aa, Homo sapiens
GDTRPRFLEYSTSECHFFNGTERVRFLDRYFYNQEEYVRFDSDVGEFRAVTELGRPDEEYWNSQKDFLEDRRAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 185 - protein, 198 aa, Homo sapiens
GDTRPRFLEYSTSECHFFNGTERVRFLDRYFHNQEENVRFDSDVGEFRAVTELGRPDAEYWNSQKDILEDERAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 186 - protein, 198 aa, Homo sapiens
GDTRPRFLEYSTSECHFFNGTERVRFLDRYFHNQEENVRFDSDVGEFRAVTELGRPDAEYWNSQKDILEDERAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 187 - protein, 198 aa, Homo sapiens
GDTRPRFLEYSTSECHFFNGTERVRFLDRYFHNQEEFVRFDSDVGEYRAVTELGRPAAEHWNSQKDLLERRRAEVDTYCRHNYGVVESFTVQRRVHPKVTVYPSKTQPLQHYNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 188 - protein, 198 aa, Homo sapiens
GDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 189 - protein, 198 aa, Homo sapiens
GDTRPRFLWQPKRECHFFNGTERVRFLDRHFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 190 - protein, 195 aa, Homo sapiens
EDIVADHVASCGVNLYQFYGPSGQYTHEFDGDEEFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTET

SEQ ID NO: 191 - protein, 199 aa, Homo sapiens
GRDSPEDFVYQFKGLCYFTNGTERVRGVTRHIYNREEYVRFDSDVGVYRAVTPQGRPVAEYWNSQKEVLEGARASVDRVCRHNYEVAYRGILQRRVEPTVTISPSRTEALNHHNLLICSVTDFYPSQIKVRWFRNDQEETAGVVSTPLIRNGDWTFQILVMLEMTPQRGDVYTCHVEHPSLQSPITVEWRAQSESAQSK

SEQ ID NO: 192 - protein, 195 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTET

SEQ ID NO: 193 - protein, 199 aa, Homo sapiens
GRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSK

SEQ ID NO: 194 - protein, 195 aa, Homo sapiens
EDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTET

SEQ ID NO: 195 - protein, 199 aa, Homo sapiens
GRDSPEDFVYQFKGMCYFTNGTERVRLVTRYIYNREEYARFDSDVGVYRAVTPLGPPAAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 196 - protein, 194 aa, Homo sapiens
EDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTET

SEQ ID NO: 197 - protein, 199 aa, Homo sapiens
GRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSK

SEQ ID NO: 198 - protein, 199 aa, Homo sapiens
GRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSK

SEQ ID NO: 199 - protein, 199 aa, Homo sapiens
GRDSPEDFVYQFKGMCYFTNGTERVRLVTRYIYNREEYARFDSDVGVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 200 - protein, 199 aa, Homo sapiens
GRDSPEDFVFQFKGMCYFTNGTERVRGVTRYIYNREEYARFDSDVGVYRAVTPLGRLDAEYWNSQKDILEEDRASVDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 201 - protein, 199 aa, Homo sapiens
GRDSPEDFVYQFKGLCYFTNGTERVRGVTRHIYNREEYVRFDSDVGVYRAVTPQGRPDAEYWNSQKEVLEGARASVDRVCRHNYEVAYRGILQRRVEPTVTISPSRTEALNHHNLLICSVTDFYPSQIKVRWFRNDQEETAGVVSTPLIRNGDWTFQILVMLEMTPQRGDVYTCHVEHPSLQSPITVEWRAQSESAQSK

SEQ ID NO: 202 - protein, 199 aa, Homo sapiens
GRDSPEDFVYQFKGMCYFTNGTERVRLVTRHIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSK

SEQ ID NO: 203 - protein, 199 aa, Homo sapiens
GRDSPEDFVYQFKGMCYFTNGTERVRLVTRHIYNREEYARFDSDVGVYRAVTPQGRPVAEYWNSQKEVLERTRAELDTVCRHNYEVGYRGILQRRVEPTVTISPSRTEALNHHNLLVCSVTDFYPGQIKVQWFRNDQEETAGVVSTPLIRNGDWTFQILVMLEMTPQRGDVYTCHVEHPSLQSPITVEWRAQSESAQSK

SEQ ID NO: 204 - protein, 9 aa, Artificial Sequence
  Description of Artificial Sequence Synthetic peptide
GILGFVFTL

SEQ ID NO: 205 - protein, 9 aa, Artificial Sequence
  Description of Artificial Sequence Synthetic peptide
NLVPMVGTV

SEQ ID NO: 206 - protein, 9 aa, Artificial Sequence
  Description of Artificial Sequence Synthetic peptide
EADPTGHSY

SEQ ID NO: 207 - protein, 10 aa, Artificial Sequence
  Description of Artificial Sequence Synthetic peptide
RYPLTFGWCF

SEQ ID NO: 208 - protein, 9 aa, Artificial Sequence
  Description of Artificial Sequence Synthetic peptide
QYDPVAALF

SEQ ID NO: 209 - protein, 11 aa, Artificial Sequence
  Description of Artificial Sequence Synthetic peptide
RPHERNGFTVL

SEQ ID NO: 210 - protein, 11 aa, Artificial Sequence
  Description of Artificial Sequence Synthetic peptide
RPHERNGFTVL

SEQ ID NO: 211 - protein, 9 aa, Artificial Sequence
  Description of Artificial Sequence Synthetic peptide
KILGFVFTV

SEQ ID NO: 212 - protein, 9 aa, Artificial Sequence
  Description of Artificial Sequence Synthetic peptide
VTEHDTLLY

SEQ ID NO: 213 - protein, 9 aa, Artificial Sequence
  Description of Artificial Sequence Synthetic peptide
TVRSHCVSK

SEQ ID NO: 214 - protein, 9 aa, Artificial Sequence
  Description of Artificial Sequence Synthetic peptide
TTFLQTMLR

SEQ ID NO: 215 - protein, 9 aa, Artificial Sequence
  Description of Artificial Sequence Synthetic peptide
IPSINVHHY

SEQ ID NO: 216 - protein, 10 aa, Artificial Sequence
  Description of Artificial Sequence Synthetic peptide
FVYGGSKTSL

SEQ ID NO: 217 - protein, 9 aa, Artificial Sequence
  Description of Artificial Sequence Synthetic peptide
FLRGRAYGL

SEQ ID NO: 218 - protein, 9 aa, Artificial Sequence
  Description of Artificial Sequence Synthetic peptide
RYRPGTVAL

SEQ ID NO: 219 - protein, 9 aa, Artificial Sequence
  Description of Artificial Sequence Synthetic peptide
QYDPVAALF

SEQ ID NO: 220 - protein, 9 aa, Artificial Sequence
  Description of Artificial Sequence Synthetic peptide
GQFLTPNSH

SEQ ID NO: 221 - protein, 9 aa, Artificial Sequence
  Description of Artificial Sequence Synthetic peptide
KEVNSQLSL

SEQ ID NO: 222 - protein, 9 aa, Artificial Sequence
  Description of Artificial Sequence Synthetic peptide
VSFIEFVGW

SEQ ID NO: 223 - protein, 9 aa, Artificial Sequence
  Description of Artificial Sequence Synthetic peptide
IAPWYAFAL

SEQ ID NO: 224 - protein, 16 aa, Artificial Sequence
  Description of Artificial Sequence Synthetic peptide
KPVSKMRMATPLLMQA

SEQ ID NO: 225 - protein, 15 aa, Artificial Sequence
  Description of Artificial Sequence Synthetic
QIYKANSKFIGITEL

SEQ ID NO: 226 - protein, 30 aa, Artificial Sequence
  Description of Artificial Sequence Synthetic polypeptide
  SITE (1)..(30): This sequence may encompass 1-6 “Gly Gly Gly Gly Ser” repeating units
GGGGSGGGGSGGGGSGGGGSGGGGSGGGGS

SEQ ID NO: 227 - protein, 11 aa, Artificial Sequence
  Description of Artificial Sequence Synthetic peptide
SSSGSSSGSAA

SEQ ID NO: 228 - protein, 5 aa, Artificial Sequence
  Description of Artificial Sequence Synthetic peptide
GGGGG

SEQ ID NO: 229 - protein, 51 aa, Artificial Sequence
  Description of Artificial Sequence Synthetic polypeptide
  SITE (2)..(51): This region may encompass 1-10 “Gly Gly Gly Gly Ser” repeating units
SGGGGSGGGGSGGGGSGGGGSGGGGSGGGGSGGGGSGGGGSGGGGSGGGGS

SEQ ID NO: 230 - protein, 20 aa, Artificial Sequence
  Description of Artificial Sequence Synthetic peptide
  SITE (1)..(20): This sequence may encompass 1-5 “Gly Gly Ser Gly” repeating units
GGSGGGSGGGSGGGSGGGSG

SEQ ID NO: 231 - protein, 4 aa, Artificial Sequence
  Description of Artificial Sequence Synthetic peptide
GSAT

SEQ ID NO: 232 - protein, 30 aa, Artificial Sequence
  Description of Artificial Sequence Synthetic polypeptide
  SITE (1)..(30): This sequence may encompass 1-5 “Gly Gly Ser Gly Gly Ser” repeating units
GGSGGSGGSGGSGGSGGSGGSGGSGGSGGS

SEQ ID NO: 233 - protein, 20 aa, Artificial Sequence
  Description of Artificial Sequence Synthetic peptide
GGGGSGGGGSGGGGSGGGGS

SEQ ID NO: 234 - protein, 14 aa, Artificial Sequence
  Description of Artificial Sequence Synthetic peptide
GSGSAGGSGSGGGS

SEQ ID NO: 235 - protein, 4 aa, Artificial Sequence
  Description of Artificial Sequence Synthetic peptide
IEGR

SEQ ID NO: 236 - protein, 24 aa, Artificial Sequence
  Description of Artificial Sequence Synthetic peptide
GGGASGGGGSIEGRGGGGSGGGGS

SEQ ID NO: 237 - protein, 5 aa, Artificial Sequence
  Description of Artificial Sequence Synthetic peptide
DDDDK

SEQ ID NO: 238 - protein, 8 aa, Artificial Sequence
  Description of Artificial Sequence Synthetic peptide
DYKDDDDK

SEQ ID NO: 239 - protein, 6 aa, Artificial Sequence
  Description of Artificial Sequence Synthetic peptide
HHHHHH

SEQ ID NO: 240 - protein, 14 aa, Artificial Sequence
  Description of Artificial Sequence Synthetic peptide
GKPIPNPLLGLDST

SEQ ID NO: 241 - protein, 8 aa, Artificial Sequence
  Description of Artificial Sequence Synthetic peptide
WSHPQFEK

SEQ ID NO: 242 - protein, 12 aa, Artificial Sequence
  Description of Artificial Sequence Synthetic peptide
EDQVDPRLIDGK

SEQ ID NO: 243 - protein, 10 aa, Artificial Sequence
  Description of Artificial Sequence Synthetic peptide
EQKLISEEDL

SEQ ID NO: 244 - protein, 28 aa, Artificial Sequence
  Description of Artificial Sequence Synthetic peptide
GLNDIFEAQKIEWHEGSGEQKLISEEDL

SEQ ID NO: 245 - protein, 34 aa, Artificial Sequence
  Description of Artificial Sequence Synthetic polypeptide
GLNDIFEAQKIEWHEGSGEQKLISEEDLHHHHHH

SEQ ID NO: 246 - protein, 40 aa, Artificial Sequence
  Description of Artificial Sequence Synthetic polypeptide
GSGSAGGGLNDIFEAQKIEWHEGSTGHHHHHHDYKDDDDK

SEQ ID NO: 247 - protein, 39 aa, Unknown
  Description of Unknown Fos sequence
LTDTLQAETDQLEDEKSALQTEIANLLKEKEKLEFILAA

SEQ ID NO: 248 - protein, 40 aa, Unknown
  Description of Unknown Jun sequence
RIARLEEKVKTLKAQNSELASTANMLREQVAQLKQKVMNH

SEQ ID NO: 249 - protein, 35 aa, Artificial Sequence
  Description of Artificial Sequence Synthetic polypeptide
TTAPSAQLEKELQALQKENAQLEWELQALEKELAQ

SEQ ID NO: 250 - protein, 35 aa, Artificial Sequence
  Description of Artificial Sequence Synthetic polypeptide
TTAPSAQLKKKLQALKKKNAQLKWKLQALKKKLAQ

SEQ ID NO: 251 - protein, 232 aa, Artificial Sequence
  Description of Artificial Sequence Synthetic polypeptide
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 232 aa, Artificial Sequence
  Description of Artificial Sequence Synthetic polypeptide
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 253 - protein, 16 aa, Artificial Sequence
  Description of Artificial Sequence Synthetic peptide
RGVPHIVMVDAYKRYK

SEQ ID NO: 254 - protein, 113 aa, Artificial Sequence
  Description of Artificial Sequence Synthetic polypeptide
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 255 - protein, 20 aa, Artificial Sequence
  Description of Artificial Sequence Synthetic peptide
MEAPAQLLFLLLLWLPDTTG

SEQ ID NO: 256 - protein, 23 aa, Homo sapiens
MNRGVPFRHLLLVLQLALLPAAT

SEQ ID NO: 257 - protein, 20 aa, Mus sp.
METDTLLLWVLLLWVPGSTG

SEQ ID NO: 258 - protein, 21 aa, Mus sp.
MVPCTLLLLLAAALAPTQTRA

SEQ ID NO: 259 - protein, 18 aa, Homo sapiens
MKWVTFISLLFLFSSAYS

SEQ ID NO: 260 - protein, 20 aa, Homo sapiens
MYRMQLLSCIALSLALVTNS

SEQ ID NO: 261 - protein, 24 aa, Homo sapiens
MAVMAPRTLLLLLSGALALTQTWA

SEQ ID NO: 262 - protein, 20 aa, Homo sapiens
MSRSVALAVLALLSLSGLEA

SEQ ID NO: 263 - protein, 159 aa, Streptomyces avidinii
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 264 - protein, 127 aa, Streptomyces avidinii
AEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 265 - protein, 126 aa, Artificial Sequence
  Description of Artificial Sequence Synthetic polypeptide
MEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAA

SEQ ID NO: 266 - protein, 119 aa, Artificial Sequence
  Description of Artificial Sequence Synthetic polypeptide
MGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKV

SEQ ID NO: 267 - protein, 10 aa, Artificial Sequence
  Description of Artificial Sequence Synthetic peptide
IALNFPGSQK

SEQ ID NO: 268 - protein, 9 aa, Artificial Sequence
  Description of Artificial Sequence Synthetic peptide
LIYRRRLMK

SEQ ID NO: 269 - protein, 9 aa, Artificial Sequence
  Description of Artificial Sequence Synthetic peptide
ILRGSVAHK

SEQ ID NO: 270 - protein, 9 aa, Artificial Sequence
  Description of Artificial Sequence Synthetic peptide
RIKEHMLKK

SEQ ID NO: 271 - protein, 10 aa, Artificial Sequence
  Description of Artificial Sequence Synthetic peptide
AVFDRKSDAK

SEQ ID NO: 272 - protein, 9 aa, Artificial Sequence
  Description of Artificial Sequence Synthetic peptide
IVTDFSVIK

SEQ ID NO: 273 - protein, 9 aa, Artificial Sequence
  Description of Artificial Sequence Synthetic peptide
AIFQSSMTK

SEQ ID NO: 274 - protein, 9 aa, Artificial Sequence
  Description of Artificial Sequence Synthetic peptide
VYGFVRACL

SEQ ID NO: 275 - protein, 10 aa, Artificial Sequence
  Description of Artificial Sequence Synthetic peptide
TPRVTGGGAM

SEQ ID NO: 276 - protein, 9 aa, Artificial Sequence
  Description of Artificial Sequence Synthetic peptide
ALKRKMMYM

SEQ ID NO: 277 - protein, 9 aa, Artificial Sequence
  Description of Artificial Sequence Synthetic peptide
HSKKKCDEL

SEQ ID NO: 278 - protein, 9 aa, Artificial Sequence
  Description of Artificial Sequence Synthetic peptide
WLSLLVPFV

SEQ ID NO: 279 - protein, 9 aa, Artificial Sequence
  Description of Artificial Sequence Synthetic peptide
VMAPRTLIL

SEQ ID NO: 280 - protein, 9 aa, Artificial Sequence
  Description of Artificial Sequence Synthetic peptide
TRATKMQVI

SEQ ID NO: 281 - protein, 9 aa, Artificial Sequence
  Description of Artificial Sequence Synthetic peptide
RQYDPVAAL

SEQ ID NO: 282 - protein, 10 aa, Artificial Sequence
  Description of Artificial Sequence Synthetic peptide
FVYGGSKTSL

SEQ ID NO: 283 - protein, 9 aa, Artificial Sequence
  Description of Artificial Sequence Synthetic peptide
VSDGGPNLY

SEQ ID NO: 284 - protein, 10 aa, Artificial Sequence
  Description of Artificial Sequence Synthetic peptide
YILGADPLRV

SEQ ID NO: 285 - protein, 8 aa, Artificial Sequence
  Description of Artificial Sequence Synthetic peptide
YHSIEWAI

SEQ ID NO: 286 - protein, 9 aa, Artificial Sequence
  Description of Artificial Sequence Synthetic peptide
RRRWRRLTV

SEQ ID NO: 287 - protein, 9 aa, Artificial Sequence
  Description of Artificial Sequence Synthetic peptide
ALFFFDIDL

SEQ ID NO: 288 - protein, 8 aa, Artificial Sequence
  Description of Artificial Sequence Synthetic peptide
FPTKDVAL

SEQ ID NO: 289 - protein, 9 aa, Artificial Sequence
  Description of Artificial Sequence Synthetic peptide
YRSGIIAVV

SEQ ID NO: 290 - protein, 9 aa, Artificial Sequence
  Description of Artificial Sequence Synthetic peptide
SYMIMEIEL

SEQ ID NO: 291 - protein, 8 aa, Artificial Sequence
  Description of Artificial Sequence Synthetic peptide
TAFTIPSI

SEQ ID NO: 292 - protein, 9 aa, Artificial Sequence
  Description of Artificial Sequence Synthetic peptide
TVCGGIMFL

SEQ ID NO: 293 - protein, 10 aa, Artificial Sequence
  Description of Artificial Sequence Synthetic peptide
SFSFGGFTFK

SEQ ID NO: 294 - protein, 9 aa, Artificial Sequence
  Description of Artificial Sequence Synthetic peptide
FEDLRVSSF

SEQ ID NO: 295 - protein, 10 aa, Artificial Sequence
  Description of Artificial Sequence Synthetic peptide
SFSFGGFTFK

SEQ ID NO: 296 - protein, 8 aa, Artificial Sequence
  Description of Artificial Sequence Synthetic peptide
SELEIKRY

SEQ ID NO: 297 - protein, 10 aa, Artificial Sequence
  Description of Artificial Sequence Synthetic peptide
CVIGGAGNNT

SEQ ID NO: 298 - protein, 9 aa, Artificial Sequence
  Description of Artificial Sequence Synthetic peptide
YLLEMLWRL

SEQ ID NO: 299 - protein, 9 aa, Artificial Sequence
  Description of Artificial Sequence Synthetic peptide
KWMRELVLY

SEQ ID NO: 300 - protein, 9 aa, Artificial Sequence
  Description of Artificial Sequence Synthetic peptide
VAFTSHEHF

SEQ ID NO: 301 - protein, 9 aa, Artificial Sequence
  Description of Artificial Sequence Synthetic peptide
AIMESGVAL

SEQ ID NO: 302 - protein, 10 aa, Artificial Sequence
  Description of Artificial Sequence Synthetic peptide
KRWIILGLNK

SEQ ID NO: 303 - protein, 9 aa, Artificial Sequence
  Description of Artificial Sequence Synthetic peptide
HSNLNDATY

SEQ ID NO: 304 - protein, 9 aa, Artificial Sequence
  Description of Artificial Sequence Synthetic peptide
VSDGGPNLY

SEQ ID NO: 305 - protein, 10 aa, Artificial Sequence
  Description of Artificial Sequence Synthetic peptide
EENLLDFVRF

SEQ ID NO: 306 - protein, 11 aa, Artificial Sequence
  Description of Artificial Sequence Synthetic peptide
KAFSPEVIPMF

SEQ ID NO: 307 - protein, 9 aa, Artificial Sequence
  Description of Artificial Sequence Synthetic peptide
FLDKGTYTL

SEQ ID NO: 308 - protein, 9 aa, Artificial Sequence
  Description of Artificial Sequence Synthetic peptide
FCRVLCCYV

SEQ ID NO: 309 - protein, 8 aa, Artificial Sequence
  Description of Artificial Sequence Synthetic peptide
TAFTIPSI

SEQ ID NO: 310 - protein, 15 aa, Artificial Sequence
  Description of Artificial Sequence Synthetic peptide
LQAKARAKKDELRRK

SEQ ID NO: 311 - protein, 9 aa, Artificial Sequence
  Description of Artificial Sequence Synthetic peptide
AENAGNDAC

SEQ ID NO: 312 - protein, 9 aa, Artificial Sequence
  Description of Artificial Sequence Synthetic peptide
HPVGEADYF

SEQ ID NO: 313 - protein, 11 aa, Artificial Sequence
  Description of Artificial Sequence Synthetic peptide
PHGPVQLSYYD

SEQ ID NO: 314 - protein, 9 aa, Artificial Sequence
  Description of Artificial Sequence Synthetic peptide
YPLHEQHGM

SEQ ID NO: 315 - protein, 9 aa, Artificial Sequence
  Description of Artificial Sequence Synthetic peptide
HERNGFTVL

SEQ ID NO: 316 - protein, 11 aa, Artificial Sequence
  Description of Artificial Sequence Synthetic peptide
KAFSPEVIPMF

SEQ ID NO: 317 - protein, 11 aa, Artificial Sequence
  Description of Artificial Sequence Synthetic peptide
FPKTTNGCSQA

SEQ ID NO: 318 - protein, 10 aa, Artificial Sequence
  Description of Artificial Sequence Synthetic peptide
EENLLDFVRF

SEQ ID NO: 319 - protein, 9 aa, Artificial Sequence
  Description of Artificial Sequence Synthetic peptide
ELKRKMMYM

SEQ ID NO: 320 - protein, 9 aa, Artificial Sequence
  Description of Artificial Sequence Synthetic peptide
HSKKKCDEL

SEQ ID NO: 321 - protein, 278 aa, Artificial Sequence
  Description of Artificial Sequence Synthetic polypeptide
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSS

SEQ ID NO: 322 - protein, 10 aa, Homo sapiens
ELAGIGILTV

SEQ ID NO: 323 - protein, 16 aa, Unknown
  Description of Unknown CLIP peptide sequence
RMATPLLMQALPMGAL

SEQ ID NO: 324 - protein, 14 aa, Unknown
  Description of Unknown CLIP peptide sequence
LMQALPMGALPQGP

SEQ ID NO: 325 - protein, 16 aa, Artificial Sequence
  Description of Artificial Sequence Synthetic peptide
SGGGGSGGGGSGGGGS

SEQ ID NO: 326 - protein, 21 aa, Artificial Sequence
  Description of Artificial Sequence Synthetic peptide
SGGGGSGGGGSGGGGSGGGGS

SEQ ID NO: 327 - protein, 4 aa, Artificial Sequence
  Description of Artificial Sequence Synthetic peptide
GSTG